(19) United States
(12) Patent Application Publication
(10) Pub. No.: US 2005/0137130 A1
(43) Pub. Date: Jun. 23, 2005

(54) Medical treatment

(76) Inventors: Mark William Bodmer, Cambridge (GB); Emmanuel Cyrille Pascal Briend, Cambridge (GB); Brian Robert Champion, Cambridge (GB); Andrew Christopher Lennard, Cambridge (GB); Grahame James Mckenzie, Cambridge (GB); Silvia Ragno, Cambridge (GB); Tamara Tugal, Cambridge (GB); Lesley Lynn Young, Cambridge (GB)

Correspondence Address: FROMMER LAWRENCE & HAUG, 745 FIFTH AVENUE- 10TH FL., NEW YORK, NY, 10151, US

(21) Appl. No.: 10/845,834

(22) Filed: May 14, 2004

Related U.S. Application Data
(63) Continuation-in-part of application No. PCT/GB02/05137, filed on Nov. 13, 2002.

(30) Foreign Application Priority Data
Nov. 14, 2001 (GB) 0127267.3
Sep. 7, 2002 (GB) 0220849.4
Sep. 10, 2002 (GB) 0220913.8
Sep. 27, 2002 (WO) PCT/GB02/04390

(51) Int. Cl. A61K 48/00; A61K 38/17

(52) U.S. Cl. 514/12; 514/44

(57) ABSTRACT

An inhibitor of the Notch signalling pathway is provided for use as an immunostimulant, for example as a vaccine adjuvant.

### CROSS-REFERENCE TO RELATED APPLICATIONS

This application is a continuation-in-part of International Application No. PCT/GB02/05137, filed on Nov. 13, 2002, published as WO 03/041735 on May 22, 2003, and claiming priority to GB application Serial Nos. 0127267.3, filed on Nov. 14, 2001, 0220849.4, filed on Sep. 7, 2002, and 0220913.8, filed on Sep. 10, 2002, and to International Application Nos. PCT/GB02/03426, filed on Jul. 25, 2002, and PCT/GB02/004390, filed on Sep. 27, 2002. Reference is made to U.S. application Ser. No. 09/310,685, filed on May 4, 1999, Ser. No. 09/870,902, filed on May 31, 2001, Ser. No. 10/013,310, filed on Dec. 7, 2001, Ser. No. 10/147,354, filed on May 16, 2002, Ser. No. 10/357,321, filed on Feb. 3, 2002, Ser. No. 10/682,230, filed on Oct. 9, 2003, Ser. No. 10/720,896, filed on Nov. 24, 2003, Ser. Nos. 10/763,362, 10/764,415 and 10/765,727, all filed on Jan. 23, 2004 and Ser. No. 10/812,144, filed on Mar. 29, 2004. Reference is also made to International Application No. PCT/GB02/05133, filed on Nov. 13, 2002, and published as WO 03/042246 on May 22, 2003.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer 's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to the modulation of immune function, in particular by use of a modulator of the Notch signalling pathway.

### BACKGROUND OF THE INVENTION

International Patent Publication No WO 98/20142 describes how manipulation of the Notch signalling pathway can be used in immunotherapy and in the prevention and/or treatment of T-cell mediated diseases. In particular, the document discusses how allergy, autoimmunity, graft rejection, tumour induced aberrations to the T-cell system and infectious diseases caused, for example, by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara, may be targeted.

It has also been shown that it is possible to generate a class of regulatory T cells which are able to transmit antigen-specific tolerance to other T cells, a process termed infectious tolerance (WO98/20142). The functional activity of these cells can be mimicked by over-expression of a Notch ligand protein on their cell surfaces or on the surface of antigen presenting cells. In particular, regulatory T cells can be generated by over-expression of a member of the Delta or Serrate family of Notch ligand proteins. Delta or Serrate induced T cells specific to one antigenic epitope are also able to transfer tolerance to T cells recognising other epitopes on the same or related antigens, a phenomenon termed “epitope spreading”.

Notch ligand expression also plays a role in cancer. Indeed, upregulated Notch ligand expression has been observed in some tumour cells. These tumour cells are capable of rendering T cells unresponsive to restimulation with a specific antigen, thus providing a possible explanation of how tumour cells prevent normal T cell responses. By downregulating Notch signalling in vivo in T cells, it may be possible to prevent tumour cells from inducing immunotolerance in those T cells that recognise tumour-specific antigens. In turn, this would allow the T cells to mount an immune response against the tumour cells (WO00/135990).

A description of the Notch signalling pathway and conditions affected by it may be found in our published PCT Applications PCT/GB97/03058 (filed on 6 Nov. 1997 and claiming priority from GB 9623236.8 filed on 7 Nov. 1996, GB 9715674.9 filed on 24 Jul. 1997 and GB 9719350.2 filed on 11 Sep. 1997; published as WO 98/20142) PCT/GB99/04233 (filed on 15 Dec. 1999 and claiming priority from GB 9827604.1 filed on 15 Dec. 1999; published as WO 00/36089) and PCT/GB00/04391 (filed on 17 Nov. 2000 and claiming priority from GB 9927328.6 filed on 18 Nov. 1999; published as WO 0135990). Each of PCT/GB97/03058 (WO 98/20142), PCT/GB99/04233 (WO 00/36089) and PCT/GB00/04391 (WO 0135990) are hereby incorporated herein by reference.

The present invention seeks to provide further methods of modulating the immune system by modification of the Notch signalling pathway, in particular for the treatment of infectious disease.

### SUMMARY OF THE INVENTION

According to a first aspect of the invention there is provided a product comprising:
i) an inhibitor of the Notch signalling pathway or a polynucleotide coding for such an inhibitor; and
ii) a pathogen antigen or antigenic determinant or a polynucleotide coding for a pathogen antigen or antigenic determinant;
as a combined preparation for simultaneous, contemporaneous, separate or sequential use for modulation of the immune system.

Preferably the agent does not act by downregulating expression of Notch or a Notch ligand.

According to a further aspect of the invention there is provided a product comprising:
i) an inhibitor of Notch signalling in the form of a Notch antagonist agent or a polynucleotide coding for such an agent; and
ii) a pathogen antigen or antigenic determinant or a polynucleotide coding for a pathogen antigen or antigenic determinant;
as a combined preparation for simultaneous, contemporaneous, separate or sequential use for modulation of the immune system.

According to a further aspect of the invention there is provided a product comprising:
i) an inhibitor of Notch signalling in the form of an agent which inhibits Notch-Notch ligand interaction or a polynucleotide coding for such an agent; and
ii) a pathogen antigen or antigenic determinant or a polynucleotide coding for a pathogen antigen or antigenic determinant;
as a combined preparation for simultaneous, contemporaneous, separate or sequential use for modulation of the immune system.

Suitably such a product may take the form of a pharmaceutical composition or kit.

Suitably such a product may take the form of a therapeutic vaccine composition or kit for treating infectious disease (including so-called “pharmaccines”).

Alternatively such a product may take the form of a prophylactic vaccine composition or kit for preventing infectious disease.

According to a further aspect of the invention there is provided the use of an inhibitor of the Notch signalling pathway in the manufacture of a medicament for use as an immunostimulant. Preferably the medicament is not for use in reversing bacteria, infection or tumour-induced immunosuppression or for the treatment of a tumour.

The term “immunostimulant” as used herein means an agent which is capable of restoring a depressed immune function, or enhancing normal immune function, or both. The term agent may boost a subject's immune system either generally or in respect of a specific antigen or antigenic determinant. Immunostimulants may be used, for example, for the treatment of conditions requiring general immune stimulation including immune deficiency conditions such as Acquired Immune Deficiency Syndrome (AIDS) and Severe Combined Immunodeficiency Disease (SCID) and in situations where antigen specific stimulation is desired, such as in vaccination.

According to a further aspect of the invention there is provided the use of an inhibitor of the Notch signalling pathway in the manufacture of a medicament for use in vaccination against a pathogen.

According to a further aspect of the invention there is provided the use of an inhibitor of the Notch signalling pathway in the manufacture of a medicament for use as an adjuvant for vaccination against a pathogen.

The term “pathogen” as used herein means a disease causing parasite which is normally a microorganism. The term includes, for example, viruses, bacteria, protozoa and fungi.

The term “pathogen antigen” as used herein means an antigen found on a pathogen or a fragment, variant or derivative of such an antigen comprising antigenic determinants (epitopes; preferably immunodominant epitopes) or epitope regions (preferably immunodominant epitope regions) of such an antigen. Preferably the antigen is immunogenic (an immunogen). Suitably the antigen is a microbial pathogen antigen.

According to a further aspect of the invention there is provided a method for stimulating the immune system by administering an inhibitor of the Notch signalling pathway which preferably does not comprise reversing bacteria, infection or tumour-induced immunosuppression or treatment of a tumour.

The terms “inhibitor of Notch signalling” and “inhibitor of the Notch signalling pathway” as used herein include any agent which is capable of reducing any one or more of the upstream or downstream events that result in, or from, (and including) activation of the Notch receptor. Preferably the inhibitor of Notch signalling does not act by downregulating expression of Notch or a Notch ligand.

According to a further aspect of the invention there is provided a method for stimulating the immune system by administering an inhibitor of the Notch signalling pathway wherein the inhibitor does not act by downregulating expression of Notch or a Notch ligand.

According to a further aspect of the invention there is provided a method for vaccination against a pathogen by administering an inhibitor of the Notch signalling pathway.

According to a further aspect of the invention there is provided a method for enhancing vaccination against a pathogen by administering an inhibitor of the Notch signalling pathway.

According to a further aspect of the invention there is provided a method for stimulating the immune system to treat or prevent an infection by administering an inhibitor of the Notch signalling pathway which does not comprise reversing bacteria, infection or tumour-induced immunosuppression or treatment of a tumour.

According to a further aspect of the invention there is provided a method for stimulating the immune system to treat or prevent an infection by administering an inhibitor of the Notch signalling pathway wherein the inhibitor of the Notch signalling pathway does not act by downregulating expression of Notch or a Notch ligand.

According to a further aspect of the invention there is provided a method for treating an acute pathogen infection by administering an inhibitor of the Notch signalling pathway.

According to a further aspect of the invention there is provided a method for treating a chronic pathogen infection by administering an inhibitor of the Notch signalling pathway.

According to a further aspect of the invention there is provided a method of increasing the immune response of a subject to a vaccine antigen or antigenic determinant comprising administering an effective amount of an inhibitor of the Notch signalling pathway to said subject simultaneously, separately or sequentially with said vaccine antigen or antigenic determinant or simultaneously, separately or sequentially with a polynucleotide coding for said vaccine antigen or antigenic determinant.

Preferably the inhibitor of Notch signalling inhibits Notch signalling in immune cells, such as APCs, B-cells or T-cells.

Suitably the inhibitor of the Notch signalling pathway may be a Notch signalling repressor or an agent which increases the expression or activity of a Notch signalling repressor.

Preferably the inhibitor of the Notch signalling pathway is an agent capable of inhibiting the activity of a Notch receptor or a Notch ligand.

Alternatively or in addition the inhibitor of the Notch signalling pathway may be an agent capable of inhibiting the activity or downregulating the expression of a downstream component of the Notch signalling pathway.

Preferably the inhibitor of the Notch signalling pathway may be an agent which interacts with, and preferably binds to a Notch receptor or a Notch ligand so as to interfere with endogenous Notch ligand-receptor interaction (also termed “Notch-Notch ligand interaction”). Such an agent may be referred to as a “Notch antagonist”. Preferably the inhibitor inhibits Notch ligand-receptor interaction in immune cells such as lymphocytes and APCs, preferably in lymphocytes, preferably in T-cells.

Suitably the inhibitor of Notch signalling may be a protein or polypeptide or a polynucleotide which codes for such a protein or polypeptide.

In one embodiment, for example, the inhibitor of Notch signalling may comprise or codes for the extracellular domain of Delta or a fragment, derivative or homologue thereof.

Suitably, for example, the inhibitor of Notch signalling comprises or codes for the extracellular domain of Serrate or Jagged or a fragment, derivative or homologue thereof.

Suitably, for example, the inhibitor of Notch signalling comprises or codes for the extracellular domain of Notch or a fragment, derivative or homologue thereof.

Suitably, for example, the inhibitor of Notch signalling comprises:
i) a protein or polypeptide which comprises a Notch ligand DSL domain and optionally a Notch ligand N-terminal domain or a heterologous amino acid sequence but which is substantially free of Notch ligand EGF-like domains;
ii) a multimer of such a protein or polypeptide (wherein each monomer may be the same or different); or
iii) a polynucleotide coding for such a protein or polypeptide.

Suitably, for example, the inhibitor of Notch signalling comprises:
i) a protein or polypeptide which comprises a Notch ligand DSL domain and at least one Notch ligand EGF-like domain;
ii) a multimer of such a protein or polypeptide (wherein each monomer may be the same or different); or
iii) a polynucleotide coding for such a protein or polypeptide.

Suitably, for example, the inhibitor of Notch signalling comprises:
i) a protein or polypeptide which comprises a Notch ligand DSL domain and at least two Notch ligand EGF-like domains;
ii) a multimer of such a protein or polypeptide (wherein each monomer may be the same or different); or
iii) a polynucleotide coding for such a protein or polypeptide.

Suitably, for example, the inhibitor of Notch signalling comprises:
i) a protein or polypeptide which comprises a Notch ligand DSL domain and either 0, 1 or 2, but no more than 2 Notch ligand EGF-like domains;
ii) a multimer of such a protein or polypeptide (wherein each monomer may be the same or different); or
iii) a polynucleotide coding for such a protein or polypeptide.

Suitably, for example, the inhibitor of Notch signalling comprises:
i) a protein or polypeptide which comprises a Notch ligand DSL domain having at least 30%, preferably at least 50% amino acid sequence similarity or identity to the DSL domain of human Delta1, Delta3 or Delta4 and at least one Notch ligand EGF-like domain having at least 30%, preferably at least 50% amino acid sequence similarity or identity to an EGF-like domain of human Delta1, Delta3 or Delta4;
ii) a multimer of such a protein or polypeptide (wherein each monomer may be the same or different); or
iii) a polynucleotide coding for such a protein or polypeptide.

Suitably, for example, the inhibitor of Notch signalling comprises:
i) a protein or polypeptide which comprises a Notch ligand DSL domain having at least 30%, preferably at least 50% amino acid sequence similarity or identity to the DSL domain of human Delta1, Delta3 or Delta4 and either 0, 1 or 2, but no more than 2 Notch ligand EGF-like domains having at least 30%, preferably at least 50% amino acid sequence similarity or identity to an EGF-like domain of human Delta1, Delta3 or Delta4;
ii) a multimer of such a protein or polypeptide (wherein each monomer may be the same or different); or
iii) a polynucleotide coding for such a protein or polypeptide.

Suitably, for example, the inhibitor of Notch signalling comprises:
i) a protein or polypeptide which comprises a Notch EGF-like domain having at least 30%, preferably at least 50% amino acid sequence similarity or identity to EGF11 of human Notch1, Notch2, Notch3 or Notch4 and a Notch EGF-like domain having at least 30%, preferably at least 50% amino acid sequence similarity or identity to EGF12 of human Notch1, Notch2, Notch3 or Notch4;
ii) a multimer of such a protein or polypeptide (wherein each monomer may be the same or different); or
iii) a polynucleotide coding for such a protein or polypeptide.

Suitably, for example, the inhibitor of Notch signalling comprises:
i) a protein or polypeptide which comprises a Notch ligand DSL domain having at least 30%, preferably at least 50% amino acid sequence similarity or identity to the DSL domain of human Jagged1 or Jagged2 and at least one Notch ligand EGF-like domain having at least 30%, preferably at least 50% amino acid sequence similarity or identity to an EGF-like domain of human Jagged 1 or Jagged2;
ii) a multimer of such a protein or polypeptide (wherein each monomer may be the same or different); or
iii) a polynucleotide coding for such a protein or polypeptide.

Suitably, for example, the inhibitor of Notch signalling comprises:
i) a protein or polypeptide which comprises a Notch ligand DSL domain having at least 30%, preferably at least 50% amino acid sequence similarity or identity to the DSL domain of human Jagged1 or Jagged2 and either 0, 1 or 2, but no more than 2 Notch ligand EGF-like domains having at least 30%, preferably at least 50% amino acid sequence similarity or identity to an EGF-like domain of human Jagged 1 or Jagged2;
ii) a multimer of such a protein or polypeptide (wherein each monomer may be the same or different); or
iii) a polynucleotide coding for such a protein or polypeptide.

Suitably, for example, the inhibitor of Notch signalling comprises:
i) a protein or polypeptide which comprises a Notch ligand DSL domain having at least 70% amino acid sequence similarity or identity to the DSL domain of human Delta1, Delta3 or Delta4 and at least one Notch ligand EGF-like domain having at least 70% amino acid sequence similarity or identity to an EGF-like domain of human Delta1, Delta3 or Delta4;
ii) a multimer of such a protein or polypeptide (wherein each monomer may be the same or different); or
iii) a polynucleotide coding for such a protein or polypeptide.

Suitably, for example, the inhibitor of Notch signalling comprises:
i) a protein or polypeptide which comprises a Notch ligand DSL domain having at least 70% amino acid sequence similarity or identity to the DSL domain of human Delta1, Delta3 or Delta4 and either 0, 1 or 2, but no more than 2 Notch ligand EGF-like domains having at least 70% amino acid sequence similarity or identity to an EGF-like domain of human Delta1, Delta3 or Delta4;
ii) a multimer of such a protein or polypeptide (wherein each monomer may be the same or different); or
iii) a polynucleotide coding for such a protein or polypeptide.

An advantage of using a protein or polypeptide having preferably no more than two Notch ligand EGF-like domains is that it provides effective inhibition of Notch signalling with little or no competing agonist activity, thus providing a more selective inhibitory effect. Such proteins and polypeptides may also be easier to produce especially, for example, in bacterial expression systems.

However, it will be appreciated that Notch signalling inhibition is also shown by constructs having more than 2 such EGF-like repeats.

Suitably, for example, the inhibitor of Notch signalling comprises:
i) a protein or polypeptide which comprises an EGF domain having at least 70% amino acid sequence similarity or identity to EGF11 of human Notch1, Notch2, Notch3 or Notch4 and an EGF domain having at least 70% amino acid sequence similarity or identity to EGF12 of human Notch1, Notch2, Notch3 or Notch4;
ii) a multimer of such a protein or polypeptide (wherein each monomer may be the same or different); or
iii) a polynucleotide coding for such a protein or polypeptide.

Suitably the protein or polypeptide may be fused to a heterologous amino acid sequence, such as an immunoglobulin Fc (IgFc) domain, for example a human IgG1 or IgG4 Fc domain.

Suitably the protein or polypeptide may further comprise a Notch ligand N-terminal domain.

Alternatively, for example, the inhibitor of Notch signalling may comprise an antibody, antibody fragment or antibody derivative or a polynucleotide which codes for an antibody, antibody fragment or antibody derivative. Suitably the antibody, antibody fragment or antibody derivative binds to a Notch receptor or a Notch ligand so as to interfere with Notch ligand-receptor interaction.

Suitably for example, the inhibitor of Notch signalling may have an IC₅₀ (preferably as measured in an assay as described herein, preferably using the Dynabeads assay of Example 12) of less than about 1000 uM, preferably less than about 100 uM, preferably less than about 10 uM, preferably less than about 1000 nM, preferably less than about 100 nM, suitably from about 0.1 to about 100 nM.

In one embodiment the modulator of the Notch signalling pathway may comprise a fusion protein comprising domains from a Notch ligand extracellular domain and an immunoglobulin Fc segment (eg IgG1 Fc or IgG4 Fc, preferably human IgG1 Fc or human IgG4 Fc) or a polynucleotide coding for such a fusion protein. Methods suitable for preparation of such fusion proteins are described, for example in Example 2 of WO 98/20142. IgG fusion proteins may be prepared as well known in the art, for example, as described in U.S. Pat. No. 5,428,130 (Genentech).

Suitably, the modulator of the Notch signalling pathway may be multimerised, preferably dimerised, for example by chemical cross-linking or formation of disulphide bonds between pairs of proteins or polypeptides. For example, where the proteins or polypeptides comprise a heterologous amino acid sequence in the form of an immunoglobulin Fc domain, these may assemble into dimers linked by disulphide bonds formed between the Fc domains (see, for example, the schematic representations of dimeric constructs as shown in the accompanying Figures).

Where the proteins or polypeptides are multimerised or dimerised in this way, the multimerised/dimerised form may contain more DSL and EGF domains than described in respect of the individual monomers. However, the ratios of DSL to EGF domains will preferably remain the same, such that there will preferably, for example be a ratio of DSL to EGF-like domains of 1:0, 1:1 or 1:2 for the multimerised aggregate as a whole.

Suitably, for example, the inhibitor of Notch signalling comprises a Notch ligand protein or polypeptide which consists essentially of the following components:
i) a Notch ligand DSL domain;
ii) optionally 1 or 2 EGF repeat domains;
iii) optionally all or part of a Notch ligand N-terminal domain; and
iv) optionally one or more heterologous amino acid sequences;
or a multimer of such a protein or polypeptide or a polynucleotide coding for such a Notch ligand protein or polypeptide.

Suitably, for example, the inhibitor of Notch signalling comprises a Notch ligand protein or polypeptide which consists essentially of the following components:
i) a Notch ligand DSL domain;
ii) optionally all or part of a Notch ligand N-terminal domain; and
iii) optionally one or more heterologous amino acid sequences;
or a multimer of such a protein or polypeptide or a polynucleotide coding for such a Notch ligand protein or polypeptide.

Suitably, for example, the inhibitor of Notch signalling comprises a Notch ligand protein or polypeptide which consists essentially of the following components:
i) a Notch ligand DSL domain;
ii) one Notch ligand EGF domain;
iii) optionally all or part of a Notch ligand N-terminal domain; and
iv) optionally one or more heterologous amino acid sequences;
or a multimer of such a protein or polypeptide or a polynucleotide coding for such a Notch ligand protein or polypeptide.

Suitably, for example, the inhibitor of Notch signalling comprises a Notch ligand protein or polypeptide which consists essentially of the following components:
i) a Notch ligand DSL domain;
ii) two Notch ligand EGF domains;
iii) optionally all or part of a Notch ligand N-terminal domain; and
iv) optionally one or more heterologous amino acid sequences;
or a multimer of such a protein or polypeptide or a polynucleotide coding for such a Notch ligand protein or polypeptide.

According to a further aspect of the invention there is provided the use of a binding agent which binds to a Notch ligand so as to interfere with binding of the ligand to a Notch receptor, or a polynucleotide which codes for such a binding agent, in the manufacture of a medicament for use as an immunostimulant.

According to a further aspect of the invention there is provided the use of an antibody or antibody derivative which binds to a Notch receptor or to a Notch ligand, or a polynucleotide which codes for such an antibody or antibody derivative, in the manufacture of a medicament for use as an immunostimulant.

According to a further aspect of the invention there is provided a method of increasing the immune response of a subject to a vaccine antigen or antigenic determinant comprising administering an effective amount of an inhibitor of the Notch signalling pathway to said subject simultaneously, separately or sequentially with said vaccine antigen.

According to a further aspect of the invention there is provided a method for stimulating the immune system by administering a binding agent which binds to a Notch receptor or Notch ligand so as to interfere with ligand-receptor interaction, or by administering a polynucleotide which codes for such a binding agent. The binding agent may, for example, comprise one or more extracellular domains from Notch or its ligands.

According to a further aspect of the invention there is provided a method for stimulating the immune system by administering an antibody or antibody derivative which binds to a Notch receptor or to a Notch ligand, or by administering a polynucleotide which codes for such an antibody or antibody derivative.

According to a further aspect of the invention there is provided an adjuvant composition comprising an inhibitor of the Notch signalling pathway.

According to a further aspect of the invention there is provided a vaccine composition comprising an adjuvant composition as described above and an antigen. Suitably the antigen may be a viral, fungal, parasitic or bacterial antigen.

According to a further aspect of the invention there is provided a method for modulating the immune system in a mammal comprising simultaneously, contemporaneously, separately or sequentially administering:
i) an effective amount of an inhibitor of the Notch signalling pathway; and
ii) a pathogen antigen or antigenic determinant or a polynucleotide coding for a pathogen antigen or antigenic determinant.

According to a further aspect of the invention there is provided a combination of:
i) an inhibitor of the Notch signalling pathway; and
ii) a pathogen antigen or antigenic determinant or a polynucleotide coding for a pathogen antigen or antigenic determinant;
for simultaneous, contemporaneous, separate or sequential use in modulating the immune system.

According to a further aspect of the invention there is provided an inhibitor of the Notch signalling pathway for use in modulating the immune system in simultaneous, contemporaneous, separate or sequential combination with a pathogen antigen or antigenic determinant or a polynucleotide coding for a pathogen antigen or antigenic determinant.

According to a further aspect of the invention there is provided the use of a combination of:
i) an inhibitor of the Notch signalling pathway; and
ii) a pathogen antigen or antigenic determinant or a polynucleotide coding for a pathogen antigen or antigenic determinant;
in the manufacture of a medicament for modulation of the immune system.

According to a further aspect of the invention there is provided the use of an inhibitor of the Notch signalling pathway in the manufacture of a medicament for modulation of the immune system in simultaneous, contemporaneous, separate or sequential combination with a pathogen antigen or antigenic determinant or a polynucleotide coding for a pathogen antigen or antigenic determinant.

According to a further aspect of the invention there is provided a pharmaceutical kit comprising an inhibitor of the Notch signalling pathway and a pathogen antigen or antigenic determinant or a polynucleotide coding for a pathogen antigen or antigenic determinant.

According to a further aspect of the invention there is provided a conjugate comprising first and second sequences, wherein the first sequence comprises a pathogen antigen or antigenic determinant or a polynucleotide sequence coding for a pathogen antigen or antigenic determinant, and the second sequence comprises a polypeptide or polynucleotide for Notch signalling modulation.

According to a further aspect of the invention there is provided a conjugate comprising first and second sequences, wherein the first sequence comprises a pathogen antigen or antigenic determinant or a polynucleotide sequence coding for a pathogen antigen or antigenic determinant, and the second sequence codes for an inhibitor of Notch signalling.

Preferably the conjugate is in the form of a vector comprising a first polynucleotide sequence coding for a modulator of the Notch signalling pathway and a second polynucleotide sequence coding for a pathogen antigen or antigenic determinant.

Preferably the conjugate is in the form of an expression vector.

Preferably in such a conjugate the first polynucleotide sequence codes for a Notch ligand or a fragment, derivative, homologue, analogue or allelic variant thereof.

Suitably the first polynucleotide sequence of the conjugate codes for a Delta or Serrate/Jagged protein or a fragment, derivative, homologue, analogue or allelic variant thereof.

Suitably the first polynucleotide sequence of the conjugate codes for a protein or polypeptide which comprises a Notch ligand DSL domain and optionally at least one Notch ligand EGF-like domain.

Suitably the first polynucleotide sequence of the conjugate codes for a protein or polypeptide which comprises a Notch ligand DSL domain and at least two Notch ligand EGF-like domains.

Suitably the first polynucleotide sequence of the conjugate codes for a protein or polypeptide which comprises a Notch ligand DSL domain and 1 or 2 but no more than 2 Notch ligand EGF-like domains.

Suitably the first and second sequences of the conjugate are each operably linked to one or more promoters.

According to a further aspect of the invention there is provided a method for increasing a TH2 immune response by administering a modulator of Notch signalling.

According to a further aspect of the invention there is provided a method for increasing a TH1 immune response by administering a modulator of Notch signalling.

According to a further aspect of the invention there is provided a method for increasing IFN-γ expression by administering an inhibitor of Notch signalling.

According to a further aspect of the invention there is provided a method for increasing IL-2 expression by administering an inhibitor of Notch signalling.

According to a further aspect of the invention there is provided a method for increasing TNFα expression by administering an inhibitor of Notch signalling.

According to a further aspect of the invention there is provided a method for increasing IL-4 expression by administering an inhibitor of Notch signalling.

According to a further aspect of the invention there is provided a method for increasing IL-5 expression by administering an inhibitor of Notch signalling.

According to a further aspect of the invention there is provided a method for increasing IL-13 expression by administering an inhibitor of Notch signalling.

According to a further aspect of the invention there is provided a method for reducing IL-10 expression by administering an inhibitor of Notch signalling.

According to a further aspect of the invention there is provided a method for increasing IL-5 expression by administering an inhibitor of Notch signalling.

According to a further aspect of the invention there is provided a method for generating an immune stimulatory cytokine profile with reduced IL-10 expression and increased IL-5 expression by administering an inhibitor of Notch signalling.

According to a further aspect of the invention there is provided a method for generating an immune stimulatory cytokine profile with increased IL-2, IFNγ, IL-5, IL-13 and TNFα expression by administering an inhibitor of Notch signalling. Suitably the cytokine profile also exhibits reduced IL-10 expression.

In one embodiment of the invention an inhibitor of Notch signalling is administered to a patient in vivo. Alternatively the inhibitor of Notch signalling may be administered to a cell ex-vivo, after which the cell may be administered to a patient.

Suitably the modulator of Notch signalling modifies cytokine expression in leukocytes, fibroblasts or epithelial cells. Preferably the modulator of Notch signalling modifies cytokine expression in dendritic cells, lymphocytes or macrophages, or their progenitors or tissue-specific derivatives.

Preferably the inhibitor of Notch signalling or the Notch signalling pathway for use in the present invention is an inhibitor of Notch-Notch ligand interaction. Suitably such an inhibitor of Notch-Notch ligand interaction is an agent which binds to a Notch receptor or Notch ligand so as to interfere with endogenous Notch-Notch ligand interaction whilst causing less activation of the Notch receptor than would result from endogenous Notch-Notch ligand interaction, or preferably no significant activation. For example, the inhibitor may bind to EGF-like domain 11 and/or EGF-like domain 12 of a Notch receptor or the DSL domain and/or EGF-like domain 1 and/or EGF-like domain 2 of a Notch ligand such as Delta, Serrate or Jagged. Thus, for example, the inhibitor may comprise EGF-like domains 11 and 12 of a Notch receptor. Alternatively the inhibitor may comprise a Notch ligand DSL domain and at least one EGF-like domain of a Notch ligand such as Delta, Serrate or Jagged. Suitably, for example, the inhibitor may comprise an extracellular domain of a Notch receptor, for example an extracellular domain of Notch1, Notch2, Notch3 or Notch4. Alternatively the inhibitor may comprise an extracellular domain of a Notch ligand such as Delta (eg a mammalian Delta1, Delta3 or Delta4), Serrate or Jagged (eg a mammalian Jagged1 or Jagged2).

Where the inhibitor binds to a Notch receptor, it may bind selectively to one Notch receptor such as Notch1, or may suitably have some degree of affinity for a range of Notch receptors or substantially all of them, due to their similar structures. Likewise, where the inhibitor binds to a Notch ligand, it may bind selectively to one Notch ligand such as Delta1, or may suitably have some degree of affinity for a range of Notch ligands or substantially all of them, due to their similar structures.

Alternatively the inhibitor may comprise an antibody which binds specifically to a Notch receptor or receptors. Preferably the antibody binds to the Notch receptor in such a way as to reduce or substantially prevent binding of native Notch ligands whilst the antibody is bound, or at least to reduce or substantially prevent activation of the Notch receptor. Suitably, for example, such an antibody may bind to EGF 11 and/or 12 of the Notch receptor (eg Notch1, Notch2, Notch3 and/or Notch4). The antibody may be selective for one Notch receptor such as Notch1, or may suitably have some degree of affinity for a range of Notch receptors or substantially all of them, due to their similar structures.

Alternatively the inhibitor may comprise an antibody which binds specifically to a Notch ligand or ligands. Preferably the antibody binds to the Notch ligand in such a way as to reduce or substantially prevent binding of the ligand to native Notch receptors whilst the antibody is bound, or at least to reduce or substantially prevent activation of the Notch receptor. Suitably, for example, such an antibody may bind to the DSL domain and/or to EGF-like domains 1 and/or 2 of a Notch ligand (eg a mammalian Delta1, Delta3, Delta4, Jagged1 or Jagged2). The antibody may be selective for one Notch ligand such as Delta1, or may suitably have some degree of affinity for a range of Notch ligands or substantially all of them, due to their similar structures.

It will be appreciated that combinations of antibodies with complementary specificities may also be used.

In an alternative embodiment, for example, the inhibitor of Notch signalling may be an inhibitor of Notch IC protease.

The term “Notch IC protease” as used herein means an enzyme or enzyme complex which acts proteolytically to cleave a Notch receptor to cause the release of all or part of the intracellular (IC) domain from the Notch receptor so as to activate the Notch signalling pathway. Enzymes which are understood to participate in such cleavage include the presenilins and gamma-secretase enzymes, and presenilin-dependent gamma-secretase enzymes or complexes.

The term “presenilin-dependent gamma-secretase” as used herein means an enzyme having gamma secretase proteolytic activity which requires presenilin for activity or activation. The presenilin may for example be required as a co-activator or as part of an enzyme complex.

Examples of presenilin proteins which may be modulated in the present invention include Presenilin-1 (PS1) and Presenilin-2 (PS2).

The modulator of Notch IC protease activity will preferably be selected from polypeptides and fragments thereof, linear peptides, cyclic peptides, and nucleic acids which encode therefor, synthetic and natural compounds including low molecular weight organic or inorganic compounds and antibodies. The modulator may for example be an agonist or an antagonist of presenilin or presenilin-dependent gamma-secretase, optionally in combination with an agent capable of respectively up-regulating or down-regulating the Notch signalling pathway respectively.

An example of an antagonist of presenilin which may be used in the present invention is 26S proteasome or a nucleic acid sequence which encodes therefor. Synthetic inhibitors include, for example, the difluoro ketone inhibitor described in Citron et al., and Wolfe et al. having the formula: 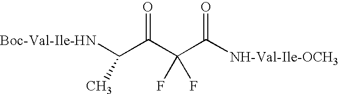
the inhibitors described in Sinha and Liederburg (2-Naphthoyl-VF-CHO, N-(2-Naphthoyl)-Val-phenylalaninal and N-Benzyloxycarbonyl-Leu-phenylalaninal Z-LF-CHO); the inhibitors described in Esler et al.; the inhibitors described in Figueiredo-Pereira et al., (N-Benzyloxycarbonyl-Leu-leucinal Z-LL-CHO); the inhibitors described in Higaki et al., (N-trans-3,5-Dimethoxycinnamoyl)-Ile-leucinal t-3,5-DMC-IL-CHO); the inhibitors described in Murphy et al., (Boc-GVV-CHO N-tert-Butyloxycarbonyl-Gly-Val-Valinal); and the inhibitors described in Riston et al., (1-(S)-endo-N-(1,3,3)-Trimethylbicyclo[2.2.1]hept-2-yl)-4-fluorophenyl Sulfonamide).

In an alternative embodiment, the inhibitor of Notch signalling is not an inhibitor of a Notch IC protease (ie is preferably not an inhibitor of presenilins and gamma-secretase enzymes, and is preferably not an inhibitor of presenilin-dependent gamma-secretase enzymes or complexes).

According to a further aspect of the invention there is provided a method for modifying an immune response by administering a Notch ligand protein or polypeptide consisting essentially of the following components:
i) a Notch ligand DSL domain;
ii) optionally 1 or 2 EGF repeat domains;
iii) optionally all or part of a Notch ligand N-terminal domain; and
iv) optionally one or more heterologous amino acid sequences;
or by administering a multimer of such a protein or polypeptide (wherein each monomer may be the same or different);
or by administering a polynucleotide coding for such a Notch ligand protein or polypeptide.

According to a further aspect of the invention there is provided a method for increasing an immune response by administering a Notch ligand protein or polypeptide consisting essentially of the following components:
i) a Notch ligand DSL domain;
ii) optionally 1 or 2 EGF repeat domains;
iii) optionally all or part of a Notch ligand N-terminal domain; and
iv) optionally one or more heterologous amino acid sequences;
or by administering a multimer of such a protein or polypeptide (wherein each monomer may be the same or different); or by administering a polynucleotide coding for such a Notch ligand protein or polypeptide.

According to a further aspect of the invention there is provided a method for reducing immune tolerance by administering a Notch ligand protein or polypeptide consisting essentially of the following components:
i) a Notch ligand DSL domain;
ii) optionally 1 or 2 EGF repeat domains;
iii) optionally all or part of a Notch ligand N-terminal domain; and
iv) optionally one or more heterologous amino acid sequences;
or by administering a multimer of such a protein or polypeptide (wherein each monomer may be the same or different);
or by administering a polynucleotide coding for such a Notch ligand protein or polypeptide.

According to a further aspect of the invention there is provided a method for modifying T cell activity by administering a Notch ligand protein or polypeptide consisting essentially of the following components:
i) a Notch ligand DSL domain;
ii) optionally 1 or 2 EGF repeat domains;
iii) optionally all or part of a Notch ligand N-terminal domain; and
iv) optionally one or more heterologous amino acid sequences;
or by administering a multimer of such a protein or polypeptide (wherein each monomer may be the same or different);
or by administering a polynucleotide coding for such a Notch ligand protein or polypeptide.

According to a further aspect of the invention there is provided a method for increasing helper (T_{H}) or cytotoxic (T_{C}) T-cell activity by administering a Notch ligand protein or polypeptide consisting essentially of the following components:
i) a Notch ligand DSL domain;
ii) optionally 1 or 2 EGF repeat domains;
iii) optionally all or part of a Notch ligand N-terminal domain; and
iv) optionally one or more heterologous amino acid sequences;
or by administering a multimer of such a protein or polypeptide (wherein each monomer may be the same or different);
or by administering a polynucleotide coding for such a Notch ligand protein or polypeptide.

According to a further aspect of the invention there is provided a method for reducing activity of regulatory T cells by administering a Notch ligand protein or polypeptide consisting essentially of the following components:
i) a Notch ligand DSL domain;
ii) optionally 1 or 2 EGF repeat domains;
iii) optionally all or part of a Notch ligand N-terminal domain; and
iv) optionally one or more heterologous amino acid sequences;
or by administering a multimer of such a protein or polypeptide (wherein each monomer may be the same or different);
or by administering a polynucleotide coding for such a Notch ligand protein or polypeptide.

Suitably the regulatory T cells are Tr1 or Th3 regulatory T-cells.

According to a further aspect of the invention there is provided a Notch ligand protein or polypeptide consisting essentially of the following components:
i) a Notch ligand DSL domain;
ii) optionally 1 or 2 EGF domains;
iii) optionally all or part of a Notch ligand N-terminal domain; and
iv) optionally one or more heterologous amino acid sequences;
or a multimer of such a protein or polypeptide (wherein each monomer may be the same or different);
or a polynucleotide coding for such a Notch ligand protein or polypeptide; for use to treat disease.

According to a further aspect of the invention there is provided a Notch ligand protein or polypeptide or polynucleotide for a use as claimed in claim 22 wherein the Notch ligand protein or polypeptide consists essentially of the following components:
i) a Notch ligand DSL domain;
ii) optionally all or part of a Notch ligand N-terminal domain; and
iii) optionally one or more heterologous amino acid sequences;
or wherein the polynucleotide codes for such a Notch ligand protein or polypeptide.

According to a further aspect of the invention there is provided the use of a Notch ligand protein or polypeptide consisting essentially of the following components:
i) a Notch ligand DSL domain;
ii) optionally 1 or 2 EGF domains;
iii) optionally all or part of a Notch ligand N-terminal domain; and
iv) optionally one or more heterologous amino acid sequences;
or a multimer of such a protein or polypeptide (wherein each monomer may be the same or different);
or a polynucleotide coding for such a Notch ligand protein or polypeptide; in the manufacture of a medicament for modification of an immune response.

According to a further aspect of the invention there is provided the use of a Notch ligand protein or polypeptide consisting essentially of the following components:
i) a Notch ligand DSL domain;
ii) optionally 1 or 2 EGF domains; and
iii) optionally one or more heterologous amino acid sequences;
or a multimer of such a protein or polypeptide (wherein each monomer may be the same or different);
or a polynucleotide coding for such a Notch ligand protein or polypeptide; in the manufacture of a medicament for modification of an immune response.

According to a further aspect of the invention there is provided the use of a Notch ligand protein or polypeptide consisting essentially of the following components:
i) a Notch ligand DSL domain;
ii) optionally 1 or 2 EGF domains;
iii) optionally all or part of a Notch ligand N-terminal domain; and
iv) optionally one or more heterologous amino acid sequences;
or a multimer of such a protein or polypeptide (wherein each monomer may be the same or different);
or a polynucleotide coding for such a Notch ligand protein or polypeptide; in the manufacture of a medicament for increasing an immune response.

According to a further aspect of the invention there is provided the use of a Notch ligand protein or polypeptide consisting essentially of the following components:
i) a Notch ligand DSL domain;
ii) optionally 1 or 2 EGF domains;
iii) optionally all or part of a Notch ligand N-terminal domain; and
iv) optionally one or more heterologous amino acid sequences;
or a multimer of such a protein or polypeptide (wherein each monomer may be the same or different);
or a polynucleotide coding for such a Notch ligand protein or polypeptide; in the manufacture of a medicament for reducing immune tolerance.

According to a further aspect of the invention there is provided the use of a Notch ligand protein or polypeptide consisting essentially of the following components:
i) a Notch ligand DSL domain;
ii) optionally 1 or 2 EGF domains;
iii) optionally all or part of a Notch ligand N-terminal domain; and
iv) optionally one or more heterologous amino acid sequences;
or a multimer of such a protein or polypeptide (wherein each monomer may be the same or different);
or a polynucleotide coding for such a Notch ligand protein or polypeptide; in the manufacture of a medicament for modification of T-cell activity.

According to a further aspect of the invention there is provided the use of a Notch ligand protein or polypeptide consisting essentially of the following components:
i) a Notch ligand DSL domain;
ii) optionally 1 or 2 EGF domains;
iii) optionally all or part of a Notch ligand N-terminal domain; and
iv) optionally one or more heterologous amino acid sequences;
or a multimer of such a protein or polypeptide (wherein each monomer may be the same or different);
or a polynucleotide coding for such a Notch ligand protein or polypeptide; in the manufacture of a medicament for increasing helper (T_{H}) or cytotoxic (T_{C}) T-cell activity.

According to a further aspect of the invention there is provided the use of a Notch ligand protein or polypeptide consisting essentially of the following components:
i) a Notch ligand DSL domain;
ii) optionally 1 or 2 EGF domains;
iii) optionally all or part of a Notch ligand N-terminal domain; and
iv) optionally one or more heterologous amino acid sequences;
or a multimer of such a protein or polypeptide (wherein each monomer may be the same or different);
or a polynucleotide coding for such a Notch ligand protein or polypeptide;
in the manufacture of a medicament for reducing activity of regulatory T cells.

According to a further aspect of the invention there is provided a pharmaceutical composition comprising a Notch ligand protein or polypeptide consisting essentially of the following components:
i) a Notch ligand DSL domain;
ii) optionally 1 or 2 EGF domains;
iii) optionally all or part of a Notch ligand N-terminal domain; and
iv) optionally one or more heterologous amino acid sequences;
or a multimer of such a protein or polypeptide (wherein each monomer may be the same or different);
or a polynucleotide coding for such a Notch ligand protein or polypeptide;
optionally in combination with a pharmaceutically acceptable carrier.

According to a further aspect of the invention there is provided a pharmaceutical composition comprising a Notch ligand protein or polypeptide consisting essentially of the following components:
i) a Notch ligand DSL domain;
ii) optionally all or part of a Notch ligand N-terminal domain; and
iii) optionally one or more heterologous amino acid sequences;
or a multimer of such a protein or polypeptide (wherein each monomer may be the same or different);
or a polynucleotide coding for such a Notch ligand protein or polypeptide;
optionally in combination with a pharmaceutically acceptable carrier.

According to a further aspect of the invention there is provided a pharmaceutical composition comprising a Notch ligand protein or polypeptide consisting essentially of the following components:
i) a Notch ligand DSL domain;
ii) one EGF repeat domain;
iii) optionally all or part of a Notch ligand N-terminal domain; and
iv) optionally one or more heterologous amino acid sequences;
or a multimer of such a protein or polypeptide (wherein each monomer may be the same or different);
or a polynucleotide coding for such a Notch ligand protein or polypeptide;
optionally in combination with a pharmaceutically acceptable carrier.

According to a further aspect of the invention there is provided a pharmaceutical composition comprising a Notch ligand protein or polypeptide consisting essentially of the following components:
i) a Notch ligand DSL domain;
ii) two EGF domains;
iii) optionally all or part of a Notch ligand N-terminal domain; and
iv) optionally one or more heterologous amino acid sequences;
or a multimer of such a protein or polypeptide (wherein each monomer may be the same or different);
or a polynucleotide coding for such a Notch ligand protein or polypeptide;
optionally in combination with a pharmaceutically acceptable carrier.

According to a further aspect of the invention there is provided a Notch ligand protein or polypeptide which consists essentially of the following components:
i) a Notch ligand DSL domain;
ii) optionally all or part of a Notch ligand N-terminal domain;
iii) an immunoglobulin Fc domain; and
iv) optionally one or more further heterologous amino acid sequences;
or a multimer of such a protein or polypeptide (wherein each monomer may be the same or different);
or a polynucleotide coding for such a Notch ligand protein or polypeptide;

According to a further aspect of the invention there is provided a Notch ligand protein or polypeptide which consists essentially of the following components:
i) a Notch ligand DSL domain;
ii) one EGF domain;
iii) optionally all or part of a Notch ligand N-terminal domain; and
iv) optionally one or more heterologous amino acid sequences;
or a multimer of such a protein or polypeptide (wherein each monomer may be the same or different);
or a polynucleotide coding for such a Notch ligand protein or polypeptide;

According to a further aspect of the invention there is provided a Notch ligand protein or polypeptide which consists essentially of the following components:
i) a Notch ligand DSL domain;
ii) two EGF domains; and
iii) optionally one or more heterologous amino acid sequences;
or a polynucleotide sequence which codes for such a Notch ligand protein or polypeptide.

The term “which consists essentially of” or “consisting essentially of” as used herein means that the construct includes the sequences and domains identified but is substantially free of other sequences or domains, and in particular is substantially free of any other Notch or Notch ligand sequences or domains.

For avoidance of doubt the term “comprising” means that any additional feature or component may be present.

According to a further aspect of the invention there is provided a vector comprising a polynucleotide coding for a Notch ligand protein or polypeptide as described above.

The invention also provides a host cell transformed or transfected with such a vector.

According to a further aspect of the invention there is provided a cell displaying a Notch ligand protein or polypeptide as described above on its surface and/or transfected with a polynucleotide coding for such a protein or polypeptide.

Suitably the protein or polypeptide is not bound to a cell. Alternatively, the protein or polypeptide may be cell-associated.

In one embodiment the protein or polypeptide may be fused to a heterologous amino acid sequence corresponding to all or part of an immunoglobulin F_{c} segment. In one embodiment, particularly where the Notch ligand protein or polypeptide comprises only two EGF repeat domains, the heterologous amino acid sequence is not a TSST sequence, or preferably is not a superantigen sequence.

Preferably the protein or polypeptide comprises at least part of a mammalian, preferably human, Notch ligand sequence.

Suitably the protein or polypeptide comprises Notch ligand domains from Delta, Serrate or Jagged or domains having at least 30% amino acid sequence similarity or identity thereto.

Suitably the protein or polypeptide comprises Notch ligand domains from Delta1, Delta 3, Delta 4, Jagged 1 or Jagged 2 or domains having at least 30% amino acid sequence similarity thereto.

Preferably the protein or polypeptide inhibits a Notch receptor. Suitably the protein or polypeptide is a Notch signalling antagonist.

According to a further aspect of the invention there is provided a polynucleotide coding for a protein or polypeptide as described above. According to further aspects of the invention there are provided a vector comprising such a polynucleotide and a host cell transformed or transfected with such a vector.

According to a further aspect of the invention there is provided a cell displaying a Notch ligand protein or polypeptide as described above on its surface and/or transfected with a polynucleotide coding for such a protein or polypeptide.

In one embodiment the modulator of the Notch signalling pathway may comprise a fusion protein comprising domains from a Notch ligand extracellular domain and an immunoglobulin F_{c} segment (eg IgG1 Fc or IgG4 Fc) or a polynucleotide coding for such a fusion protein. Methods suitable for preparation of such fusion proteins are described, for example in Example 2 of WO 98/20142. IgG fusion proteins may be prepared as well known in the art, for example, as described in U.S. Pat. No. 5,428,130 (Genentech).

According to a further aspect of the invention there is provided a method for increasing TNFα expression by administering a protein, polypeptide or polynucleotide as described above.

According to a further aspect of the invention there is provided a method for reducing IL-10 expression by administering a protein, polypeptide or polynucleotide as described above.

According to a further aspect of the invention there is provided a method for increasing IL-5 expression by administering a protein, polypeptide or polynucleotide as described above.

According to a further aspect of the invention there is provided a method for increasing IL-13 expression by administering a protein, polypeptide or polynucleotide as described above.

Suitably the protein, polypeptide or polynucleotide modifies cytokine expression in leukocytes (such as lymphocytes or macrophages), fibroblasts or epithelial cells or their progenitors or tissue-specific derivatives.

According to a further aspect of the invention there is provided a method for generating an immune stimulatory cytokine profile with reduced IL-10 expression and increased TNFα expression by administering a protein, polypeptide or polynucleotide as described above.

According to a further aspect of the invention there is provided a method for generating an immune stimulatory cytokine profile with reduced IL-10 expression and increased IL-5 expression by administering a protein, polypeptide or polynucleotide as described above.

According to a further aspect of the invention there is provided a method for generating an immune stimulatory cytokine profile with reduced IL-10 expression and increased IL-13 expression by administering a protein, polypeptide or polynucleotide as described above.

According to a further aspect of the invention there is provided a method for generating an immune stimulatory cytokine profile with increased IL-5, IL-13 and TNFα expression by administering a protein, polypeptide or polynucleotide as described above.

According to a further aspect of the invention there is provided a method for generating an immune stimulatory cytokine profile with increased IL-2, IFNγ, IL-5, IL-13 and TNFα expression by administering a protein, polypeptide or polynucleotide as described above.

Suitably the cytokine profile also exhibits reduced IL-10 expression.

According to a further aspect of the invention there is provided a method for increasing a TH2 immune response by administering a protein, polypeptide or polynucleotide as described above.

According to a further aspect of the invention there is provided a method for increasing a TH1 immune response by administering a protein, polypeptide or polynucleotide as described above.

### BRIEF DESCRIPTION OF THE DRAWINGS

Various preferred features and embodiments of the present invention will now be described in more detail by way of non-limiting example and with reference to the accompanying drawings, in which:

FIG. 1 shows a schematic representation of Notch/Ligand interaction;

FIG. 2 shows a schematic representation of the Notch signalling pathway;

FIG. 3 shows a schematic representation of Notch 1-4;

FIG. 4 shows a schematic representation of Notch ligands Jagged and Delta;

FIG. 5 shows aligned amino acid sequences of DSL domains from various Drosophila and mammalian Notch ligands;

FIG. 6 shows amino acid sequences of human Delta-1, Delta-3 and Delta-4;

FIG. 7 shows amino acid sequences of human Jagged-1 and Jagged-2;

FIG. 8 shows an amino acid sequence of human Notch-1;

FIG. 9 shows an amino acid sequence of human Notch-2;

FIG. 10 shows a schematic representation of Notch ligand/IgFc fusion proteins suitable for use in the present invention;

FIG. 11 shows a schematic representation of a nucleic acid expression construct according to the present invention;

FIG. 12 shows the amino acid sequence and domain structure of the fusion protein of Example 1;

FIG. 13 shows the results of Example 2;

FIG. 14 shows the results of Example 3;

FIG. 15 shows the results of Example 4;

FIG. 16 shows the results of Example 5;

FIG. 17 shows the results of Example 6;

FIG. 18 shows the results of Example 8;

FIG. 19 shows the results of Example 9;

FIG. 20 shows the results of Example 10;

FIG. 21 shows the results of Example 11;

FIG. 21 shows the results of Example 12;

FIG. 22 shows the results of Example 13;

FIG. 23 shows the results of Example 14;

FIG. 24 shows the results of Example 15;

FIG. 25 shows the results of Example 16;

FIG. 26 shows the results of Example 17;

FIG. 27 shows the results of Example 18;

FIGS. 28 and 29 show the results of Example 19;

FIGS. 30 and 31 show the results of Example 21;

FIGS. 32 and 33 shows the results of Example 22; and

FIG. 34 shows the results of Example 23.

### DETAILED DESCRIPTION

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; and J. E. Coligan, A. M. Kruisbeek, D. H. Margulies, E. M. Shevach and W. Strober (1992 and periodic supplements; Current Protocols in Immunology, John Wiley & Sons, New York, N.Y.). Each of these general texts is herein incorporated by reference.

For the avoidance of doubt, Drosophila and vertebrate names are used interchangeably and all homologues are included within the scope of the invention.

Notch Signalling

As used herein, the expression “Notch signalling” is synonymous with the expression “the Notch signalling pathway” and refers to any one or more of the upstream or downstream events that result in, or from, (and including) activation of the Notch receptor.

Preferably, by “Notch signalling” we refer to any event directly upstream or downstream of Notch receptor activation or inhibition including activation or inhibition of Notch/Notch ligand interactions, upregulation or downregulation of Notch or Notch ligand expression or activity and activation or inhibition of Notch signalling transduction including, for example, proteolytic cleavage of Notch and upregulation or downregulation of the Ras-Jnk signalling pathway.

Thus, by “Notch signalling” we refer to the Notch signalling pathway as a signal tranducing pathway comprising elements which interact, genetically and/or molecularly, with the Notch receptor protein. For example, elements which interact with the Notch protein on both a molecular and genetic basis are, by way of example only, Delta, Serrate and Deltex. Elements which interact with the Notch protein genetically are, by way of example only, Mastermind, Hairless, Su(H) and Presenilin.

In one aspect, Notch signalling includes signalling events taking place extracellularly or at the cell membrane. In a further aspect, it includes signalling events taking place intracellularly, for example within the cell cytoplasm or within the cell nucleus.

Modulators of Notch Signalling

The term “modulate” as used herein refers to a change or alteration in the biological activity of the Notch signalling pathway or a target signalling pathway thereof. The term “modulator” preferably refers to antagonists or inhibitors of Notch signalling, i.e. compounds which block, at least to some extent, the normal biological activity of the Notch signalling pathway. Conveniently such compounds may be referred to herein as inhibitors or antagonists. Preferably the modulator is an antagonist of Notch signalling, and preferably an antagonist of the Notch receptor (eg an antagonist of the Notch1, Notch2, Notch3 and/or Notch4 receptor).

An antagonist of the Notch receptor is preferably an agent which binds to the extracellular domain of Notch to reduce or inhibit activation of signalling. Preferably an antagonist of the Notch receptor binds to Notch in immune cells, such as APCs, B-cells or T-cells.

Alternatively, an inhibitor of Notch signalling may bind to Notch ligands to reduce their ability to bind to and/or activate a Notch receptor. Preferably such an inhibitor binds to Notch ligands in immune cells, such as APCs, B-cells or T-cells.

The active agent of the present invention may be an organic compound or other chemical. In one embodiment, a modulator will be an organic compound comprising two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. The candidate modulator may comprise at least one cyclic group. The cyclic group may be a polycyclic group, such as a non-fused polycyclic group. For some applications, the agent comprises at least the one of said cyclic groups linked to another hydrocarbyl group.

In one preferred embodiment, the modulator will be an amino acid sequence or a chemical derivative thereof, or a combination thereof. In another preferred embodiment, the modulator will be a nucleotide sequence—which may be a sense sequence or an anti-sense sequence. The modulator may also be an antibody.

Modulators may be synthetic compounds or natural isolated compounds.

A very important component of the Notch signalling pathway is Notch receptor/Notch ligand interaction. Thus Notch signalling may involve changes in expression, nature, amount or activity of Notch ligands or receptors or their resulting cleavage products. In addition, Notch signalling may involve changes in expression, nature, amount or activity of Notch signalling pathway membrane proteins or G-proteins or Notch signalling pathway enzymes such as proteases, kinases (e.g. serine/threonine kinases), phosphatases, ligases (e.g. ubiquitin ligases) or glycosyltransferases. Alternatively the signalling may involve changes in expression, nature, amount or activity of DNA binding elements such as transcription factors.

In a preferred form of the invention the Notch signalling is specific signalling, meaning that the signal detected results substantially or at least predominantly from the Notch signalling pathway, and preferably from Notch/Notch ligand interaction, rather than any other significant interfering or competing cause, such as for example cytokine signalling. Thus, in a preferred embodiment the term “Notch signalling” as used herein excludes cytokine signalling. Preferably therefore the modulator or inhibitor of Notch signalling is not a cytokine and is preferably not a mitogen.

Preferably the modulator of Notch signalling is not an agent which acts primarily by inhibiting or downregulating the expression of a Notch ligand such as Delta and/or Serrate. Thus, it will be appreciated that although such inhibition or downregulation may occur as a result of the main mode of action of the modulator of Notch signalling, preferably this is not the primary mode of action of the modulator. Preferably the primary mode of action of the modulator of Notch signalling is to modulate (preferably inhibit) interactions between Notch and Notch ligands which are already expressed on immune cells.

Thus, preferably the modulator of Notch signalling is not a Toll protein or BMP and is preferably not an agent which decreases or interferes with the production of Noggin, Chordin, Follistatin, Xnr3, FGF or Fringe as described, for example in WO98/20142.

The Notch signalling pathway is described in more detail below.

Key targets for Notch-dependent transcriptional activation are genes of the Enhancer of split complex (E[spl]). Moreover these genes have been shown to be direct targets for binding by the Su(H) protein and to be transcriptionally activated in response to Notch signalling. By analogy with EBNA2, a viral coactivator protein that interacts with a mammalian Su(H) homologue CBF1 to convert it from a transcriptional repressor to a transcriptional activator, the Notch intracellular domain, perhaps in association with other proteins may combine with Su(H) to contribute an activation domain that allows Su(H) to activate the transcription of E(spl) as well as other target genes. It should also be noted that Su(H) is not required for all Notch-dependent decisions, indicating that Notch mediates some cell fate choices by associating with other DNA-binding transcription factors or by employing other mechanisms to transduce extracellular signals.

In one embodiment, the active agent may be a Notch ligand, or a polynucleotide encoding a Notch ligand. Notch ligands of use in the present invention include endogenous Notch ligands which are typically capable of binding to a Notch receptor polypeptide present in the membrane of a variety of mammalian cells, for example hemapoietic stem cells.

The term “Notch ligand” as used herein means an agent capable of interacting with a Notch receptor to cause a biological effect. The term includes naturally occurring protein ligands such as Delta and Serrate, and artificial/modified constructs having equivalent activity.

Particular examples of mammalian Notch ligands identified to date include the Delta family, for example Delta or Delta-like 1 (Genbank Accession No. AF003522—Homo sapiens), Delta-3 (Genbank Accession No. AF084576—Rattus norvegicus) and Delta-like 3 (Mus musculus) (Genbank Accession No. NM_{—}016941—Homo sapiens) and U.S. Pat. No. 6,121,045 (Millennium), Delta-4 (Genbank Accession Nos. AB043894 and AF 253468—Homo sapiens) and the Serrate family, for example Serrate-1 and Serrate-2 (WO97/01571, WO96/27610 and WO92/19734), Jagged-1 (Genbank Accession No. U73936—Homo sapiens) and Jagged-2 (Genbank Accession No. AF029778—Homo sapiens), and LAG-2. Homology between family members is extensive.

Further homologues of known mammalian Notch ligands may be identified using standard techniques. By a “homologue” it is meant a gene product that exhibits sequence homology, either amino acid or nucleic acid sequence homology, to any one of the known Notch ligands, for example as mentioned above. Typically, a homologue of a known Notch ligand will be at least 20%, preferably at least 30%, identical at the amino acid level to the corresponding known Notch ligand over a sequence of at least 10, preferably at least 20, preferably at least 50, suitably at least 100 amino acids, or over the entire length of the Notch ligand. Techniques and software for calculating sequence homology between two or more amino acid or nucleic acid sequences are well known in the art (see for example programs available through the National Center for Biotechnology Information of the National Institutes of Health and Ausubel et al., Current Protocols in Molecular Biology (1995), John Wiley & Sons, Inc.)

Notch ligands identified to date have a diagnostic DSL domain (D. Delta, S. Serrate, L. Lag2) comprising 20 to 22 amino acids at the amino terminus of the protein and up to 14 or more EGF-like repeats on the extracellular surface. It is therefore preferred that homologues of Notch ligands also comprise a DSL domain at the N-terminus and up to 14 or more EGF-like repeats on the extracellular surface.

In addition, suitable homologues will be capable of binding to a Notch receptor. Binding may be assessed by a variety of techniques known in the art including in vitro binding assays.

Homologues of Notch ligands can be identified in a number of ways, for example by probing genomic or cDNA libraries with probes comprising all or part of a nucleic acid encoding a Notch ligand under conditions of medium to high stringency (for example 0.03M sodium chloride and 0.03M sodium citrate at from about 50° C. to about 60° C.). Alternatively, homologues may also be obtained using degenerate PCR which will generally use primers designed to target sequences within the variants and homologues encoding conserved amino acid sequences. The primers will contain one or more degenerate positions and will be used at stringency conditions lower than those used for cloning sequences with single sequence primers against known sequences.

Inhibition of Notch signalling may also be achieved by mimicking or enhancing activity or expression of inhibitors of the Notch signalling pathway. As such, polypeptides for Notch signalling inhibition include molecules capable of mimicking or enhancing activity or expression of any Notch signalling inhibitors. Preferably the molecule will be a polypeptide, or a polynucleotide encoding such a polypeptide, that increases the production or activity of compounds that are capable of producing a decrease in the expression or activity of Notch, Notch ligands, or any downstream components of the Notch signalling pathway. Such molecules include the Toll-like receptor protein family, and growth factors such as the bone morphogenetic protein (BMP), BMP receptors and activins, derivatives, fragments, variants and homologues thereof.

By a protein which is for Notch signalling inhibition or a polynucleotide encoding such a protein, we mean a molecule which is capable of inhibiting Notch, the Notch signalling pathway or any one or more of the components of the Notch signalling pathway.

In one embodiment, the molecule may be capable of reducing or preventing Notch or Notch ligand expression. Such a molecule may be a nucleic acid sequence capable of reducing or preventing Notch or Notch ligand expression.

Suitably the nucleic acid sequence encodes a polypeptide selected from Toll-like receptor protein family or a growth factor such as a bone morphogenetic protein (BMP), a BMP receptor and activins. Preferably the agent is a polypeptide, or a polynucleotide encoding such a polypeptide, that decreases or interferes with the production of compounds that are capable of producing an increase in the expression of Notch ligand, such as Noggin, Chordin, Follistatin, Xnr3, fibroblast growth factors and derivatives, fragments, variants and homologues thereof.

Alternatively, the nucleic acid sequence may be an antisense construct derived from a sense nucleotide sequence encoding a polypeptide selected from a Notch ligand and a polypeptide capable of upregulating Notch ligand expression, such as Noggin, Chordin, Follistatin, Xnr3, fibroblast growth factors and derivatives, fragments, variants and homologues thereof.

Preferably, however, an inhibitor of Notch signalling will be a molecule which is capable of inhibiting Notch-Notch ligand interactions. A molecule may be considered to modulate Notch-Notch ligand interactions if it is capable of inhibiting the interaction of Notch with its naturally occurring ligands, preferably to an extent sufficient to provide therapeutic efficacy.

Agents which modulate Notch-Notch ligand interaction may, for example be antibodies, antibody fragments or derivatives, peptides, small organic molecules, peptidomimetics or the like. Antibodies are preferred agents. Such antibodies may be polyclonal or monoclonal, intact or truncated, and may for example be xenogeneic, allogeneic or syngeneic.

For example, antibodies capable of binding to Notch receptors or Notch ligands may be used to inhibit normal Notch-Notch ligand interactions in accordance with the present invention.

The expression “Notch-Notch ligand interaction” (which may be used interchangeably with the term “Notch ligand-receptor interaction”) as used herein means the interaction between a Notch family member and a ligand capable of binding to one or more such member.

An agent may be considered to inhibit Notch-Notch ligand interactions if it is capable of inhibiting the interaction of Notch with its ligands, preferably to an extent sufficient to provide therapeutic efficacy.

Whilst oligopeptides and peptides may be preferred agents, other sources such as combinatorial libraries provide compounds other than oligopeptides that have the necessary binding characteristics.

Non-peptide agents include numerous chemical types, though typically they are organic molecules, preferably small organic compounds having a molecular weight of between about 50 and about 2,500 daltons. Suitable agents include functional groups necessary for structural interaction with proteins, particularly hydrogen bonding, and frequently include at least one group selected from, for example, an amine, carbonyl, carboxyl, hydroxyl, or sulfhydryl group, preferably at least two such functional chemical groups. Compounds may, for example be cyclic or heterocyclic structures and/or aromatic or polyaromatic structures substituted with one or more such functional groups.

Suitably the agents block binding of human Notch to human Delta and/or Serrate by at least about 10%, 20%, 30%, 40%, 50%, 60%, 70%, 80%, 90%, 99%, or 100%.

Preferably when the inhibitor is a receptor or a nucleic acid sequence encoding a receptor, the receptor is activated. Thus, for example, when the agent is a nucleic acid sequence, the receptor is preferably constitutively active when expressed.

Inhibitors of Notch signalling also include downstream inhibitors of the Notch signalling pathway, compounds that prevent expression of Notch target genes or induce expression of genes repressed by the Notch signalling pathway. Examples of such proteins include Dsh or Numb and dominant negative versions of Notch IC or Deltex. Proteins for Notch signalling inhibition will also include variants of the wild-type components of the Notch signalling pathway which have been modified in such a way that their presence blocks rather than transduces the signalling pathway. An example of such a compound would be a Notch receptor which has been modified such that proteolytic cleavage of its intracellular domain is no longer possible.

Notch signalling may also be inhibited by inhibiting Notch signalling transduction.

Notch Signalling Transduction

The Notch signalling pathway directs binary cell fate decisions in the embryo. Notch was first described in Drosophila as a transmembrane protein that functions as a receptor for two different ligands, Delta and Serrate. Vertebrates express multiple Notch receptors and ligands (discussed below). At least four Notch receptors (Notch-1, Notch-2, Notch-3 and Notch-4) have been identified to date in human cells (see for example GenBank Accession Nos. AF308602, AF308601 and U95299—Homo sapiens).

Notch proteins are synthesized as single polypeptide precursors that undergo cleavage via a Furin-like convertase that yields two polypeptide chains that are further processed to form the mature receptor. The Notch receptor present in the plasma membrane comprises a heterodimer of two Notch proteolytic cleavage products, one comprising an N-terminal fragment consisting of a portion of the extracellular domain, the transmembrane domain and the intracellular domain, and the other comprising the majority of the extracellular domain. The proteolytic cleavage step of Notch to activate the receptor occurs in the Golgi apparatus and is mediated by a furin-like convertase.

Notch receptors are inserted into the membrane as heterodimeric molecules consisting of an extracellular domain containing up to 36 epidermal growth factor (EGF)-like repeats [Notch 1/2=36, Notch 3=34 and Notch 4=29], 3 Cysteine Rich Repeats (Lin-Notch (L/N) repeats) and a transmembrane subunit that contains the cytoplasmic domain. The cytoplasmic domain of Notch contains six ankyrin-like repeats, a polyglutamine stretch (OPA) and a PEST sequence. A further domain termed RAM23 lies proximal to the ankyrin repeats and is involved in binding to a transcription factor, known as Suppressor of Hairless [Su(H)] in Drosophila and CBF1 in vertebrates (Tamura K, et al. (1995) Curr. Biol. 5:1416-1423 (Tamura)). The Notch ligands also display multiple EGF-like repeats in their extracellular domains together with a cysteine-rich DSL (Delta-Serrate Lag2) domain that is characteristic of all Notch ligands (Artavanis-Tsakomas et al. (1995) Science 268:225-232, Artavanis-Tsakomas et al. (1999) Science 284:770-776).

The Notch receptor is activated by binding of extracellular ligands, such as Delta, Serrate and Scabrous, to the EGF-like repeats of Notch's extracellular domain. Delta requires cleavage for activation. It is cleaved by the ADAM disintegrin metalloprotease Kuzbanian at the cell surface, the cleavage event releasing a soluble and active form of Delta. An oncogenic variant of the human Notch-1 protein, also known as TAN-1, which has a truncated extracellular domain, is constitutively active and has been found to be involved in T-cell lymphoblastic leukemias.

The cdc10/ankyrin intracellular-domain repeats mediate physical interaction with intracellular signal transduction proteins. Most notably, the cdc10/ankyrin repeats interact with Suppressor of Hairless [Su(H)]. Su(H) is the Drosophila homologue of C-promoter binding factor-1 [CBF-1], a mammalian DNA binding protein involved in the Epstein-Barr virus-induced immortalization of B-cells. It has been demonstrated that, at least in cultured cells, Su(H) associates with the cdc10/ankyrin repeats in the cytoplasm and translocates into the nucleus upon the interaction of the Notch receptor with its ligand Delta on adjacent cells. Su(H) includes responsive elements found in the promoters of several genes and has been found to be a critical downstream protein in the Notch signalling pathway. The involvement of Su(H) in transcription is thought to be modulated by Hairless.

The intracellular domain of Notch (NotchIC) also has a direct nuclear function (Lieber et al. (1993) Genes Dev 7(10):1949-65 (Lieber)). Recent studies have indeed shown that Notch activation requires that the six cdc10/ankyrin repeats of the Notch intracellular domain reach the nucleus and participate in transcriptional activation. The site of proteolytic cleavage on the intracellular tail of Notch has been identified between gly1743 and val 1744 (termed site 3, or S3) (Schroeter, E. H. et al. (1998) Nature 393(6683):382-6 (Schroeter)). It is thought that the proteolytic cleavage step that releases the cdc 10/ankyrin repeats for nuclear entry is dependent on Presenilin activity.

The intracellular domain has been shown to accumulate in the nucleus where it forms a transcriptional activator complex with the CSL family protein CBF1 (suppressor of hairless, Su(H) in Drosophila, Lag-2 in C. elegans) (Schroeter; Struhl, G. et al. (1998) Cell 93(4):649-60 (Struhl)). The NotchIC-CBF1 complexes then activate target genes, such as the bHLH proteins HES (hairy-enhancer of split like) 1 and 5 (Weinmaster G. (2000) Curr. Opin. Genet. Dev. 10:363-369 (Weinmaster)). This nuclear function of Notch has also been shown for the mammalian Notch homologue (Lu, F. M. et al. (1996) Proc Natl Acad Sci 93(11):5663-7 (Lu)).

S3 processing occurs only in response to binding of Notch ligands Delta or Serrate/Jagged. The post-translational modification of the nascent Notch receptor in the Golgi (Munro S, Freeman M. (2000) Curr. Biol. 10:813-820 (Munro); Ju B J, et al. (2000) Nature 405:191-195 (Ju)) appears, at least in part, to control which of the two types of ligand is expressed on a cell surface. The Notch receptor is modified on its extracellular domain by Fringe, a glycosyl transferase enzyme that binds to the Lin/Notch motif. Fringe modifies Notch by adding O-linked fucose groups to the EGF-like repeats (Moloney D J, et al. (2000) Nature 406:369-375 (Moloney), Brucker K, et al. (2000) Nature 406:411-415 (Brucker)). This modification by Fringe does not prevent ligand binding, but may influence ligand induced conformational changes in Notch. Furthermore, recent studies suggest that the action of Fringe modifies Notch to prevent it from interacting functionally with Serrate/Jagged ligands but allow it to preferentially bind Delta (Panin V M, et al. (1997) Nature 387:908-912 (Panin), Hicks C, et al. (2000) Nat. Cell. Biol. 2:515-520 (Hicks)). Although Drosophila has a single Fringe gene, vertebrates are known to express multiple genes (Radical, Manic and Lunatic Fringes) (Irvine KD (1999) Curr. Opin. Genet. Devel. 9:434-441 (Irvine)).

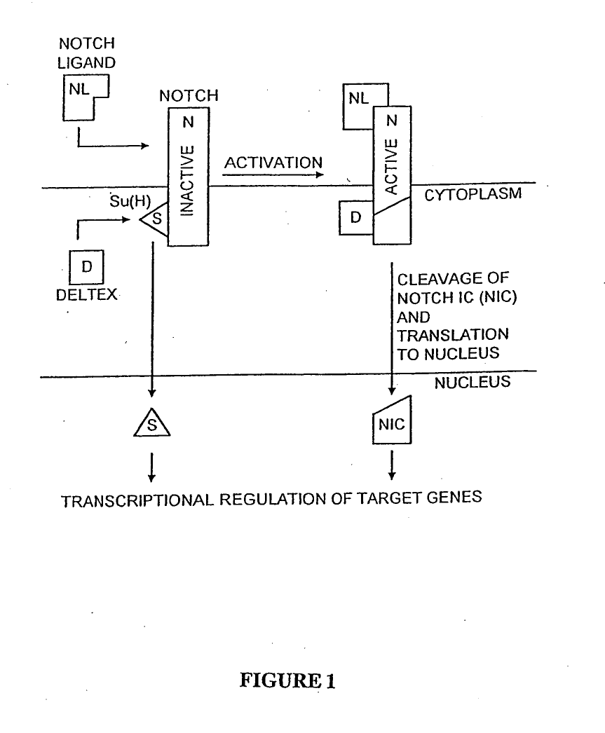

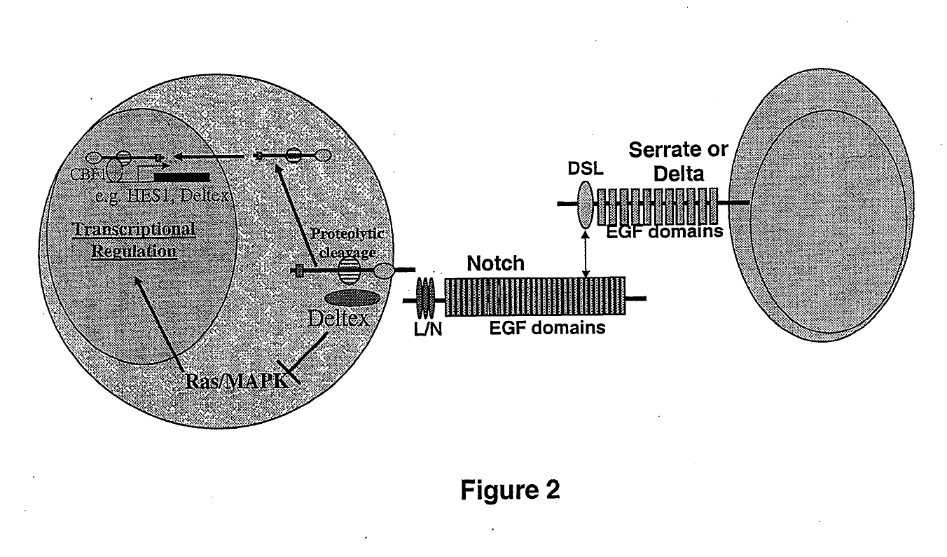

Signal transduction from the Notch receptor can occur via two different pathways (FIG. 1). The better defined pathway involves proteolytic cleavage of the intracellular domain of Notch (Notch IC) that translocates to the nucleus and forms a transcriptional activator complex with the CSL family protein CBF1 (suppressor of Hairless, Su(H) in Drosophila, Lag-2 in C. elegans). NotchIC-CBF1 complexes then activate target genes, such as the bHLH proteins HES (hairy-enhancer of split like) 1 and 5. Notch can also signal in a CBF1-independent manner that involves the cytoplasmic zinc finger containing protein Deltex. Unlike CBF1, Deltex does not move to the nucleus following Notch activation but instead can interact with Grb2 and modulate the Ras-JNK signalling pathway.

Target genes of the Notch signalling pathway include Deltex, genes of the Hes family (Hes-1 in particular), Enhancer of Split [E(spl)] complex genes, IL-10, CD-23, CD-4 and Dll-1.

Deltex, an intracellular docking protein, replaces Su(H) as it leaves its site of interaction with the intracellular tail of Notch. Deltex is a cytoplasmic protein containing a zinc-finger (Artavanis-Tsakomas et al. (1995) Science 268:225-232; Artavanis-Tsakomas et al. (1999) Science 284:770-776; Osborne B, Miele L. (1999) Immunity 11:653-663 (Osborne)). It interacts with the ankyrin repeats of the Notch intracellular domain. Studies indicate that Deltex promotes Notch pathway activation by interacting with Grb2 and modulating the Ras-JNK signalling pathway (Matsuno et al. (1995) Development 121(8):2633-44; Matsuno K, et al. (1998) Nat. Genet. 19:74-78). Deltex also acts as a docking protein which prevents Su(H) from binding to the intracellular tail of Notch (Matsuno). Thus, Su(H) is released into the nucleus where it acts as a transcriptional modulator. Recent evidence also suggests that, in a vertebrate B-cell system, Deltex, rather than the Su(H) homologue CBF1, is responsible for inhibiting E47 function (Ordentlich et al. (1998) Mol. Cell. Biol. 18:2230-2239 (Ordentlich)). Expression of Deltex is upregulated as a result of Notch activation in a positive feedback loop. The sequence of Homo sapiens Deltex (DTX1) mRNA may be found in GenBank Accession No. AF053700.

Hes-1 (Hairy-enhancer of Split-1) (Takebayashi K. et al. (1994) J Biol Chem 269(7):150-6 (Takebayashi)) is a transcriptional factor with a basic helix-loop-helix structure. It binds to an important functional site in the CD4 silencer leading to repression of CD4 gene expression. Thus, Hes-1 is strongly involved in the determination of T-cell fate. Other genes from the Hes family include Hes-5 (mammalian Enhancer of Split homologue), the expression of which is also upregulated by Notch activation, and Hes-3. Expression of Hes-1 is upregulated as a result of Notch activation. The sequence of Mus musculus Hes-1 can be found in GenBank Accession No. D16464.

The E(spl) gene complex [E(spl)-C] (Leimeister C. et al. (1999) Mech Dev 85(1-2):173-7 (Leimeister)) comprises seven genes of which only E(spl) and Groucho show visible phenotypes when mutant. E(spl) was named after its ability to enhance Split mutations, Split being another name for Notch. Indeed, E(spl)-C genes repress Delta through regulation of achaete-scute complex gene expression. Expression of E(spl) is upregulated as a result of Notch activation.

Interleukin-10 (IL-10) was first characterised in the mouse as a factor produced by Th2 cells which was able to suppress cytokine production by Th1 cells. It was then shown that IL-10 was produced by many other cell types including macrophages, keratinocytes, B cells, Th0 and Th1 cells. It shows extensive homology with the Epstein-Barr bcrf1 gene which is now designated viral IL-10. Although a few immunostimulatory effects have been reported, it is mainly considered as an immunosuppressive cytokine. Inhibition of T cell responses by IL-10 is mainly mediated through a reduction of accessory functions of antigen presenting cells. IL-10 has notably been reported to suppress the production of numerous pro-inflammatory cytokines by macrophages and to inhibit co-stimulatory molecules and MHC class II expression. IL-10 also exerts anti-inflammatory effects on other myeloid cells such as neutrophils and eosinophils. On B cells, IL-10 influences isotype switching and proliferation. More recently, IL-10 was reported to play a role in the induction of regulatory T cells and as a possible mediator of their suppressive effect. Although it is not clear whether it is a direct downstream target of the Notch signalling pathway, its expression has been found to be strongly up-regulated coincident with Notch activation. The mRNA sequence of IL-10 may be found in GenBank ref. No. GI1041812.

CD-23 is the human leukocyte differentiation antigen CD23 (FCE2) which is a key molecule for B-cell activation and growth. It is the low-affinity receptor for IgE. Furthermore, the truncated molecule can be secreted, then functioning as a potent mitogenic growth factor. The sequence for CD-23 may be found in GenBank ref. No. GI1783344.

CTLA4 (cytotoxic T-lymphocyte activated protein 4) is an accessory molecule found on the surface of T-cells which is thought to play a role in the regulation of airway inflammatory cell recruitment and T-helper cell differentiation after allergen inhalation. The promoter region of the gene encoding CTLA4 has CBF 1 response elements and its expression is upregulated as a result of Notch activation. The sequence of CTLA4 can be found in GenBank Accession No. L15006.

Dlx-1 (distalless-1) (McGuinness T. Et al (1996) Genomics 35(3):473-85 (McGuiness)) expression is downregulated as a result of Notch activation. Sequences for Dlx genes may be found in GenBank Accession Nos. U51000-3.

CD-4 expression is downregulated as a result of Notch activation. A sequence for the CD-4 antigen may be found in GenBank Accession No. XM006966.

Other genes involved in the Notch signaling pathway, such as Numb, Mastermind and Dsh, and all genes the expression of which is modulated by Notch activation, are included in the scope of this invention.

As described above the Notch receptor family participates in cell-cell signalling events that influence T cell fate decisions. In this signalling NotchIC localises to the nucleus and functions as an activated receptor. Mammalian NotchIC interacts with the transcriptional repressor CBF1. It has been proposed that the NotchIC cdc10/ankyrin repeats are essential for this interaction. Hsieh et al (Hsieh et al. (1996) Molecular & Cell Biology 16(3):952-959) suggests rather that the N-terminal 114 amino acid region of mouse NotchIC contains the CBF1 interactive domain. It is also proposed that NotchIC acts by targeting DNA-bound CBF1 within the nucleus and abolishing CBF1-mediated repression through masking of the repression domain. It is known that Epstein Barr virus (EBV) immortalizing protein EBNA” also utilises CBF1 tethering and masking of repression to upregulate expression of CBF1-repressed B-cell genes. Thus, mimicry of Notch signal transduction is involved in EBV-driven immortalization. Strobl et al (Strobl et al. (2000) J Virol 74(4): 1727-35) similarly reports that “EBNA2 may hence be regarded as a functional equivalent of an activated Notch receptor”. Other EBV proteins which fall in this category include BARF0 (Kusano and Raab-Truab (2001) J Virol 75(1):384-395 (Kusano and Raab-Traub)) and LMP2A.

Any one or more of appropriate targets—such as an amino acid sequence and/or nucleotide sequence—may be used for identifying a compound capable of modulating the Notch signalling pathway and/or a targeting molecule in any of a variety of drug screening techniques. The target employed in such a test may be free in solution, affixed to a solid support, borne on a cell surface, or located intracellularly.

Techniques for drug screening may be based on the method described in Geysen, European Patent No. 0138855, published on Sep. 13, 1984. In summary, large numbers of different small peptide candidate modulators or targeting molecules are synthesized on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in drug screening techniques. Plates of use for high throughput screening (HTS) will be multi-well plates, preferably having 96, 384 or over 384 wells/plate. Cells can also be spread as “lawns”. Alternatively, non-neutralising antibodies can be used to capture the peptide and immobilise it on a solid support. High throughput screening, as described above for synthetic compounds, can also be used for identifying organic candidate modulators and targeting molecules.

This invention also contemplates the use of competitive drug screening assays in which neutralising antibodies capable of binding a target specifically compete with a test compound for binding to a target.

Techniques are well known in the art for the screening and development of agents such as antibodies, peptidomimetics and small organic molecules which are capable of binding to components of the Notch signalling pathway. These include the use of phage display systems for expressing signalling proteins, and using a culture of transfected E. coli or other microorganism to produce the proteins for binding studies of potential binding compounds (see, for example, G. Cesarini, FEBS Letters, 307(1):66-70 (July 1992); H. Gram et al., J. Immunol. Meth., 161:169-176 (1993); and C. Summer et al., Proc. Natl. Acad. Sci., USA, 89:3756-3760 (May 1992)). Further library and screening techniques are described, for example, in U.S. Pat. No. 6,281,344 (Phylos).

Polypeptides, Proteins and Amino Acid Sequences

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”.

“Peptide” usually refers to a short amino acid sequence that is 10 to 40 amino acids long, preferably 10 to 35 amino acids.

The amino acid sequence may be prepared and isolated from a suitable source, or it may be made synthetically or it may be prepared by use of recombinant DNA techniques.

Nucleotide Sequences

As used herein, the term “nucleotide sequence” is synonymous with the term “polynucleotide”.

The nucleotide sequence may be DNA or RNA of genomic or synthetic or of recombinant origin. They may also be cloned by standard techniques. The nucleotide sequence may be double-stranded or single-stranded whether representing the sense or antisense strand or combinations thereof.

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector. In general, primers will be produced by synthetic means, involving a step wise manufacture of the desired nucleic acid sequence one nucleotide at a time. Techniques for accomplishing this using automated techniques are readily available in the art.

“Polynucleotide” refers to a polymeric form of nucleotides of at least 10 bases in length and up to 10,000 bases or more, either ribonucleotides or deoxyribonucleotides or a modified form of either type of nucleotide. The term includes single and double stranded forms of DNA and also derivatised versions such as protein nucleic acid (PNA).

These may be constructed using standard recombinant DNA methodologies. The nucleic acid may be RNA or DNA and is preferably DNA. Where it is RNA, manipulations may be performed via cDNA intermediates. Generally, a nucleic acid sequence encoding the first region will be prepared and suitable restriction sites provided at the 5′ and/or 3′ ends. Conveniently the sequence is manipulated in a standard laboratory vector, such as a plasmid vector based on pBR322 or pUC19 (see below). Reference may be made to Molecular Cloning by Sambrook et al. (Cold Spring Harbor, 1989) or similar standard reference books for exact details of the appropriate techniques.

Sources of nucleic acid may be ascertained by reference to published literature or databanks such as GenBank. Nucleic acid encoding the desired first or second sequences may be obtained from academic or commercial sources where such sources are willing to provide the material or by synthesising or cloning the appropriate sequence where only the sequence data are available. Generally this may be done by reference to literature sources which describe the cloning of the gene in question.

Alternatively, where limited sequence data is available or where it is desired to express a nucleic acid homologous or otherwise related to a known nucleic acid, exemplary nucleic acids can be characterised as those nucleotide sequences which hybridise to the nucleic acid sequences known in the art.

For some applications, preferably, the nucleotide sequence is DNA. For some applications, preferably, the nucleotide sequence is prepared by use of recombinant DNA techniques (e.g. recombinant DNA). For some applications, preferably, the nucleotide sequence is cDNA. For some applications, preferably, the nucleotide sequence may be the same as the naturally occurring form.

Alternatively, where limited sequence data are available or where it is desired to express a nucleic acid homologous or otherwise related to a known nucleic acid, exemplary nucleic acids can be characterised as those nucleotide sequences which hybridise to the nucleic acid sequences known in the art.

It will be understood by a skilled person that numerous different nucleotide sequences can encode the same protein used in the present invention as a result of the degeneracy of the genetic code. In addition, it is to be understood that skilled persons may, using routine techniques, make nucleotide substitutions that do not affect the protein encoded by the nucleotide sequence of the present invention to reflect the codon usage of any particular host organism in which the target protein or protein for Notch signalling modulation of the present invention is to be expressed.

Variants, Derivatives, Analogues, Homologues and Fragments

In addition to the specific amino acid sequences and nucleotide sequences mentioned herein, the present invention also encompasses the use of variants, derivatives, analogues, homologues and fragments thereof.

In the context of the present invention, a variant of any given sequence is a sequence in which the specific sequence of residues (whether amino acid or nucleic acid residues) has been modified in such a manner that the polypeptide or polynucleotide in question retains at least one of its endogenous functions. A variant sequence can be modified by addition, deletion, substitution modification replacement and/or variation of at least one residue present in the naturally-occurring protein.

The term “derivative” as used herein, in relation to proteins or polypeptides of the present invention includes any substitution of, variation of, modification of, replacement of, deletion of and/or addition of one (or more) amino acid residues from or to the sequence providing that the resultant protein or polypeptide retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Within the definitions of “proteins” and “polypeptides” useful in the present invention, the specific amino acid residues may be modified in such a manner that the protein in question retains at least one of its endogenous functions, such modified proteins are referred to as “variants”. A variant protein can be modified by addition, deletion and/or substitution of at least one amino acid present in the naturally-occurring protein.

Typically, amino acid substitutions may be made, for example from 1, 2 or 3 to 10 or 20 substitutions provided that the modified sequence retains the required target activity or ability to modulate Notch signalling. Amino acid substitutions may include the use of non-naturally occurring analogues.

Proteins of use in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity, and/or the amphipathic nature of the residues as long as the target or modulation function is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

For ease of reference, the one and three letter codes for the main naturally occurring amino acids (and their associated codons) are set out below:

| | | | |
|---|---|---|---|
| Symbol | 3-letter | Meaning | Codons |

| | | | |
|---|---|---|---|
| A | Ala | Alanine | GCT, GCC, GCA, GCG |
| B | Asp, Asn | Aspartic, | GAT, GAC, AAT, AAC |
| | | Asparagine | |
| C | Cys | Cysteine | TGT, TGC |
| D | Asp | Aspartic | GAT, GAC |
| E | Glu | Glutamic | GAA, GAG |
| F | Phe | Phenylalanine | TTT, TTC |
| G | Gly | Glycine | GGT, GGC, GGA, GGG |
| H | His | Histidine | CAT, CAC |
| I | Ile | Isoleucine | ATT, ATC, ATA |
| K | Lys | Lysine | AAA, AAG |
| L | Leu | Leucine | TTG, TTA, CTT, CTC, |
| | | | CTA, CTG |
| M | Met | Methionine | ATG |
| N | Asn | Asparagine | AAT, AAC |
| P | Pro | Proline | CCT, CCC, CCA, CCG |
| Q | Gln | Glutamine | CAA, CAG |
| R | Arg | Arginine | CGT, CGC, CGA, CGG, |
| | | | AGA, AGG |
| S | Ser | Serine | TCT, TCC, TCA, TCG, |
| | | | AGT, AGC |
| T | Thr | Threonine | ACT, ACC, ACA, ACG |
| V | Val | Valine | GTT, GTC, GTA, GTG |
| W | Trp | Tryptophan | TGG |
| X | Xxx | Unknown | |
| Y | Tyr | Tyrosine | TAT, TAC |
| Z | Glu, Gln | Glutamic, | GAA, GAG, CAA, CAG |
| | | Glutamine | |
| * | End | Terminator | TAA, TAG, TGA |

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

As used herein, the term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds. The terms subunit and domain may also refer to polypeptides and peptides having biological function.

A peptide useful in the invention will at least have a target or signalling modulation capability. “Fragments” are also variants and the term typically refers to a selected region of the protein that is of interest in a binding assay and for which a binding partner is known or determinable. “Fragment” thus refers to an amino acid sequence that is a portion of a full-length polypeptide, for example between about 8 and about 1500 amino acids in length, preferably between about 8 and about 745 amino acids in length, preferably about 8 to about 300, more preferably about 8 to about 200 amino acids, and even more preferably about 10 to about 50 or 100 amino acids in length. “Peptide” refers to a short amino acid sequence that is 10 to 40 amino acids long, preferably 10 to 35 amino acids.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Variants of the nucleotide sequence may also be made. Such variants will preferably comprise codon optimised sequences. Codon optimisation is known in the art as a method of enhancing RNA stability and therefore gene expression. The redundancy of the genetic code means that several different codons may encode the same amino-acid. For example, leucine, arginine and serine are each encoded by six different codons. Different organisms show preferences in their use of the different codons. Viruses such as HIV, for instance, use a large number of rare codons. By changing a nucleotide sequence such that rare codons are replaced by the corresponding commonly used mammalian codons, increased expression of the sequences in mammalian target cells can be achieved. Codon usage tables are known in the art for mammalian cells, as well as for a variety of other organisms.

Where the active agent is a nucleotide sequences it may suitably be codon optimised for expression in mammalian cells. Preferably, at least part of the sequence is codon optimised. Even more preferably, the sequence is codon optimised in its entirety.

Sequence Homology, Similarity and Identity

As used herein, the term “homology” can be equated with “identity”. An homologous sequence will be taken to include an amino acid sequence which may be at least 75, 85 or 90% identical, preferably at least 95 or 98% identical. In particular, homology should typically be considered with respect to those regions of the sequence (such as amino acids at positions 51, 56 and 57) known to be essential for an activity. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefor firstly requires the production of an optimal alignment, taking into consideration gap penalties. A suitable computer program for carrying out such an alignment is the GCG Wisconsin Bestfit package (University of Wisconsin, U.S.A.; Devereux). Examples of other software than can perform sequence comparisons include, but are not limited to, the BLAST package, FASTA (Atschul et al. (1990) J. Mol. Biol. 403-410 (Atschul)) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

The five BLAST programs, available online through the National Center for Biotechnology Information of the National Institutes of Health, perform the following tasks:

blastp—compares an amino acid query sequence against a protein sequence database.

blastn—compares a nucleotide query sequence against a nucleotide sequence database.

blastx—compares the six-frame conceptual translation products of a nucleotide query sequence (both strands) against a protein sequence database.

tblastn—compares a protein query sequence against a nucleotide sequence database dynamically translated in all six reading frames (both strands).

tblastx—compares the six-frame translations of a nucleotide query sequence against the six-frame translations of a nucleotide sequence database.

BLAST uses the following search parameters:

HISTOGRAM—Display a histogram of scores for each search; default is yes. (See parameter H in the BLAST Manual).

DESCRIPTIONS—Restricts the number of short descriptions of matching sequences reported to the number specified; default limit is 100 descriptions. (See parameter V in the manual page).

EXPECT—The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF—Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

ALIGNMENTS—Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

MATRIX—Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND—Restrict a TBLASTN search to just the top or bottom strand of the database sequences; or restrict a BLASTN, BLASTX or TBLASTX search to just reading frames on the top or bottom strand of the query sequence.

FILTER—Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Clayerie & States (1993) Computers and Chemistry 17:191-201, or, for BLASTN, by the DUST program of Tatusov and Lipman (see the website of the National Center for Biotechnology Information of the National Institutes of Health). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi—Causes NCBI gi identifiers to be shown in the output, in addition to the accession and/or locus name.

Most preferably, sequence comparisons are conducted using the simple BLAST search algorithm provided by the National Center for Biotechnology Information of the National Institutes of Health.

In some aspects of the present invention, no gap penalties are used when determining sequence identity.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

Nucleotide sequences which are homologous to or variants of sequences of use in the present invention can be obtained in a number of ways, for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the reference nucleotide sequence under conditions of medium to high stringency. Similar considerations apply to obtaining species homologues and allelic variants of the amino acid and/or nucleotide sequences useful in the present invention.

Variants and strain/species homologues may also be obtained using degenerate PCR which will use primers designed to target sequences within the variants and homologues encoding conserved amino acid sequences within the sequences of use in the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Variants and strain/species homologues may also be obtained using degenerate PCR which will use primers designed to target sequences within the variants and homologues encoding conserved amino acid sequences within the sequences of use in the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

PCR technology as described e.g. in section 14 of Sambrook et al., 1989, requires the use of oligonucleotide probes that will hybridise to nucleic acid. Strategies for selection of oligonucleotides are described below.

As used herein, a probe is e.g. a single-stranded DNA or RNA that has a sequence of nucleotides that includes between 10 and 50, preferably between 15 and 30 and most preferably at least about 20 contiguous bases that are the same as (or the complement of) an equivalent or greater number of contiguous bases. The nucleic acid sequences selected as probes should be of sufficient length and sufficiently unambiguous so that false positive results are minimised. The nucleotide sequences are usually based on conserved or highly homologous nucleotide sequences or regions of polypeptides. The nucleic acids used as probes may be degenerate at one or more positions.

Preferred regions from which to construct probes include 5′ and/or 3′ coding sequences, sequences predicted to encode ligand binding sites, and the like. For example, either the full-length cDNA clone disclosed herein or fragments thereof can be used as probes. Preferably, nucleic acid probes of the invention are labelled with suitable label means for ready detection upon hybridisation. For example, a suitable label means is a radiolabel. The preferred method of labelling a DNA fragment is by incorporating α32P dATP with the Klenow fragment of DNA polymerase in a random priming reaction, as is well known in the art. Oligonucleotides are usually end-labelled with γ³²P-labelled ATP and polynucleotide kinase. However, other methods (e.g. non-radioactive) may also be used to label the fragment or oligonucleotide, including e.g. enzyme labelling, fluorescent labelling with suitable fluorophores and biotinylation.

Preferred are such sequences, probes which hybridise under high-stringency conditions.

Alternatively, such nucleotide sequences may be obtained by site directed mutagenesis of characterised sequences. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the polynucleotide or encoded polypeptide.

In general, the terms “variant”, “homologue” or “derivative” in relation to the nucleotide sequence used in the present invention includes any substitution of, variation of, modification of, replacement of, deletion of or addition of one (or more) nucleic acid from or to the sequence providing the resultant nucleotide sequence codes for a target protein or protein for T cell signalling modulation.

As indicated above, with respect to sequence homology, preferably there is at least 75%, more preferably at least 85%, more preferably at least 90% homology to the reference sequences. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

Hybridisation

The present invention also encompasses nucleotide sequences that are capable of hybridising selectively to the reference sequences, or any variant, fragment or derivative thereof, or to the complement of any of the above. Nucleotide sequences are preferably at least 15 nucleotides in length, more preferably at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction (PCR) technologies.

Nucleotide sequences useful in the invention capable of selectively hybridising to the nucleotide sequences presented herein, or to their complement, will be generally at least 75%, preferably at least 85 or 90% and more preferably at least 95% or 98% homologous to the corresponding nucleotide sequences presented herein over a region of at least 20, preferably at least 25 or 30, for instance at least 40, 60 or 100 or more contiguous nucleotides. Preferred nucleotide sequences of the invention will comprise regions homologous to the nucleotide sequence, preferably at least 80 or 90% and more preferably at least 95% homologous to the nucleotide sequence.

The term “selectively hybridizable” means that the nucleotide sequence used as a probe is used under conditions where a target nucleotide sequence of the invention is found to hybridize to the probe at a level significantly above background. The background hybridization may occur because of other nucleotide sequences present, for example, in the cDNA or genomic DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical nucleotide sequences while an intermediate (or low) stringency hybridization can be used to identify or detect similar or related polynucleotide sequences. In a preferred aspect, the present invention covers nucleotide sequences that can hybridise to the nucleotide sequence of the present invention under stringent conditions (e.g. 65° C. and 0.1×SSC {1×SSC=0.15 M NaCl, 0.015 M Na₃ Citrate pH 7.0). Where the nucleotide sequence of the invention is double-stranded, both strands of the duplex, either individually or in combination, are encompassed by the present invention. Where the nucleotide sequence is single-stranded, it is to be understood that the complementary sequence of that nucleotide sequence is also included within the scope of the present invention.

Stringency of hybridisation refers to conditions under which polynucleic acids hybrids are stable. Such conditions are evident to those of ordinary skill in the field. As known to those of skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridisation reaction is performed under conditions of higher stringency, followed by washes of varying stringency.

As used herein, high stringency preferably refers to conditions that permit hybridisation of only those nucleic acid sequences that form stable hybrids in 1 M Na⁺ at 65-68° C. High stringency conditions can be provided, for example, by hybridisation in an aqueous solution containing 6×SSC, 5× Denhardt's, 1% SDS (sodium dodecyl sulphate), 0.1 Na⁺ pyrophosphate and 0.1 mg/ml denatured salmon sperm DNA as non specific competitor. Following hybridisation, high stringency washing may be done in several steps, with a final wash (about 30 min) at the hybridisation temperature in 0.2-0.1×SSC, 0.1% SDS.

It is understood that these conditions may be adapted and duplicated using a variety of buffers, e.g. formamide-based buffers, and temperatures. Denhardt's solution and SSC are well known to those of skill in the art as are other suitable hybridisation buffers (see, e.g. Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). Optimal hybridisation conditions have to be determined empirically, as the length and the GC content of the hybridising pair also play a role.

Cloning and Expression

Nucleotide sequences which are not 100% homologous to the sequences of the present invention but fall within the scope of the invention can be obtained in a number of ways. Other variants of the sequences described herein may be obtained for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the reference nucleotide sequence under conditions of medium to high stringency. Similar considerations apply to obtaining species homologues and allelic variants of the amino acid and/or nucleotide sequences useful in the present invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the target protein or protein for T cell signalling modulation encoded by the nucleotide sequences.

The nucleotide sequences such as a DNA polynucleotides useful in the invention may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

The present invention also relates to vectors which comprise a polynucleotide useful in the present invention, host cells which are genetically engineered with vectors of the invention and the production of polypeptides useful in the present invention by such techniques.

For recombinant production, host cells can be genetically engineered to incorporate expression systems or polynucleotides of the invention. Introduction of a polynucleotide into the host cell can be effected by methods described in many standard laboratory manuals, such as Davis et al and Sambrook et al, such as calcium phosphate transfection, DEAE-dextran mediated transfection, transfection, microinjection, cationic lipid-mediated transfection, electroporation, transduction, scrape loading, ballistic introduction and infection. It will be appreciated that such methods can be employed in vitro or in vivo as drug delivery systems.

Representative examples of appropriate hosts include bacterial cells, such as streptococci, staphylococci, E. coli, streptomyces and Bacillus subtilis cells; fungal cells, such as yeast cells and Aspergillus cells; insect cells such as Drosophila S2 and Spodoptera Sf9 cells; animal cells such as CHO, COS, NSO, HeLa, C127, 3T3, BHK, 293 and Bowes melanoma cells; and plant cells.

A great variety of expression systems can be used to produce a polypeptide useful in the present invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al.

For secretion of the translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Proteins or polypeptides may be in the form of the “mature” protein or may be a part of a larger protein such as a fusion protein or precursor. For example, it is often advantageous to include an additional amino acid sequence which contains secretory or leader sequences or pro-sequences (such as a HIS oligomer, immunoglobulin Fc, glutathione S-transferase, FLAG etc) to aid in purification. Likewise such an additional sequence may sometimes be desirable to provide added stability during recombinant production. In such cases the additional sequence may be cleaved (eg chemically or enzymatically) to yield the final product. In some cases, however, the additional sequence may also confer a desirable pharmacological profile (as in the case of IgFc fusion proteins) in which case it may be preferred that the additional sequence is not removed so that it is present in the final product as administered.

Proteins or polypeptides may be in the form of the “mature” protein or may be a part of a larger protein such as a fusion protein or precursor. For example, it is often advantageous to include an additional amino acid sequence which contains secretory or leader sequences or pro-sequences (such as a HIS oligomer, immunoglobulin Fc, glutathione S-transferase, FLAG etc) to aid in purification. Likewise such an additional sequence may sometimes be desirable to provide added stability during recombinant production. In such cases the additional sequence may be cleaved (eg chemically or enzymatically) to yield the final product. In some cases, however, the additional sequence may also confer a desirable pharmacological profile (as in the case of IgFc fusion proteins) in which case it may be preferred that the additional sequence is not removed so that it is present in the final product as administered.

Also included within the invention are mammalian and microbial host cells comprising such vectors or other polynucleotides encoding the fusion proteins, and their production and use.

Active agents for use in the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding protein may be employed to regenerate active conformation when the polypeptide is denatured during isolation and/or purification.

Various preferred features and embodiments of the present invention will now be described in more detail by way of non-limiting examples.

Substances that may be used to modulate Notch signalling by inhibiting Notch ligand expression include nucleic acid sequences encoding polypeptides that affect the expression of genes encoding Notch ligands. For instance, for Delta expression, binding of extracellular BMPs (bone morphogenetic proteins, Wilson and Hemmati-Brivanlou; Hemmati-Brivanlou and Melton) to their receptors leads to down-regulated Delta transcription due to the inhibition of the expression of transcription factors of the achaete/scute complex. This complex is believed to be directly involved in the regulation of Delta expression. Thus, any polypeptide that upregulates BMP expression and/or stimulates the binding of BMPs to their receptors may be capable of producing a decrease in the expression of Notch ligands such as Delta and/or Serrate. Examples may include nucleic acids encoding BMPs themselves. Furthermore, any substance that inhibits expression of transcription factors of the achaete/scute complex may also downregulate Notch ligand expression.

Members of the BMP family include BMP1 to BMP6, BMP7 also called OP1, OP2 (BMP8) and others. BMPs belong to the transforming growth factor beta (TGF-beta) superfamily, which includes, in addition to the TGF-betas, activins/inhibins (e.g., alpha-inhibin), mullerian inhibiting substance, and glial cell line-derived neurotrophic factor.

Other examples of polypeptides that inhibit the expression of Delta and/or Serrate include the Toll-like receptor (Medzhitov) or any other receptors linked to the innate immune system (for example CD 14, complement receptors, scavenger receptors or defensin proteins), and other polypeptides that decrease or interfere with the production of Noggin (Valenzuela), Chordin (Sasai), Follistatin (lemura), Xnr3, and derivatives and variants thereof. Noggin and Chordin bind to BMPs thereby preventing activation of their signalling cascade which leads to decreased Delta transcription. Consequently, reducing Noggin and Chordin levels may lead to decreased Notch ligand, in particular Delta, expression.

In more detail, in Drosophila, the Toll transmembrane receptor plays a central role in the signalling pathways that control amongst other things the innate nonspecific immune response. This Toll-mediated immune response reflects an ancestral conserved signalling system that has homologous components in a wide range of organisms. Human Toll homologues have been identified amongst the Toll-like receptor (TLR) genes and Toll/interleukin-1 receptor-like (TIL) genes and contain the characteristic Toll motifs: an extracellular leucine-rich repeat domain and a cytoplasmic interleukin-1 receptor-like region. The Toll-like receptor genes (including TIL genes) now include TLR4, TIL3, TIL4, and 4 other identified TLR genes.

Other suitable sequences that may be used to downregulate Notch ligand expression include those encoding immune costimulatory molecules (for example CD80, CD86, ICOS, SLAM) and other accessory molecules that are associated with immune potentiation (for example CD2, LFA-1).

Other suitable substances that may be used to downregulate Notch ligand expression include nucleic acids that inhibit the effect of transforming growth factors such as members of the fibroblast growth factor (FGF) family. The FGF may be a mammalian basic FGF, acidic FGF or another member of the FGF family such as an FGF-1, FGF-2, FGF-3, FGF-4, FGF-5, FGF-6, FGF-7. Preferably the FGF is not acidic FGF (FGF-1; Zhao et al., 1995). Most preferably, the FGF is a member of the FGF family which acts by stimulating the upregulation of expression of a Serrate polypeptide on APCs. It has been shown that members of the FGF family can upregulate Serrate-1 gene expression in APCs.

Inhibition of Notch Signalling by Use of Anti-Sense Constructs

Suitable nucleic acid sequences may include anti-sense constructs, for example nucleic acid sequences encoding antisense Notch ligand constructs or antisense sequences corresponding to other components of the Notch signalling pathway as discussed above. The antisense nucleic acid may be an oligonucleotide such as a synthetic single-stranded DNA. However, more preferably, the antisense is an antisense RNA produced in the patient's own cells as a result of introduction of a genetic vector. The vector is responsible for production of antisense RNA of the desired specificity on introduction of the vector into a host cell.

Antisense nucleic acids can be oligonucleotides that are double-stranded or single-stranded, RNA or DNA or a modification or derivative thereof, which can be directly administered to a cell, or which can be produced intracellularly by transcription of exogenous, introduced sequences.

For example, as described in U.S. Pat. No. 2,002,0119540 inhibitory antisense or double stranded oligonucleotides can additionally comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluraci-1, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyamoinomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

An antisense oligonucleotide may also comprise one or more modified sugar moieties such as, for example, arabinose, 2-fluoroarabinose, xylulose, or hexose.

In yet another embodiment, the antisense oligonucleotide may if desired comprise at least one modified phosphate backbone such as, for example, a phosphorothioate, a phosphorodithioate, a phosphoramidothioate, a phosphoramidate, a phosphordiamidate, a methylphosphonate, an alkyl phosphotriester, or a formacetal or analog thereof. Alternatively another polymeric backbone such as a modified polypeptide backbone may be used (eg protein nucleic acid: PNA).

In yet another embodiment, the antisense oligonucleotide may be an alpha-anomeric oligonucleotide. An alpha-anomeric oligonucleotide forms specific double-stranded hybrids with complementary RNA in which, contrary to the usual beta-units, the strands run parallel to each other (Gautier et al., 1987, Nucl. Acids Res. 15:6625-6641). The oligonucleotide may for example be a 2′-O-methylribonucleotide (Inoue et al., 1987, Nucl. Acids Res. 15:6131-6148), or a chimeric RNA-DNA analogue (Inoue et al., 1987, FEBS Lett. 215:327-330). Oligonucleotides may be synthesized by standard methods known in the art, e.g. by use of an automated DNA synthesizer (such as are commercially available from Biosearch, Applied Biosystems, etc.). Merely as examples, phosphorothioate oligonucleotides can be synthesized by the method of Stein et al. (1988, Nucl. Acids Res. 16:3209), and methylphosphonate oligonucleotides can be prepared by use of controlled pore glass polymer supports (Sarin et al., 1988, Proc. Natl. Acad. Sci. U.S.A. 85:7448-7451), etc.

Preferably, the nucleic acid sequence for use in the present invention is capable of inhibiting Serrate and Delta, preferably Serrate 1 and Serrate 2 as well as Delta 1, Delta 3 and Delta 4 expression in APCs such as dendritic cells. In particular, the nucleic acid sequence may be capable of inhibiting Serrate expression but not Delta expression, or Delta but not Serrate expression in APCs or T cells. Alternatively, the nucleic acid sequence for use in the present invention is capable of inhibiting Delta expression in T cells such as CD4+ helper T cells or other cells of the immune system that express Delta (for example in response to stimulation of cell surface receptors). In particular, the nucleic acid sequence may be capable of inhibiting Delta expression but not Serrate expression in T cells. In a particularly preferred embodiment, the nucleic acid sequence is capable of inhibiting Notch ligand expression in both T cells and APC, for example Serrate expression in APCs and Delta expression in T cells.

Preferred suitable substances that may be used to downregulate Notch ligand expression include growth factors and cytokines. More preferably soluble protein growth factors may be used to inhibit Notch or Notch ligand expression. For instance, Notch ligand expression may be reduced or inhibited by the addition of BMPs or activins (a member of the TGF-β superfamily). In addition, T cells, APCs or tumour cells could be cultured in the presence of inflammatory type cytokines including IL-12, IFN-γ, IL-18, TNF-α, either alone or in combination with BMPs.

Molecules for inhibition of Notch signalling will also include polypeptides, or polynucleotides which encode therefore, capable of modifying Notch-protein expression or presentation on the cell membrane or signalling pathways. Molecules that reduce or interfere with its presentation as a fully functional cell membrane protein may include MMP inhibitors such as hydroxymate-based inhibitors.

Other substances which may be used to reduce interaction between Notch and Notch ligands are exogenous Notch or Notch ligands or functional derivatives thereof. For example, Notch ligand derivatives would preferably have the DSL domain at the N-terminus and between 1 to 8, suitably from 2 to 5, EGF-like repeats on the extracellular surface. A peptide corresponding to the Delta/Serrate/LAG-2 domain of hJagged1 and supernatants from COS cells expressing a soluble form of the extracellular portion of hJagged1 was found to mimic the effect of Jagged1 in inhibiting Notch1 (Li).

In one embodiment a Notch ligand derivative may be a fusion protein, for example, a fusion protein comprising a segment of a Notch ligand extracellular domain and an immunoglobulin F_{c} segment such as IgGF_{c} or IgMF_{c}.

Alternatively, the modulator may comprise all or part of the extracellular domain of a Notch receptor (eg Notch1, Notch2, Notch3, Notch4 or homologues thereof), which can bind to Notch ligands and so reduce interactions with endogenous Notch receptors. Preferably, such a modulator may comprise at least the 11th and 12th domains of Notch (EGF11 and EGF12), as these are believed to be important for Notch ligand interaction.

For example, a rat Notch-1/Fc fusion protein is available from R& D Systems Inc (Minneapolis, USA and Abingdon, Oxon, UK: Catalog No 1057-TK). This comprises the 12 amino terminal EGF domains of rat Notch-1 (amino acid residues Met 1 to Glu 488) fused to the Fc region of human IgG (Pro 100 to Lys 330) via a polypeptide linker (IEGRMD).

Other Notch signalling pathway antagonists include antibodies which inhibit interactions between components of the Notch signalling pathway, e.g. antibodies to Notch or Notch ligands.

The term “antibody” includes intact molecules as well as fragments thereof, such as Fab, Fab′, F(ab′)₂, Fv and scFv which are capable of binding the epitopic determinant. These antibody fragments retain some ability to selectively bind with its antigen or receptor and include, for example:
(i) Fab, the fragment which contains a monovalent antigen-binding fragment of an antibody molecule can be produced by digestion of whole antibody with the enzyme papain to yield an intact light chain and a portion of one heavy chain;
(ii) Fab′, the fragment of an antibody molecule can be obtained by treating whole antibody with pepsin, followed by reduction, to yield an intact light chain and a portion of the heavy chain; two Fab′ fragments are obtained per antibody molecule;
(iii) (Fab′)₂, the fragment of the antibody that can be obtained by treating whole antibody with pepsin without subsequent reduction; F(ab′) 2 is a dimer of two Fab′ fragments held together by two disulfide bonds;
(iv) Fv, defined as a genetically engineered fragment containing the variable genetically fused single chain molecule; and
(v) fragments consisting of essentially only a variable (VH or VL), antigen-binding domain of the antibody (so-called “domain antibodies”).

General methods of making antibodies are known in the art. (See for example, Harlow and Lane, Antibodies: A Laboratory Manual, Cold Spring Harbor Laboratory, New York (1988), the text of which is incorporated herein by reference). Antibodies may be monoclonal or polyclonal but are preferably monoclonal.

Suitably, the binding affinity (equilibrium association constant (Ka)) may be at least about. 10⁶ M⁻¹, at least about 10⁷ M⁻¹, at least about 10⁸ M⁻¹ or at least about 10⁹ M⁻¹.

Suitably the antibody, derivative or fragment binds to one or more DSL, EGF or N-terminal domains of a Notch ligand or to one or more EGF or Lin/Notch (L/N) domains of Notch (for example to EGF repeats 11 and 12 of Notch).

In one embodiment the agent may be an antibody, derivative or fragment which binds to Notch.

In a further embodiment the agent may be an antibody, derivative or fragment which binds to Delta.

In a further embodiment the agent may be an antibody, derivative or fragment which binds to Serrate or Jagged.

Suitable antibodies for use as blocking agents are obtained by immunizing a host animal with peptides comprising all or a portion of Notch or a Notch ligand such as Delta or Serrate/Jagged.

The peptide used may comprise the complete protein or a fragment or derivatives thereof. Preferred immunogens comprise all or a part of the extracellular domain of human Notch, Delta or Serrate/Jagged, where these residues contain any post-translation modifications, such as glycosylation, found in the native proteins. Immunogens comprising the extracellular domain may be produced by a number of techniques which are well known in the art such as expression of cloned genes using conventional recombinant methods and/or isolation from T cells or cell populations expressing high levels of Notch or Notch ligands.

Monoclonal antibodies may be produced by means well known in the art. Generally, the spleen and/or lymph nodes of an immunized host animal provide a source of plasma cells. The plasma cells are immortalized by fusion with myeloma cells to produce hybridoma cells. Culture supernatant from individual hybridomas is screened using standard techniques to identify those producing antibodies with the desired specificity. The antibody may be purified from the hybridoma cell supernatants or ascites fluid by conventional techniques, such as affinity chromatography using Notch, Notch ligands or fragments thereof bound to an insoluble support, protein A sepharose, or the like.

For example, antibodies against Notch and Notch ligands are described in U.S. Pat. No. 5,648,464, U.S. Pat. No. 5,849,869 and U.S. Pat. No. 6,004,924 (Yale University/Imperial Cancer Technology), the texts of which are herein incorporated by reference.

Antibodies generated against the Notch receptor are also described in WO 0020576 (the text of which is also incorporated herein by reference). For example, this document discloses generation of antibodies against the human Notch-1 EGF-like repeats 11 and 12. For example, in particular embodiments, WO 0020576 discloses a monoclonal antibody secreted by a hybridoma designated A6 having the ATCC Accession No. HB 12654, a monoclonal antibody secreted by a hybridoma designated Cll having the ATCC Accession No. HB 12656 and a monoclonal antibody secreted by a hybridoma designated F3 having the ATCC Accession No. HB12655.

Preferably, antibodies for use to treat human patients will be chimeric or humanised antibodies. Antibody “humanisation” techniques are well known in the art. These techniques typically involve the use of recombinant DNA technology to manipulate DNA sequences encoding the polypeptide chains of the antibody molecule.

As described in U.S. Pat. No. 5,859,205 early methods for humanising monoclonal antibodies (Mabs) involved production of chimeric antibodies in which an antigen binding site comprising the complete variable domains of one antibody is linked to constant domains derived from another antibody. Such chimerisation procedures are described in EP-A-0120694 (Celltech Limited), EP-A-0125023 (Genentech Inc. and City of Hope), EP-A-0 171496 (Res. Dev. Corp. Japan), EP-A-0 173 494 (Stanford University), and WO 86/01533 (Celltech Limited). For example, WO 86/01533 discloses a process for preparing an antibody molecule having the variable domains from a mouse MAb and the constant domains from a human immunoglobulin.

In an alternative approach, described in EP-A-0239400 (Winter), the complementarity determining regions (CDRS) of a mouse MAb are grafted onto the framework regions of the variable domains of a human immunoglobulin by site directed mutagenesis using long oligonucleotides. Such CDR-grafted humanised antibodies are much less likely to give rise to an anti-antibody response than humanised chimeric antibodies in view of the much lower proportion of non-human amino acid sequence which they contain. Examples in which a mouse MAb recognising lysozyme and a rat MAb recognising an antigen on human T-cells were humanised by CDR-grafting have been described by Verhoeyen et al (Science, 239, 1534-1536, 1988) and Riechmann et al (Nature, 332, 323-324, 1988) respectively. The preparation of CDR-grafted antibody to the antigen on human T cells is also described in WO 89/07452 (Medical Research Council).

In WO 90/07861 Queen et al propose four criteria for designing humanised immunoglobulins. The first criterion is to use as the human acceptor the framework from a particular human immunoglobulin that is unusually homologous to the non-human donor immunoglobulin to be humanised, or to use a consensus framework from many human antibodies. The second criterion is to use the donor amino acid rather than the acceptor if the human acceptor residue is unusual and the donor residue is typical for human sequences at a specific residue of the framework. The third criterion is to use the donor framework amino acid residue rather than the acceptor at positions immediately adjacent to the CDRs. The fourth criterion is to use the donor amino acid residue at framework positions at which the amino acid is predicted to have a side chain atom within about 3 A of the CDRs in a three-dimensional immunoglobulin model and to be capable of interacting with the antigen or with the CDRs of the humanised immunoglobulin. It is proposed that criteria two, three or four may be applied in addition or alternatively to criterion one, and may be applied singly or in any combination.

The choice of isotype will be guided by the desired effector functions, such as complement fixation, or activity in antibody-dependent cellular cytotoxicity. Suitable isotypes include IgG 1, IgG3 and IgG4. Suitably, either of the human light chain constant regions, kappa or lambda, may be used.

Chemical Linking

Chemically coupled sequences can be prepared (where required) from individual proteins sequences and coupled using known chemically coupling techniques. The conjugate can be assembled using conventional solution- or solid-phase peptide synthesis methods, affording a fully protected precursor with only the terminal amino group in deprotected reactive form. This function can then be reacted directly with a protein for T cell signalling modulation or a suitable reactive derivative thereof. Alternatively, this amino group may be converted into a different functional group suitable for reaction with a cargo moiety or a linker. Thus, e.g. reaction of the amino group with succinic anhydride will provide a selectively addressable carboxyl group, while further peptide chain extension with a cysteine derivative will result in a selectively addressable thiol group. Once a suitable selectively addressable functional group has been obtained in the delivery vector precursor, a protein for T cell signalling modulation or a derivative thereof may be attached through e.g. amide, ester, or disulphide bond formation. Cross-linking reagents which can be utilized are discussed, for example, in Neans, G. E. and Feeney, R. E., Chemical Modification of Proteins, Holden-Day, 1974, pp. 39-43.

As discussed above the target protein and protein for T cell signalling modulation may be linked directly or indirectly via a cleavable linker moiety. Direct linkage may occur through any convenient functional group on the protein for T cell signalling modulation such as a hydroxy, carboxy or amino group. Indirect linkage which is preferable, will occur through a linking moiety. Suitable linking moieties include bi- and multi-functional alkyl, aryl, aralkyl or peptidic moieties, alkyl, aryl or aralkyl aldehydes acids esters and anyhdrides, sulphydryl or carboxyl groups, such as maleimido benzoic acid derivatives, maleimido proprionic acid derivatives and succinimido derivatives or may be derived from cyanuric bromide or chloride, carbonyldiimidazole, succinimidyl esters or sulphonic halides and the like. The functional groups on the linker moiety used to form covalent bonds between linker and protein for T cell signalling modulation on the one hand, as well as linker and target protein on the other hand, may be two or more of, e.g., amino, hydrazino, hydroxyl, thiol, maleimido, carbonyl, and carboxyl groups, etc. The linker moiety may include a short sequence of from 1 to 4 amino acid residues that optionally includes a cysteine residue through which the linker moiety bonds to the target protein.

Notch Ligand Domains

As discussed above, naturally occurring Notch ligands typically comprise a number of distinctive domains. Some predicted/potential domain locations for various naturally occurring human Notch ligands (based on amino acid numbering in the precursor proteins) are shown below:

| | | |
|---|---|---|
| Component | Amino acids | Proposed function/domain |

Human Delta 1

| | | |
|---|---|---|
| SIGNAL | 1-17 | SIGNAL |
| CHAIN | 18-723 | DELTA-LIKE PROTEIN 1 |
| DOMAIN | 18-545 | EXTRACELLULAR |
| TRANSMEM | 546-568 | TRANSMEMBRANE |
| DOMAIN | 569-723 | CYTOPLASMIC |
| DOMAIN | 159-221 | DSL |
| DOMAIN | 226-254 | EGF-LIKE 1 |
| DOMAIN | 257-285 | EGF-LIKE 2 |
| DOMAIN | 292-325 | EGF-LIKE 3 |
| DOMAIN | 332-363 | EGF-LIKE 4 |
| DOMAIN | 370-402 | EGF-LIKE 5 |
| DOMAIN | 409-440 | EGF-LIKE 6 |
| DOMAIN | 447-478 | EGF-LIKE 7 |
| DOMAIN | 485-516 | EGF-LIKE 8 |

Human Delta 3

| | | |
|---|---|---|
| DOMAIN | 158-248 | DSL |
| DOMAIN | 278-309 | EGF-LIKE 1 |
| DOMAIN | 316-350 | EGF-LIKE 2 |
| DOMAIN | 357-388 | EGF-LIKE 3 |
| DOMAIN | 395-426 | EGF-LIKE 4 |
| DOMAIN | 433-464 | EGF-LIKE 5 |

Human Delta 4

| | | |
|---|---|---|
| SIGNAL | 1-26 | SIGNAL |
| CHAIN | 27-685 | DELTA-LIKE PROTEIN 4 |
| DOMAIN | 27-529 | EXTRACELLULAR |
| TRANSMEM | 530-550 | TRANSMEMBRANE |
| DOMAIN | 551-685 | CYTOPLASMIC |
| DOMAIN | 155-217 | DSL |
| DOMAIN | 218-251 | EGF-LIKE 1 |
| DOMAIN | 252-282 | EGF-LIKE 2 |
| DOMAIN | 284-322 | EGF-LIKE 3 |
| DOMAIN | 324-360 | EGF-LIKE 4 |
| DOMAIN | 362-400 | EGF-LIKE 5 |
| DOMAIN | 402-438 | EGF-LIKE 6 |
| DOMAIN | 440-476 | EGF-LIKE 7 |
| DOMAIN | 480-518 | EGF-LIKE 8 |

Human Jagged 1

| | | |
|---|---|---|
| SIGNAL | 1-33 | SIGNAL |
| CHAIN | 34-1218 | JAGGED 1 |
| DOMAIN | 34-1067 | EXTRACELLULAR |
| TRANSMEM | 1068-1093 | TRANSMEMBRANE |
| DOMAIN | 1094-1218 | CYTOPLASMIC |
| DOMAIN | 167-229 | DSL |
| DOMAIN | 234-262 | EGF-LIKE 1 |
| DOMAIN | 265-293 | EGF-LIKE 2 |
| DOMAIN | 300-333 | EGF-LIKE 3 |
| DOMAIN | 340-371 | EGF-LIKE 4 |
| DOMAIN | 378-409 | EGF-LIKE 5 |
| DOMAIN | 416-447 | EGF-LIKE 6 |
| DOMAIN | 454-484 | EGF-LIKE 7 |
| DOMAIN | 491-522 | EGF-LIKE 8 |
| DOMAIN | 529-560 | EGF-LIKE 9 |
| DOMAIN | 595-626 | EGF-LIKE 10 |
| DOMAIN | 633-664 | EGF-LIKE 11 |
| DOMAIN | 671-702 | EGF-LIKE 12 |
| DOMAIN | 709-740 | EGF-LIKE 13 |
| DOMAIN | 748-779 | EGF-LIKE 14 |
| DOMAIN | 786-817 | EGF-LIKE 15 |
| DOMAIN | 824-855 | EGF-LIKE 16 |
| DOMAIN | 863-917 | VON WILLEBRAND |
| | | FACTOR C |

Human Jagged 2

| | | |
|---|---|---|
| SIGNAL | 1-26 | SIGNAL |
| CHAIN | 27-1238 | JAGGED 2 |
| DOMAIN | 27-1080 | EXTRACELLULAR |
| TRANSMEM | 1081-1105 | TRANSMEMBRANE |
| DOMAIN | 1106-1238 | CYTOPLASMIC |
| DOMAIN | 178-240 | DSL |
| DOMAIN | 249-273 | EGF-LIKE 1 |
| DOMAIN | 276-304 | EGF-LIKE 2 |
| DOMAIN | 311-344 | EGF-LIKE 3 |
| DOMAIN | 351-382 | EGF-LIKE 4 |
| DOMAIN | 389-420 | EGF-LIKE 5 |
| DOMAIN | 427-458 | EGF-LIKE 6 |
| DOMAIN | 465-495 | EGF-LIKE 7 |
| DOMAIN | 502-533 | EGF-LIKE 8 |
| DOMAIN | 540-571 | EGF-LIKE 9 |
| DOMAIN | 602-633 | EGF-LIKE 10 |
| DOMAIN | 640-671 | EGF-LIKE 11 |
| DOMAIN | 678-709 | EGF-LIKE 12 |
| DOMAIN | 716-747 | EGF-LIKE 13 |
| DOMAIN | 755-786 | EGF-LIKE 14 |
| DOMAIN | 793-824 | EGF-LIKE 15 |
| DOMAIN | 831-862 | EGF-LIKE 16 |
| DOMAIN | 872-949 | VON WILLEBRAND |
| | | FACTOR C |

DSL Domain

A typical DSL domain may include most or all of the following consensus amino acid sequence:

Cys Xaa Xaa Xaa Xaa Xaa Xaa Xaa Xaa Cys Xaa Xaa
Xaa Cys Xaa Xaa Xaa Xaa Xaa Xaa Xaa Xaa Xaa Xaa
Xaa Cys Xaa Xaa Xaa Xaa Xaa Xaa Xaa Cys Xaa Xaa
Xaa Xaa Xaa Xaa Xaa Xaa Cys

Preferably the DSL domain may include most or all of the following consensus amino acid sequence:

Cys Xaa Xaa Xaa ARO ARO Xaa Xaa Xaa Cys Xaa Xaa
Xaa Cys BAS NOP BAS ACM ACM Xaa ARO NOP ARO Xaa
Xaa Cys Xaa Xaa Xaa NOP Xaa Xaa Xaa Cys Xaa Xaa
NOP ARO Xaa NOP Xaa Xaa Cys

wherein:
ARO is an aromatic amino acid residue, such as tyrosine, phenylalanine, tryptophan or histidine;
NOP is a non-polar amino acid residue such as glycine, alanine, proline, leucine, isoleucine or valine;
BAS is a basic amino acid residue such as arginine or lysine; and
ACM is an acid or amide amino acid residue such as aspartic acid, glutamic acid, asparagine or glutamine.

Preferably the DSL domain may include most or all of the following consensus amino acid sequence:

Cys Xaa Xaa Xaa Tyr Tyr Xaa Xaa Xaa Cys Xaa Xaa
Xaa Cys Arg Pro Arg Asx Asp Xaa Phe Gly His Xaa
Xaa Cys Xaa Xaa Xaa Gly Xaa Xaa Xaa Cys Xaa Xaa
Gly Trp Xaa Gly Xaa Xaa Cys

(wherein Xaa may be any amino acid and Asx is either aspartic acid or asparagine).

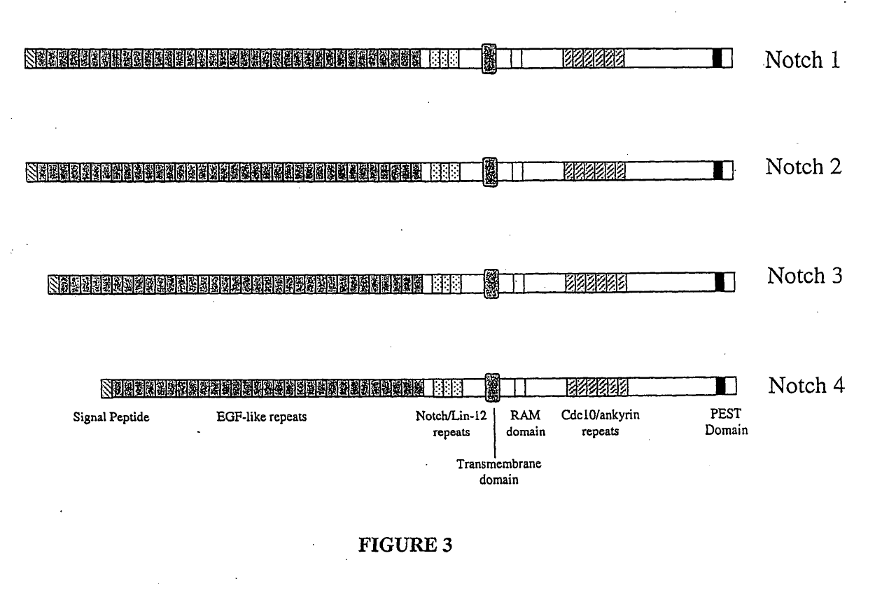

An alignment of DSL domains from Notch ligands from various sources is shown in FIG. 3.

The DSL domain used may be derived from any suitable species, including for example Drosophila, Xenopus, rat, mouse or human. Preferably the DSL domain is derived from a vertebrate, preferably a mammalian, preferably a human Notch ligand sequence.

It will be appreciated that the term “DSL domain” as used herein includes sequence variants, fragments, derivatives and mimetics having activity corresponding to naturally occurring domains.

Suitably, for example, a DSL domain for use in the present invention may have at least 30%, preferably at least 50%, preferably at least 60%, preferably at least 70%, preferably at least 80%, preferably at least 90%, preferably at least 95% amino acid sequence identity to the DSL domain of human Jagged 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Jagged 2.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 3.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 4.

EGF-Like Domain

The EGF-like motif has been found in a variety of proteins, as well as EGF and Notch and Notch ligands, including those involved in the blood clotting cascade (Furie and Furie, 1988, Cell 53: 505-518). For example, this motif has been found in extracellular proteins such as the blood clotting factors 1× and X (Rees et al., 1988, EMBO J. 7:2053-2061; Furie and Furie, 1988, Cell 53: 505-518), in other Drosophila genes (Knust et al., 1987 EMBO J. 761-766; Rothberg et al., 1988, Cell 55:1047-1059), and in some cell-surface receptor proteins, such as thrombomodulin (Suzuki et al., 1987, EMBO J. 6:1891-1897) and LDL receptor (Sudhofet al., 1985, Science 228:815-822). A protein binding site has been mapped to the EGF repeat domain in thrombomodulin and urokinase (Kurosawa et al., 1988, J. Biol. Chem 263:5993-5996; Appella et al., 1987, J. Biol. Chem. 262:4437-4440).

As reported by PROSITE a typical EGF domain may include six cysteine residues which have been shown (in EGF) to be involved in disulfide bonds. The main structure is proposed, but not necessarily required, to be a two-stranded beta-sheet followed by a loop to a C-terminal short two-stranded sheet. Subdomains between the conserved cysteines strongly vary in length as shown in the following schematic representation of a typical EGF-like domain: 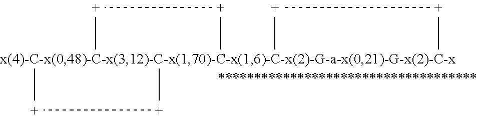
wherein:
‘C’: conserved cysteine involved in a disulfide bond.
‘G’: often conserved glycine
‘a’: often conserved aromatic amino acid
‘*’: position of both patterns.
‘x’: any residue

The region between the 5th and 6th cysteines contains two conserved glycines of which at least one is normally present in most EGF-like domains.

The EGF-like domain used may be derived from any suitable species, including for example Drosophila, Xenopus, rat, mouse or human. Preferably the EGF-like domain is derived from a vertebrate, preferably a mammalian, preferably a human Notch ligand sequence.

It will be appreciated that the term “EGF domain” as used herein includes sequence variants, fragments, derivatives and mimetics having activity corresponding to naturally occurring domains.

Suitably, for example, an EGF-like domain for use in the present invention may have at least 30%, preferably at least 50%, preferably at least 60%, preferably at least 70%, preferably at least 80%, preferably at least 90%, preferably at least 95% amino acid sequence identity to an EGF-like domain of human Jagged 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Jagged 2.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 3.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 4.

As a practical matter, whether any particular amino acid sequence is at least X % identical to another sequence can be determined conventionally using known computer programs. For example, the best overall match between a query sequence and a subject sequence, also referred to as a global sequence alignment, can be determined using a program such as the FASTDB computer program based on the algorithm of Brutlag et al. (Comp. App. Biosci. (1990) 6:237-245). In a sequence alignment the query and subject sequences are either both nucleotide sequences or both amino acid sequences. The result of the global sequence alignment is given as percent identity.

The term “Notch ligand N-terminal domain” means the part of a Notch ligand sequence from the N-terminus to the start of the DSL domain. It will be appreciated that this term includes sequence variants, fragments, derivatives and mimetics having activity corresponding to naturally occurring domains.

The term “heterologous amino acid sequence” or “heterologous nucleotide sequence” as used herein means a sequence which is not found in the native sequence (eg in the case of a Notch ligand sequence is not found in the native Notch ligand sequence) or its coding sequence. Preferably any such heterologous amino acid sequence is not a TSST sequence, and preferably it is not a superantigen sequence.

Whether a substance can be used for activating Notch may be determined using suitable screening assays, for example, as described in our co-pending International Patent Application claiming priority from GB 0118153.6, and the examples herein.

Screening Assays

Whether a substance can be used for modulating Notch signalling may be determined using suitable screening assays (see for example, the Examples herein).

Notch signalling can be monitored either through protein assays or through nucleic acid assays. Activation of the Notch receptor leads to the proteolytic cleavage of its cytoplasmic domain and the translocation thereof into the cell nucleus. The “detectable signal” referred to herein may be any detectable manifestation attributable to the presence of the cleaved intracellular domain of Notch. Thus, increased Notch signalling can be assessed at the protein level by measuring intracellular concentrations of the cleaved Notch domain. Activation of the Notch receptor also catalyses a series of downstream reactions leading to changes in the levels of expression of certain well defined genes. Thus, increased Notch signalling can be assessed at the nucleic acid level by say measuring intracellular concentrations of specific mRNAs. In one preferred embodiment of the present invention, the assay is a protein assay. In another preferred embodiment of the present invention, the assay is a nucleic acid assay.

The advantage of using a nucleic acid assay is that they are sensitive and that small samples can be analysed.

The intracellular concentration of a particular mRNA, measured at any given time, reflects the level of expression of the corresponding gene at that time. Thus, levels of mRNA of downstream target genes of the Notch signalling pathway can be measured in an indirect assay of the T-cells of the immune system. In particular, an increase in levels of Deltex, Hes-1 and/or IL-10 mRNA may, for instance, indicate induced anergy while an increase in levels of Dll-1 or IFN-γ mRNA, or in the levels of mRNA encoding cytokines such as IL-2, IL-5 and IL-13, may indicate improved responsiveness.

Various nucleic acid assays are known. Any convention technique which is known or which is subsequently disclosed may be employed. Examples of suitable nucleic acid assay are mentioned below and include amplification, PCR, RT-PCR, RNase protection, blotting, spectrometry, reporter gene assays, gene chip arrays and other hybridization methods.

In particular, gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe. Those skilled in the art will readily envisage how these methods may be modified, if desired.

PCR was originally developed as a means of amplifying DNA from an impure sample. The technique is based on a temperature cycle which repeatedly heats and cools the reaction solution allowing primers to anneal to target sequences and extension of those primers for the formation of duplicate daughter strands. RT-PCR uses an RNA template for generation of a first strand cDNA with a reverse transcriptase. The cDNA is then amplified according to standard PCR protocol. Repeated cycles of synthesis and denaturation result in an exponential increase in the number of copies of the target DNA produced. However, as reaction components become limiting, the rate of amplification decreases until a plateau is reached and there is little or no net increase in PCR product. The higher the starting copy number of the nucleic acid target, the sooner this “end-point” is reached. Primers can be designed using standard procedures in the art, for example the Taqman™ technique.

Real-time PCR uses probes labeled with a fluorescent tag and differs from end-point PCR for quantitative assays in that it is used to detect PCR products as they accumulate rather than for the measurement of product accumulation after a fixed number of cycles. The reactions are characterized by the point in time during cycling when amplification of a target sequence is first detected through a significant increase in fluorescence. An advantage of real-time PCR is its accuracy in determining the amounts if target sequences in a sample. Suitable protocols are described, for example, in Meuer S. et al (2000).

The ribonuclease protection (RNase protection) assay is an extremely sensitive technique for the quantitation of specific RNAs in solution. The ribonuclease protection assay can be performed on total cellular RNA or poly(A)-selected mRNA as a target. The sensitivity of the ribonuclease protection assay derives from the use of a complementary in vitro transcript probe which is radiolabeled to high specific activity. The probe and target RNA are hybridized in solution, after which the mixture is diluted and treated with ribonuclease (RNase) to degrade all remaining single-stranded RNA. The hybridized portion of the probe will be protected from digestion and can be visualized via electrophoresis of the mixture on a denaturing polyacrylamide gel followed by autoradiography. Since the protected fragments are analyzed by high resolution polyacrylamide gel electrophoresis, the ribonuclease protection assay can be employed to accurately map mRNA features. If the probe is hybridized at a molar excess with respect to the target RNA, then the resulting signal will be directly proportional to the amount of complementary RNA in the sample.

Gene expression may also be detected using a reporter system. Such a reporter system may comprise a readily identifiable marker under the control of an expression system, e.g. of the gene being monitored. Fluorescent markers, which can be detected and sorted by FACS, are preferred. Especially preferred are GFP and luciferase. Another type of preferred reporter is cell surface markers, i.e. proteins expressed on the cell surface and therefore easily identifiable.

In general, reporter constructs useful for detecting Notch signalling by expression of a reporter gene may be constructed according to the general teaching of Sambrook et al (1989). Typically, constructs according to the invention comprise a promoter by the gene of interest, and a coding sequence encoding the desired reporter constructs, for example of GFP or luciferase. Vectors encoding GFP and luciferase are known in the art and available commercially.

Sorting of cells, based upon detection of expression of genes, may be performed by any technique known in the art, as exemplified above. For example, cells may be sorted by flow cytometry or FACS. For a general reference, see Flow Cytometry and Cell Sorting: A Laboratory Manual (1992) A. Radbruch (Ed.), Springer Laboratory, New York.

Flow cytometry is a powerful method for studying and purifying cells. It has found wide application, particularly in immunology and cell biology: however, the capabilities of the FACS can be applied in many other fields of biology. The acronym F.A.C.S. stands for Fluorescence Activated Cell Sorting, and is used interchangeably with “flow cytometry”. The principle of FACS is that individual cells, held in a thin stream of fluid, are passed through one or more laser beams, causing light to be scattered and fluorescent dyes to emit light at various frequencies. Photomultiplier tubes (PMT) convert light to electrical signals, which are interpreted by software to generate data about the cells. Sub-populations of cells with defined characteristics can be identified and automatically sorted from the suspension at very high purity (˜100%).

FACS can be used to measure gene expression in cells transfected with recombinant DNA encoding polypeptides. This can be achieved directly, by labelling of the protein product, or indirectly by using a reporter gene in the construct. Examples of reporter genes are β-galactosidase and Green Fluorescent Protein (GFP). β-galactosidase activity can be detected by FACS using fluorogenic substrates such as fluorescein digalactoside (FDG). FDG is introduced into cells by hypotonic shock, and is cleaved by the enzyme to generate a fluorescent product, which is trapped within the cell. One enzyme can therefore generate a large amount of fluorescent product. Cells expressing GFP constructs will fluoresce without the addition of a substrate. Mutants of GFP are available which have different excitation frequencies, but which emit fluorescence in the same channel. In a two-laser FACS machine, it is possible to distinguish cells which are excited by the different lasers and therefore assay two transfections at the same time.

Alternative means of cell sorting may also be employed. For example, the invention comprises the use of nucleic acid probes complementary to mRNA. Such probes can be used to identify cells expressing mRNA for polypeptides individually, such that they may subsequently be sorted either manually, or using FACS sorting. Nucleic acid probes complementary to mRNA may be prepared according to the teaching set forth above, using the general procedures as described by Sambrook et al (1989).

In a preferred embodiment, the invention comprises the use of an antisense nucleic acid molecule, complementary to a mRNA, conjugated to a fluorophore which may be used in FACS cell sorting.

Methods have also been described for obtaining information about gene expression and identity using so-called gene chip arrays or high density DNA arrays (Chee). These high density arrays are particularly useful for diagnostic and prognostic purposes. Use may also be made of In vivo Expression Technology (IVET) (Camilli). UVET identifies genes up-regulated during say treatment or disease when compared to laboratory culture.

The advantage of using a protein assay is that Notch activation can be directly measured.

Assay techniques that can be used to determine levels of a polypeptide are well known to those skilled in the art. Such assay methods include radioimmunoassays, competitive-binding assays, Western Blot analysis, antibody sandwich assays, antibody detection, FACS and ELISA assays.

The modulator of Notch signalling may also be an immune cell which has been treated to modulate expression or interaction of Notch, a Notch ligand or the Notch signalling pathway. Such cells may readily be prepared, for example, as described in WO 00/36089 in the name of Lorantis Ltd, the text of which is herein incorporated by reference.

Pharmaceutical Compositions

Suitably active agents are administered in combination with a pharmaceutically acceptable diluent, carrier, or excipient (ie as a pharmaceutical composition). The pharmaceutical compositions may be for human or animal usage in human and veterinary medicine.

Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s). Preservatives, stabilizers, dyes and even flavoring agents may also be provided in the pharmaceutical composition as appropriate. Examples of preservatives include sodium benzoate, sorbic acid and esters of p-hydroxybenzoic acid. Antioxidants and suspending agents may be also used.

For some applications, active agents may be administered orally in the form of tablets containing excipients such as starch or lactose, or in capsules or ovules either alone or in admixture with excipients, or in the form of elixirs, solutions or suspensions containing flavouring or colouring agents.

Alternatively or in addition, active agents may be administered by inhalation, intranasally or in the form of aerosol, or in the form of a suppository or pessary, or they may be applied topically in the form of a lotion, solution, cream, ointment or dusting powder. An alternative means of transdermal administration is by use of a skin patch. For example, they can be incorporated into a cream consisting of an aqueous emulsion of polyethylene glycols or liquid paraffin. They can also be incorporated, at a concentration of between 1 and 10% by weight, into an ointment consisting of a white wax or white soft paraffin base together with such stabilisers and preservatives as may be required.

Active agents such as polynucleotides and proteins/polypeptides may also be administered by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes. Active agents may be adminstered by conventional DNA delivery techniques, such as DNA vaccination etc., or injected or otherwise delivered with needleless systems, such as ballistic delivery on particles coated with the DNA for delivery to the epidermis or other sites such as mucosal surfaces.

Typically, the physician will determine the actual dosage which will be most suitable for an individual patient and it will vary with the age, weight and response of the particular patient. The above dosages are exemplary of the average case. There can, of course, be individual instances where higher or lower dosage ranges are merited, and such are within the scope of this invention.

In general, a therapeutically effective oral or intravenous dose is likely to range from 0.01 to 50 mg/kg body weight of the subject to be treated, preferably 0.1 to 20 mg/kg. The conjugate may also be administered by intravenous infusion, at a dose which is likely to range from 0.001-10 mg/kg/hr.

Tablets or capsules of the conjugates may be administered singly or two or more at a time, as appropriate. It is also possible to administer the conjugates in sustained release formulations.

Active agents may also be injected parenterally, for example intracavernosally, intravenously, intramuscularly, intradermally or subcutaneously.

For parenteral administration, active agents may be used in the form of a sterile aqueous solution which may contain other substances, for example enough salts or monosaccharides to make the solution isotonic with blood.

For buccal or sublingual administration, agents may be administered in the form of tablets or lozenges which can be formulated in a conventional manner.

For oral, parenteral, buccal and sublingual administration to subjects (such as patients), the dosage level of active agents and their pharmaceutically acceptable salts and solvates may typically be from 10 to 500 mg (in single or divided doses). Thus, and by way of example, tablets or capsules may contain from 5 to 100 mg of active agent for administration singly, or two or more at a time, as appropriate. As indicated above, the physician will determine the actual dosage which will be most suitable for an individual patient and it will vary with the age, weight and response of the particular patient. It is to be noted that whilst the above-mentioned dosages are exemplary of the average case there can, of course, be individual instances where higher or lower dosage ranges are merited and such dose ranges are within the scope of this invention.

The routes of administration and dosages described are intended only as a guide since a skilled practitioner will be able to determine readily the optimum route of administration and dosage for any particular patient depending on, for example, the age, weight and condition of the patient.

The term treatment or therapy as used herein should be taken to encompass diagnostic and prophylatic applications.

The treatment of the present invention includes both human and veterinary applications.

Active agents may also be administered by any suitable means including, but not limited to, traditional syringes, needleless injection devices, or “microprojectile bombardment gene guns”. Alternatively, active agents such as polynucleotides may be introduced by various means into cells that are removed from an individual. Such means include, for example, ex vivo transfection, electroporation, nucleoporation, microinjection and microprojectile bombardment. After an agent has been taken up by the cells, they may be reimplanted into an individual. It is also contemplated that otherwise non-immunogenic cells that have gene constructs incorporated therein can be implanted into an individual even if the vaccinated cells were originally taken from another individual.

According to some preferred embodiments of the present invention, the active agent may be administered to an individual using a needleless injection device. For example, an active agent may be administered to an individual intradermally, subcutaneously and/or intramuscularly using a needleless injection device, or similarly delivered to mucosal tissues of, for example, the respiratory, gastrointestinal or urinogenital tracts. Needleless injection devices are well known and widely available. Needleless injection devices are especially well suited to deliver genetic material to tissues. They are particularly useful to deliver genetic material to skin and muscle cells. In some embodiments, for example, a needleless injection device may be used to propel a liquid that contains DNA molecules toward the surface of the individual's skin. The liquid is propelled at a sufficient velocity such that upon impact with the skin the liquid penetrates the surface of the skin and permeates the skin and/or muscle tissue beneath. Thus, the genetic material is simultaneously or selectively administered intradermally, subcutaneously and intramuscularly. In some embodiments, a needleless injection device may be used to deliver genetic material to tissue of other organs in order to introduce a nucleic acid molecule to cells of that organ.

Preferably the pharmaceutical preparations according to the present invention are provided sterile and pyrogen free.

Pharmaceutical Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

It will be appreciated that in one embodiment the therapeutic agents used in the present invention may be administered directly to patients in vivo. Alternatively or in addition, the agents may be administered to immune cells such as T cells and/or APCs in an ex vivo manner. For example, leukocytes such as T cells or APCs may be obtained from a patient or donor in known manner, treated/incubated ex vivo in the manner of the present invention, and then administered to a patient.

In general, a therapeutically effective daily dose of the conjugate of the active agent according to the invention may for example range from 0.01 to 50 mg/kg body weight of the subject to be treated, preferably 0.1 to 20 mg/kg.

A skilled practitioner will be able to determine readily the optimum route of administration and dosage for any particular patient depending on, for example, the age, weight and condition of the patient. Preferably the pharmaceutical compositions are in unit dosage form. The present invention includes both human and veterinary applications.

By “simultaneously” is meant that the modulator of the Notch signalling pathway and the pathogen antigen, antigenic determinant or the polynucleotide coding for the pathogen antigen or antigenic determinant are administered at substantially the same time, and preferably together in the same formulation.

By “contemporaneously” it is meant that the modulator of the Notch signalling pathway and the pathogen antigen, antigenic determinant or the polynucleotide coding for the pathogen antigen or antigenic determinant are administered closely in time, e.g., the the pathogen antigen, antigenic determinant or the polynucleotide coding for the pathogen antigen or antigenic determinant is administered within from about one minute to within about one day before or after the modulator of the Notch signalling pathway is administered. Any contemporaneous time is useful. However, it will often be the case that when not administered simultaneously, the modulator of the Notch signalling pathway and the pathogen antigen, antigenic determinant or the polynucleotide coding for the pathogen antigen or antigenic determinant will be administered within about one minute to within about eight hours, and preferably within less than about one to about four hours. When administered contemporaneously, the modulator of the Notch signalling pathway and the pathogen antigen, antigenic determinant or the polynucleotide coding for the pathogen antigen or antigenic determinant are preferably administered at the same site on the animal. The term “same site” includes the exact location, but can be within about 0.5 to about 15 centimeters, preferably from within about 0.5 to about 5 centimeters.

The term “separately” as used herein means that the modulator of the Notch signalling pathway and the pathogen antigen, antigenic determinant or the polynucleotide coding for the pathogen antigen or antigenic determinant are administered at an interval, for example at an interval of about a day to several weeks or months. The active agents may be administered in either order.

Likewise, the modulator of the Notch signalling pathway may be administered more frequently than the pathogen antigen, antigenic determinant or the polynucleotide coding for the pathogen antigen or antigenic determinant or vice versa.

The term “sequentially” as used herein means that the modulator of the Notch signalling pathway and the pathogen antigen, antigenic determinant or the polynucleotide coding for the pathogen antigen or antigenic determinant are administered in sequence, for example at an interval or intervals of minutes, hours, days or weeks. If appropriate the active agents may be administered in a regular repeating cycle.

Vaccine Compositions

Vaccine compositions and preparations made in accordance with the present invention may be used to protect or treat a mammal susceptible to, or suffering from disease, by means of administering said vaccine via a mucosal route, such as the oral/bucal/intestinal/vaginal/rectal or nasal route. Such administration may be in a droplet, spray, or dry powdered form. Nebulised or aerosolised vaccine formulations may also be used where appropriate.

Enteric formulations such as gastro resistant capsules and granules for oral administration, suppositories for rectal or vaginal administration may also be used. The present invention may also be used to enhance the immunogenicity of antigens applied to the skin, for example by intradermal, transdermal or transcutaneous delivery. In addition, the adjuvants of the present invention may be parentally delivered, for example by intramuscular or subcutaneous administration.

Depending on the route of administration, a variety of administration devices may be used. For example, for intranasal administration a spray device such as the commercially available Accuspray (Becton Dickinson) may be used.

Preferred spray devices for intranasal use are devices for which the performance of the device is not dependent upon the pressure applied by the user. These devices are known as pressure threshold devices. Liquid is released from the nozzle only when a threshold pressure is attained. These devices make it easier to achieve a spray with a regular droplet size. Pressure threshold devices suitable for use with the present invention are known in the art and are described for example in WO 91/13281 and EP 311 863 B. Such devices are commercially available from Pfeiffer GmbH.

For certain vaccine formulations, other vaccine components may be included in the formulation. For example the adjuvant formulations of the present invention may also comprise a bile acid or derivative of cholic acid. Suitably the derivative of cholic acid is a salt thereof, for example a sodium salt thereof. Examples of bile acids include cholic acid itself, deoxycholic acid, chenodeoxy colic acid, lithocholic acid, taurodeoxycholate ursodeoxycholic acid, hyodeoxycholic acid and derivatives like glyco-, tauro-, amidopropyl-1-propanesulfonic- and amidopropyl-2-hydroxy-1-propanesulfonic-derivatives of the above bile acids, or N,N-bis (3DGluconoamidopropyl) deoxycholamide.

Suitably, the adjuvant formulation of the present invention may be in the form of an aqueous solution or a suspension of non-vesicular forms. Such formulations are convenient to manufacture, and also to sterilise (for example by terminal filtration through a 450 or 220 nm pore membrane).

Suitably, the route of administration to said host is via the skin, intramuscular or via a mucosal surface such as the nasal mucosa. When the admixture is administered via the nasal mucosa, the admixture may for example be administered as a spray. The methods to enhance an immune response may be either a priming or boosting dose of the vaccine.

The term “adjuvant” as used herein includes an agent having the ability to enhance the immune response of a vertebrate subject's immune system to an antigen or antigenic determinant.

The term “immune response” includes any response to an antigen or antigenic determinant by the immune system of a subject. Immune responses include for example humoral immune responses (e.g. production of antigen-specific antibodies) and cell-mediated immune responses (e.g. lymphocyte proliferation).

The term “cell-mediated immune response” includes the immunological defence provided by lymphocytes, such as the defence provided by T cell lymphocytes when they come into close proximity with their victim cells.

When “lymphocyte proliferation” is measured, the ability of lymphocytes to proliferate in response to specific antigen may be measured. Lymphocyte proliferation includes B cell, T-helper cell or CTL cell proliferation.

Compositions of the present invention may be used to formulate vaccines containing antigens derived from a wide variety of sources. For example, antigens may include human, bacterial, or viral nucleic acid, pathogen derived antigen or antigenic preparations, host-derived antigens, including GNRH and IgE peptides, recombinantly produced protein or peptides, and chimeric fusion proteins.

Preferably the vaccine formulations of the present invention contain an antigen or antigenic composition capable of eliciting an immune response against a human pathogen. The antigen or antigens may, for example, be peptides/proteins, polysaccharides and lipids and may be derived from pathogens such as viruses, bacteria and parasites/fingi as follows:

Viral Antigens

Viral antigens or antigenic determinants may be derived, for example, from:

Cytomegalovirus (especially Human, such as gB or derivatives thereof); Epstein Barr virus (such as gp350); flaviviruses (e.g. Yellow Fever Virus, Dengue Virus, Tick-borne encephalitis virus, Japanese Encephalitis Virus); hepatitis virus such as hepatitis B virus (for example Hepatitis B Surface antigen such as the PreS1, PreS2 and S antigens described in EP-A-414 374; EP-A-0304 578, and EP-A-198474), hepatitis A virus, hepatitis C virus and hepatitis E virus; HIV-1, (such as tat, nef, gp120 or gp160); human herpes viruses, such as gD or derivatives thereof or Immediate Early protein such as ICP27 from HSV1 or HSV2; human papilloma viruses (for example HPV6, 11, 16, 18); Influenza virus (whole live or inactivated virus, split influenza virus, grown in eggs or MDCK cells, or Vero cells or whole flu virosomes (as described by Gluck, Vaccine, 1992, 10, 915-920) or purified or recombinant proteins thereof, such as NP, NA, HA, or M proteins); measles virus; mumps virus; parainfluenza virus; rabies virus; Respiratory Syncytial virus (such as F and G proteins); rotavirus (including live attenuated viruses); smallpox virus; Varicella Zoster Virus (such as gpI, II and IE63); and the HPV viruses responsible for cervical cancer (for example the early proteins E6 or E7 in fusion with a protein D carrier to form Protein D-E6 or E7 fusions from HPV 16, or combinations thereof; or combinations of E6 or E7 with L2 (see for example WO 96/26277).

Bacterial Antigens

Bacterial antigens or antigenic determinants may be derived, for example, from:

Bacillus spp., including B. anthracis (eg botulinum toxin); Bordetella spp, including B. pertussis (for example pertactin, pertussis toxin, filamenteous hemagglutinin, adenylate cyclase, fimbriae); Borrelia spp., including B. burgdorferi (eg OspA, OspC, DbpA, DbpB), B. garinii (eg OspA, OspC, DbpA, DbpB), B. afzelii (eg OspA, OspC, DbpA, DbpB), B. andersonii (eg OspA, OspC, DbpA, DbpB), B. hermsii; Campylobacter spp, including C. jejuni (for example toxins, adhesins and invasins) and C. coli; Chlamydia spp., including C. trachomatis (eg MOMP, heparin-binding proteins), C. pneumonie (eg MOMP, heparin-binding proteins), C. psittaci; Clostridium spp., including C. tetani (such as tetanus toxin), C. botulinum (for example botulinum toxin), C. difficile (eg clostridium toxins A or B); Corynebacterium spp., including C. diphtheriae (eg diphtheria toxin); Ehrlichia spp., including E. equi and the agent of the Human Granulocytic Ehrlichiosis; Rickettsia spp, including R. rickettsii; Enterococcus spp., including E. faecalis, E. faecium; Escherichia spp, including enterotoxic E. coli (for example colonization factors, heat-labile toxin or derivatives thereof, or heat-stable toxin), enterohemorragic E. coli, enteropathogenic E. coli (for example shiga toxin-like toxin); Haemophilus spp., including H. influenzae type B (eg PRP), non-typable H. influenzae, for example OMP26, high molecular weight adhesins, P5, P6, protein D and lipoprotein D, and fimbrin and fimbrin derived peptides (see for example U.S. Pat. No. 5,843,464); Helicobacter spp, including H. pylori (for example urease, catalase, vacuolating toxin); Pseudomonas spp, including P. aeruginosa; Legionella spp, including L pneumophila; Leptospira spp., including L. interrogans; Listeria spp., including L. monocytogenes; Moraxella spp, including M catarrhalis, also known as Branhamella catarrhalis (for example high and low molecular weight adhesins and invasins); Morexella Catarrhalis (including outer membrane vesicles thereof, and OMP106 (see for example WO97/41731)); Mycobacterium spp., including M. tuberculosis (for example ESAT6, Antigen 85A, -B or -C), M. bovis, M. leprae, M. avium, M. paratuberculosis, M. smegmatis; Neisseria spp, including N. gonorrhea and N. meningitidis (for example capsular polysaccharides and conjugates thereof, transferrin-binding proteins, lactoferrin binding proteins, PilC, adhesins); Neisseria mengitidis B (including outer membrane vesicles thereof, and NspA (see for example WO 96/29412); Salmonella spp, including S. typhi, S. paratyphi, S. choleraesuis, S. enteritidis; Shigella spp, including S. sonnei, S. dysenteriae, S. flexnerii; Staphylococcus spp., including S. aureus, S. epidermidis; Streptococcus spp, including S. pneumonie (eg capsular polysaccharides and conjugates thereof, PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (see for example WO 90/06951; WO 99/03884); Treponema spp., including T. pallidum (eg the outer membrane proteins), T. denticola, T. hyodysenteriae; Vibrio spp, including V. cholera (for example cholera toxin); and Yersinia spp, including Y. enterocolitica (for example a Yop protein), Y. pestis, Y. pseudotuberculosis.

Parasite/Fungal Antigens

Parasitic/fungal antigens or antigenic determinants may be derived, for example, from:

Babesia spp., including B. microti; Candida spp., including C. albicans; Cryptococcus spp., including C. neoformans; Entamoeba spp., including E. histolytica; Giardia spp., including; G. lamblia; Leshmania spp., including L. major; Plasmodium. faciparum (MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, Pfs230 and their analogues in Plasmodium spp.); Pneumocystis spp., including P.; carinii; Schisostoma spp., including S. mansoni; Trichomonas spp., including T. vaginalis; Toxoplasma spp., including T. gondii (for example SAG2, SAG3, Tg34); Trypanosoma spp., including T. cruzi.

Approved/licensed vaccines include, for example anthrax vaccines such as Biothrax (BioPort Corp); tuberculosis (BCG) vaccines such as TICE BCG (Organon Teknika Corp) and Mycobax (Aventis Pasteur, Ltd); diphtheria & tetanus toxoid and acellular pertussis (DTP) vaccines such as Tripedia (Aventis Pasteur, Inc), Infanrix (GlaxoSmithKline), and DAPTACEL (Aventis Pasteur, Ltd); Haemophilus b conjugate vaccines (eg diphtheria CRM197 protein conjugates such as HibTITER from Lederle Lab Div, American Cyanamid Co; meningococcal protein conjugates such as PedvaxHIB from Merck & Co, Inc; and tetanus toxoid conjugates such as ActHIB from Aventis Pasteur, SA); Hepatitis A vaccines such as Havrix (GlaxoSmithKline) and VAQTA (Merck & Co, Inc); combined Hepatitis A and Hepatitis B (recombinant) vaccines such as Twinrix (GlaxoSmithKline); recombinant Hepatitis B vaccines such as Recombivax HB (Merck & Co, Inc) and Engerix-B (GlaxoSmithKline); influenza virus vaccines such as Fluvirin (Evans Vaccine), FluShield (Wyeth Laboratories, Inc) and Fluzone (Aventis Pasteur, Inc); Japanese Encephalitis virus vaccine such as JE-Vax (Research Foundation for Microbial Diseases of Osaka University); Measles virus vaccines such as Attenuvax (Merck & Co, Inc); measles and mumps virus vaccines such as M-M-Vax (Merck & Co, Inc); measles, mumps, and rubella virus vaccines such as M-M-R II (Merck & Co, Inc); meningococcal polysaccharide vaccines (Groups A, C, Y and W-135 combined) such as Menomune-A/C/Y/W-135 (Aventis Pasteur, Inc); mumps virus vaccines such as Mumpsvax (Merck & Co, Inc); pneumococcal vaccines such as Pneumovax (Merck & Co, Inc) and Pnu-Imune (Lederle Lab Div, American Cyanamid Co); Pneumococcal 7-valent conjugate vaccines (eg diphtheria CRM197 Protein conjugates such as Prevnar from Lederle Lab Div, American Cyanamid Co); poliovirus vaccines such as Poliovax (Aventis Pasteur, Ltd); poliovirus vaccines such as IPOL (Aventis Pasteur, SA); rabies vaccines such as Imovax (Aventis Pasteur, SA) and RabAvert (Chiron Behring GmbH & Co); rubella virus vaccines such as Meruvax II (Merck & Co, Inc); Typhoid Vi polysaccharide vaccines such as TYPHIM Vi (Aventis Pasteur, SA); Varicella virus vaccines such as Varivax (Merck & Co, Inc) and Yellow Fever vaccines such as YF-Vax (Aventis Pasteur, Inc).

It will be appreciated that in accordance with this aspect of the present invention antigens and antigenic determinants may be used in many different forms. For example, antigens or antigenic determinants may be present as isolated proteins or peptides (for example in so-called “subunit vaccines”) or, for example, as cell-associated or virus-associated antigens or antigenic determinants (for example in either live or killed pathogen strains). Live pathogens will preferably be attenuated in known manner. Alternatively, antigens or antigenic determinants may be generated in situ in the subject by use of a polynucleotide coding for an antigen or antigenic determinant (as in so-called “DNA vaccination”, although it will be appreciated that the polynucleotides which may be used with this approach are not limited to DNA, and may also include RNA and modified polynucleotides as discussed above).

As used herein, the term “genetic vaccine” refers to a pharmaceutical preparation that comprises a polynucleotide (eg DNA) construct. Genetic vaccines include pharmaceutical preparations useful to invoke a prophylactic and/or therapeutic immune response. Therapeutic vaccines may also be referred to as “Pharmacines”.

As discussed, for example, in U.S. Pat. No. 6,025,341 and elsewhere, direct injection of polynucleotides such as DNA is a promising method for delivering antigens for immunization (Barry, et al., Bio Techniques, 1994, 16, 616-619; Davis, et al., Hum. Mol. Genet., 1993, 11, 1847-1851; Tang, et al., Nature, 1992, 356, 152-154; Wang, et al., J. Virol., 1993, 67, 3338-3344; and Wolff, et al., Science, 1990, 247, 1465-1468). This approach has been successfully used to generate protective immunity against influenza virus in mice and chickens, against bovine herpes virus 1 in mice and cattle and against rabies virus in mice (Cox, et al., J. Virol., 1993, 67, 5664-5667; Fynan, et al., DNA and Cell Biol., 1993, 12, 785-789; Ulmer, et al., Science, 1993, 259, 1745-1749; and Xiang, et al., Virol., 1994, 199, 132-140). In most cases, strong, yet highly variable, antibody and cytotoxic T-cell responses were associated with control of infection. Indeed, the potential to generate long-lasting memory CTLs without using a liver vector makes this approach particularly attractive compared with those involving killed-virus vaccines and generating a CTL response that not only protects against acute infection but also may have benefits in eradicating persistent viral infection (Wolff, et al., Science, 1990, 247, 1465-1468; Wolff, et al., Hum. Mol. Genet., 1992, 1, 363-369; Manthorpe, et al., Human Gene Therapy, 1993, 4, 419-431; Ulmer, et al., Science, 1993, 259, 1745-1749; Yankauckas, et al., DNA and Cell Biol., 1993, 12, 777-783; Montgomery, et al., DNA and Cell Biol., 1993, 12, 777-783; Fynan, et al., DNA and Cell Biol., 1993, 12, 785-789; Wang, et al., Proc. Natl. Acad. Sci. USA, 1993, 90, 4156-4160; Wang, et al., DNA and Cell Biol., 1993, 12, 799-805; Xiang, et al., Virol., 1994, 199, 132-140; and Davis, et al., Hum. Mol. Genet., 1993, 11, 1847-1851) of which HCV and HBV are important human diseases of world wide significance.

Genetic vaccines suitable for use according to the present invention may for example comprise from about 1 nanogram to about 1000 micrograms of a polynucleotide such as DNA, suitably from about about 10 nanograms to about 800 micrograms, suitably from about 0.1 to about 500 micrograms, suitably from about 1 to about 350 micrograms, suitably from about 25 to about 250 micrograms of a polynucleotide such as DNA.

The amount of protein in a vaccine dose is selected as an amount which induces an immunoprotective response without significant, adverse side effects in typical recipients. Such amount will vary depending upon which specific immunogen is employed and how it is presented. Typically, it is expected that each dose will comprise 1-1000 μg of protein, preferably 1-500 μg, preferably 1-100 μg, most preferably 1 to 50 μg. After an initial vaccination, subjects may receive one or several booster immunisations suitably spaced.

The vaccines of the present invention may also be administered via the oral route. In such cases the pharmaceutically acceptible excipient may also include alkaline buffers, or enteric capsules or microgranules. The vaccines of the present invention may also be administered by the vaginal route. In such cases, the pharmaceutically acceptable excipients may also include emulsifiers, polymers such as CARBOPOL, and other known stablilisers of vaginal creams and suppositories. The vaccines of the present invention may also be administered by the rectal route. In such cases the excipients may also include waxes and polymers known in the art for forming rectal suppositories.

The formulations of the present invention may be used for both prophylactic and therapeutic purposes. Accordingly, the present invention provides for a method of treating a mammal susceptible to or suffering from an infectious disease. In a further aspect of the present invention there is provided an adjuvant combination and a vaccine as herein described for use in medicine. Vaccine preparation is generally described in New Trends and Developments in Vaccines, edited by Voller et al., University Park Press, Baltimore, Md., U.S.A. 1978. It will be appreciated that the adjuvants of the present invention may further be combined with other adjuvants including, for example: Cholera toxin and its B subunit; E. Coli heat labile enterotoxin LT, its B subunit LTB and detoxified versions thereof such as mLT; immunologically active saponin fractions e.g. Quil A derived from the bark of the South American tree Quillaja Saponaria Molina and derivatives thereof (for example QS21, as described in U.S. Pat. No. 5,057,540); the oligonucleotide adjuvant system CpG (as described in WO 96/02555), especially 5′TCG TCG TTT TGT CGT TTT GTC GTT3 (SEQ ID NO: 1); and Monophosphoryl Lipid A and its non-toxic derivative 3-O-deacylated monophosphoryl lipid A (3D-MPL, as described in GB 2,220,211).

The present invention provides an increased magnitude and/or increased duration of immune response. Preferably the invention provides an increased protective immune response.

The present invention also contemplates generating selective Th1 or Th2 immunity. In general, T cells can act in different subpopulations that show different effector functions. T cell responses can be pro-inflammatory T helper 1 type (Th1) characterized by the secretion of interferon gamma (IFN-gamma.) and interleukin 2 (IL-2). Th1 cells are the helper cells for the cellular defence but provide little help for antibody secretion. The other class of T cell responses is generally anti-inflammatory, and is mediated by Th2 cells that produce IL-4, IL-5 and IL-10, but little or no IL-2 or IFN-gamma. Th2 cells are the helper cells for antibody production. CD4+ and CD8+ cells both occur in these subpopulations: Th1/Th2:CD4, Tc1/Tc2:CD8.

For each type of pathogen/infection there may be an “appropriate” (and different) type of T cell response (e.g., Th1 vs. Th2, CD4+ vs. CD8+) that combats the infectious agent but does not cause excessive tissue damage in the subject. It may be detrimental to the subject if an “inappropriate” type of T cell response is induced (Th1 instead of Th2, or vice versa). Generally, one would want to induce the Th1 response to clear an intracellular pathogen such as a virus or intracellular bacterium and a Th2 response to clear an extracellular pathogen. It will be appreciated that the present invention may be used in both so-called prophylactic and so-called therapeutic vaccines.

For example, prophylactic vaccines may be used to provide protective immunity in an uninfected subject to provide protection against future establishment of infection.

Conversely, therapeutic vaccines may be used, for example, after an infection has become established (for example as either an acute or chronic infection) in order to increase the immune response against the infection. Suitably, therapeutic vaccines may be used to combat chronic infections which may for example be bacterial infections (such as tuberculosis), parasitic infections such as malarial infections or viral infections (such as HPV, HCV, HBV or HIV infections).

Examples of chronic infections associated with significant morbidity and early death include human hepatitis viruses such as hepatitis A, B, C, D and E, for example hepatitis B virus (HBV) and hepatitis C virus (HCV) which cause chronic hepatitis, cirrhosis and liver cancer (see U.S. Pat. No. 5,738,852).

Additional examples of chronic infections caused by viral infectious agents include those caused by the human retroviruses: human immunodeficiency viruses (HIV-1 and HIV-2), which cause acquired immune deficiency syndrome (AIDS); and human T lymphotropic viruses (HTLV-1 and HTLV-2) which cause T cell leukemia and myelopathies. Many other infections such as human herpes viruses including the herpes simplex virus (HSV) types 1 and 2, Epstein Barr virus (EBV), cytomegalovirus (CMV), varicella-zoster virus (VZV) and human herpes virus 6 (HHV-6) are often not eradicated by host mechanisms, but rather become chronic and in this state may cause disease. Chronic infection with human papilloma viruses is associated with cervical carcinoma. Numerous other viruses and other infectious agents replicate intracellularly and may become chronic when host defense mechanisms fail to eliminate them. These include pathogenic protozoa (e.g., Pneumocystis carinii, Trypanosoma, Leishmania, Plasmodium (responsible for Malaria) and Toxoplasma gondii), bacteria (e.g., mycobacteria (eg Mycobacterium tuberculosis responsible for tuberculosis), salmonella and listeria), and fungi (e.g., candida and aspergillus).

The pathogen antigen is suitably an antigen that is naturally encoded in the pathogen against which an enhanced or augmented immune response is desired.

The nucleotide sequences of a large number of bacteria, protozoans and viruses, including different species, strains, and isolates are known in the art (see, for example Levy, Microbiological Reviews, 57:183-289 (1993) (HIV); and Choo et al., Seminars in Liver Disease, 12:279-288 (1992) (HCV)). Particularly suitable target antigens are those which induce a T cell response, and particularly a CTL-response during infection. These may include, for example, from HBV, the core antigen (HBcAg) the E antigen, and the surface antigen (HBsAg). Polynucleotide sequences for HBsAg including the pre-S 1, pre-S2 and S regions from a variety of surface antigen subtypes are well known in the art (see, for example, Okamoto et al., J. Gen. Virol., 67:1383-1389 (1986); GenBank Accession numbers D00329 and D00330). The polynucleotide sequences encoding HIV glycoprotein gp160 and other antigenic HIV regions are known in the art (Lautenberger et al., Nature, 313:277-284 (1985); Starcich et al., Cell, 45:637-648 (1986); Wiley et al., Proc. Natl. Acad. Sci. USA, 83:5038-5042 (1986); and Modrow et al., J. Virol., 61:570-578 (1987)).

For example, the genome for Human immunodeficiency virus type 1 (HXB2; HIV1/HTLV-III/LAV reference genome) is provided at GenBank Accession No K03455, which reports sequences for various HIV antigenic proteins.

Numerous genome sequences for HAV, HBV and HCV strains (including sequences for antigenic proteins) are provided on GenBank, for example AY057948 (Hepatitis B virus isolate Tibet127, complete genome); AY057947 (Hepatitis B virus isolate Tibet705, complete genome); NC_{—}003977 (Hepatitis B virus, complete genome); NC_{—}004102 (Hepatitis C virus, complete genome); AF139594 (Hepatitis C virus strain HCV-N, complete genome); M16632 (Hepatitis A virus (HM-175 strain; attenuated)).

In one embodiment the modulator/inhibitor of Notch signalling increases cytotoxic (CD8+) T cell responses to antigen.

Conjugates

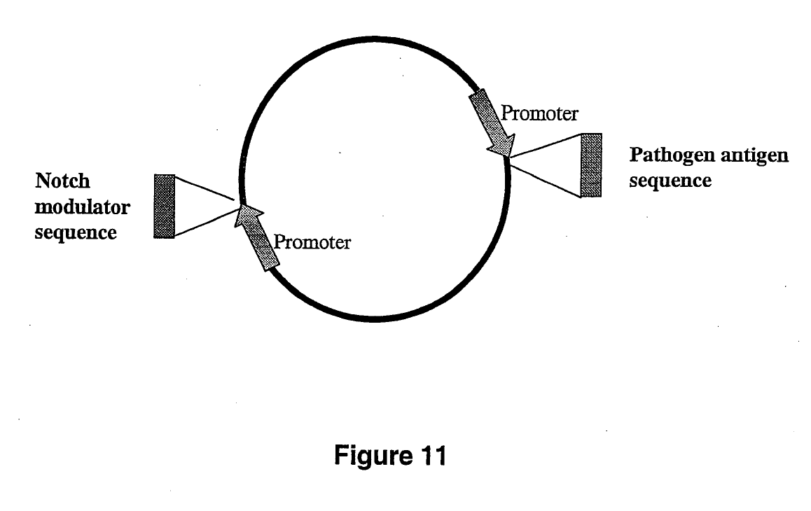

As noted above, the invention further provides a conjugate comprising first and second sequences, wherein the first sequence comprises a pathogen antigen or a polynucleotide sequence coding for such an antigen and the second sequence comprises a polypeptide or polynucleotide for Notch signalling modulation. The conjugates of the present invention may be protein/polypeptide or polynucleotide conjugates.

Where the conjugate is a polynucleotide conjugate, it may suitably take the form of a polynucleotide vector such as a plasmid comprising a polynucleotide sequence coding for a pathogen antigen or antigenic determinant and a polynucleotide sequence coding for a modulator of the Notch signalling pathway, wherein preferably each sequence is operably linked to regulatory elements necessary for expression in eukaryotic cells. A schematic representation of one such form of vector is shown in FIG. 11.

Suitably the polynucleotide sequence coding for the modulator of the Notch signalling pathway may be a nucleotide sequence coding for a Notch ligand such as Delta1, Delta3, Delta4, Jagged1 or Jagged 2, or a biologically active fragment, derivative or homologue of such a sequence. Where intended for human therapy, suitably sequences based on human sequences may be used.

Preferably the polynucleotide sequence coding for the modulator of the Notch signalling pathway may be a nucleotide sequence coding for a Notch ligand DSL domain and at least 1 to 20, suitably at least 2 to 15, suitably at least 2 to 10, for example at least 3 to 8 EGF-like domains. Suitably the DSL and EGF-like domain sequences are or correspond to mammalian sequences. Suitably the polynucleotide sequence coding for the modulator of the Notch signalling pathway may further comprise a transmembrane domain and, suitably, a Notch ligand intracellular domain. Preferred sequences include human sequences such as human Delta1, Delta3, Delta4, Jagged1 or Jagged2 sequences.

If desired, the polynucleotide sequence that encodes the pathogen antigen or antigenic determinant may further include a nucleotide sequence that encodes a signal sequence which directs trafficking of the antigen or antigenic determinant within a cell to which it is administered. For example, such a signal sequence may direct the antigen or antigenic determinant to be secreted or to be localized to the cytoplasm, the cell membrane, the endoplasmic reticulum, or a lysosome.

Regulatory elements for DNA expression include a promoter and a polyadenylation signal. In addition, other elements, such as a Kozak region, may also be included if desired. Initiation and termination signals are regulatory elements which are often considered part of the coding sequence.

Examples of suitable promoters include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from human genes such as human Actin, human Myosin, human Hemoglobin, human muscle creatine and human metalothionein. Tissue-specific promoters specific for lymphocytes, dendritic cells, skin, brain cells and epithelial cells within the eye are particularly preferred, for example the CD2, CD11c, keratin 14, Wnt-1 and Rhodopsin promoters respectively. Suitably an epithelial cell promoter such as SPC may be used.

Examples of suitable polyadenylation signals include but are not limited to SV40 polyadenylation signals and LTR polyadenylation signals. For example, the SV40 polyadenylation signal used in plasmid pCEP4 (Invitrogen, San Diego Calif.), referred to as the SV40 polyadenylation signal, may be used.

In addition to the regulatory elements required for DNA expression, other elements may also be included in the conjugate. Such additional elements include enhancers which may, for example, be selected from human Actin, human Myosin, human Hemoglobin, human muscle creatine and viral enhancers such as those from CMV, RSV and EBV.

When administered to and taken up by a cell, the nucleotide conjugate may for example remain present in the cell as a functioning extrachromosomal molecule and/or integrate into the cell's chromosomal DNA. DNA may be introduced into cells where it remains as separate genetic material in the form of a plasmid or plasmids. Alternatively, linear DNA which can integrate into the chromosome may be introduced into the cell. When introducing DNA into the cell, reagents which promote DNA integration into chromosomes may be added. DNA sequences which are useful to promote integration may also be included in the DNA molecule. Alternatively, RNA may be administered to the cell. It is also possible, for example, to provide the conjugate in the form of a minichromosome including a centromere, telomeres and an origin of replication.

If desired, conjugates may be provided with mammalian origin of replication in order to maintain the construct extrachromosomally and produce multiple copies of the construct in the cell. For example, plasmids pCEP4 and pREP4 from Invitrogen (San Diego, Calif.) contain the Epstein Barr virus origin of replication and nuclear antigen EBNA-1 coding region which produces high copy episomal replication without integration.

In order to maximize protein production, regulatory sequences may be selected which are well suited for gene expression in the type of cells the construct is to be administered to. Moreover, codons may be selected which are most efficiently transcribed in the cell.

Such conjugates may be used either in vivo or ex-vivo with a “genetic vaccination” approach to provide expression of both an inhibitor of Notch signalling and a pathogen antigen or antigenic determinant in cells or tissues.

Facilitating Agents

In some embodiments, polynucleotides may be delivered in conjunction with administration of a facilitating agent. Facilitating agents which are administered in conjunction with nucleic acid molecules may be administered as a mixture with the nucleic acid molecule or administered separately simultaneously, before or after administration of nucleic acid molecules. Examples of facilitators include benzoic acid esters, anilides, amidines, urethans and the hydrochloride salts thereof such as those of the family of local anesthetics.

Examples of esters include: benzoic acid esters such as piperocaine, meprylcaine and isobucaine; para-aminobenzoic acid esters such as procaine, tetracaine, butethamine, propoxycaine and chloroprocaine; meta-aminobenzoic acid esters including metabuthamine and primacaine; and para-ethoxybenzoic acid esters such as parethoxycaine. Examples of anilides include lidocaine, etidocaine, mepivacaine, bupivacaine, pyrrocaine and prilocalne. Other examples of such compounds include dibucaine, benzocaine, dyclonine, pramoxine, proparacaine, butacaine, benoxinate, carbocaine, methyl bupivacaine, butasin picrate, phenacaine, diothan, luccaine, intracaine, nupercaine, metabutoxycaine, piridocaine, biphenamine and the botanically-derived bicyclics such as cocaine, cinnamoylcocaine, truxilline and cocaethylene and all such compounds complexed with hydrochloride.

The facilitating agent may be administered prior to, simultaneously with or subsequent to the genetic construct. The facilitating agent and the genetic construct may be formulated in the same composition.

Bupivacaine-HCl is chemically designated as 2-piperidinecarboxamide, 1-butyl-N-(2,6-dimethylphenyl)-monohydrochloride, monohydrate and is widely available commercially for pharmaceutical uses from many sources including from Astra Pharmaceutical Products Inc. (Westboro, Mass.) and Sanofi Winthrop Pharmaceuticals (New York, N.Y.), Eastman Kodak (Rochester, N.Y.). Bupivacaine is commercially formulated with and without methylparaben and with or without epinephrine. Any such formulation may be used. It is commercially available for pharmaceutical use in concentration of 0.25%, 0.5% and 0.75% which may be used on the invention. Alternative concentrations, particularly those between 0.05%-1.0% which elicit desirable effects may be prepared if desired. Suitably, for example, about 250 μg to about 10 mg of bupivacaine may be administered.

Antigen Presenting Cells

Where required, antigen-presenting cells (APCs) may be “professional” antigen presenting cells or may be another cell that may be induced to present antigen to T cells. Alternatively a APC precursor may be used which differentiates or is activated under the conditions of culture to produce an APC. An APC for use in the ex vivo methods of the invention is typically isolated from a tumour or peripheral blood found within the body of a patient. Preferably the APC or precursor is of human origin. However, where APCs are used in preliminary in vitro screening procedures to identify and test suitable nucleic acid sequences, APCs from any suitable source, such as a healthy patient, may be used.

APCs include dendritic cells (DCs) such as interdigitating DCs or follicular DCs, Langerhans cells, PBMCs, macrophages, B-lymphocytes, or other cell types such as epithelial cells, fibroblasts or endothelial cells, activated or engineered by transfection to express a MHC molecule (Class I or II) on their surfaces. Precursors of APCs include CD34⁺ cells, monocytes, fibroblasts and endothelial cells. The APCs or precursors may be modified by the culture conditions or may be genetically modified, for instance by transfection of one or more genes encoding proteins which play a role in antigen presentation and/or in combination of selected cytokine genes which would promote to immune potentiation (for example IL-2, IL-12, IFN-γ, TNF-α, IL-18 etc.). Such proteins include MHC molecules (Class I or Class II), CD80, CD86, or CD40. Most preferably DCs or DC-precursors are included as a source of APCs.

Dendritic cells (DCs) can be isolated/prepared by a number of means, for example they can either be purified directly from peripheral blood, or generated from CD34⁺ precursor cells for example after mobilisation into peripheral blood by treatment with GM-CSF, or directly from bone marrow. From peripheral blood, adherent precursors can be treated with a GM-CSF/IL-4 mixture (Inaba K, et al. (1992) J. Exp. Med. 175: 1157-1167 (Inaba)), or from bone marrow, non-adherent CD34⁺ cells can be treated with GM-CSF and TNF-a (Caux C, et al. (1992) Nature 360: 258-261 (Caux)). DCs can also be routinely prepared from the peripheral blood of human volunteers, similarly to the method of Sallusto and Lanzavecchia (Sallusto F and Lanzavecchia A (1994) J. Exp. Med. 179: 1109-1118) using purified peripheral blood mononucleocytes (PBMCs) and treating 2 hour adherent cells with GM-CSF and IL-4. If required, these may be depleted of CD19⁺ B cells and CD3⁺, CD2⁺ T cells using magnetic beads (Coffin R S, et al. (1998) Gene Therapy 5: 718-722 (Coffin)). Culture conditions may include other cytokines such as GM-CSF or IL-4 for the maintenance and, or activity of the dendritic cells or other antigen presenting cells.

Thus, it will be understood that the term “antigen presenting cell or the like” are used herein is not intended to be limited to APCs. The skilled man will understand that any vehicle capable of presenting to the T cell population may be used, for the sake of convenience the term APCs is used to refer to all these. As indicated above, preferred examples of suitable APCs include dendritic cells, L cells, hybridomas, fibroblasts, lymphomas, macrophages, B cells or synthetic APCs such as lipid membranes.

T Cells

Where required, T cells from any suitable source, such as a healthy patient, may be used and may be obtained from blood or another source (such as lymph nodes, spleen, or bone marrow). They may optionally be enriched or purified by standard procedures. The T cells may be used in combination with other immune cells, obtained from the same or a different individual. Alternatively whole blood may be used or leukocyte enriched blood or purified white blood cells as a source of T cells and other cell types. It is particularly preferred to use helper T cells (CD4⁺). Alternatively other T cells such as CD8⁺ cells may be used. It may also be convenient to use cell lines such as T cell hybridomas.

Thus, it will be understood that the term “antigen presenting cell or the like” are used herein is not intended to be limited to APCs. The skilled man will understand that any vehicle capable of presenting to the T cell population may be used, for the sake of convenience the term APCs is used to refer to all these. As indicated above, preferred examples of suitable APCs include dendritic cells, L cells, hybridomas, fibroblasts, lymphomas, macrophages, B cells or synthetic APCs such as lipid membranes.

Exposure of Agent to APCs and T Cells

T cells/APCs/tumour cells may be cultured as described above. The APCs/T cells/tumour cells may be incubated/exposed to substances which are capable of interferring with or downregulating Notch or Notch ligand expression. The resulting T cells/APCs/tumour cells that have downregulated Notch or Notch ligand expression are now ready for use. For example, they may be prepared for administration to a patient or incubated with T cells in vitro (ex vivo).

For example, tumour material may be isolated and transfected with a nucleic acid sequence which encodes for, e.g., a Toll-like receptor or BMP receptor and/or costimulatory molecules (suitable costimulants are mentioned above) and/or treated with cytokines, e.g. IFN-γ, TNF-α, IL-12, and then used in vitro to prime TRL and/or TIL cells.

Where treated ex-vivo, modified cells of the present invention are preferably administered to a host by direct injection into the lymph nodes of the patient. Typically from 10⁴ to 10⁸ treated cells, preferably from 10⁵ to 10⁷ cells, more preferably about 10⁶ cells are administered to the patient. Preferably, the cells will be taken from an enriched cell population.

As used herein, the term “enriched” as applied to the cell populations of the invention refers to a more homogeneous population of cells which have fewer other cells with which they are naturally associated. An enriched population of cells can be achieved by several methods known in the art. For example, an enriched population of T-cells can be obtained using immunoaffinity chromatography using monoclonal antibodies specific for determinants found only on T-cells.

Enriched populations can also be obtained from mixed cell suspensions by positive selection (collecting only the desired cells) or negative selection (removing the undesirable cells). The technology for capturing specific cells on affinity materials is well known in the art (Wigzel, et al., J. Exp. Med., 128:23, 1969; Mage, et al., J. Immunol. Meth., 15:47, 1977; Wysocki, et al., Proc. Natl. Acad. Sci. U.S.A., 75:2844, 1978; Schrempf-Decker, et al., J. Immunol Meth., 32:285, 1980; Muller-Sieburg, et al., Cell, 44:653, 1986).

Monoclonal antibodies against antigens specific for mature, differentiated cells have been used in a variety of negative selection strategies to remove undesired cells, for example, to deplete T-cells or malignant cells from allogeneic or autologous marrow grafts, respectively (Gee, et al., J.N.C.I. 80:154, 1988). Purification of human hematopoietic cells by negative selection with monoclonal antibodies and immunomagnetic microspheres can be accomplished using multiple monoclonal antibodies (Griffin, et al., Blood, 63:904, 1984).

Procedures for separation of cells may include magnetic separation, using antibody coated magnetic beads, affinity chromatography, cytotoxic agents joined to a monoclonal antibody or used in conjunction with a monoclonal antibody, for example, complement and cytotoxins, and “panning” with antibodies attached to a solid matrix, for example, plate, or other convenient technique. Techniques providing accurate separation include fluorescence activated cell sorters, which can have varying degrees of sophistication, for example, a plurality of color channels, low angle and obtuse light scattering detecting channels, impedance channels, etc.

It will be appreciated that in one embodiment the therapeutic agents used in the present invention may be administered directly to patients in vivo. Alternatively or in addition, the agents may be administered to cells such as T cells and/or APCs in an ex vivo manner. For example, leukocytes such as T cells or APCs may be obtained from a patient or donor in known manner, treated/incubated ex vivo in the manner of the present invention, and then administered to a patient. In addition, it will be appreciated that a combination of routes of administration may be employed if desired. For example, where appropriate one component (such as the modulator of Notch signalling) may be administered ex-vivo and the other may be administered in vivo, or vice versa.

Introduction of Nucleic Acid Sequences into APCs and T-Cells

T-cells and APCs as described above are cultured in a suitable culture medium such as DMEM or other defined media, optionally in the presence of fetal calf serum.

Polypeptide substances may be administered to T-cells and/or APCs by introducing nucleic acid constructs/viral vectors encoding the polypeptide into cells under conditions that allow for expression of the polypeptide in the T-cell and/or APC. Similarly, nucleic acid constructs encoding antisense constructs may be introduced into the T-cells and/or APCs by transfection, viral infection or viral transduction.

In a preferred embodiment, nucleotide sequences encoding the modulator(s) of Notch signalling will be operably linked to control sequences, including promoters/enhancers and other expression regulation signals. The term “operably linked” means that the components described are in a relationship permitting them to function in their intended manner. A regulatory sequence “operably linked” to a coding sequence is peferably ligated in such a way that expression of the coding sequence is achieved under condition compatible with the control sequences.

The promoter is typically selected from promoters which are functional in mammalian cells, although prokaryotic promoters and promoters functional in other eukaryotic cells may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of a-actin, b-actin, tubulin) or, alternatively, a tissue-specific manner (such as promoters of the genes for pyruvate kinase). Tissue-specific promoters specific for lymphocytes, dendritic cells, skin, brain cells and epithelial cells within the eye are particularly preferred, for example the CD2, CD11c, keratin 14, Wnt-1 and Rhodopsin promoters respectively. Preferably the epithelial cell promoter SPC is used. They may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

It may also be advantageous for the promoters to be inducible so that the levels of expression of the heterologous gene can be regulated during the life-time of the cell. Inducible means that the levels of expression obtained using the promoter can be regulated.

Any of the above promoters may be modified by the addition of further regulatory sequences, for example enhancer sequences. Chimeric promoters may also be used comprising sequence elements from two or more different promoters.

Alternatively (or in addition), the regulatory sequences may be cell specific such that the gene of interest is only expressed in cells of use in the present invention. Such cells include, for example, APCs and T-cells.

The resulting T-cells and/or APCs that comprise nucleic acid constructs capable of up-regulating Notch ligand expression are now ready for use. If required, a small aliquot of cells may be tested for up-regulation of Notch ligand expression as described above. The cells may be prepared for administration to a patient or incubated with T-cells in vitro (ex vivo).

Any of the assays described above (see “Assays”) can be adapted to monitor or to detect reactivity in immune cells for use in clinical applications. Such assays will involve, for example, detecting Notch-ligand activity in host cells or monitoring Notch cleavage in donor cells. Further methods of monitoring immune cell activity are set out below.

Immune cell activity may be monitored by any suitable method known to those skilled in the art. For example, cytotoxic activity may be monitored. Natural killer (NK) cells will demonstrate enhanced cytotoxic activity after activation. Therefore any drop in or stabilisation of cytotoxicity will be an indication of reduced reactivity.

Once activated, leukocytes express a variety of new cell surface antigens. NK cells, for example, will express transferrin receptor, HLA-DR and the CD25 IL-2 receptor after activation. Reduced reactivity may therefore be assayed by monitoring expression of these antigens.

Hara et al. Human T-cell Activation: III, Rapid Induction of a Phosphorylated 28 kD/32 kD Disulfide linked Early Activation Antigen (EA-1) by 12-O-tetradecanoyl Phorbol-13-Acetate, Mitogens and Antigens, J. Exp. Med., 164:1988 (1986), and Cosulich et al. Functional Characterization of an Antigen (MLR3) Involved in an Early Step of T-Cell Activation, PNAS, 84:4205 (1987), have described cell surface antigens that are expressed on T-cells shortly after activation. These antigens, EA-1 and MLR3 respectively, are glycoproteins having major components of 28 kD and 32 kD. EA-1 and MLR3 are not HLA class II antigens and an MLR3 Mab will block IL-1 binding. These antigens appear on activated T-cells within 18 hours and can therefore be used to monitor immune cell reactivity.

Additionally, leukocyte reactivity may be monitored as described in EP 0325489, which is incorporated herein by reference. Briefly this is accomplished using a monoclonal antibody (“Anti-Leu23”) which interacts with a cellular antigen recognised by the monoclonal antibody produced by the hybridoma designated as ATCC No. HB-9627.

Anti-Leu 23 recognises a cell surface antigen on activated and antigen stimulated leukocytes. On activated NK cells, the antigen, Leu 23, is expressed within 4 hours after activation and continues to be expressed as late as 72 hours after activation. Leu 23 is a disulfide-linked homodimer composed of 24 kD subunits with at least two N-linked carbohydrates.

Because the appearance of Leu 23 on NK cells correlates with the development of cytotoxicity and because the appearance of Leu 23 on certain T-cells correlates with stimulation of the T-cell antigen receptor complex, Anti-Leu 23 is useful in monitoring the reactivity of leukocytes.

Further details of techniques for the monitoring of immune cell reactivity may be found in: ‘The Natural Killer Cell’ Lewis C. E. and J. O'D. McGee 1992. Oxford University Press; Trinchieri G. ‘Biology of Natural Killer Cells’ Adv. Immunol. 1989 vol 47 pp 187-376; ‘Cytokines of the Immune Response’ Chapter 7 in “Handbook of Immune Response Genes”. Mak T. W. and J. J. L. Simard 1998, which are incorporated herein by reference.

Preparation of Primed APCs and Lymphocytes

According to one aspect of the invention immune cells may be used to present antigens or allergens and/or may be treated to modulate expression or interaction of Notch, a Notch ligand or the Notch signalling pathway. Thus, for example, Antigen Presenting Cells (APCs) may be cultured in a suitable culture medium such as DMEM or other defined media, optionally in the presence of a serum such as fetal calf serum. Optimum cytokine concentrations may be determined by titration. One or more substances capable of up-regulating or down-regulating the Notch signalling pathway are then typically added to the culture medium together with the antigen of interest. The antigen may be added before, after or at substantially the same time as the substance(s). Cells are typically incubated with the substance(s) and antigen for at least one hour, preferably at least 3 hours, at 37° C. If required, a small aliquot of cells may be tested for modulated target gene expression as described above. Alternatively, cell activity may be measured by the inhibition of T cell activation by monitoring surface markers, cytokine secretion or proliferation as described in WO98/20142. APCs transfected with a nucleic acid construct directing the expression of, for example Serrate, may be used as a control.

As discussed above, polypeptide substances may be administered to APCs by introducing nucleic acid constructs/viral vectors encoding the polypeptide into cells under conditions that allow for expression of the polypeptide in the APC. Similarly, nucleic acid constructs encoding antigens may be introduced into the APCs by transfection, viral infection or viral transduction. The resulting APCs that show increased levels of a Notch signalling are now ready for use.

Tolerisation Assays

Any of the assays described above (see “Assays”) can be adapted to monitor or to detect the degree of reactivity and tolerisation in immune cells for use in clinical applications. Such assays will involve, for example, detecting decreased Notch signalling activity in host cells or monitoring Notch cleavage in donor cells. Further methods of monitoring immune cell activity are set out below.

Immune cell activity may be monitored by any suitable method known to those skilled in the art. For example, cytotoxic activity may be monitored. Natural killer (NK) cells will demonstrate enhanced cytotoxic activity after activation. Therefore any drop in or stabilisation of cytotoxicity will be an indication of reduced reactivity.

Once activated, leukocytes express a variety of new cell surface antigens. NK cells, for example, will express transferrin receptor, HLA-DR and the CD25 IL-2 receptor after activation. Reduced reactivity may therefore be assayed by monitoring expression of these antigens.

Hara et al. Human T-cell Activation: III, Rapid Induction of a Phosphorylated 28 kD/32 kD Disulfide linked Early Activation Antigen (EA-1) by 12-O-tetradecanoyl Phorbol-13-Acetate, Mitogens and Antigens, J. Exp. Med., 164:1988 (1986), and Cosulich et al. Functional Characterization of an Antigen (MLR3) Involved in an Early Step of T-Cell Activation, PNAS, 84:4205 (1987), have described cell surface antigens that are expressed on T-cells shortly after activation. These antigens, EA-1 and MLR3 respectively, are glycoproteins having major components of 28 kD and 32 kD. EA-1 and MLR3 are not HLA class II antigens and an MLR3 Mab will block IL-1 binding. These antigens appear on activated T-cells within 18 hours and can therefore be used to monitor immune cell reactivity.

Additionally, leukocyte reactivity may be monitored as described in EP 0325489, which is incorporated herein by reference. Briefly this is accomplished using a monoclonal antibody (“Anti-Leu23”) which interacts with a cellular antigen recognised by the monoclonal antibody produced by the hybridoma designated as ATCC No. HB-9627.

Anti-Leu 23 recognises a cell surface antigen on activated and antigen stimulated leukocytes. On activated NK cells, the antigen, Leu 23, is expressed within 4 hours after activation and continues to be expressed as late as 72 hours after activation. Leu 23 is a disulfide-linked homodimer composed of 24 kD subunits with at least two N-linked carbohydrates.

Because the appearance of Leu 23 on NK cells correlates with the development of cytotoxicity and because the appearance of Leu 23 on certain T-cells correlates with stimulation of the T-cell antigen receptor complex, Anti-Leu 23 is useful in monitoring the reactivity of leukocytes.

Further details of techniques for the monitoring of immune cell reactivity may be found in: ‘The Natural Killer Cell’ Lewis C. E. and J. O'D. McGee 1992. Oxford University Press; Trinchieri G. ‘Biology of Natural Killer Cells’ Adv. Immunol. 1989 vol 47 pp 187-376; ‘Cytokines of the Immune Response’ Chapter 7 in “Handbook of Immune Response Genes”. Mak T. W. and J. J. L. Simard 1998, which are incorporated herein by reference.

Various preferred features and embodiments of the present invention will now be described in more detail by way of non-limiting examples.

### EXAMPLES

### Example 1

### Preparation of Inhibitor of Notch Signalling (hDelta1-IgG4Fc Fusion Protein

A fusion protein comprising the extracellular domain of human Delta1 fused to the Fc domain of human IgG4 (“hDelta1-IgG4Fc”) was prepared by inserting a nucleotide sequence coding for the extracellular domain of human Delta1 (see, eg Genbank Accession No AF003522) into the expression vector pCONγ (Lonza Biologics, Slough, UK) and expressing the resulting construct in CHO cells.

i) Cloning

A 1622 bp extracellular (EC) fragment of human Delta-like ligand 1 (hECDLL-1; see GenBank Accession No AF003522) was gel purified using a Qiagen QIAquick™ Gel Extraction Kit (cat 28706) according to the manufacturer's instructions. The fragment was then ligated into a pCR Blunt cloning vector (Invitrogen, UK) cut HindIII-BsiWI, thus eliminating a HindIII, BsiWI and ApaI site.

The ligation was transformed into DH5α cells, streaked onto LB+Kanamycin (30 ug/ml) plates and incubated at 37° C. overnight. Colonies were picked from the plates into 3 ml LB+Kanamycin (30 ugml⁻¹) and grown up overnight at 37° C. Plasmid DNA was purified from the cultures using a Qiagen Qiaquick Spin Miniprep kit (cat 27106) according to the manufacturer's instructions, then diagnostically digested with HindIII. A clone was chosen and streaked onto an LB+Kanamycin (30 ug/ml) plate with the glycerol stock of modified pCRBlunt-hECDLL-1 and incubated at 37° C. overnight. A colony was picked off this plate into 60 ml LB+Kanamycin (30 ug/ml) and incubated at 37° C. overnight. The culture was maxiprepped using a Clontech Nucleobond Maxi Kit (cat K3003-2) according to the manufacturer's instructions, and the final DNA pellet was resuspended in 300 ul dH₂O and stored at −20° C.

5 ug of modified pCR Blunt-hECDLL-1 vector was linearised with HindIII and partially digested with ApaI. The 1622 bp hECDLL-1 fragment was then gel purified using a Clontech Nucleospin® Extraction Kit (K3051-1) according to the manufacturer's instructions. The DNA was then passed through another Clontech Nucleospin® column and followed the isolation from PCR protocol, concentration of sample was then checked by agarose gel analysis ready for ligation.

Plasmid pconγ (Lonza Biologics, UK) was cut with HindIII-ApaI and the following oligos were ligated in (SEQ ID NO: 2):
agcttgcggc cgcgggccca gcggtggtgg acctcactga gaagctagag gcttccacca aaggcc acgccg gcgcccgggt cgccaccacc tggagtgact cttcgatctc cgaaggtggt tt

The ligation was transformed into DH5α cells and LB+Amp (100 ug/ml) plates were streaked with 200 ul of the transformation and incubated at 37° C. overnight. The following day 12 clones were picked into 2×YT+Ampicillin (100 ugml⁻¹) and grown up at 37° C. throughout the day. Plasmid DNA was purified from the cultures using a Qiagen Qiaquick Spin Miniprep kit (cat 27106) and diagnostically digested with NotI. A clone (designated “pDev41”) was chosen and an LB+Amp (100 ug/ml) plate was streaked with the glycerol stock of pDev41 and incubated at 37° C. overnight. The following day a clone was picked from this plate into 60 ml LB+Amp (10 ug/ml) and incubated with shaking at 37° C. overnight. The clone was maxiprepped using a Clontech Nucleobond Maxi Kit (cat K3003-2) according to the manufacturer's instructions and stored at −20° C.

The pDev41 clone 5 maxiprep was then digested with ApaI-EcoRI to generate the IgG4Fc fragment (1624 bp). The digest was purified on a 1% agarose gel and the main band was cut out and purified using a Clontech Nucleospin Extraction Kit (K3051-1).

The polynucleotide was then cloned into the polylinker region of pEE14.4 (Lonza Biologics, UK) downstream of the strong hCMV promoter enhancer region (hCMV-MIE) and upstream of SV40 polyadenylation signal (encodes the GS gene required for selection in glutamine free media; contains the GS minigene—GS cDNA which includes the last intron and polylinker adenylation signals of the wild type hamster GS gene) which is under the control of the late SV40 promoter, has the hCMV promoter to drive transcription of the desired gene. 5 ug of the maxiprep of pEE14.4 was digested with HindIII-EcoRI, and the product was gel extracted and treated with alkaline phosphatase.

ii) Generation of Expression Constructs

A 3 fragment ligation was set up with pEE14.4 cut HindIII-EcoRI, ECDLL-1 from modified pCR Blunt (HindIII-ApaI) and the IgG4Fc fragment cut from pDev41 (ApaI-EcoRI). This was transformed into DH5α cells and LB+Amp (100 ug/ml) plates were streaked with 200 ul of the transformation and incubated at 37 C overnight. The following day 12 clones were picked into 2×YT+Amp (100 ug/ml) and mimpreps were grown up at 37° C. throughout the day. Plasmid DNA was purified from the preps using a Qiagen Qiaquick spin miniprep kit (Cat No 27106), diagnostically digested (with EcoRI and HindIII) and a clone (clone 8; designated “pDev44”) was chosen for maxiprepping. The glycerol stock of pDev44 clone 8 was streaked onto an LB+Amp (100 ugml⁻¹) plate and incubated at 0.37° C. overnight. The following day a colony was picked into 60 ml LB+Amp (100 ugml⁻¹) broth and incubated at 37° C. overnight. The plasmid DNA was isolated using a Clontech Nucleobond Maxiprep Kit (Cat K3003-2).
iii) Addition of optimal KOZAK Sequence

A Kozak sequence was inserted into the expression construct as follows. Oligonucleotides were kinase treated and annealed to generate the following sequences:

| | |
|---|---|
| AGCTTGCCGCCACCATGGGCAGTCGGTGCGCGCTGGCCCTGGCGGTGCTC | (SEQ ID NO: 3) |
| ACGGCGGTGGTACCCGTCAGCCACGCGCGACCGGGACCGC | |
| TCGGCCTTGCTGTGTCAGGTCTGGAGCTCTGGGGTGTT | (SEQ ID NO: 4) |
| CACGAGAGCCGGAACGACACAGTCCAGACCTCGAGACCCCACAAGC | |

pDev44 was digested with HindIII-BstBI, gel purified and treated with alkaline phosphatase. The digest was ligated with the oligos, transformed into DH5α cells by heat shock. 200 ul of each transformation were streaked onto LB+Amp plates (100 ug/ml) and incubated at 37° C. overnight. Minipreps were grown up in 3 ml 2×YT+Ampicillin (100 ugml⁻). Plasmid DNA was purified from the minipreps using a Qiagen Qiaquick spin miniprep kit (Cat No 27106) and diagnostically digested with NcoI. A clone (pDev46) was selected and the sequence was confirmed. The glycerol stock was streaked, broth grown up and the plasmid maxiprepped.

iv) Transfection

Approx 100 ug pDev46 Clone 1 DNA was linearised with restriction enzyme Pvu I. The resulting DNA preparation was cleaned up using phenol/chlorofom/IAA extraction followed by ethanol wash and precipitation. The pellets were resuspended in sterile water and linearisation and quantification was checked by agarose gel electrophoresis and UV spectrophotometry.

40 ug linearised DNA (pDev46 Clone 1) and 1×10⁷ CHO-K1 cells were mixed in serum free DMEM in a 4 mm cuvette, at room temp. The cells were then electroporated at 975 uF 280 volts, washed out into non-selective DMEM, diluted into 96 well plates and incubated. After 24 hours media were removed and replaced with selective media (25 uM L-MSX). After 6 weeks media were removed and analysed by IgG4 sandwich ELISA.

Selective media were replaced. Positive clones were identified and passaged in selective media 25 um L-MSX.

v) Expression

Cells were grown in selective DMEM (25 um L-MSX) until semi-confluent. The media was then replaced with serum free media (UltraCHO) for 3-5 days. Protein (hDelta1-IgG4Fc fusion protein) was purified from the resulting media by HPLC.

The amino acid sequence of the resulting expressed fusion protein was as follows (SEQ ID NO: 5):

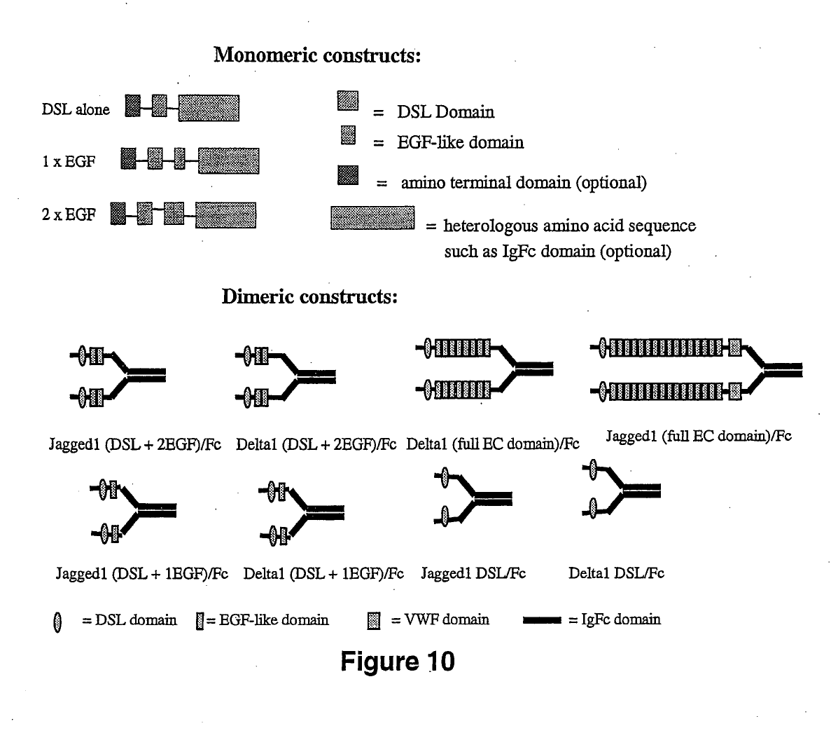

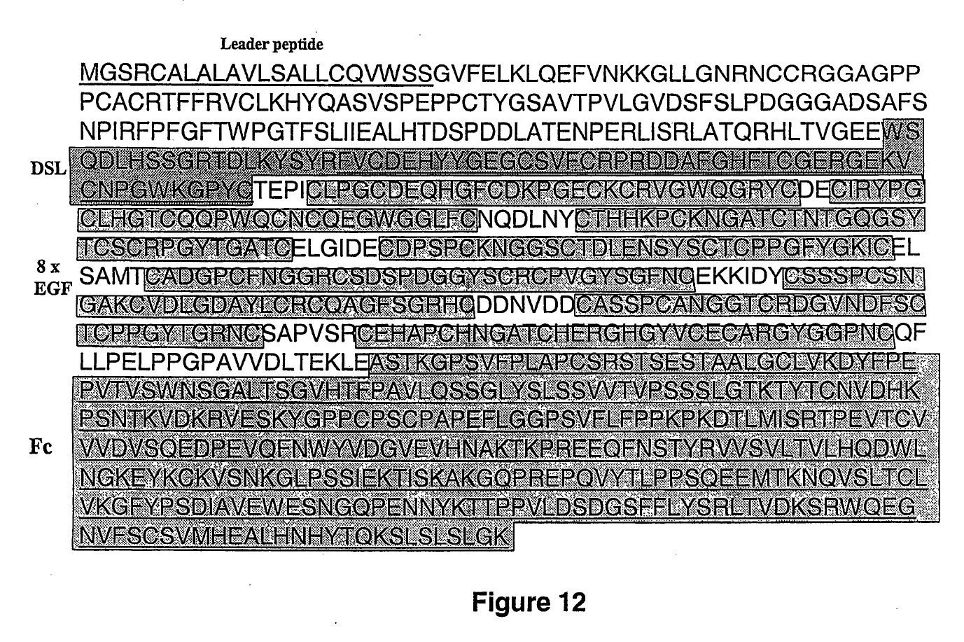

MGSRCALALAVLSALLCQVWSSGVFELKLQEFVNKKGLLGNRNCCRGGAG
PPPCACRTFFRVCLKHYQASVSPEPPCTYGSAVTPVLGVDSFSLPDGGGA
DSAFSNPIRFPFGFTWPGTFSLIIEALHTDSPDDLATENPERLISRLATQ
RHLTVGEEWSQDLHSSGRTDLKYSYRFVCDEHYYGEGCSVFCRPRDDAFG
HFTCGERGEKVCNPGWKGPYCTEPICLPGCDEQHGFCDKPGECKCRVGWQ
GRYCDECIRYPGCLHGTCQQPWQCNCQEGWGGLFCNQDLNYCTHHKPCKN
GATCTNTGQGSYTCSCRPGYTGATCELGIDECDPSPCKNGGSCTDLENSY
SCTCPPGFYGKICELSAMTCADGPCFNGGRCSDSPDGGYSCRCPVGYSGF
NCEKKIDYCSSSPCSNGAKCVDLGDAYLCRCQAGFSGRHCDDNVDDCASS
PCANGGTCRDGVNDFSCTCPPGYTGRNCSAPVSRCEHAPCHNGATCHERG
HGYVCECARGYGGPNCQFLLPELPPGPAVVDLTEKLEASTKGPSVFPLAP
CSRSTSESTAALGCLVKDYFPEPVTVSWNSGALTSGVHTFPAVLQSSGLY
SLSSVVTVPSSSLGTKTYTCNVDHKPSNTKVDKRVESKYGPPCPSCPAPE
FLGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIE
KTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALH
NHYTQKSLSLSLGK

Wherein the first underlined sequence is the signal peptide (cleaved from the mature protein) and the second underlined sequence is the IgG4 Fc sequence. The protein normally exists as a dimer linked by cysteine disulphide bonds (see eg schematic representation in FIG. 10). The domain structure of the expressed fusion protein is shown in more detail in FIG. 12.

### Example 2

### Notch Signalling Inhibitor Enhances Immune Response to Flu Antigen

Flushield™ flu vaccine (5 micrograms; Roche USA) was emulsified in incomplete Freund's adjuvant with or without 100 micrograms of hDelta1-IgG4Fc (from Example 1 above). 6-8 weeks old BALB/c mice (eight per group) were immunized subcutaneously at the base of the tail and 14 days later the mice were challenged in the right ear with 1.8 micrograms of Flushield flu vaccine in saline. Ear responses (ear thickness measured with callipers) were measured at 1, 2 and 6 days thereafter.

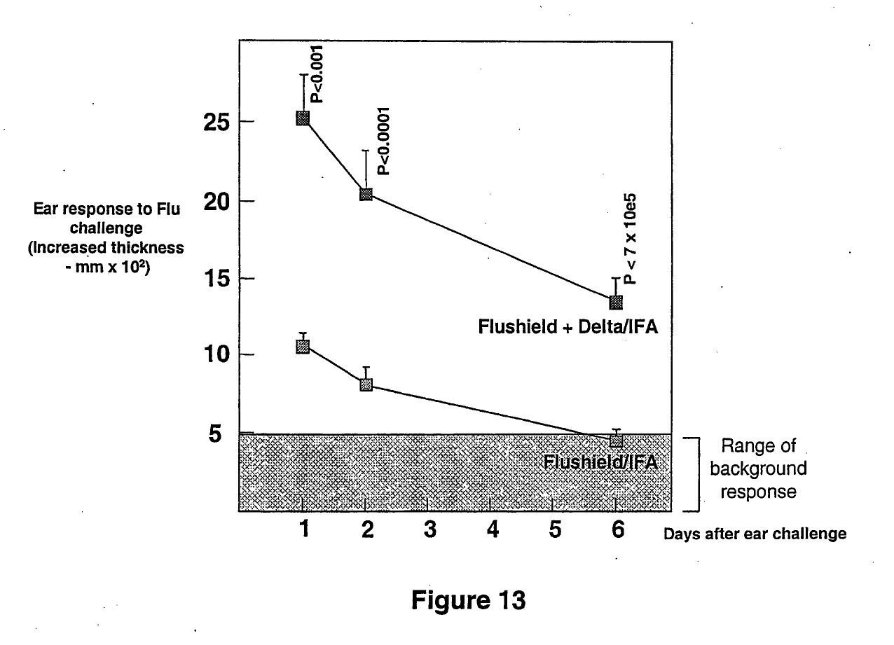

Results expressed as increase (right ear-left ear) in ear swelling are shown in FIG. 13.

### Example 3

### Notch Signalling Inhibitor Enhances Immune Response to KLH

6-8 weeks old BALB/c mice (eight per group) were immunized subcutaneously at the base of the tail with keyhole limpet haemocyanin (KLH) from Pierce at 50 ng or 0.5 ng per mouse emulsified in incomplete Freund's adjuvant (IFA) with or without hDelta1-IgG4Fc protein from Example 1 above (100 micrograms). Some mice also received additional hdeltal-IgG4Fc (400 micrograms) at an adjacent s.c. site one day later. 14 days after the initial KLH priming, mice were challenged in the right ear with 20 micrograms KLH and the ear immune response was measured with callipers as an increase in ear thickness due to the induced inflammatory reaction after 24 hours.

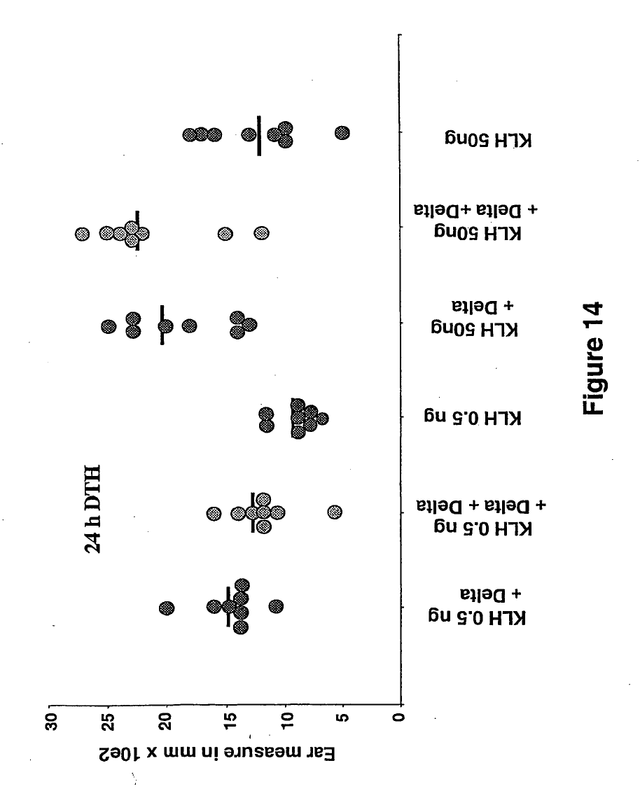

Results are shown in FIG. 14.

### Example 4

### Notch Signalling Inhibitor Enhances Immune Response to Flu Vaccine

6-8 weeks old BALB/c mice (eight per group) were immunized subcutaneously at the base of the tail with Flushield™ flu vaccine at 5 μg per mouse emulsified in incomplete Freund's adjuvant (IFA) with hDelta1-IgG4Fc protein from Example 1 above (100 micrograms) or isotype control hIgG4 (Sigma, UK) 100 μg/IFA control. 14 days after the initial Flushield™ flu vaccine priming, mice were challenged in the right ear with Flushield™ and the ear immune response was measured with callipers as an increase in ear thickness due to the induced inflammatory reaction after 24 hours.

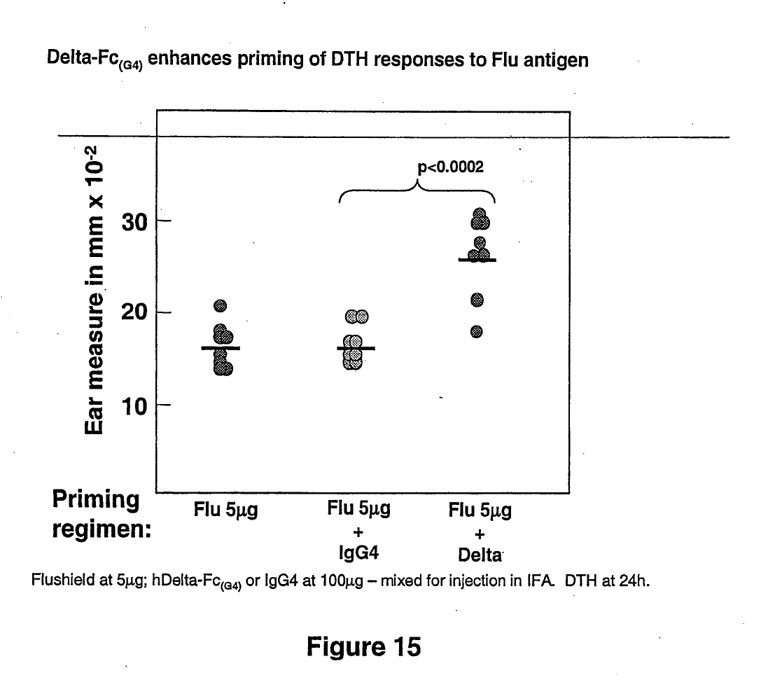

Results are shown in FIG. 15.

### Example 5

### The Modulation of Cytokine Production Induced by Delta1 Beads is Inhibited by the Addition of Soluble hDelta1-IgG4Fc

i) Preparation of Beads Coated with hDelta1-IgG4Fc Fusion Proteins

M450 Streptavidin Dynabead™ magnetic beads (Dynal, USA) were coated with an anti-human-IgG4 biotinylated monoclonal antibody (BD Bioscience, 555879) by rotating them in the presence of the antibody for 30 minutes at room temperature. Beads were washed three times with PBS (1 ml). They were further incubated with hDelta1-hIgG4 (see Example 1 above) for 2 hours at room temperature and then washed three times with PBS (1 ml).

ii) Investigation of Notch Signalling by ELISA

Human peripheral blood mononuclear cells (PBMC) were purified from blood using Ficoll-Paque separation medium (Pharmacia). Briefly, 28 ml of blood were overlaid on 21 ml of Ficoll-Paque separation medium and centrifuged at 18-20° C. for 40 minutes at 400 g. PBMC were recovered from the interface and washed 3 times before use for CD4+ T cell purification.

The CD4+ T cells were incubated in triplicates in a 96-well-plate (flat bottom) at 10⁵ CD4/well/200 μl in RPMI medium containing 10% FCS, glutamine, penicillin, streptomycin and β₂-mercaptoethanol.

Cytokine production was induced by stimulating the cells with anti-CD3/CD28 T cell expander beads from Dynal at a 1:1 ratio (bead/cell) in the presence of beads coated with hDelta1-IgG4Fc fusion protein (Example 1 above) at a 5:1 ratio (beads/cell). In some wells, increasing amounts of soluble hdelta1-IgG4Fc fusion protein were also added.

The supernatants were removed after 3 days of incubation at 37° C./5% CO₂/humidified atmosphere and cytokine production was evaluated by ELISA using Pharmingen kits OptEIA Set human IL10 (catalog No. 555157), OptEIA Set human IL-5 (catalog No. 555202) for IL-10 and IL-5 respectively according to the manufacturer's instructions.

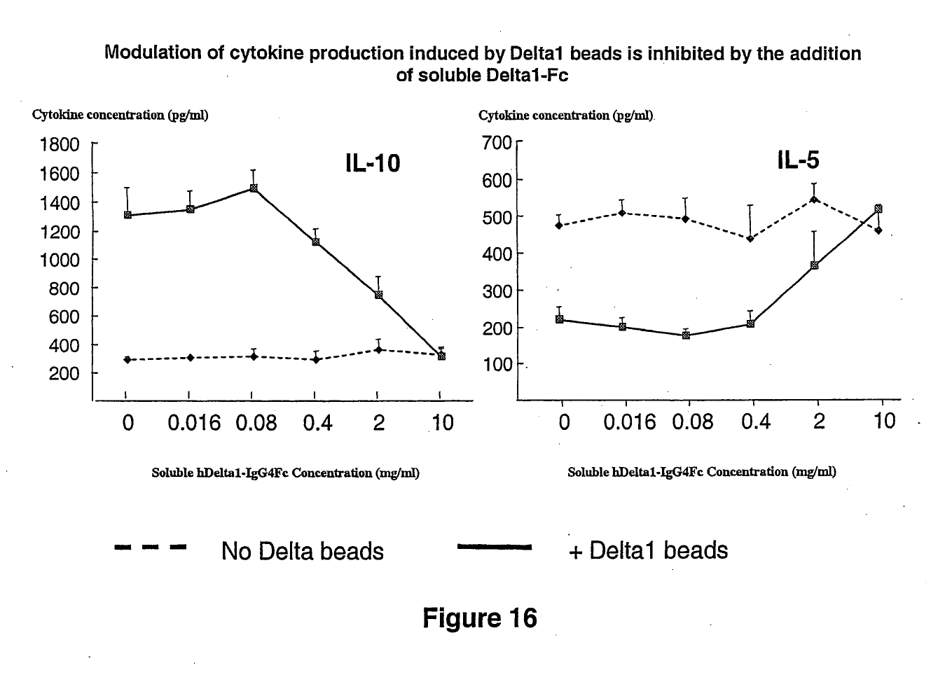

Results showing the effect of increasing concentrations of added soluble hDelta1-IgG4Fc are shown in FIG. 16.

As can be seen from these results, bead-immobilised human Delta1 enhances IL-10 production by activated human CD4+ T cells. This effect was inhibited when soluble hDelta1-IgG4Fc was added into the culture medium.

### Example 6

### The Modulation of Cytokine Production Induced by Delta1 Beads is Inhibited by the Addition of Soluble Notch1 EC Domain/Fc Fusion Protein

Human peripheral blood mononuclear cells (PBMC) were purified from blood using Ficoll-Paque separation medium (Pharmacia). Briefly, 28 ml of blood were overlaid on 21 ml of Ficoll-Paque separation medium and centrifuged at 18-20° C. for 46 minutes at 400 g. PBMC were recovered from the interface and washed 3 times before use for CD4+ T cell purification.

The CD4+ T cells were incubated in triplicates in a 96-well-plate (flat bottom) at 10⁵ CD4/well/200 μl in RPMI medium containing 10% FCS, glutamine, penicillin, streptomycin and β₂-mercaptoethanol.

Cytokine production was induced by stimulating the cells with anti-CD3/CD28 T cell expander beads from Dynal at a 1:1 ratio (bead/cell) in the presence of beads coated with hDelta1-IgG4Fc fusion protein (Example 1 above) at a 5:1 ratio (beads/cell). In some wells, increasing amounts of soluble rat Notch1 extracellular domain-hIgG1 fusion protein (R&D Systems, Catalog No 1057-TK) were also added.

The supernatants were removed after 3 days of incubation at 37° C./5% CO₂/humidified atmosphere and cytokine production was evaluated by ELISA using Pharmingen kits OptEIA Set human IL10 (Catalog No. 555157), OptEIA Set human IL-5 (Catalog No. 555202) for IL-10 and IL-5 respectively according to the manufacturer's instructions.

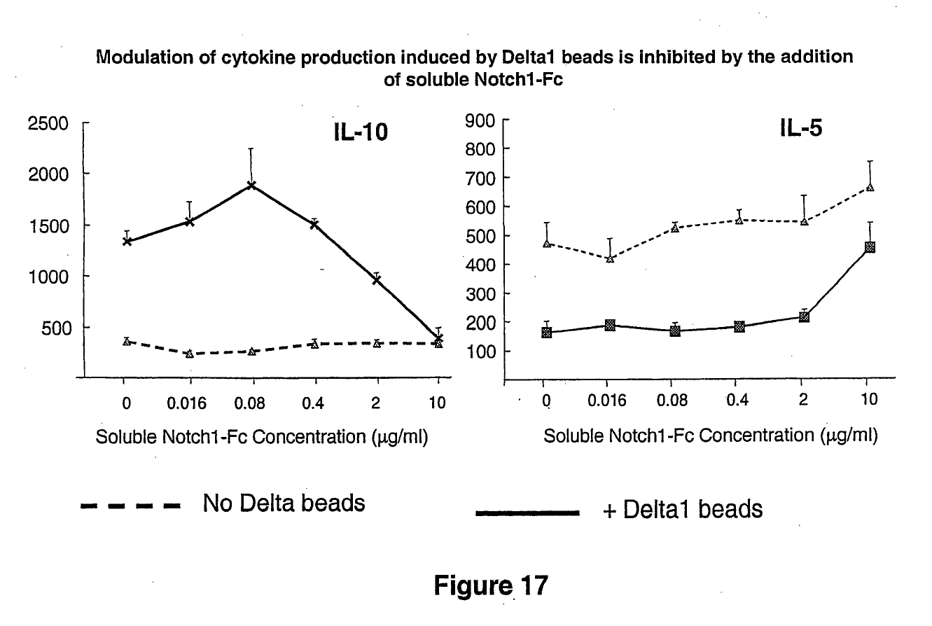

Results showing the effect of increasing concentrations of added soluble rat Notch1 EC-hIgG1Fc fusion protein are shown in FIG. 17.

As can be seen from these results, bead-immobilised human Delta1-Fc enhances IL-10 production by activated human CD4+ T cells. This effect was inhibited when soluble rat Notch1-hIgG1Fc was added into the culture medium.

Example 7

### Preparation of Inhibitor of Notch Signalling: Truncated Human Jagged1 Fusion Protein (hJagged1EGF1&2-IgG4Fc)

A fusion protein capable of acting as an inhibitor of Notch signalling comprising human jagged1 sequence up to the end of EGF2 (leader sequence, amino terminal, DSL, EGF1+2) fused to the Fc domain of human IgG4 (“hJagged1(EGF1+2)-IgG4Fc”) was prepared by inserting a nucleotide sequence coding for human Jagged1 from ATG through to the end of the second EGF repeat (EGF2) into the expression vector pCONγ (Lonza Biologics, Slough, UK) to add the IgG4 Fc tag. The full fusion protein was then shuttled into the Glutamine Synthetase (GS) selection system vector pEE14.4 (Lonza Biologics). The resulting construct was transfected and expressed in CHO-K1 cells (Lonza Biologics).

1. Cloning

i) Preparation of DNA—pDEV 47 and pDEV20

Human Jagged1 was cloned into pcDNA3.1 (Invitrogen) to give plasmid pLOR47. The Jagged 1 sequence from pLOR47 was aligned against full length human jagged1 (GenBank U61276) and found to have only a small number of apparently silent changes.

Plasmid pLOR47 was then modified to remove one of two DraIII sites (whilst maintaining and replacing the amino acid sequence for full extracellular hJagged1) and add a BsiWI site after for ease of subsequent cloning. The resulting plasmid was named pDEV20.

Plasmid pLOR47 was cut with DraIII. This removed a 1.7 kb fragment comprising the 3′ end of the extracellular, the transmembrane and intracellular regions of hJagged1 as well as part of the vector sequence leaving a larger fragment of 7.3 kbp of the main vector backbone with almost all of the extracellular region (EC) of hJagged1. The cut DNA was run out on an agarose gel, the larger fragment excised and gel purified using a Qiagen QIAquick™ Gel Extraction Kit (cat 28706) according to the manufacturer's instructions.

A pair of oligonucleotides were ordered such that when ligated together gave a double stranded piece of DNA that had a compatible sticky end for DraIII at the 5′ end and recreated the original restriction site. This sequence was followed by a BsiWI site then another compatible sticky end for DraIII at the 3′ end that did not recreate the restriction site.

| ie | DraIII BsiWI DraIII | |
|---|---|---|
| | gtg ctg tta ccc gta cgg ta | (SEQ ID NO: |
| | gaa cac gac aat ggg cat gc | 6) |

This oligo pair was then ligated into the DraII cut pLOR47 thus maintaining the 5′ DraIII site, inserting a BsiWI and eliminating the 3′DraIII site. The resulting plasmid was named pDEV20.

ii) Preparing hJagged1 IgG4 FC Fusion DNA:

A three fragment ligation was necessary to reassemble full hJagged1 EC sequence with addition of a modified 5′ Kozak sequence and 5′ end repair together with repair of 3′end.

Fragment 1: EC hJagged Sequence

pDev 20 was cut RsrII-DraIII giving rise to 3 fragments; 1270+2459+3621 bp. The fragments were run out on an agarose gel, the 2459 bp band excised and the DNA gel purified using a Qiagen QIAquick™ Gel Extraction Kit (cat 28706) according to the manufacturer's instructions. This contained hJagged1 sequence—with loss of 3′ sequence (up to the RsrII site) and loss of some 5′sequence at the end of the EC region.

Fragment 2: Modified Kozak Sequence

pUC19 (Invitrogen) was modified to insert new restriction enzyme sites and also introduce a modified Kozak with 5′ hJagged1 sequence. The new plasmid was named pLOR49. pLOR49 was created by cutting pUC19 vector HindIII EcoRI and ligating in 4 oligonucleotides (2 oligo pairs).

One pair has a HindIII cohesive end followed by an optimal Kozac and 5′hJagged1 sequence followed by RsrII cohesive end.

| | | |
|---|---|---|
| ie | HindIII optimal Kozak + 5′ hJagged1 sequence RsrII | |
| | ag ctt gcc gcc acc atg ggt tcc cca cgg aca cgc ggc cg | (SEQ ID NO: 7) |
| | a cgg cgg tgg tac cca agg ggt gcc tgt gcg ccg gcc ag | |

The other pair has a cohesive RsrII end then DraIII, KpnI, BsiWI sites followed by a cohesive EcoRI site.

| | | |
|---|---|---|
| ie | RsrII DraIII KpnI BsiWI EcoRI | |
| | gtc cgc acc ttg tgg gta ccc gta cgg | (SEQ ID NO: 8) |
| | gcg tgg aac acc cat ggg cat gcc tta a | |

pLOR49 thus is a pUC19 back bone with the HindIII site followed by optimal Kozac and 5′hJagged1 sequence and introduced unique RsrII, DraIII, KpnI, BsiWI sites before recreating the EcorI site.

Plasmid pLOR49 was then cut RsrII-BsiWI to give a 2.7 kbp vector backbone fragment that was run out on an agarose gel, the band excised and the DNA gel purified using a Qiagen QIAquick™ Gel Extraction Kit (cat 28706) according to the manufacturer's instructions.

Fragment 3: Generation of 3′ hJagged1 EC with BsiWI Site PCR Fragment

pLOR47 was used as a template for PCR to amplify up hJagged1 EC and add a 3′ BsiWI site.
5′ primer from RsrII site of hJagged I
3′ site up to end of hJagged1 EC with BsiWI site stitched on 3′

The resulting fragment was cut with DraIII and BsiWI to give a fragment around 600 bp. This was run out on an agarose gel, the band excised and the DNA gel purified using a Qiagen QIAquick™ Gel Extraction Kit (cat 28706) according to the manufacturer's instructions.

The three fragments described above;
1) 2459 bp h Jagged1 fragment from pDev 20 cut RsrII-DraIII
2) 2.7 kbp optimised Kozak and 5′ hJagged1 from Lor 49 cut RsrII-BsiWI
3) 600 bp 3′EC hJagged1 PCR fragment cut DraIII-BsiWI
   were then ligated together to give plasmid pDEV21.
   iii) Further ligation (PDEV10):

To exclude any extraneous sequences a further 3 fragment ligation was carried out to drop straight into the vector pCONγ 4 (Lonza Biologics, Slough, UK).

Fragment 1: Plasmid pDEV21-4 was cut HindIII-BglII to give 4958 bp+899 bp fragments. These were run out on an agarose gel, the smaller 889 bp fragment band was excised and the DNA gel purified using a Qiagen QIAquick™ Gel Extraction Kit (cat 28706) according to the manufacturer's instructions.

Fragment 2: pCONγ 4 (Lonza Biologics) was cut Hind 1′-ApaI to give a 6602 bp vector fragment—missing the first 5 amino acids of IgG4 FC. The fragment band was excised and the DNA gel purified using a Qiagen QIAquick™ Gel Extraction Kit (cat 28706) according to the manufacturer's instructions.

Fragment 3: A linker oligonucleotide pair was ordered to give a tight junction between the end of hJagged1 EGF2 and the 3′ start of IgG4 FC, with no extra amino acids introduced.

| | | |
|---|---|---|
| ie BglII D L A S T K G ApaI | | DL = hJagged1 sequence |
| gat ctc gct tcc acc aag ggc c | (SEQ ID NO: 9) | remainder = IgG4 FC sequence |
| ag cga agg tgg ttc | | |

The three fragments described above;
1. 899 bp hJagged1 fragment pDEV21-4 cut HindIII-BglII
2. 6602 bp pConGamma vector backbone cut HindIII ApaI
3. oligo linker BglII-ApaI
   were ligated together to give plasmid pDEV10.

Ligated DNA was transformed into competent DH5alpha (Invitrogen), plated onto LB amp paltes and incubated at 37 degres overnight. A good ratio was evident between control and vector plus insert pates therefore only 8 colonies were picked into 10 ml LB amp broth and incubated at 37 overnight. Glycerol broths were made and the bacterial pellets were frozen at −20 degrees. Later plasmid DNA was extracted using Qiagen miniprep spin kit and were diagnostically digested with ScaI. Clones 2, 4, and 5 looked correct so clone 2 was steaked onto LB Amp plates and inoculate 1/100 into 120 ml LB+amp broth. Plates and broths were incubated at 37 degrees overnight. Glycerol broths were made from the broths and pellets frozen to maxiprep later. Plasmid DNA was extracted Clontech Maxiprep, diagnostic digests were set up with ScaI and the DNA was diluted for quantification and quality check by UV spectrophotometry.

iv) pDev11 Cloning:

The coding sequence for hJagged1 EGF1+2 IgG4 FC fusion was shuttled out of pCONγ 4 (Lonza Biologics) into pEE 14.4 (Lonza Biologics) downstream of the hCMV promoter region (hCMV-MIE) and upstream of SV40 polyadenylation signal, to enable stable cell lines to be selected using the GS system (Lonza Biologics).

Plasmid pEE14.4 contains the GS minigene—(GS cDNA which includes the last intron and polylinker adenylation signals of the wild type hamster GS gene under the control of the late SV40 promoter) which encodes the GS gene required for selection in glutamine free media.

v) Insert:

pDEV10 clone 2 was cut HindIII-EcoRI giving rise to 2 fragment s 5026 bp+2497 bp. The 2497 bp contained the coding sequence for hJagged1 EGF1+2 IgG4 FC fusion and so was excised from an agarose gel and the DNA gel purified using a Qiagen QIAquick™ Gel Extraction Kit (cat 28706) according to the manufacturer's instructions.

vi) Vector:

pEE14.4 (Lonza Biologics) was cut HindIII-EcoRI to remove the IgG4 FC sequence giving 2 fragments 5026 bp+1593 bp. The larger 5026 bp fragment was excised from an agarose gel and the DNA gel purified using a Qiagen QIAquick™ Gel Extraction Kit (cat 28706) according to the manufacturer's instructions.

The pEE14.4 vector backbone and the hJagged1 EGF1+2 IgG4 FC fusion insert were ligated to give the final transfection plasmid pDEV11.

The ligation was transformed into DH5 a cells, streaked onto LB+Ampicillin (100 ug/ml) plates and incubated at 37° C. overnight. Colonies were picked from the plates into 7 ml LB+Ampicillin (100 ug/ml) and grown up shaking overnight at 37° C. Glycerol broths were made and the plasmid DNA was purified from the cultures using a Qiagen Qiaquick Spin Miniprep kit (cat 27106) according to the manufacturer's instructions. The DNA was then diagnostically digested with SapI.

vii) Maxiprep for Transfection:

A correct clone (clone 1) was chosen and 100 ul of the glycerol stock was inoculated into 100 ml LB+Ampicillin (100 ug/ml), and also streaked out onto LB+Ampicillin (100 ug/ml) plates. Both plate and broth were incubated at 37° C. overnight.

The plates showed pure growth; therefore the culture was maxi-prepped using a Clontech Nucleobond Maxi Kit (cat K3003-2) according to the manufacturer's instructions. The final DNA pellet was resuspended in 500 ul dH₂O.

A sample of pLOR11 clone 1 DNA was then diluted and the concentration and quality of DNA assessed by UV spectrophotometry. A sample was also diagnostically digested with SapI, and gave bands of the correct size.

viii) Linearisation of DNA:

Approx 100 ug pDev11 Clone 1 DNA was linearised with restriction enzyme Pvu I.

The resulting DNA preparation was cleaned up using phenol/chloroform/IAA extraction followed by ethanol wash and precipitation inside a laminar flow hood. The pellets were resuspended in sterile water. Linearisation was checked by agarose gel electrophoresis while quantification and quality were assessed by UV spectrophotometry at 260 and 280 nm.

2. Transfection

40 ug linearised DNA (pDev11 Clone 1) and 1×10⁷ CHO-K1 cells (Lonza) were mixed in 500 ul of serum free DMEM in a 4 mm cuvette, at room temp. The cells were then electroporated at 975 uF 280 volts, washed out into 60 ml of non-selective DMEM (DMEM/glut/10% FCS).

From this dilution 6×96 well pates were inoculated with 50 ul per well. A ¼ dilution of the original stock was made and from this 8×96 well pates were inoculated with 50 ul per well. A further 1/10 dilution was made from the second stock, and from this 12×96 well pates were inoculated with 50 ul per well.

Plates were incubated at 37 degrees C. 5% CO₂ overnight. After 24 hours the media was removed and replaced with 200 ul of selective media (25 uM L-MSX).

Between 4-6 weeks post transfection media was removed from the plates for analysis by IgG4 sandwich ELISA. Selective media were replaced. Positive clones were identified, passaged and expanded in selective media 25 um L-MSX.

3. Expression Cells were grown in selective DMEM (25 um L-MSX) until semi-confluent. The media was then replaced with serum free media (UltraCHO; BioWhittaker) for 3-5 days. Protein (hJagged1EGF1+2-IgG4Fc fusion protein) was purified from the resulting media by FPLC.

Amino Acid Sequence of the Expressed Fusion Protein (hJagged1 EGF1+2 IgG4 FC):

| | | | | | | | |
|---|---|---|---|---|---|---|---|
| 1 | mrsprtrgrs | grplslllal | lcalrakvcg | asgqfeleil | smqnvngelq | ngnccggarn | (SEQ ID NO:10) |
| 61 | pgdrkctrde | cdtyfkvclk | eyqsrvtagg | pcsfgsgstp | viggntfnlk | asrgndpnri | |
| 121 | vlpfsfawpr | sytllveawd | ssndtvqpds | iiekashsgm | inpsrqwqtl | kqntgvahfe | |
| 181 | yqirvtcddy | yygfgcnkfc | rprddffghy | acdqngnktc | megwmgpecn | raicrqgcsp | |
| 241 | khgscklpgd | crcqygwqgl | ycdkciphpg | cvhgicnepw | qclcetnwgg | qlcdkdlvra | |
| 301 | stkgpsvfpl | apcsrstses | taalgclvkd | yfpepvtvsw | nsgaltsgvh | tfpavlqssg | |
| 361 | lyslssvvtv | pssslgtkty | tcnvdhkpsn | tkvdkrvesk | ygppcpscpa | peflggpsvf | |
| 421 | lfppkpkdtl | misrtpevtc | vvvdvsqedp | evqfnwyvdg | vevhnaktkp | reeqfnstyr | |
| 481 | vvsvltvlhq | dwlngkeykc | kvsnkglpss | iektiskakg | qprepqvytl | ppsqeemtkn | |
| 541 | qvsltclvkg | fypsdiavew | esngqpenny | kttppvldsd | gsfflysrlt | vdksrwqegn | |
| 601 | vfscsvmhea | lhnhytqksl | slslgk | | | | |

Bold = hJagged1 extracellular domain leader sequence, amino terminal region, DSL and EGF 1 + 2, Underlined = IgG4 Fc sequence

The protein is believed to exist as a dimer linked by cysteine disulphide bonds, with cleavage of the signal peptide.

### Example 8

### The Modulation of Cytokine Production Induced by Delta1 Beads is Inhibited by the Addition of Soluble Jagged1 (2EGF Truncation)/Fc Fusion Protein

Human peripheral blood mononuclear cells (PBMC) were purified from blood using Ficoll-Paque separation medium (Pharmacia). Briefly, 28 ml of blood were overlaid on 21 ml of Ficoll-Paque separation medium and centrifuged at 18-20° C. for 40 minutes at 400 g. PBMC were recovered from the interface and washed 3 times before use for CD4+ T cell purification.

The CD4+ T cells were incubated in triplicates in a 96-well-plate (flat bottom) at 10⁵ CD4/well/2001 μl in RPMI medium containing 10% FCS, glutamine, penicillin, streptomycin and β₂-mercaptoethanol.

Cytokine production was induced by stimulating the cells with anti-CD3/CD28 T cell expander beads from Dynal at a 1:1 ratio (bead/cell) in the presence of beads coated with hDelta1-IgG4Fc fusion protein (Example 1 above) at a 5:1 ratio (beads/cell). In some wells, increasing amounts of soluble Jagged-1 (2EGF)-hIgG1 fusion protein (hJagged1EGF1&2-IgG4Fc; prepared as described above) were also added.

The supernatants were removed after 3 days of incubation at 37° C./5% CO₂/humidified atmosphere and cytokine production was evaluated by ELISA using Pharmingen kits OptEIA Set human IL10 (Catalog No. 555157), OptEIA Set human IL-5 (Catalog No. 555202) for IL-10 and IL-5 respectively according to the manufacturer's instructions.

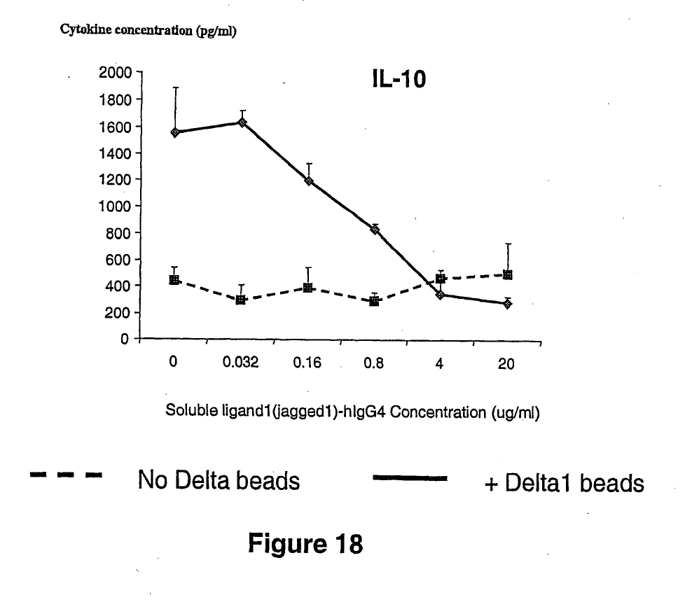

Results showing the effect of increasing concentrations of added soluble hJagged1EGF1&2-IgG4Fc are shown in FIG. 18.

As can be seen from these results, bead-immobilised human Delta1-Fc enhances IL-10 production by activated human CD4+ T cells. This effect was inhibited when soluble hJagged1EGF1&2-IgG4Fc fusion protein (hJ1E2Fc) was added into the culture medium.

### Example 9

### ELISA Assay Method for Detecting Notch Signalling Modulator Activity in Mouse CD4+ Cells

(i) CD4+ Cell Purification

Spleens were removed from female Balb/c mice 8-10 weeks old and passed through a 0.2 μM cell strainer into 20 ml R10F medium (R10F-RPMI 1640 media (Gibco Cat No 22409) plus 2 mM L-glutamine, 50 μg/ml Penicillin, 50 μg/ml Streptomycin, 5×10⁻⁵ M β-mercapto-ethanol in 10% fetal calf serum). The cell suspension was spun (1150 rpm 5 min) and the media removed.

The cells were incubated for 4 minutes with 5 ml ACK lysis buffer (0.15M NH₄Cl, 1.0M KHCO₃, O. 1 mM Na₂EDTA in double distilled water) per spleen (to lyse red blood cells). The cells were then washed once with R10F medium and counted. CD4+ cells were purified from the suspensions by positive selection on a Magnetic Associated Cell Sorter (MACS) column (Miltenyi Biotec, Bisley, UK: Cat No 130-042-401) using CD4 (L3T4) beads (Miltenyi Biotec Cat No 130-049-201), according to the manufacturer's directions.

(ii) Antibody Coating

The following protocol was used for coating 96 well flat-bottomed plates with antibodies.

The plates were coated with DPBS plus 1 μg/ml anti-hamsterIgG antibody (Pharmingen Cat No 554007) plus 1 μg/ml anti-IgG4 antibody. 100 μl of coating mixture was added per well. Plates were incubated overnight at 4° C. then washed with DPBS. Each well then received either 100 μl DPBS plus anti-CD3 antibody (1 μg/ml) or, 100 μl DPBS plus anti-CD3 antibody (1 μg/ml) plus hDelta1-IgG4Fc fusion protein (10 μg/ml). The plates were incubated for 2-3 hours at 37° C. then washed again with DPBS before cells (prepared as described above) were added.

iii) Investigation of Notch Signaling Inhibition

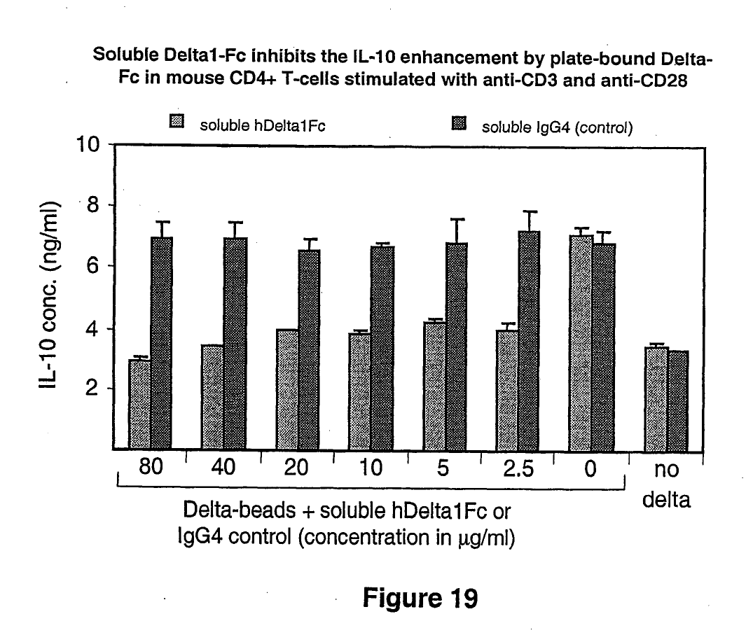

Mouse CD4+T-cells (prepared as above) were cultured at 2×10⁵/well on anti-CD3 coated plates with or without plate-bound hDelta1-IgG4Fc fusion protein (prepared as described above) and soluble anti-CD28. (Pharmingen, Cat No 553294, Clone No 37.51) at a final concentration of 2 μg/ml. Soluble hDelta1-IgG4Fc fusion protein was added into culture at the start at the concentrations shown and IL-10 was measured in supernatants on day 3 by ELISA using antibody pairs from R & D Systems (Abingdon, UK). The results (shown in FIG. 19) show that the increased IL-10 release induced by plate-bound hDelta1-IgG4Fc fusion protein is substantially reversed by all concentrations of soluble hDelta1-IgG4Fc fusion protein tested.

### Example 10

### CHO-N2 (N27) Luciferase Reporter Assay

A) Construction of Luciferase Reporter Plasmid 10xCBF1-Luc (pLOR91)

An adenovirus major late promoter TATA-box motif with BglII and HindIII cohesive ends was generated as follows:

BglII HindIII

| | |
|---|---|
| GATCTGGGGGGCTATAAAAGGGGGTA | (SEQ ID NO:11) |
| ACCCCCCGATATTTTCCCCCATTCGA | |

This was cloned into plasmid pGL3-Basic (Promega) between the BgiII and HindIII sites to generate plasmid pGL3-AdTATA.

A TP1 promoter sequence (TP1; equivalent to 2 CBF1 repeats) with BamH1 and BglII cohesive ends was generated as follows:

| BamH1 BglII | |
|---|---|
| 5′ GATCCCGACTCGTGGGAAAATGGGCGGAAGGGCACCGTGGGAAAATAGTA 3′ | (SEQ ID NO:12) |
| 3′ GGCTGAGCACCCTTTTACCCGCCTTCCCGTGGCACCCTTTTATCATCTAG 5′ | |

This sequence was pentamerised by repeated insertion into a BglII site and the resulting TP1 pentamer (equivalent to 10 CBF1 repeats) was inserted into pGL3-AdTATA at the BglII site to generate plasmid pLOR91.

B) Generation of a Stable CHO Cell Reporter Cell Line Expressing Full Length Notch2 and the 10xCBF1-Luc Reporter Cassette

A cDNA clone spanning the complete coding sequence of the human Notch2 gene (see, eg GenBank Accession No AF315356) was constructed as follows. A 3′ cDNA fragment encoding the entire intracellular domain and a portion of the extracellular domain was isolated from a human placental cDNA library (OriGene Technologies Ltd., USA) using a PCR-based screening strategy. The remaining 5′ coding sequence was isolated using a RACE (Rapid Amplification of cDNA Ends) strategy and ligated onto the existing 3′ fragment using a unique restriction site common to both fragments (Cla I). The resulting full-length cDNA was then cloned into the mammalian expression vector pcDNA3.1-V5-HisA (Invitrogen) without a stop codon to generate plasmid pLOR92. When expressed in mammalian cells, pLOR92 thus expresses the full-length human Notch2 protein with V5 and His tags at the 3′ end of the intracellular domain.

Wild-type CHO-K1 cells (eg see ATCC No CCL 61) were transfected with pLOR92 (pcDNA3.1-FLNotch2-V5-His) using Lipfectamine 2000™ (Invitrogen) to generate a stable CHO cell clone expressing full length human Notch2 (N2). Transfectant clones were selected in Dulbecco's Modified Eagle Medium (DMEM) plus 10% heat inactivated fetal calf serum ((HI)FCS) plus glutamine plus Penicillin-Streptomycin (P/S) plus 1 mg/ml G418 (Geneticin™-Invitrogen) in 96-well plates using limiting dilution. Individual colonies were expanded in DMEM plus 10%(HI)FCS plus glutamine plus P/S plus 0.5 mg/ml G418. Clones were tested for expression of N2 by Western blots of cell lysates using an anti-V5 monoclonal antibody (Invitrogen). Positive clones were then tested by transient transfection with the reporter vector pLOR91 (10xCBF1-Luc) and co-culture with a stable CHO cell clone (CHO-Delta) expressing full length human delta-like ligand 1 (DLL1; eg see GenBank Accession No AF196571). CHO-Delta cells were prepared in the same way as the CHO Notch 2 clone, but with human DLL1 used in place of Notch 2. A strongly positive clone was selected by Western blots of cell lysates with anti-V5 mAb.

One CHO-N2 stable clone, N27, was found to give high levels of induction when transiently transfected with pLOR91 (10xCBF1-Luc) and co-cultured with the stable CHO cell clone expressing fill length human DLL1 (CHO-Delta1). A hygromycin gene cassette (obtainable from pcDNA3.1/hygro, Invitrogen) was inserted into pLOR91 (10xCBF1-Luc) using BamHI and Sal1 and this vector (10xCBF1-Luc-hygro) was transfected into the CHO-N2 stable clone (N27) using Lipfectamine 2000 (Invitrogen). Transfectant clones were selected in DMEM plus 10%(HI)FCS plus glutamine plus P/S plus 0.4 mg/ml hygromycin B (Invitrogen) plus 0.5 mg/ml G418 (Invitrogen) in 96-well plates using limiting dilution. Individual colonies were expanded in DMEM plus 10%(HI)FCS plus glutamine plus P/S+0.2 mg/ml hygromycin B plus 0.5 mg/ml G418 (Invitrogen).

Clones were tested by co-culture with a CHO Delta (expressing full length human Delta1 (DLL1)). Three stable reporter cell lines were produced N27#11, N27#17 and N27#36. N27#11 was selected for further use because of its low background signal in the absence of Notch signalling, and hence high fold induction when signalling is initiated. Assays were set up in 96-well plates with 2×10⁴ N27#11 cells per well in 100 μl per well of DMEM plus 10%(HI)FCS plus glutamine plus P/S.

CHO-Delta cells (as described above) were maintained in DMEM plus 10% (HI)FCS plus glutamine plus P/S plus 0.5 mg/ml G418. Just prior to use the cells were removed from a T80 flask using 0.02% EDTA solution (Sigma), spun down and resuspended in 10 ml DMEM plus 10%(HI)FCS plus glutamine plus P/S. 10 μl of cells were counted and the cell density was adjusted to 5.0×10⁵ cells/ml with fresh DMEM plus 10%(HI)FCS plus glutamine plus P/S.

To set up the CHO-Delta antagonist assay, N27#11 cells (T₈₀ flask) were removed using 0.02% EDTA solution (Sigma), spun down and resuspended in 10 ml DMEM plus 10%(HI)FCS plus glutamine plus P/S. 10 μl of cells were counted and the cell density was adjusted to 2.0×10⁵ cells/ml with fresh DMEM plus 10%(HI)FCS plus glutamine plus P/S. The reporter cells were plated out at 100 μl per well of a 96-well plate (i.e. 2×10⁴ cells per well) and were placed in an incubator to settle down for at least 30 minutes.

hDelta1-IgG4Fc (soluble ligand inhibitor of Notch signalling) prepared as described above was diluted in complete DMEM to 5× final concentration required in the assay and 50 μl of diluted ligand was added to the 100 μl of N27#11 cells in a 96-well plate. Then 100 μl of CHO-Delta cells at 5×10⁵ cells/ml was added to initiate the signalling—giving a final volume of 250 μl in each well. The plate was then placed at 37° C. in an incubator overnight.

The following day 150 μl of supernatant was then removed from all the wells, 100 μl of SteadyGlo™ luciferase assay reagent (Promega) was added and the resulting mixture left at room temperature for 5 minutes. The mixture was then pipetted up and down 2 times to ensure cell lysis and the contents from each well were transferred to a white 96-well plate (Nunc). Luminescence was then read in a TopCount™ (Packard) counter.

Identical assays were performed using IgG4 as a control.

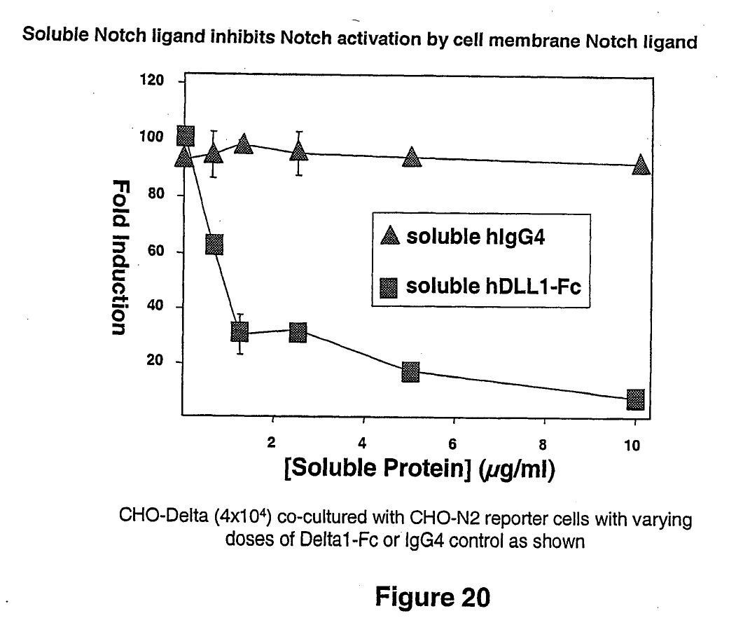

Results are shown in FIG. 20.

### Example 11

### Soluble hJagged1[2EGF]-IgG4Fc Antagonizes Notch Activation in CHO-N2 Cells

Antagonist Assay of Notch Signalling from CHO-Delta Cells

The procedure of Example 8 was repeated with use hJagged1 EGF 1 &2-IgG4Fc in place of hDelta1-IgG4Fc. Corresponding experiments were performed using hDelta1-IgG4Fc for comparison.

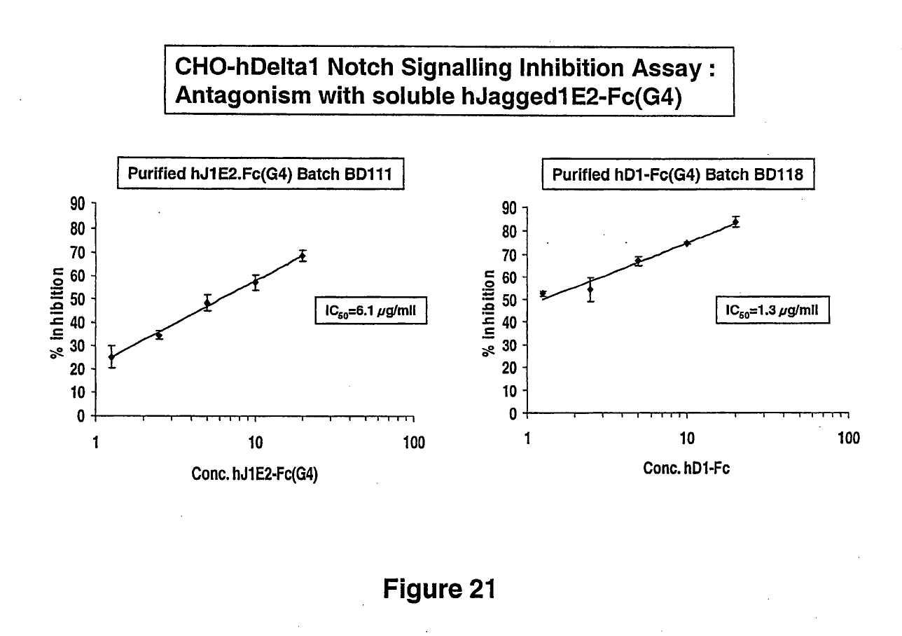

Results are shown in FIG. 21. It can be seen that the truncated Jagged protein with just 2 EGF repeats (hJagged1EGF1&2-IgG4Fc) provided substantially the same inhibition of Notch signalling as a corresponding protein comprising a full length human Delta1 extracellular domain (hDelta1-IgG4Fc).

### Example 12

### Antagonist Assays of Notch Signalling from mDLL1-Fc-Coated Dynabeads

A fusion protein was prepared corresponding to hDelta1-IgG4Fc as described above but using mouse Delta1 instead of human Delta1 (“mDelta1-IgG4Fc”).

Fc tagged Notch signalling modulators were immobilised on Streptavidin-Dynabeads (CELLection Biotin Binder Dynabeads [Cat. No. 115.21] at 4.0×10⁸ beads/ml from Dynal (UK) Ltd; “beads”) in combination with biotinylated α-IgG-4 (clone JDC14 at 0.5 mg/ml from Pharmingen [Cat. No. 555879]) as follows:

A volume of Dynabeads beads corresponding to the total number required was removed from a stock of beads at 4.0×10⁸ beads/ml. This was washed twice with 1 ml of PBS, and resuspended in a final volume of 100 μl of PBS containing a biotinylated anti-IgG4 antibody (clone JDC14 at 0.5 mg/ml from Pharmingen [Cat. No. 555879]) in a sterile Eppendorf tube and placed on shaker at room temperature for 30 minutes. The amount of biotinylated anti-IgG4 antibody needed to coat the beads was calculated relative to the fact that 1×10⁷ streptavidin Dynabeads bind a maximum of 2 μg of antibody.

After coating the beads with antibody they were washed 3 times with 1 ml of PBS and finally resuspended in mDelta1-IgG4Fc protein diluted in PBS. Beads were coated in a solution of 2 ug/ml protein (usually 5 μg of mDelta1-IgG4Fc protein was added per 10⁷ beads to be coated) and the ligand was allowed to bind to the beads in a 1 ml volume for 2 h at room temperature (or 4° C. overnight) on a rotary shaker to keep the beads in suspension. After coating the beads with mDelta1-IgG4Fc the beads were washed 3 times with 1 ml of PBS and finally resuspended complete DMEM at 2×10⁷ beads per ml so that addition of 100 μl of this to a well of 2×10⁴ reporter cells gave a ratio of 100 beads:cell.

To set up the bead antagonist assay, N27#11 cells (T₈₀ flask) were removed using 0.02% EDTA solution (Sigma), spun down and resuspended in 10 ml DMEM plus 10%(HI) FCS plus glutamine plus P/S. Ten μl of cells were counted and the cell density was adjusted to 2.0×10⁵ cells/ml with fresh DMEM plus 10%(HI) FCS plus glutamine plus P/S. The reporter cells were plated out at 100 μl per well of a 96-well plate (i.e. 2×10⁴ cells per well) and were placed in an incubator to settle down for at least 30 minutes.

Purified mDelta1-IgG4Fc was diluted in complete DMEM to 5×final concentration required in the assay and 50 μl of diluted ligand was added to the 100 μl of N27#11 cells in a 96-well plate. Then 100 μl of mDelta1-IgG4Fc Dynabeads at 2×10⁷ beads/ml was added to initiate the signalling—giving a final volume of 250 μl in each well. The plate was then placed at 37° C. in an incubator overnight.

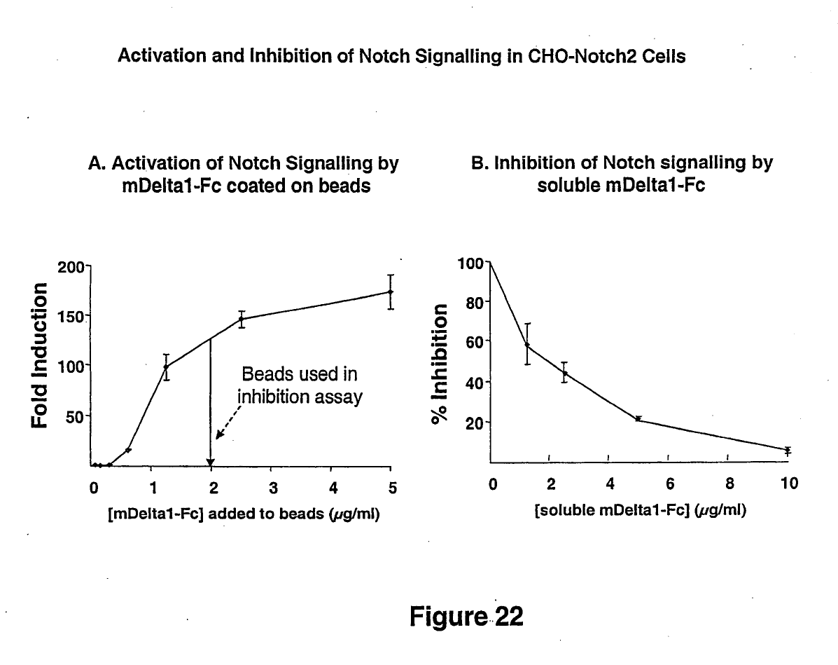

The following day 150 μl of supernatant was then removed from all the wells, 100 μl of SteadyGlo™ luciferase assay reagent (Promega) was added and the resulting mixture left at room temperature for 5 minutes. The mixture was then pipetted up and down 2 times to ensure cell lysis and the contents from each well were transferred to a 96 well plate (with V-shaped wells) and spun in a plate holder for 5 minutes at 1000 rpm at room temperature. The cleared supernatant was then transferred to a white 96-well plate (Nunc) leaving the beads pellet behind. Luminescence was then read in a TopCount™ (Packard) counter. Results are shown in FIG. 22.

### Example 13

### Soluble hJagged1EGF1&2-IgG4Fc Antagonizes Notch Activation in CHO-N2 Cells

Antagonist Assay of Notch Signalling from Delta Beads

The procedure of Example 8B was repeated with use of hJagged1EGF1 &2-IgG4Fc in place of mDelta1-IgG4Fc. Corresponding experiments were performed using hDelta1-IgG4Fc for comparison and using IgG4Fc as a control.

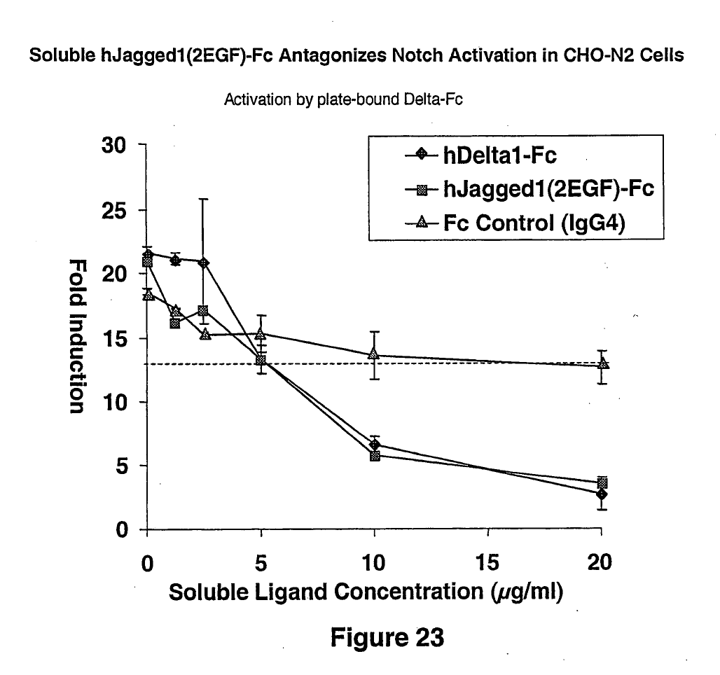

Results are shown in FIG. 23. It can be seen that the truncated Jagged protein with just 2 EGF repeats (hJagged1EGF1&2-IgG4Fc) provided substantially the same inhibition of Notch signalling as a corresponding protein comprising a full length human Delta1 extracellular domain (hDelta1-IgG4Fc). In both cases there was significant inhibition compared to control.

Example 14

### Reporter Assay Using Jurkat Cell Line

As Jurkat cells cannot be cloned by simple limiting dilution a methylcellulose-containing medium (ClonaCell™ TCS) was used with these cells.

Jurkat E6.1 cells (lymphoblast cell line; ATCC No TIB-152) were cloned using ClonaCell™ Transfected Cell Selection (TCS) medium (StemCell Technologies, Vancouver, Canada and Meylan, France) according to the manufacturer's guidelines.

Plasmid pLOR92 (prepared as described above) was electroporated into the Jurkat E6.1 cells with a Biorad Gene Pulser II electroporator as follows:

Actively dividing cells were spun down and resuspended in ice-cold RPMI medium containing 10% heat-inactivated FCS plus glutamine plus penicillin/streptomycin (complete RPMI) at 2.0×10⁷ cells per ml. After 10 min on ice, 0.5 ml of cells (ie 1×10⁷ cells) was placed into a pre-cooled 4 mm electroporation cuvette containing 20 μg of plasmid DNA (Endo-free Maxiprep DNA dissolved in sterile water). The cells were electroporated at 300 v and 950° F. and then quickly removed into 0.5 ml of warmed complete RPMI medium in an Eppendorf tube. The cells were spun for at 3000 rpm for 1 min in a microfuge and placed at 37° C. for 15 min to recover from being electroporated. The supernatant was then removed and the cells were plated out into a well of a 6-well dish in 4 ml of complete RPMI and left at 37° C. for 48 h to allow for expression of the antibiotic resistance marker.

After 48 h the cells were spun down and resupended in to 10 ml fresh complete RPMI. This was then divided into 10×15 ml Falcon tubes and 8 ml of pre-warmed ClonaCell-TCS medium was added followed by 1 ml of a 10×final concentration of the antibiotic being used for selection. For G418 selection the final concentration of G418 was 1 mg/ml so a 10 mg/ml solution in RPMI was prepared and 1 ml of this was added to each tube. The tubes were mixed well by inversion and allowed to settle for 15 min at room temperature before being plated out into 10 cm tissue culture dishes. These were then placed in a CO₂ incubator for 14 days when that were examined for visible colonies.

Macroscopically visible colonies were picked off the plates and these colonies were expanded through 96-well plates to 24-well plates to T25 flasks.

A clone was selected and transiently transfected with pLOR91 reporter contruct using Lipofectamine 2000 reagent and then plated out onto a 96-well plate containing plate-bound immobilised hDLL1-Fc (plates were coated by adding 10 μg of purified Notch ligand protein to each plate in sterile PBS; sealing the lid of the plate with parafilm and incubating at 4° C. overnight or at 37° C. for 2 hours and washing the plate with 200 μl of PBS before use).

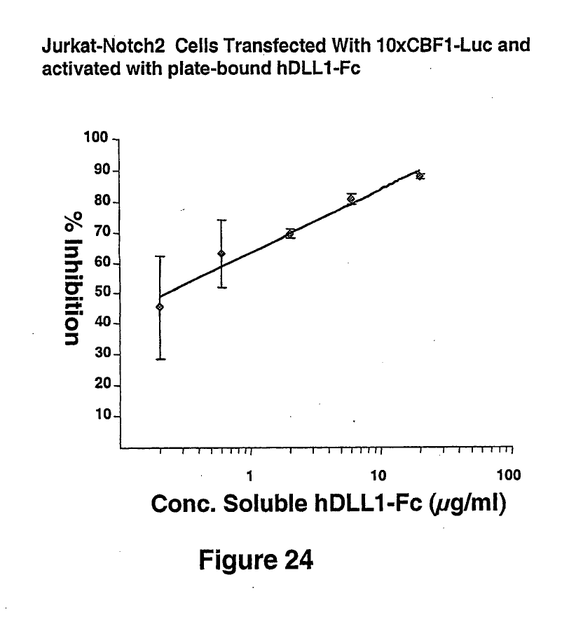

Luciferase assays were then conducted generally as described above. Results are shown in FIG. 24.

### Example 15

### Antagonism of A20-Delta and A20-Jagged Notch Signalling with Soluble hDLL-1 Fc

A20-Delta and A20-Jagged Cells

The IVS, IRES, Neo and pA elements were removed from plasmid pIRESneo2 (Clontech, USA) and inserted into a pUC cloning vector downstream of a chicken beta-actin promoter (eg see GenBank Accession No E02199). Mouse Delta-1 cDNA (eg see GenBank Accession No NM_{—}007865) was inserted between the actin promoter and IVS elements and a sequence with multiple stop codons in all three reading frames was inserted between the Delta and IVS elements.

The resulting construct was transfected into A20 cells using electroporation and G418 to provide A20 cells expressing mouse Delta1 on their surfaces (A20-Delta).

Corresponding cells (A20-Jagged) were prepared using human Jagged1 cDNA (see e.g. GenBank Accession No U61276).

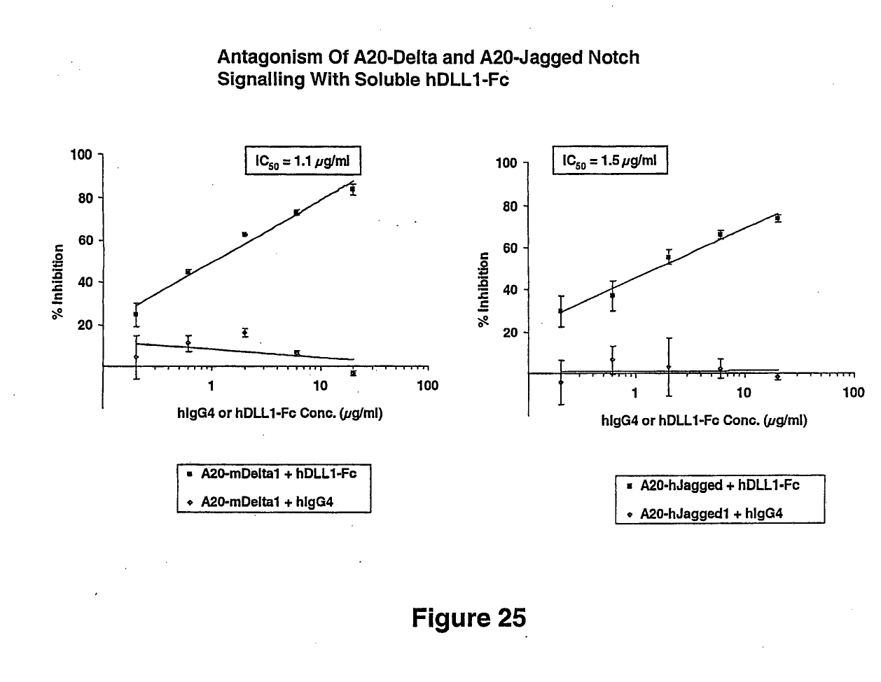

The procedure of Example was repeated using A20-Delta or A20-Jagged cells (1×10⁵ per well) in place of CHO-Delta cells. IgG4 was used as a control. Results are shown in FIG. 25. The results show that hDelta1-IgG4Fc was able to inhbit Notch signalling from Jagged1 as well as from Delta.

### Example 16

A fusion protein was prepared corresponding to hDelta1-IgG4Fc as described above but using human Jagged1 instead of human Delta1 (hJagged1-IgG4Fc).

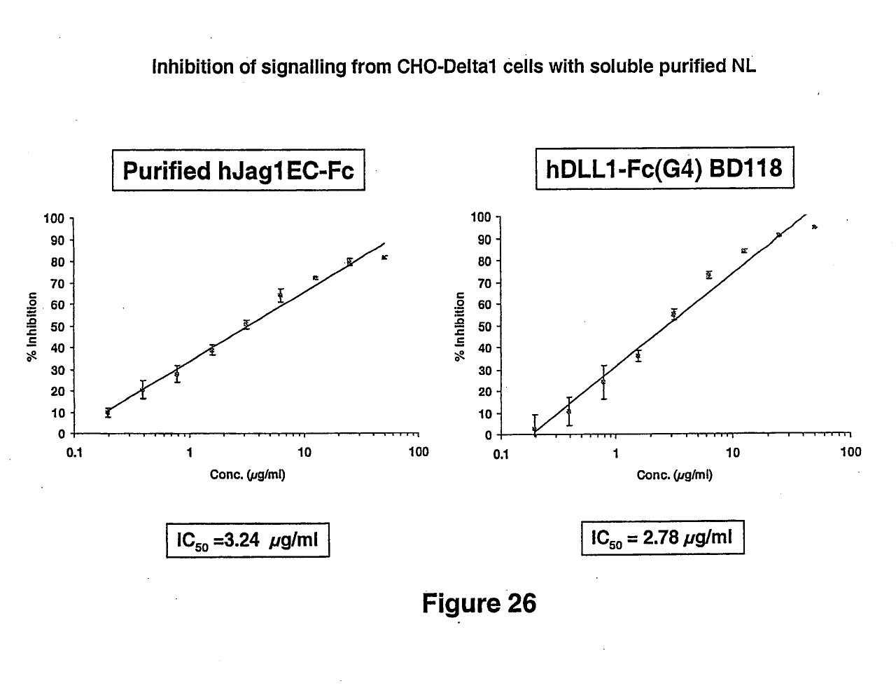

The procedure of Example 8 was repeated using hJagged1-IgG4Fc instead of hDelta1-IgG4Fc, and a corresponding repeat experiment was performed using hDelta1-IgG4Fc for comparison. Results are shown in FIG. 26.

### Example 17

### Notch Signalling Inhibitor Reduces Induction of Tolerance to KLH

BALB/c mice (eight per group) were treated intranasally with i) PBS, ii) KLH (10 mg) alone or iii) KLH (10 mg) plus hDelta1-IgG4Fc (100 mg). After 14 days, the mice were given KLH 50 mg/IFA s.c. 28 days after the initial KLH priming, mice were challenged in the ear with KLH 50 mg/IFA s.c and the ear immune response was measured with callipers as an increase in ear thickness due to the induced inflammatory reaction after 48 hours.

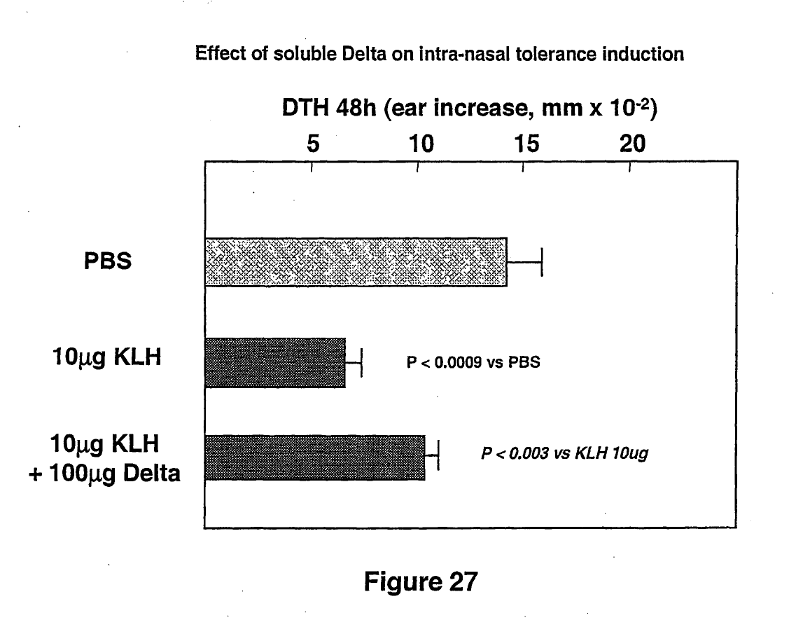

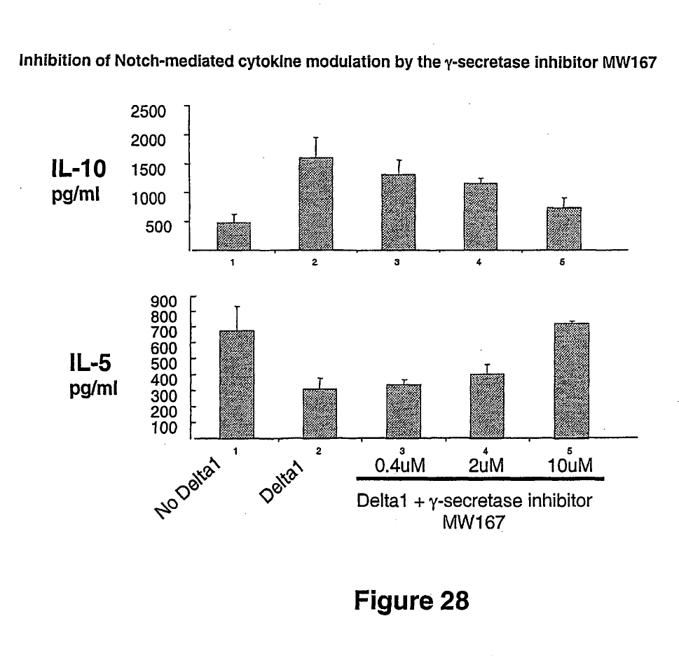

Results are shown in FIG. 27.

### Example 18

### Modulation of Cytokine Production by γ-Secretase Inhibitor in Human CD4+ T Cells

Human peripheral blood mononuclear cells (PBMC) were purified from blood using Ficoll-Paque separation medium (Pharmacia). Briefly, 28 ml of blood were overlaid on 21 ml of Ficoll-Paque separation medium and centrifuged at 18-20° C. for 40 minutes at 400 g. PBMC were recovered from the interface and washed 3 times before use for CD4+ T cell purification.

Human CD4+ T cells were isolated by positive selection using anti-CD4 microbeads from Miltenyi Biotech according to the manufacturer's instructions.

The CD4+ T cells were incubated in triplicates in a 96-well-plate (flat bottom) at 10⁵ CD4/well/200 ml in RPMI medium containing 10% FCS, glutamine, penicillin, streptomycin and b₂-mercaptoethanol.

Cytokine production was induced by stimulating the cells with anti-CD3/CD28 T cell expander beads from Dynal at a 1:1 ratio (bead/cell). Dynal beads coated with hDelta1-IgG4Fc fusion protein or control beads were added in some of the wells at a 5:1 ratio (beads/cell) and the γ-secretase inhibitor MW 167 (Calbiochem γ-secretase inhibitor II, Cat. No. 565755) was added variously (in DMSO) to final concentrations of 0, 0.4 mM, 2 mM and 10 mM.

The supernatants were removed after 3 days of incubation at 37° C./5% CO₂/humidified atmosphere and cytokine production was evaluated by ELISA using Pharmingen kits OptEIA Set human IL10 (catalog No. 555157), OptEIA Set human IL-5 (catalog No. 555202) for IL-10 and IL-5 respectively according to the manufacturer's instructions.

Results are shown in FIG. 22 from which it can be seen that the γ-secretase inhibitor substantially reversed a Delta-mediated increase in IL-10 expression and also substantially reversed a Delta-mediated reduction in IL-5 expression.

### Example 19

### Effect of γ-Secretase Inhibitor on Delta-Mediated Activation of Notch Signalling in Jurkat-N2 Cells

As Jurkat cells cannot be cloned by simple limiting dilution a methylcellulose-containing medium (ClonaCell™ TCS) was used with these cells.

Jurkat E6.1 cells (lymphoblast cell line; ATCC No TIB-152) were cloned using ClonaCell™ Transfected Cell Selection (TCS) medium (StemCell Technologies, Vancouver, Canada and Meylan, France) according to the manufacturer's guidelines.

Plasmid pLOR92 (prepared as described above) was electroporated into the Jurkat E6.1 cells with a Biorad Gene Pulser II electroporator as follows:

Actively dividing cells were spun down and resuspended in ice-cold RPMI medium containing 10% heat-inactivated FCS plus glutamine plus penicillin/streptomycin (complete RPMI) at 2.0×10⁷ cells per ml. After 10 min on ice, 0.5 ml of cells (ie 1×10⁷ cells) was placed into a pre-cooled 4 mm electroporation cuvette containing 20 μg of plasmid DNA (Endo-free Maxiprep DNA dissolved in sterile water). The cells were electroporated at 300 v and 950 μF and then quickly removed into 0.5 ml of warmed complete RPMI medium in an Eppendorf tube. The cells were spun for at 3000 rpm for 1 min in a microfuge and placed at 37° C. for 15 min to recover from being electroporated. The supernatant was then removed and the cells were plated out into a well of a 6-well dish in 4 ml of complete RPMI and left at 37° C. for 48 h to allow for expression of the antibiotic resistance marker.

After 48 h the cells were spun down and resupended into 10 ml fresh complete RPMI. This was then divided into 10×15 ml Falcon tubes and 8 ml of pre-warmed ClonaCell-TCS medium was added followed by 1 ml of a 10×final concentration of the antibiotic being used for selection. For G418 selection the final concentration of G418 was 1 mg/ml so a 10 mg/ml solution in RPMI was prepared and 1 ml of this was added to each tube. The tubes were mixed well by inversion and allowed to settle for 15 min at room temperature before being plated out into 10 cm tissue culture dishes. These were then placed in a CO₂ incubator for 14 days when that were examined for visible colonies.

Macroscopically visible colonies were picked off the plates and these colonies were expanded through 96-well plates to 24-well plates to T25 flasks—in complete RPMI containing 1 mg/ml G418.

The resulting clones were each transiently transfected with pLOR91 using Lipofectamine 2000 reagent (according to manufacturer's protocol) and then plated out onto a 96-well plate containing plate-bound immobilised hDelta1-IgG4Fc (prepared as described below). A well-performing clone (#24) was selected and used for further study.

10 μg of purified hDelta1-IgG4Fc fusion protein was added to sterile PBS in a sterile Eppendorf tube to give a final volume of 1 ml and 100 μl was added to wells of a 96-well tissue culture plate. The lid of the plate was sealed with parafilm and the plate was left at 4° C. overnight or at 37° C. for 2 hours. The protein was then removed and the plate was washed twice with 200 μl of PBS.

Assays were set up in the coated 96-well plates with 2×1 Jurkat cells per well in 100 μl per well of DMEM plus 10%(HI)FCS plus glutamine plus P/S. MW167 was diluted to 20 μM final concentration in complete RPMI from a 10 mM stock solution in DMSO. Control wells were set up with an equivalent dilution of DMSO alone. Plates were left in a CO₂ incubator overnight.

Supernatant was removed from all wells leaving 100 μl of cells plus medium behind and 100 μl of SteadyGlo™ luciferase assay reagent (Promega) was added and the cells were left at room temperature for 5 minutes. The mixture was pipetted up and down 2 times to ensure cell lysis and contents from each well were transferred into a white 96-well OptiPlate™ (Packard). Luminescence was measured in a TopCount™ counter (Packard).

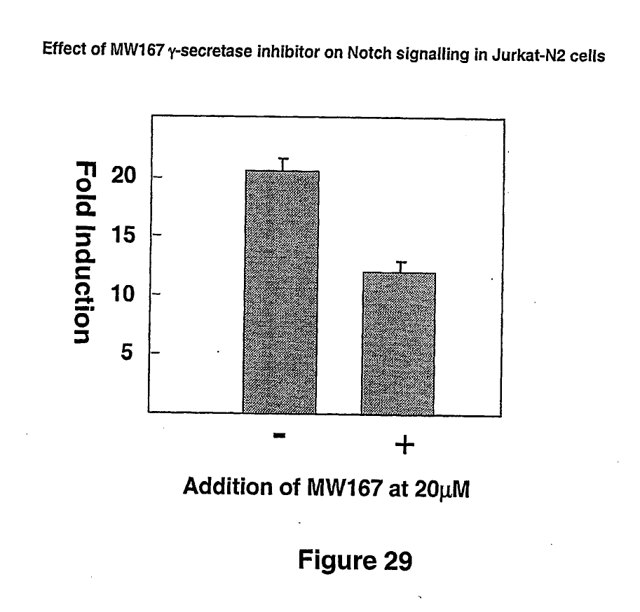

Results of sample assays using the Jurkat cells described above with plate-immobilised hDelta1-IgG4Fc fusion protein, are shown in FIG. 29 (expressed as fold activation of reporter activity compared to cells cultured in the absence of Delta).

### Example 20

### Preparation of Notch Inhibitor Construct with Human Jagged 1 DSL Domain Plus EGF Repeats 1-2 (“hJagged1[2EGF]-IgG4Fc”)

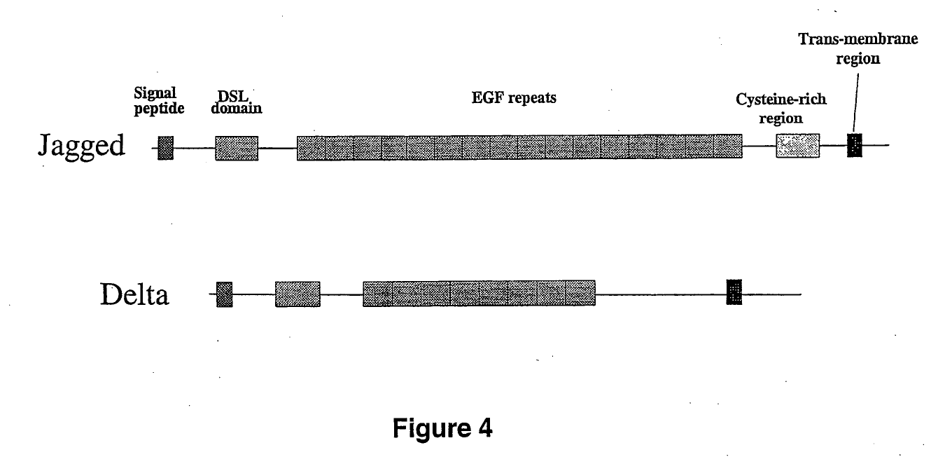

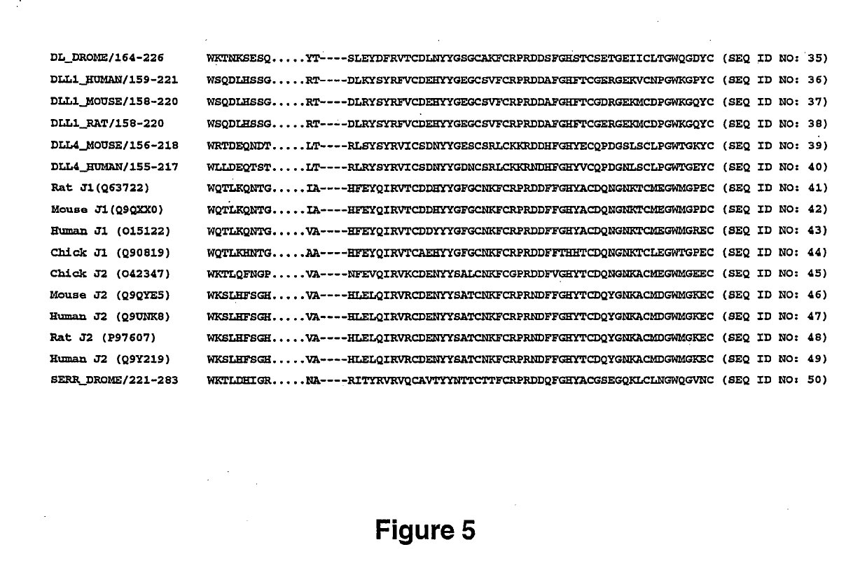

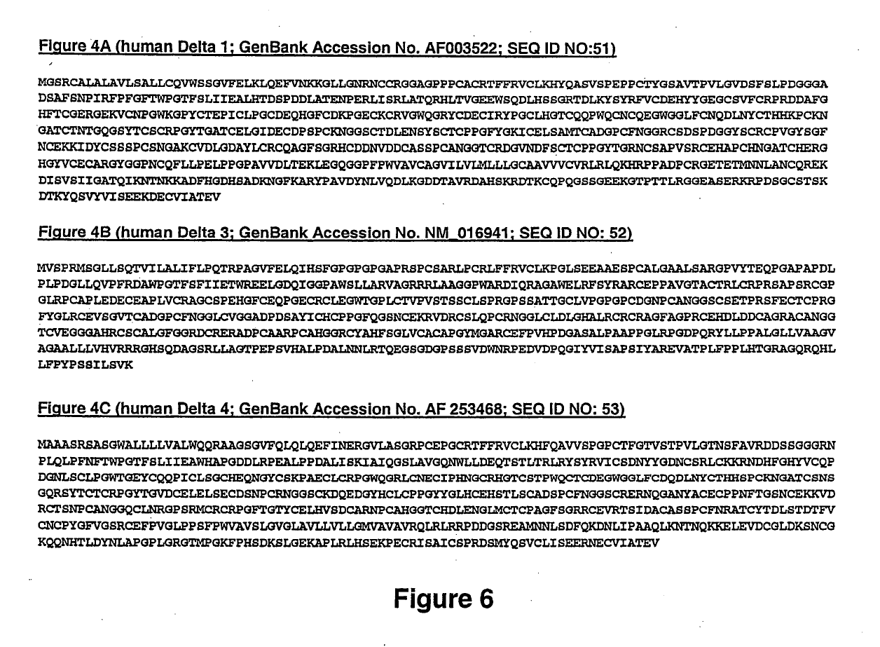

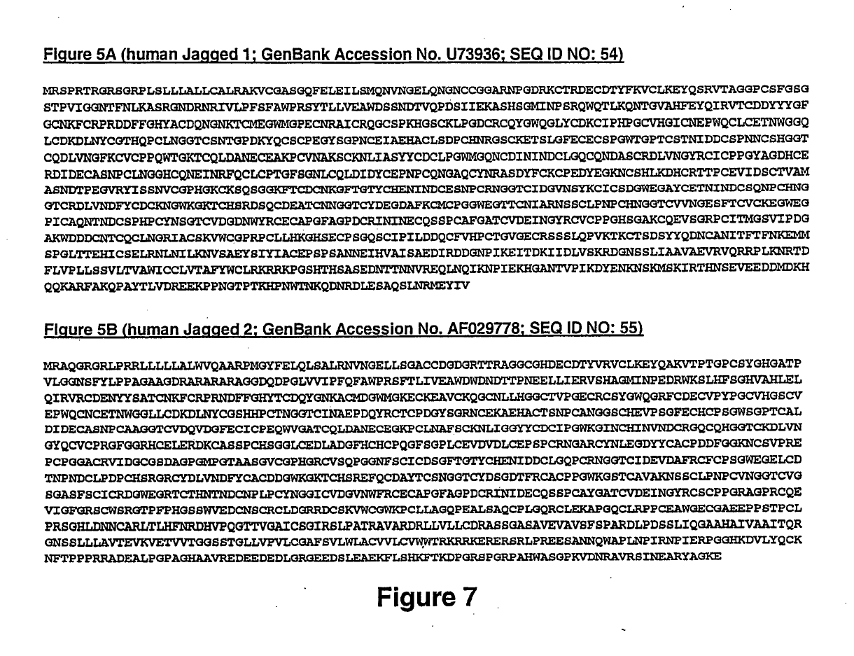

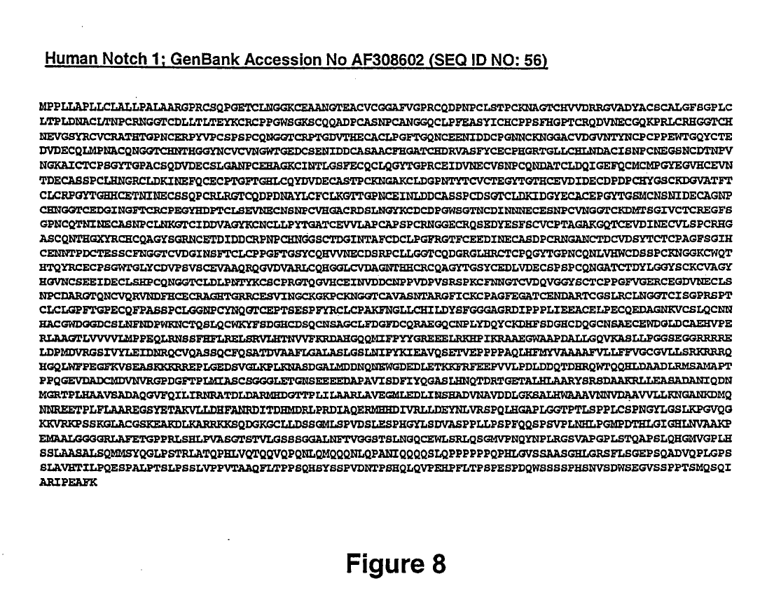

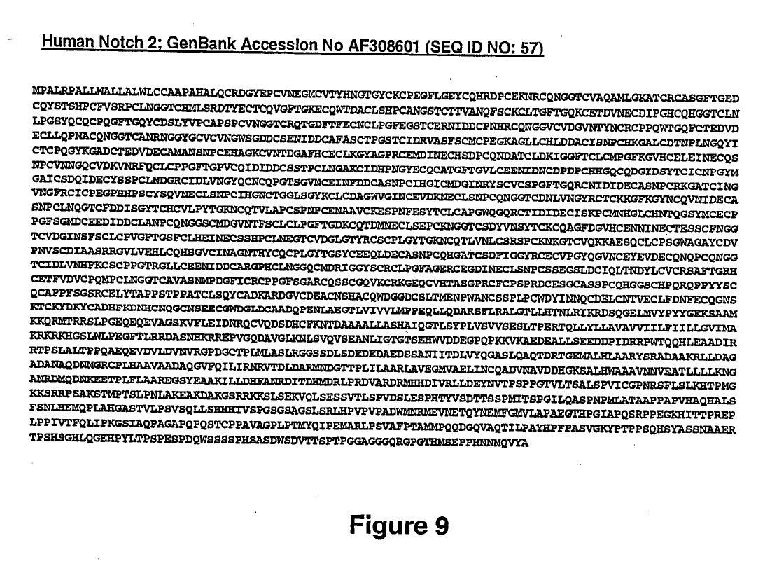

A human Jagged 1 (JAG-1) deletion coding for the DSL domain and the first two only of the naturally occurring EGF repeats (ie omitting EGF repeats 3 to 16 inclusive) was generated by PCR from a JAG-1 clone (for the sequence of the human JAG-1 see FIG. 4 and, for example, Genbank Accession No. U73936) using a primer pair as follows:

| | |
|---|---|
| EN0102f: | |
| CCAGGCAAGCTTATGGGTTCCCCACGGACGCGC | (SEQ ID NO:13) and |
| J1E2Fc4rev: | |
| CAGCTCTGTGACAAAGATCTCAATTACCTCGAGATCG | (SEQ ID NO:14) |

These primers generate a sequence that changes aa. 2 of the leader peptide region from R to G.

PCR conditions were:
1 cycle at 95° C./2 minutes;
18 cycles of (95° C./30 seconds, 60° C./30 seconds, 72° C./1½ minutes); and
1 cycle at 72° C./10 minutes.

The DNA was then isolated from a 1% agarose gel in 1×TBE (Tris/borate/EDTA) buffer.

pCONγ (Lonza Biologics, UK) was cut with HindIII and ApaI and the following adaptor oligonucleotide sequence was ligated to introduce a XhoI site then subsequently cloned in DH5α cells:

(SEQ ID NO:15)

AGCTTTCAGTTCTCGAGGGATCGGCTTCCACCAAGGGCC

pCONγX was cut with HindIII and XhoI then treated with shrimp alkaline phosphatase (Roche) and gel purified. The purified JAG-1 PCR product was cut with HindIII and XhoI and ligated into restricted pCONγX then subsequently cloned in DH5α cells (InVitrogen). Plasmid DNA was generated using a Qiagen Minprep kit (QIAprep™) according to the manufacturer's instructions and the identity of the PCR product was confirmed by sequencing.

The resulting construct (pCONγ hJ1E2) coded for the following JAG-1 amino acid sequence (SEQ ID NO: 16) fused to the IgG Fc domain encoded by the pCONγ vector.

MGSPRTRGRSGRPLSLLLALLCALRAKVCGASGQFELEILSMQNVNGELQNGNCCGGAR
NPGDRKCTRDECDTYFKVCLKEYQSRVTAGGPCSFGSGSTPVIGGNTFNLKASRGNDRN
RIVLPFSFAWPRSYTLLVEAWDSSNDTVQPDSIIEKASHSGMINPSRQWQTLKQNTGVA
HFEYQIRVTCDDYYYGFGCNKFCRPRDDFFGHYACDQNGNKTCMEGWMGPECNRAICRQ
GCSPKHGSCKLPGDCRCQYGWQGLYCDKCIPHPGCVHGICNEPWQCLCETNWGGQLCDK
DLNYEGS

(wherein the emboldened portion of the sequence which is single underlined is the DSL domain and the emboldened portions of the sequence which are double underlined are EGF repeats 1 and 2 respectively and the linker/hinge in italic).

DNA encoding the J1E2.Fc4 sequence was excised with EcoRI and HindIII and ligated into EcoRI and HindIII restricted pEE14.4. The resulting plasmid, pEE14.J1E2.Fc4, was cloned in DH5α (Invitrogen). Plasmid DNA was generated using a Qiagen Endofree Maxiprep kit (QIAprep™) according to the manufacturer's instructions and the identity of the product was confirmed by sequencing.

### Example 21

A series of truncations based on human Delta1 comprising varying numbers of EGF repeats was prepared as follows:

A) Delta 1 DSL Domain Plus EGF Repeats 1-2

A human Delta 1 (DLL-1) deletion coding for the DSL domain and the first two only of the naturally occurring EGF repeats (i.e. omitting EGF repeats 3 to 8 inclusive) was generated by PCR from a DLL-1 extracellular (EC) domain/VSHis clone (for the sequence of the human DLL-1 EC domain see Figures and, for example, Genbank Accession No. AF003522) using a primer pair as follows:

| | |
|---|---|
| DLac13: | |
| CACCAT GGGCAG TCGGTG CGCGCT GG | (SEQ ID NO:17) and |
| DLL1d3-8: | |
| GTAGTT CAGGTC CTGGTT GCAG | (SEQ ID NO:18) |

PCR conditions were:
1 cycle at 95° C./3 minutes;
18 cycles of (95° C./1 minute, 60° C./1 minute, 72° C./2 minutes); and
1 cycle at 72° C./2 minutes.

The DNA was then isolated from a 1% agarose gel in 1×U/V-Safe TAE (Tris/acetate/EDTA) buffer (MWG-Biotech, Ebersberg, Germany) and used as a template for PCR with the following primers:

| | |
|---|---|
| FcDL.4: | |
| CACCAT GGGCAG TCGGTG CGCGCT GG | (SEQ ID NO:19) and |
| FcDLLd3-8: | |
| GGATAT GGGCCC TTGGTG GAAGCG | (SEQ ID NO:20) |
| TAGTTC AGGTCC TGGTTG CAG | |

PCR conditions were:
1 cycle at 94° C./3 minutes;
18 cycles of (94° C./11 minute, 68° C./1 minute, 72° C./2 minutes); and
1 cycle at 72° C./10 minutes.

The fragment was ligated into pCRbluntII.TOPO (Invitrogen) and cloned in TOP10 cells (Invitrogen). Plasmid DNA was generated using a Qiagen Minprep kit (QIAprep™) according to the manufacturer's instructions and the identity of the PCR products was confirmed by sequencing.

An IgFc fusion vector pCONγ (Lonza Biologics, UK) was cut with ApaI and HindIII then treated with shrimp alkaline phosphatase (Roche) and gel purified.

The DLL-1 deletions cloned in pCRbluntII were cut with HindIII (and EcoRV to aid later selection of the desired DNA product) followed by ApaI partial restriction. The sequences were then gel purified and ligated into the pCONγ vector which was cloned into TOP10 cells.

Plasmid DNA was generated using a Qiagen Minprep kit (QIAprep™) according to the manufacturer's instructions.

The resulting construct (pCONγ hDLL1 EGF1-2) coded for the following DLL-1 amino acid sequence (SEQ ID NO: 21) fused to the IgG Fc domain encoded by the pCONγ vector.

MGSRCALALAVLSALLCQVWSSGVFELKLQEFVNKKGLLGNRNCCRGGAGPPPCACR
TFFRVCLKHYQASVSPEPPCTYGSAVTPVLGVDSFSLPDGGGADSAFSNPIRFPFGF
TWPGTFSLIIEALHTDSPDDLATENPERLISRLATQRHLTVGEEWSQDLHSSGRTDL
KYSYRFVCDEHYYGEGCSVFCRPRDDAFGHFTCGERGEKVCNPGWKGPYCTEPICLP
GCDEQHGFCDKPGECKCRVGWQGRYCDECIRYPGCLHGTCQQPWQCNCQEGWGGLFC
NQDLNY

(wherein the emboldened portion of the sequence which is single underlined is the DSL domain and the emboldened portions of the sequence which are double underlined are EGF repeats 1 and 2 respectively).
B) Delta 1 DSL Domain Plus EGF Repeats 1-3

A human Delta 1 (DLL-1) deletion coding for the DSL domain and the first three only of the naturally occurring EGF repeats (ie omitting EGF repeats 4 to 8 inclusive) was generated by PCR from a DLL-1 DSL plus EGF repeats 1-4 clone using a primer pair as follows:

| | |
|---|---|
| DLac13: | |
| CACCATGGGCAGTCGGTGCGCGCTGG | (SEQ ID NO:22) |
| and | |
| FcDLLd4-8: | |
| GGA TAT GGG CCC TTG | (SEQ ID NO:23) |
| GTG GAA GCC TCG TCA | |
| ATC CCC AGC TCG CAG | |

PCR conditions were:
1 cycle at 94° C./3 minutes;
18 cycles of (94° C./1 minute, 68° C./1 minute, 72° C./2.5 minutes); and
1 cycle at 72° C./10 minutes The DNA was then isolated from a 1% agarose gel in 1×U/V-Safe TAE (Tris/acetate/EDTA) buffer (MWG-Biotech, Ebersberg, Germany) and ligated into pCRbluntII.TOPO and cloned in TOP10 cells (Invitrogen). Plasmid DNA was generated using a Qiagen Minprep kit (QIAprep™) according to the manufacturer's instructions and the identity of the PCR products was confirmed by sequencing.

An IgFc fusion vector pCONγ (Lonza Biologics, UK) was cut with ApaI and HindIII then treated with shrimp alkaline phosphatase (Roche) and gel purified.

The DLL-1 deletions cloned in pCRbluntII were cut with HindIII followed by ApaI partial restriction. The sequences were then gel purified and ligated into the pCONγ vector which was cloned into TOP10 cells.

Plasmid DNA was generated using a Qiagen Minprep kit (QIAprep™) according to the manufacturer's instructions and the identity of the PCR products was confirmed by sequencing.

The resulting construct (pCONγ hDLL1 EGF1-3) coded for the following DLL-1 sequence (SEQ ID NO: 24) fused to the IgG Fc domain coded by the pCONγ vector.

MGSRCALALAVLSALLCQVWSSGVFELKLQEFVNKKGLLGNRNCCRGGAGPPPCACR
TFFRVCLKHYQASVSPEPPCTYGSAVTPVLGVDSFSLPDGGGADSAFSNPIRFPFGF
TWPGTFSLIIEALHTDSPDDLATENPERLISRLATQRHLTVGEEWSQDLHSSGRTDL
KYSYRFVCDEHYYGEGCSVFCRPRDDAFGHFTCGERGEKVCNPGWKGPYCTEPICLP
GCDEQHGFCDKPGECKCRVGWQGRYCDECIRYPGCLHGTCQQPWQCNCQEGWGGLFC
NQDLNYCTHHKPCKNGATCTNTGQGSYTCSCRPGYTGATCELGIDE

(wherein the emboldened portion of the sequence which is single underlined is the DSL domain and the emboldened portions of the sequence which are double underlined are EGF repeats 1 to 3 respectively).
C) Delta 1 DSL Domain Plus EGF Repeats 14

A human Delta 1 (DLL-1) deletion coding for the DSL domain and the first four only of the naturally occurring EGF repeats (ie omitting EGF repeats 5 to 8 inclusive) was generated by PCR from a DLL-1 EC domain/V5His clone using a primer pair as follows:

| | |
|---|---|
| DLac13: | |
| CACCAT GGGCAG TCGGTG CGCGCT GG | (SEQ ID NO:25) |
| and | |
| DLL1d5-8: | |
| GGTCAT GGCACT CAATTC ACAG | (SEQ ID NO:26) |

PCR conditions were:
1 cycle at 95° C./3 minutes;
18 cycles of (95° C./1 minute, 60° C./1 minute, 72° C./2.5 minutes); and
1 cycle at 72° C./10 minutes.

The DNA was then isolated from a 1% agarose gel in 1×U/V-Safe TAE (Tris/acetate/EDTA) buffer (MWG-Biotech, Ebersberg, Germany) and used as a template for PCR using the following primers:

| | |
|---|---|
| FcDL.4: | |
| CACCAT GGGCAG TCGGTG CGCGCT GG; | (SEQ ID NO:27) |
| and | |
| FcDLLd5-8: | |
| GGATAT GGGCCC TTGGTG GAAGCG GTCATG | (SEQ ID NO:28) |
| GCACTC AATTCA CAG | |

PCR conditions were:
1 cycle at 94° C./3 minutes;
18 cycles of (94° C./1 minute, 68° C./1 minute, 72° C./2.5 minutes); and
1 cycle at 72° C./10 minutes.

The fragment was ligated into pCRbluntII.TOPO and cloned in TOP10 cells (Invitrogen). Plasmid DNA was generated using a Qiagen Minprep kit (QIAprep™) according to the manufacturer's instructions and the identity of the PCR products was confirmed by sequencing.

An IgFc fusion vector pCONγ (Lonza Biologics, UK) was cut with ApaI and HindIII then treated with shrimp alkaline phosphatase (Roche) and gel purified.

The DLL-1 deletions cloned in pCRbluntII were cut with HindIII (and EcoRV to aid later selection of the desired DNA product) followed by ApaI partial restriction. The sequences were then gel purified and ligated into the pCONγ vector which was cloned into TOP10 cells.

Plasmid DNA was generated using a Qiagen Minprep kit (QIAprep™) according to the manufacturer's instructions and the identity of the PCR products was confirmed by sequencing.

The resulting construct (pCONγ hDLL1 EGF1-4) coded for the following DLL-1 sequence (SEQ ID NO: 29) fused to the IgG Fc domain coded by the pCONγ vector.

MGSRCALALAVLSALLCQVWSSGVFELKLQEFVNKKGLLGNRNCCRGGAGPPPCACR
TFFRVCLKHYQASVSPEPPCTYGSAVTPVLGVDSFSLPDGGGADSAFSNPIRFPFGF
TWPGTFSLIIEALHTDSPDDLATENPERLISRLATQRHLTVGEEWSQDLHSSGRTDL
KYSYRFVCDEHYYGEGCSVFCRPRDDAFGHFTCGERGEKVCNPGWKGPYCTEPICLP
GCDEQHGFCDKPGECKCRVGWQGRYCDECIRYPGCLHGTCQQPWQCNCQEGWGGLFC
NQDLNYCTHHKPCKNGATCTNTGQGSYTCSCRPGYTGATCELGIDECDPSPCKNGGS
CTDLENSYSCTCPPGFYGKICELSAMT

(wherein the emboldened portion of the sequence which is single underlined is the DSL domain and the emboldened portions of the sequence which are double underlined are EGF repeats 1 to 4 respectively).
D) Delta 1 DSL Domain Plus EGF Repeats 1-7

A human Delta 1 (DLL-1) deletion coding for the DSL domain and the first seven of the naturally occurring EGF repeats (ie omitting EGF repeat 8) was generated by PCR from a DLL-1 EC domain/V5His clone using a primer pair as follows:

| | |
|---|---|
| DLac13: | |
| CACCAT GGGCAG TCGGTG CGCGCT GG; | (SEQ ID NO:30) and |
| DLL1d8: | |
| CCTGCT GACGGG GGCACT GCAGTT C | (SEQ ID NO:31) |

PCR conditions were:
1 cycle at 95° C./3 minutes;
18 cycles of (95° C./1 minute, 68° C./1 minute, 72° C./3 minutes); and
1 cycle at 72° C./10 minutes.

The DNA was then isolated from a 1% agarose gel in 1×U/V-Safe TAE (Tris/acetate/EDTA) buffer (MWG-Biotech, Ebersberg, Germany) and used as a template for PCR using the following primers:

| | |
|---|---|
| FcDL.4: | |
| CACCAT GGGCAG TCGGTG CGCGCT GG; | (SEQ ID NO:32) and |
| FCDLLd8: | |
| GGATAT GGGCCC TTGGTG GAAGCC CTGCTG | (SEQ ID NO:33) |
| ACGGGG GCACTG CAGTTC | |

PCR conditions were:
1 cycle at 94° C./3 minutes;
18 cycles of (94° C./1 minute, 68° C./1 minute, 72° C./3 minutes); and
1 cycle at 72° C./10 minutes.

The fragment was ligated into pCRbluntII.TOPO and cloned in TOP10 cells (Invitrogen). Plasmid DNA was generated using a Qiagen Minprep kit (QIAprep™) according to the manufacturer's instructions and the identity of the PCR products was confirmed by sequencing.

An IgFc fusion vector pCONγ (Lonza Biologics, UK) was cut with ApaI and HindIII then treated with shrimp alkaline phosphatase (Roche) and gel purified.

The DLL-1 deletions cloned in pCRbluntII were cut with HindIII (and EcoRV to aid later selection of the desired DNA product) followed by ApaI partial restriction. The sequences were then gel purified and ligated into the pCONγ vector which was cloned into TOP10 cells.

Plasmid DNA was generated using a Qiagen Minprep kit (QIAprep™) according to the manufacturer's instructions and the PCR products were sequenced.

The resulting construct (pCONγ hDLL1 EGF1-7) coded for the following DLL-1 sequence (SEQ ID NO: 34) fused to the IgG Fc domain coded by the pCONγ vector.

MGSRCALALAVLSALLCQVWSSGVFELKLQEFVNKKGLLGNRNCCRGGAGPPPCACR
TFFRVCLKHYQASVSPEPPCTYGSAVTPVLGVDSFSLPDGGGADSAFSNPIRFPFGF
TWPGTFSLIIEALHTDSPDDLATENPERLISRLATQRHLTVGEEWSQDLHSSGRTDL
KYSYRFVCDEHYYGEGCSVFCRPRDDAFGHFTCGERGEKVCNPGWKGPYCTEPICLP
GCDEQHGFCDKPGECKCRVGWQGRYCDECIRYPGCLHGTCQQPWQCNCQEGWGGLFC
NQDLNYCTHHKPCKNGATCTNTGQGSYTCSCRPGYTGATCELGIDECDPSPCKNGGS
CTDLENSYSCTCPPGFYGKICELSAMTCADGPCFNGGRCSDSPDGGYSCRCPVGYSG
FNCEKKIDYCSSSPCSNGAKCVDLGDAYLCRCQAGFSGRHCDDNVDDCASSPCANGG
TCRDGVNDFSCTCPPGYTGRNCSAPVSR

(wherein the emboldened portion of the sequence which is single underlined is the DSL domain and the emboldened portions of the sequence which are double underlined are EGF repeats 1 to 7 respectively).
E) Transfection and Expression
i) Transfection and Expression of Constructs of Constructs A, C and D

Cos 1 cells were separately transfected with each of the expression constructs from Examples 1, 3 and 4 above (viz pCONγ hDLL1 EGF1-2, pCONγ hDLL1 EGF1-4, pCONγ hDLL1 EGF1-7) and pCONγ control as follows:

In each case 3×10⁶ cells were plated in a 10 cm dish in Dulbecco's Modified Eagle's Medium (DMEM)+10% Fetal Calf Serum (FCS) and cells were left to adhere to the plate overnight. The cell monolayer was washed twice with 5 ml phosphate-buffered saline (PBS) and cells left in 8 ml OPTIMEM™ medium (Gibco/Invitrogen). 12 μg of the relevant construct DNA was diluted into 810 μl OPTIMEM medium and 14 μl Lipofectamine2000™ cationic lipid transfection reagent (Invitrogen) was diluted in 810 μl OPTIMEM medium. The DNA-containing and Lipofectamine2000 reagent-containing solutions were then mixed and incubated at room temperature for a minimum of 20 minutes, and then added to the cells ensuring an even distribution of the transfection mix within the dish. The cells were incubated with the transfection reagent for 6 hours before the media was removed and replaced with 20 ml DMEM+10% FCS. Supernatant containing secreted protein was collected from the cells after 5 days and dead cells suspended in the supernatant were removed by centrifugation (4,500 rpm for 5 minutes). The resulting expression products were designated: hDLL1 EGF1-2 Fc (from pCONγ hDLL1 EGF1-2), hDLL1 EGF1-4 Fc (from pCONγ hDLL1 EGF1-4) and hDLL1 EGF1-7 Fc (from pCONγ hDLL1 EGF1-7). Expression of the Fc fusion proteins was assessed by western blot. The protein in 10 μl of supernatant was separated by 12% SDS-PAGE and blotted by semi dry apparatus on to Hybond™-ECL (Amersham Pharmacia Biotech) nitrocellulose membrane (17 V for 28 minutes). The presence of Fc fusion proteins was detected by Western blot using JDC14 anti-human IgG4 antibody diluted 1:500 in blocking solution (5% non-fat Milk solids in Tris-buffered saline with Tween 20 surfactant; TBS-T). The blot was incubated in this solution for 1 hour before being washed in TBS-T. After 3 washes of 5 minutes each, the presence of mouse anti-human IgG4 antibodies was detected using anti mouse IgG-HPRT conjugate antiserum diluted 1:10,000 in blocking solution. The blot was incubated in this solution for 1 hour before being washed in TBS-T (3 washes of 5 minutes each). The presence of Fc fusion proteins was then visualised using ECL™ detection reagent (Amersham Pharmacia Biotech).

The amount of protein present in 10 ml supernatant was assessed by comparing to Kappa chain standards containing 10 ng (7), 30 ng (8) and 100 ng (9) protein.

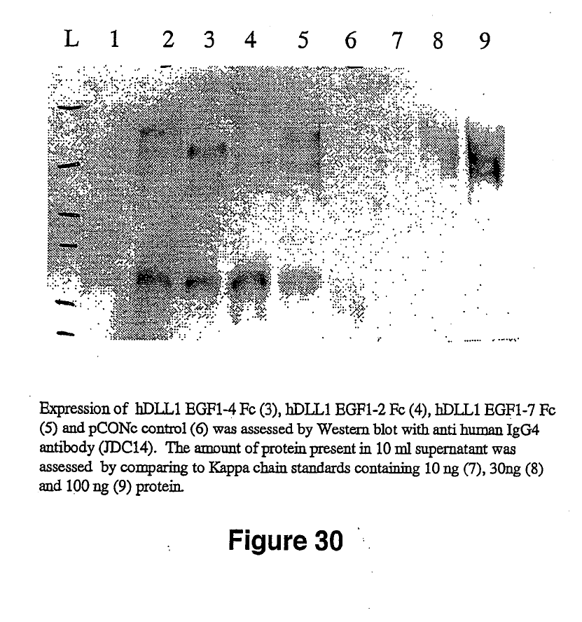

The blot results are shown in FIG. 30.

ii) Transfection and Expression of Constructs of Construct B

Cos 1 cells were transfected with the expression construct from Example 2 above (viz pCONγ hDLL1 EGF1-3) as follows:

7.1×10⁵ cells were plated in a T25 flask in Dulbecco's Modified Eagle's Medium (DMEM)+10% Fetal Calf Serum (FCS) and cells were left to adhere to the plate overnight. The cell monolayer was washed twice with 5 ml phosphate-buffered saline (PBS) and cells left in 1.14 ml OPTIMEM™ medium (Gibco/Invitrogen). 2.85 μg of the relevant construct DNA was diluted into 143 μl OPTIMEM medium and 14.3 μl Lipofectamine2000 cationic lipid transfection reagent (Invitrogen) was diluted in 129 μl OPTIMEM medium and incubated at room temperature for 45 minutes. The DNA-containing and Lipofectamine2000 reagent-containing solutions were then mixed and incubated at room temperature for 15 minutes, and then added to the cells ensuring an even distribution of the transfection mix within the flask. The cells were incubated with the transfection reagent for 18 hours before the media was removed and replaced with 3 ml DMEM+10% FCS. Supernatant containing secreted protein was collected from the cells after 4 days and dead cells suspended in the supernatant were removed by centrifugation (1,200 rpm for 5 minutes). The resulting expression product was designated: hDLL1 EGF1-3 Fc (from pCONγ hDLL1 EGF1-3).

F) Luciferase Reporter Assay

The Fc-tagged Notch ligand expression products from A to D above (hDLL1 EGF1-2 Fc, hDLL1 EGF1-4 Fc and hDLL1 EGF1-7 Fc) were each separately immobilised on Streptavidin-Dynabeads (CELLection Biotin Binder Dynabeads [Cat. No. 115.21] at 4.0×10⁸ beads/ml from Dynal (UK) Ltd; “beads”) in combination with biotinylated α-IgG-4 (clone JDC14 at 0.5 mg/ml from Pharmingen [Cat. No. 555879]) as follows:

1×10⁷ beads (25 μl of beads at 4.0×10⁸ beads/ml) and 2 μg biotinylated α-IgG4 was used for each sample assayed. PBS was added to the beads to 1 ml and the mixture was spun down at 13,000 rpm for 1 minute. Following washing with a further 1 ml of PBS the mixture was spun down again. The beads were then resuspended in a final volume of 100 μl of PBS containing the biotinylated α-IgG4 in a sterile Eppendorf tube and placed on shaker at room temperature for 30 minutes. PBS to was added to 1 ml and the mixture was spun down at 13,000 rpm for 1 minute and then washed twice more with 1 ml of PBS.

The mixture was then spun down at 13,000 rpm for 1 minute and the beads were resupsended in 50 μl PBS per sample. 50 μl of biotinylated a-IgG4-coated beads were added to each sample and the mixture was incubated on a rotary shaker at 4° C. overnight. The tube was then spun at 1000 rpm for 5 minutes at room temperature.

The beads then were washed with 10 ml of PBS, spun down, resupended in 1 ml of PBS, transferred to a sterile Eppendorf tube, washed with a further 2×1 ml of PBS, spun down and resuspended in a final volume of 100 μl of DMEM plus 10%(HI)FCS plus glutamine plus P/S, i.e. at 1.0×10⁵ beads/μl.

Stable N27#11 cells (T80 flask) were removed using 0.02% EDTA solution (Sigma), spun down and resuspended in 10 ml DMEM plus 10%(HI)FCS plus glutamine plus P/S. 10 μl of cells were counted and the cell density was adjusted to 1.0×10⁵ cells/ml with fresh DMEM plus 10%(HI)FCS plus glutamine plus P/S. 1.0×1 of the cells were plated out per well of a 24-well plate in a 1 ml volume of DMEM plus 10%(HI)FCS plus glutamine plus P/S and cells were placed in an incubator to settle down for at least 30 minutes.

20 μl of beads were then added in duplicate to a pair of wells to give 2.0×10⁶ beads/well (100 beads/cell). The plate was left in a CO₂ incubator overnight.

Supernatant was then removed from all the wells, 100 μl of SteadyGlo™ luciferase assay reagent (Promega) was added and the resulting mixture left at room temperature for 5 minutes.

The mixture was then pipetted up and down 2 times to ensure cell lysis and the contents from each well were transferred to a 96 well plate (with V-shaped wells) and spun in a plate holder for 5 minutes at 1000 rpm at room temperature.

175 μl of cleared supernatant was then transferred to a white 96-well plate (Nunc) leaving the beads pellet behind.

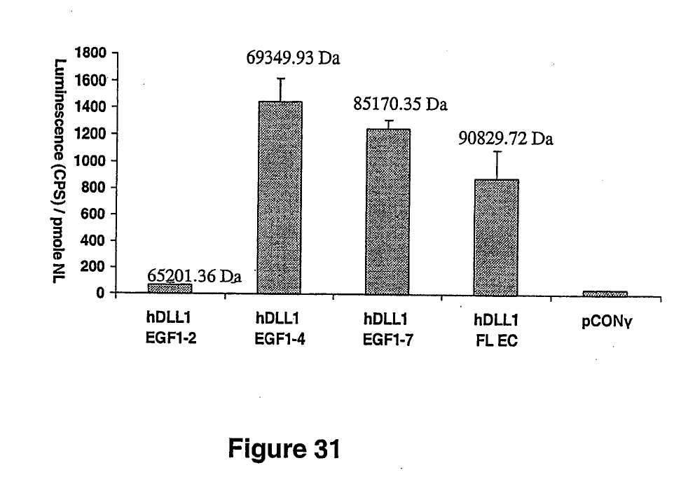

Luminescence was then read in a TopCount™ (Packard) counter. Results are shown in FIG. 31 (where activity from fusion protein comprising a full Dll1 EC domain (hDelta1-IgG4Fc) is also shown for comparison).

### Example 22

### Jagged Truncations

A similar series of truncations based on human Jagged1 comprising varying numbers of EGF repeats was prepared as follows:

In a similar manner to that described in Example 21, nucleotide sequences coding for the human Jagged1 (hJag1) DSL domain and the first two, three, four and sixteen respectively of the naturally occurring Jagged EGF repeats were generated by PCR from a human Jagged-1 (see eg GenBank Accession No U61276) cDNA. The sequences were then purified, ligated into a pCONγ expression vector coding for an immunogolbulin Fc domain, expressed and coated onto microbeads. The expressed proteins comprised the DSL domain and the first two (hJag1 EGF1-2), three (hJag1 EGF1-3), four (hJag1 EGF1-4) and sixteen (hJag1 EGF1-16) respectively of the Jagged EGF repeats fused to the IgG Fc domain encoded by the pCONγ vector.

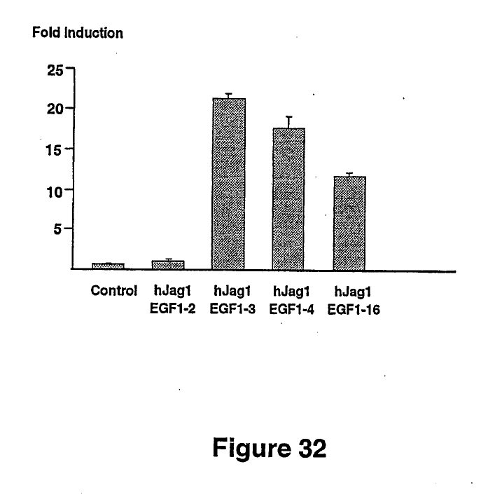

Beads coated with each of the expressed proteins were then tested for activity in the Notch signalling reporter assay as described above (Example 21). The activity data obtained is shown in FIG. 32.

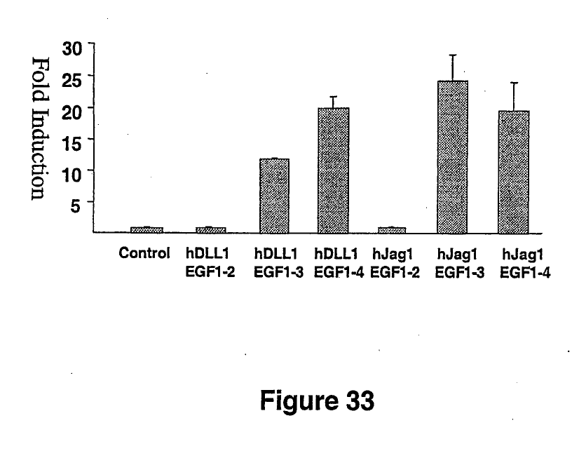

Similar assays were conducted with expressed Jagged proteins alongside corresponding Delta proteins, for more ready comparison. Results are shown in FIG. 33.

### Example 23

### Assay of Jagged EGF1-2 with Increased Sensitivity

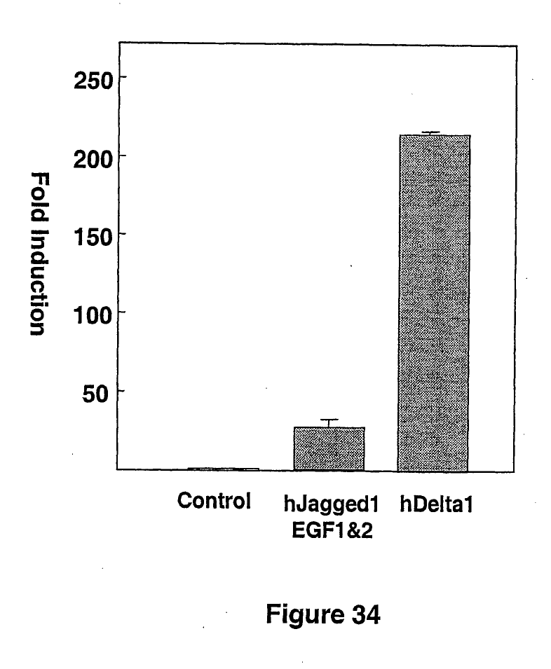

In a further experiment purified protein comprising human Jagged1 DSL domain plus the first two EGF repeats (hJagged1EGF1&2-IgG4Fc) from Example 7 was coated onto beads and tested for activity in a Notch reporter assay as described above, at a higher protein load, to give greater sensitivity. The activity data obtained is shown in FIG. 34 (activity from a fusion protein comprising a full Dll1 EC domain (hDelta1-IgG4Fc) is also shown for comparison).

The invention is further described by the following numbered paragraphs:
1. A product comprising:
i) an inhibitor of the Notch signalling pathway or a polynucleotide coding for such an inhibitor; and
ii) a pathogen antigen or antigenic determinant or a polynucleotide coding for a pathogen antigen or antigenic determinant;
as a combined preparation for simultaneous, contemporaneous, separate or sequential use for modulation of the immune system.
2. A product as described in paragraph 1 wherein the inhibitor of Notch signalling does not act by downregulating expression of Notch or a Notch ligand.
3. A product comprising:
i) an inhibitor of Notch signalling in the form of a Notch antagonist or a polynucleotide coding for such an antagonist; and
ii) a pathogen antigen or antigenic determinant or a polynucleotide coding for a pathogen antigen or antigenic determinant;
as a combined preparation for simultaneous, contemporaneous, separate or sequential use for modulation of the immune system.
4. A product comprising:
i) an inhibitor of Notch signalling in the form of an agent which inhibits Notch-Notch ligand interaction or a polynucleotide coding for such an agent; and
ii) a pathogen antigen or antigenic determinant or a polynucleotide coding for a pathogen antigen or antigenic determinant;
as a combined preparation for simultaneous, contemporaneous, separate or sequential use for modulation of the immune system.
5. A product as described in paragraph 4 wherein the inhibitor of Notch signalling binds to a Notch ligand or Notch receptor so as to interfere with Notch-Notch ligand interaction.
6. A product as described in any one of the preceding paragraphs in the form of a pharmaceutical composition or kit.
7. A product as described in any one of the preceding paragraphs in the form of a therapeutic vaccine composition or kit for treating infectious disease.
8. A product as described in any one of paragraphs 1 to 6 in the form of a prophylactic vaccine composition or kit for preventing infectious disease.
9. A product as described in any one of the preceding paragraphs wherein the inhibitor of Notch signalling is an agent capable of inhibiting the activity of a Notch receptor or a Notch ligand.
10. A product as described in any one of the preceding paragraphs wherein the inhibitor of Notch signalling is an agent capable of inhibiting the activity or downregulating the expression of a downstream component of the Notch signalling pathway.
11. A product as described in any one of the preceding paragraphs wherein the inhibitor of Notch signalling is a protein or polypeptide or a polynucleotide which codes for such a protein or polypeptide.
12. A product as described in any one of the preceding paragraphs wherein the inhibitor of Notch signalling comprises or codes for the extracellular domain of Delta or a fragment thereof.
12. A product as described in any one of paragraphs 1 to 11 wherein the inhibitor of Notch signalling comprises or codes for the extracellular domain of Serrate or Jagged or a fragment thereof.
13. A product as described in any one of paragraphs 1 to 11 wherein the inhibitor of Notch signalling comprises or codes for the extracellular domain of Notch or a fragment thereof.
14. A product as described in any one of paragraphs 1 to 11 wherein the inhibitor of Notch signalling comprises:
i) a protein or polypeptide which comprises a Notch ligand DSL domain and optionally a Notch ligand N-terminal domain or a heterologous amino acid sequence but which is substantially free of Notch ligand EGF-like domains;
ii) a multimer of such a protein or polypeptide (wherein each monomer may be the same or different); or
iii) a polynucleotide coding for such a protein or polypeptide.
15. A product as described in any one of paragraphs 1 to 11 wherein the inhibitor of Notch signalling comprises:
i) a protein or polypeptide which comprises a Notch ligand DSL domain and at least one Notch ligand EGF-like domain;
ii) a multimer of such a protein or polypeptide (wherein each monomer may be the same or different); or
iii) a polynucleotide coding for such a protein or polypeptide.
16. A product as described in any one of paragraphs 1 to 11 wherein the inhibitor of Notch signalling comprises:
i) a protein or polypeptide which comprises a Notch ligand DSL domain and at least two Notch ligand EGF-like domains;
ii) a multimer of such a protein or polypeptide (wherein each monomer may be the same or different); or
iii) a polynucleotide coding for such a protein or polypeptide.
17. A product as described in any one of paragraphs 1 to 11 wherein the inhibitor of Notch signalling comprises:
i) a protein or polypeptide which comprises a Notch ligand DSL domain and either 1 or 2, but no more than 2 Notch ligand EGF-like domains;
ii) a multimer of such a protein or polypeptide (wherein each monomer may be the same or different); or
iii) a polynucleotide coding for such a protein or polypeptide.
18. A product as described in any one of paragraphs 1 to 11 wherein the inhibitor of Notch signalling comprises:
i) a protein or polypeptide which comprises a Notch ligand DSL domain having at least 50% amino acid sequence identity to the DSL domain of human Delta1, Delta3 or Delta4 and at least one Notch ligand EGF-like domain having at least 50% amino acid sequence identity to an EGF-like domain of human Delta1, Delta3 or Delta4;
ii) a multimer of such a protein or polypeptide (wherein each monomer may be the same or different); or
iii) a polynucleotide coding for such a protein or polypeptide.
19. A product as described in any one of paragraphs 1 to 11 wherein the inhibitor of Notch signalling comprises:
i) a protein or polypeptide which comprises a Notch ligand DSL domain having at least 50% amino acid sequence identity to the DSL domain of human Delta1, Delta3 or Delta4 and either 1 or 2, but no more than 2 Notch ligand EGF-like domains having at least 50% amino acid sequence identity to an EGF-like domain of human Delta1, Delta3 or Delta4;
ii) a multimer of such a protein or polypeptide (wherein each monomer may be the same or different); or
iii) a polynucleotide coding for such a protein or polypeptide.
20. A product as described in any one of paragraphs 1 to 11 wherein the inhibitor of Notch signalling comprises:
i) a protein or polypeptide which comprises a Notch EGF-like domain having at least 50% amino acid sequence identity to EGF11 of human Notch1, Notch2, Notch3 or Notch4 and a Notch EGF-like domain having at least 50% amino acid sequence identity to EGF12 of human Notch1, Notch2, Notch3 or Notch4;
ii) a multimer of such a protein or polypeptide (wherein each monomer may be the same or different); or
iii) a polynucleotide coding for such a protein or polypeptide.
21. A product as described in any one of paragraphs 1 to 11 wherein the inhibitor of Notch signalling comprises:
i) a protein or polypeptide which comprises a Notch ligand DSL domain having at least 50% amino acid sequence identity to the DSL domain of human Jagged1 or Jagged2 and at least one Notch ligand EGF-like domain having at least 50% amino acid sequence identity to an EGF-like domain of human Jagged 1 or Jagged2;
ii) a multimer of such a protein or polypeptide (wherein each monomer may be the same or different); or
iii) a polynucleotide coding for such a protein or polypeptide.
22. A product as described in any one of paragraphs 1 to 11 wherein the inhibitor of Notch signalling comprises:
i) a protein or polypeptide which comprises a Notch ligand DSL domain having at least 50% amino acid sequence identity to the DSL domain of human Jagged1 or Jagged2 and either 0, 1 or 2, but no more than 2 Notch ligand EGF-like domains having at least 50% amino acid sequence identity to an EGF-like domain of human Jagged 1 or Jagged2;
ii) a multimer of such a protein or polypeptide (wherein each monomer may be the same or different); or
iii) a polynucleotide coding for such a protein or polypeptide.
23. A product as described in any one of paragraphs 1 to 11 wherein the inhibitor of Notch signalling comprises:
i) a protein or polypeptide which comprises a Notch ligand DSL domain having at least 70% amino acid sequence identity to the DSL domain of human Delta1, Delta3 or Delta4 and at least one Notch ligand EGF-like domain having at least 70% amino acid sequence identity to an EGF-like domain of human Delta1, Delta3 or Delta4;
ii) a multimer of such a protein or polypeptide (wherein each monomer may be the same or different); or
iii) a polynucleotide coding for such a protein or polypeptide.
24. A product as described in any one of paragraphs 1 to 11 wherein the inhibitor of Notch signalling comprises:
i) a protein or polypeptide which comprises a Notch ligand DSL domain having at least 70% amino acid sequence identity to the DSL domain of human Delta1, Delta3 or Delta4 and either 0, 1 or 2, but no more than 2 Notch ligand EGF-like domains having at least 70% amino acid sequence identity to an EGF-like domain of human Delta1, Delta3 or Delta4;
ii) a multimer of such a protein or polypeptide (wherein each monomer may be the same or different); or
iii) a polynucleotide coding for such a protein or polypeptide.
25. A product as described in any one of paragraphs 1 to 11 wherein the inhibitor of Notch signalling comprises:
i) a protein or polypeptide which comprises an EGF domain having at least 70% amino acid sequence identity to EGF11 of human Notch1, Notch2, Notch3 or Notch4 and an EGF domain having at least 70% amino acid sequence identity to EGF12 of human Notch1, Notch2, Notch3 or Notch4;
ii) a multimer of such a protein or polypeptide (wherein each monomer may be the same or different); or
iii) a polynucleotide coding for such a protein or polypeptide.
26. A product as described in any one of paragraphs 12 to 25 wherein the protein or polypeptide is fused to a heterologous amino acid sequence.
27. A product as described in paragraph 26 wherein the protein or polypeptide is fused to an immunoglobulin Fc (IgFc) domain.
28. A product as described in paragraph 27 wherein the IgFc domain is a human IgG1 or IgG4 Fc domain.
29. A product as described in any one of paragraphs 12 to 28 wherein the protein or polypeptide further comprises a Notch ligand N-terminal domain.
30. A product as described in any one of paragraphs 1 to 11 wherein the inhibitor of Notch signalling is an antibody, antibody fragment or antibody derivative or a polynucleotide which codes for an antibody, antibody fragment or antibody derivative.
31. A product as described in paragraph 30 wherein the antibody, antibody fragment or antibody derivative binds to a Notch receptor or a Notch ligand so as to interfere with Notch ligand-receptor interaction.
32. The use of an inhibitor of the Notch signalling pathway in the manufacture of a medicament for use as an immunostimulant wherein the medicament is not for use in reversing bacteria, infection or tumour-induced immunosuppression or for the treatment of a tumour.
33. The use of an inhibitor of the Notch signalling pathway in the manufacture of a medicament for use as an immunostimulant wherein the inhibitor does not act by downregulating expression of Notch or a Notch ligand.
34. The use of an inhibitor of the Notch signalling pathway in the manufacture of a medicament for use in vaccination against a pathogen.
35. The use of an inhibitor of the Notch signalling pathway in the manufacture of a medicament for use as an adjuvant for vaccination against a pathogen.
36. A use as described in any one of paragraphs 30 to 35 wherein the inhibitor of the Notch signalling pathway is a Notch signalling repressor or an agent which increases the expression or activity of a Notch signalling repressor.
37. A use as described in any one of paragraphs 30 to 35 wherein the inhibitor of the Notch signalling pathway is an agent capable of inhibiting the activity of a Notch receptor or a Notch ligand.
38. A use as described in any one of paragraphs 30 to 35 wherein the inhibitor of the Notch signalling pathway is an agent capable of inhibiting the activity or downregulating the expression of a downstream component of the Notch signalling pathway.
39. A use as described in any one of paragraphs 30 to 35 wherein the inhibitor of the Notch signalling pathway is an agent which binds to a Notch receptor or to a Notch ligand so as to interfere with Notch ligand-receptor interaction.
40. A use as described in any one of paragraphs 30 to 39 wherein the inhibitor of the Notch signalling pathway is a protein or polypeptide or a polynucleotide which codes for such a protein or polypeptide.
41. A use as described in paragraph 40 wherein the agent comprises or codes for the extracellular domain of Delta or a fragment thereof.
42. A use as described in paragraph 40 wherein the agent comprises or codes for the extracellular domain of Serrate or Jagged or a fragment thereof.
43. A use as described in paragraph 40 wherein the agent comprises or codes for the extracellular domain of Notch or a fragment thereof.
44. A use as described in any one of paragraphs 30 to 40 wherein the inhibitor of the Notch signalling pathway comprises:
i) a protein or polypeptide which comprises a Notch ligand DSL domain and at least one Notch ligand EGF-like domain;
ii) a multimer of such a protein or polypeptide; or
iii) a polynucleotide coding for such a protein or polypeptide.
45. A use as described in any one of paragraphs 30 to 40 wherein the inhibitor of the Notch signalling pathway comprises:
i) a protein or polypeptide which comprises a Notch ligand DSL domain and at least two Notch ligand EGF-like domains;
ii) a multimer of such a protein or polypeptide; or
iii) a polynucleotide coding for such a protein or polypeptide.
46. A use as described in any one of paragraphs 30 to 40 wherein the inhibitor of the Notch signalling pathway comprises:
i) a protein or polypeptide which comprises a Notch ligand DSL domain and either 0, 1 or 2, but no more than 2 Notch ligand EGF-like domains;
ii) a multimer of such a protein or polypeptide (wherein each monomer may be the same or different); or
iii) a polynucleotide coding for such a protein or polypeptide.
47. A use as described in any one of paragraphs 30 to 40 wherein the inhibitor of the Notch signalling pathway comprises:
i) a protein or polypeptide which comprises a Notch ligand DSL domain having at least 50% amino acid sequence identity to the DSL domain of human Delta1, Delta3 or Delta4 and at least one Notch ligand EGF-like domain having at least 50% amino acid sequence identity to an EGF-like domain of human Delta1, Delta3 or Delta4;
ii) a multimer of such a protein or polypeptide (wherein each monomer may be the same or different); or
iii) a polynucleotide coding for such a protein or polypeptide.
48. A use as described in any one of paragraphs 30 to 40 wherein the inhibitor of the Notch signalling pathway comprises:
i) a protein or polypeptide which comprises a Notch ligand DSL domain having at least 50% amino acid sequence identity to the DSL domain of human Delta1, Delta3 or Delta4 and either 1 or 2, but no more than 2 Notch ligand EGF-like domains having at least 50% amino acid sequence identity to an EGF-like domain of human Delta1, Delta3 or Delta4;
ii) a multimer of such a protein or polypeptide (wherein each monomer may be the same or different); or
iii) a polynucleotide coding for such a protein or polypeptide.
49. A use as described in any one of paragraphs 30 to 40 wherein the inhibitor of the Notch signalling pathway comprises:
i) a protein or polypeptide which comprises an EGF domain having at least 50% amino acid sequence identity to EGF11 of human Notch1, Notch2, Notch3 or Notch4 and an EGF domain having at least 50% amino acid sequence identity to EGF12 of human Notch1, Notch2, Notch3 or Notch4;
ii) a multimer of such a protein or polypeptide (wherein each monomer may be the same or different); or
iii) a polynucleotide coding for such a protein or polypeptide.
50. A use as described in any one of paragraphs 30 to 40 wherein the inhibitor of the Notch signalling pathway comprises:
i) a protein or polypeptide which comprises a Notch ligand DSL domain having at least 50% amino acid sequence identity to the DSL domain of human Jagged1 or Jagged2 and at least one Notch ligand EGF-like domain having at least 50% amino acid sequence identity to an EGF-like domain of human Jagged 1 or Jagged2;
ii) a multimer of such a protein or polypeptide (wherein each monomer may be the same or different); or
iii) a polynucleotide coding for such a protein or polypeptide.
51. A use as described in any one of paragraphs 30 to 40 wherein the inhibitor of the Notch signalling pathway comprises:
i) a protein or polypeptide which comprises a Notch ligand DSL domain having at least 50% amino acid sequence identity to the DSL domain of human Jagged1 or Jagged2 and either 1 or 2, but no more than 2 Notch ligand EGF-like domains having at least 50% amino acid sequence identity to an EGF-like domain of human Jagged 1 or Jagged2;
ii) a multimer of such a protein or polypeptide (wherein each monomer may be the same or different); or
iii) a polynucleotide coding for such a protein or polypeptide.
52. A use as described in any one of paragraphs 30 to 40 wherein the inhibitor of the Notch signalling pathway comprises:
i) a protein or polypeptide which comprises a Notch ligand DSL domain having at least 70% amino acid sequence identity to the DSL domain of human Delta1, Delta3 or Delta4 and at least one Notch ligand EGF-like domain having at least 70% amino acid sequence identity to an EGF-like domain of human Delta1, Delta3 or Delta4;
ii) a multimer of such a protein or polypeptide (wherein each monomer may be the same or different); or
iii) a polynucleotide coding for such a protein or polypeptide.
53. A use as described in any one of paragraphs 30 to 40 wherein the inhibitor of the Notch signalling pathway comprises:
i) a protein or polypeptide which comprises a Notch ligand DSL domain having at least 70% amino acid sequence identity to the DSL domain of human Delta1, Delta3 or Delta4 and either 1 or 2, but no more than 2 Notch ligand EGF-like domains having at least 70% amino acid sequence identity to an EGF-like domain of human Delta1, Delta3 or Delta4;
ii) a multimer of such a protein or polypeptide (wherein each monomer may be the same or different); or
iii) a polynucleotide coding for such a protein or polypeptide.
54. A use as described in any one of paragraphs 30 to 40 wherein the inhibitor of the Notch signalling pathway comprises:
i) a protein or polypeptide which comprises an EGF domain having at least 70% amino acid sequence identity to EGF11 of human Notch 1, Notch2, Notch3 or Notch4 and an EGF domain having at least 70% amino acid sequence identity to EGF12 of human Notch1, Notch2, Notch3 or Notch4;
ii) a multimer of such a protein or polypeptide (wherein each monomer may be the same or different); or
iii) a polynucleotide coding for such a protein or polypeptide.
55. A use as described in any one of paragraphs 40 to 54 wherein the protein or polypeptide is fused to a heterologous amino acid sequence.
56. A use as described in paragraph 55 wherein the protein or polypeptide is fused to an immunoglobulin Fc (IgFc) domain.
57. A use as described in paragraph 56 wherein the IgFc domain is a human IgG1 or IgG4 Fc domain.
58. A use as described in any one of paragraphs 32 to 39 wherein the inhibitor of the Notch signalling pathway is an antibody, antibody fragment or antibody derivative or a polynucleotide which codes for an antibody, antibody fragment or antibody derivative.
59. A use as described in paragraph 58 wherein the antibody, antibody fragment or antibody derivative binds to a Notch receptor or a Notch ligand so as to interfere with Notch ligand-receptor interaction.
60. The use of a binding agent which binds to a Notch ligand so as to interfere with binding of the ligand to a Notch receptor, or a polynucleotide which codes for such a binding agent, in the manufacture of a medicament for use as an immunostimulant.
61. The use of an antibody or antibody derivative which binds to a Notch receptor or to a Notch ligand, or a polynucleotide which codes for such an antibody or antibody derivative, in the manufacture of a medicament for use as an immunostimulant.
62. A method for stimulating the immune system by administering an inhibitor of the Notch signalling pathway which does not comprise reversing bacteria, infection or tumour-induced immunosuppression or treatment of a tumour.
63. A method for stimulating the immune system by administering an inhibitor of the Notch signalling pathway wherein the inhibitor does not act by downregulating expression of Notch or a Notch ligand.
64. A method for stimulating the immune system to treat or prevent an infection by administering an inhibitor of the Notch signalling pathway which does not comprise reversing bacteria, infection or tumour-induced immunosuppression or treatment of a tumour.
65. A method for stimulating the immune system to treat or prevent an infection by administering an inhibitor of the Notch signalling pathway wherein the inhibitor of the Notch signalling pathway does not act by downregulating expression of Notch or a Notch ligand.
66. A method for vaccination against a pathogen by administering an inhibitor of the Notch signalling pathway.
67. A method for enhancing vaccination against a pathogen by administering an inhibitor of the Notch signalling pathway.
68. A method for treating a chronic pathogen infection by administering an inhibitor of the Notch signalling pathway.
69. A method of increasing the immune response of a subject to a vaccine antigen or antigenic determinant comprising administering an effective amount of an inhibitor of the Notch signalling pathway to said subject simultaneously, separately or sequentially with said vaccine antigen or antigenic determinant or simultaneously, separately or sequentially with a polynucleotide coding for said vaccine antigen or antigenic determinant.
70. A method as described in any one of paragraphs 62 to 69 wherein the inhibitor of the Notch signalling pathway comprises a protein or polypeptide or a polynucleotide which codes for such a protein or polypeptide.
71. A method as described in any one of paragraphs 62 to 69 wherein the agent comprises or codes for the extracellular domain of Delta or a fragment thereof.
72. A method as described in any one of paragraphs 62 to 69 wherein the inhibitor of the Notch signalling pathway comprises or codes for the extracellular domain of Serrate or Jagged or a fragment thereof.
73. A method as described in any one of paragraphs 62 to 69 wherein the inhibitor of the Notch signalling pathway comprises or codes for the extracellular domain of Notch or a fragment thereof.
74. A method as described in any one of paragraphs 62 to 69 wherein the inhibitor of Notch signalling comprises:
i) a protein or polypeptide which comprises a Notch ligand DSL domain and at least one Notch ligand EGF-like domain;
ii) a multimer of such a protein or polypeptide; or
iii) a polynucleotide coding for such a protein or polypeptide.
75. A method as described in any one of paragraphs 62 to 69 wherein the inhibitor of Notch signalling comprises:
i) a protein or polypeptide which comprises a Notch ligand DSL domain and at least two Notch ligand EGF-like domains;
ii) a multimer of such a protein or polypeptide; or
iii) a polynucleotide coding for such a protein or polypeptide.
76. A method as described in any one of paragraphs 62 to 69 wherein the inhibitor of Notch signalling comprises:
i) a protein or polypeptide which comprises a Notch ligand DSL domain and either 0, 1 or 2, but no more than 2 Notch ligand EGF-like domains;
ii) a multimer of such a protein or polypeptide (wherein each monomer may be the same or different); or
iii) a polynucleotide coding for such a protein or polypeptide.
77. A method as described in any one of paragraphs 62 to 69 wherein the inhibitor of Notch signalling comprises:
i) a protein or polypeptide which comprises a Notch ligand DSL domain having at least 50% amino acid sequence identity to the DSL domain of human Delta1, Delta3 or Delta4 and at least one Notch ligand EGF-like domain having at least 50% amino acid sequence identity to an EGF-like domain of human Delta1, Delta3 or Delta4;
ii) a multimer of such a protein or polypeptide (wherein each monomer may be the same or different); or
iii) a polynucleotide coding for such a protein or polypeptide.
78. A method as described in any one of paragraphs 62 to 69 wherein the inhibitor of Notch signalling comprises:
i) a protein or polypeptide which comprises a Notch ligand DSL domain having at least 50% amino acid sequence identity to the DSL domain of human Delta1, Delta3 or Delta4 and either 0, 1 or 2, but no more than 2 Notch ligand EGF-like domains having at least 50% amino acid sequence identity to an EGF-like domain of human Delta1, Delta3 or Delta4;
ii) a multimer of such a protein or polypeptide (wherein each monomer may be the same or different); or
iii) a polynucleotide coding for such a protein or polypeptide.
79. A method as described in any one of paragraphs 62 to 69 wherein the inhibitor of Notch signalling comprises:
i) a protein or polypeptide which comprises an EGF domain having at least 50% amino acid sequence identity to EGF11 of human Notch1, Notch2, Notch3 or Notch4 and an EGF domain having at least 50% amino acid sequence identity to EGF12 of human Notch1, Notch2, Notch3 or Notch4;
ii) a multimer of such a protein or polypeptide (wherein each monomer may be the same or different); or
iii) a polynucleotide coding for such a protein or polypeptide.
80. A method as described in any one of paragraphs 62 to 69 wherein the inhibitor of Notch signalling comprises:
i) a protein or polypeptide which comprises a Notch ligand DSL domain having at least 50% amino acid sequence identity to the DSL domain of human Jagged1 or Jagged2 and at least one Notch ligand EGF-like domain having at least 50% amino acid sequence identity to an EGF-like domain of human Jagged 1 or Jagged2;
ii) a multimer of such a protein or polypeptide (wherein each monomer may be the same or different); or
iii) a polynucleotide coding for such a protein or polypeptide.
81. A method as described in any one of paragraphs 62 to 69 wherein the inhibitor of Notch signalling comprises:
i) a protein or polypeptide which comprises a Notch ligand DSL domain having at least 50% amino acid sequence identity to the DSL domain of human Jagged1 or Jagged2 and either 0, 1 or 2, but no more than 2 Notch ligand EGF-like domains having at least 50% amino acid sequence identity to an EGF-like domain of human Jagged 1 or Jagged2;
ii) a multimer of such a protein or polypeptide (wherein each monomer may be the same or different); or
iii) a polynucleotide coding for such a protein or polypeptide.
82. A method as described in any one of paragraphs 62 to 69 wherein the inhibitor of Notch signalling comprises:
i) a protein or polypeptide which comprises a Notch ligand DSL domain having at least 70% amino acid sequence identity to the DSL domain of human Delta1, Delta3 or Delta4 and at least one Notch ligand EGF-like domain having at least 70% amino acid sequence identity to an EGF-like domain of human Delta1, Delta3 or Delta4;
ii) a multimer of such a protein or polypeptide (wherein each monomer may be the same or different); or
iii) a polynucleotide coding for such a protein or polypeptide.
83. A method as described in any one of paragraphs 62 to 69 wherein the inhibitor of Notch signalling comprises:
i) a protein or polypeptide which comprises a Notch ligand DSL domain having at least 70% amino acid sequence identity to the DSL domain of human Delta1, Delta3 or Delta4 and either 1 or 2, but no more than 2 Notch ligand EGF-like domains having at least 70% amino acid sequence identity to an EGF-like domain of human Delta1, Delta3 or Delta4;
ii) a multimer of such a protein or polypeptide (wherein each monomer may be the same or different); or
iii) a polynucleotide coding for such a protein or polypeptide.
84. A method as described in any one of paragraphs 62 to 69 wherein the inhibitor of Notch signalling comprises:
i) a protein or polypeptide which comprises an EGF domain having at least 70% amino acid sequence identity to EGF11 of human Notch1, Notch2, Notch3 or Notch4 and an EGF domain having at least 70% amino acid sequence identity to EGF12 of human Notch1, Notch2, Notch3 or Notch4;
ii) a multimer of such a protein or polypeptide (wherein each monomer may be the same or different); or
iii) a polynucleotide coding for such a protein or polypeptide.
85. A method as described in any one of paragraphs 62 to 69 wherein the protein or polypeptide is fused to a heterologous amino acid sequence.
86. A method as described in paragraph 85 wherein the protein or polypeptide is fused to an immunoglobulin Fc (IgFc) domain.
87. A method as described in paragraph 86 wherein the IgFc domain is a human IgG4 Fc domain.
88. A method as described in any one of paragraphs 62 to 69 wherein the inhibitor of the Notch signalling pathway is a Notch signalling repressor or an agent which increases the expression or activity of a Notch signalling repressor.
89. A method as described in any one of paragraphs 62 to 69 wherein the inhibitor of the Notch signalling pathway is an agent capable of inhibiting the activity of a Notch receptor or a Notch ligand.
90. A method as described in any one of paragraphs 62 to 69 wherein the inhibitor of the Notch signalling pathway is an agent capable of inhibiting the activity or downregulating the expression of a downstream component of the Notch signalling pathway.
91. A method as described in any one of paragraphs 62 to 69 wherein the inhibitor of the Notch signalling pathway is an agent which binds to a Notch receptor or a Notch ligand so as to interfere with Notch-Notch ligand interaction.
92. A method as described in paragraph 91 wherein the agent is a protein or polypeptide or a polynucleotide which codes for such a protein or polypeptide.
93. A method as described in any one of paragraphs 62 to 69 wherein the inhibitor of the Notch signalling pathway is an antibody, antibody fragment or antibody derivative or a polynucleotide which codes for an antibody, antibody fragment or antibody derivative.
94. A method as described in paragraph 93 wherein the antibody, antibody fragment or antibody derivative binds to a Notch receptor or a Notch ligand so as to interfere with Notch-Notch ligand interaction.
95. A method for stimulating the immune system by administering an antibody or antibody derivative which binds to a Notch receptor or to a Notch ligand, or by administering a polynucleotide which codes for such an antibody or antibody derivative.
96. An adjuvant composition comprising an inhibitor of the Notch signalling pathway.
97. A composition as described in paragraph 96 wherein the inhibitor of the Notch signalling pathway is a Notch signalling repressor or an agent which increases the expression or activity of a Notch signalling repressor.
98. A composition as described in paragraph 96 wherein the inhibitor of the Notch signalling pathway is an agent capable of inhibiting the activity of a Notch receptor or a Notch ligand.
99. A composition as described in paragraph 96 wherein the inhibitor of the Notch signalling pathway is an agent capable of inhibiting the activity or downregulating the expression of a downstream component of the Notch signalling pathway.
100. A composition as described in paragraph 96 wherein the inhibitor of the Notch signalling pathway is an agent which binds to a Notch receptor or a Notch ligand so as to interfere with Notch-Notch ligand interaction.
101. A composition as described in paragraph 96 wherein the agent is a protein or polypeptide or a polynucleotide which codes for such a protein or polypeptide.
102. A composition as described in paragraph 101 wherein the inhibitor of the Notch signalling pathway is an antibody, antibody fragment or antibody derivative or a polynucleotide which codes for an antibody, antibody fragment or antibody derivative.
103. A composition as described in paragraph 102 wherein the antibody, antibody fragment or antibody derivative binds to a Notch receptor or a Notch ligand so as to interfere with Notch-Notch ligand interaction.
104. A composition as described in paragraph 96 wherein the agent comprises or codes for the extracellular domain of Delta or a fragment thereof.
105. A composition as described in paragraph 96 wherein the agent comprises or codes for the extracellular domain of Serrate or Jagged or a fragment thereof.
106. A composition as described in paragraph 96 wherein the agent comprises or codes for the extracellular domain of Notch or a fragment thereof.
107. A vaccine composition comprising an adjuvant composition as described in any one of paragraphs 94 to 106 and a pathogen antigen or antigenic determinant or a polynucleotide coding for a pathogen antigen or antigenic determinant.
108. A vaccine composition as described in paragraph 107 comprising a viral, fungal, parasitic or bacterial antigen or antigenic determinant or a polynucleotide coding for a viral, fungal, parasitic or bacterial antigen or antigenic determinant.
109. A product comprising:
i) an inhibitor of the Notch signalling pathway; and
ii) a pathogen antigen or antigenic determinant or a polynucleotide coding for a pathogen antigen or antigenic determinant;
as a combined preparation for simultaneous, contemporaneous, separate or sequential use for modulation of the immune system.
110. A product comprising:
i) a Notch antagonist; and
ii) a pathogen antigen or antigenic determinant or a polynucleotide coding for a pathogen antigen or antigenic determinant;
as a combined preparation for simultaneous, contemporaneous, separate or sequential use for modulation of the immune system.
111. A product as described in paragraph 109 or paragraph 110 for increasing effector T cell activity.
112. A method for modulating the immune system in a mammal comprising simultaneously, contemporaneously, separately or sequentially administering:
i) an effective amount of an inhibitor of the Notch signalling pathway; and
ii) a pathogen antigen or antigenic determinant or a polynucleotide coding for a pathogen antigen or antigenic determinant.
113. A combination of:
i) an inhibitor of the Notch signalling pathway; and
ii) a pathogen antigen or antigenic determinant or a polynucleotide coding for a pathogen antigen or antigenic determinant;
for simultaneous, contemporaneous, separate or sequential use in modulating the immune system.
114. An inhibitor of the Notch signalling pathway for use in modulating the immune system in simultaneous, contemporaneous, separate or sequential combination with a pathogen antigen or antigenic determinant or a polynucleotide coding for a pathogen antigen or antigenic determinant.
115. The use of a combination of:
i) an inhibitor of the Notch signalling pathway; and
ii) a pathogen antigen or antigenic determinant or a polynucleotide coding for a pathogen antigen or antigenic determinant; in the manufacture of a medicament for modulation of the immune system.
116. The use of an inhibitor of the Notch signalling pathway in the manufacture of a medicament for modulation of the immune system in simultaneous, contemporaneous, separate or sequential combination with a pathogen antigen or antigenic determinant or a polynucleotide coding for a pathogen antigen or antigenic determinant.
117. A pharmaceutical kit comprising an inhibitor of the Notch signalling pathway and a pathogen antigen or antigenic determinant or a polynucleotide coding for a pathogen antigen or antigenic determinant.
118. A conjugate comprising first and second sequences, wherein the first sequence comprises a pathogen antigen or antigenic determinant or a polynucleotide sequence coding for a pathogen antigen or antigenic determinant, and the second sequence comprises a polypeptide or polynucleotide for Notch signalling modulation.
119. A conjugate as described in paragraph 118 in the form of a vector comprising a first polynucleotide sequence coding for a modulator of the Notch signalling pathway and a second polynucleotide sequence coding for a pathogen antigen or antigenic determinant.
120. A conjugate as described in paragraph 119 in the form of an expression vector.
121. A conjugate as described in any one of paragraphs 1.18 to 120 wherein the first polynucleotide sequence codes for a Notch ligand or a fragment, derivative, homologue, analogue or allelic variant thereof.
122. A conjugate as described in paragraph 121 wherein the first polynucleotide sequence codes for a Delta or Serrate/Jagged protein or a fragment, derivative, homologue, analogue or allelic variant thereof.
123. A conjugate as described in any one of paragraphs 118 to 122 wherein the first polynucleotide sequence codes for a protein or polypeptide which comprises a Notch ligand DSL domain and at least one Notch ligand EGF-like domain.
124. A conjugate as described in paragraph 123 wherein the first polynucleotide sequence codes for a protein or polypeptide which comprises a Notch ligand DSL domain and at least two Notch ligand EGF-like domains.
125. A conjugate as described in paragraph 123 wherein the first polynucleotide sequence codes for a protein or polypeptide which comprises a Notch ligand DSL domain and 1 or 2 but no more than 2 Notch ligand EGF-like domains.
126. A conjugate as described in any one of paragraphs 118 to 125 wherein the first and second sequences are operably linked to one or more promoters.
127. A method for increasing the immune response to a pathogen antigen or antigenic determinant by administering in any order:
i) an inhibitor of the Notch signalling pathway; and
ii) a pathogen antigen or antigenic determinant or a polynucleotide coding for a pathogen antigen or antigenic determinant.
128. A method for increasing the immune response to a pathogen antigen or antigenic determinant by administering in any order:
i) an agent which binds to Notch or a Notch ligand to inhibit Notch-Notch ligand interactions; and
ii) a pathogen antigen or antigenic determinant or a polynucleotide coding for a pathogen antigen or antigenic determinant.
129. A method as described in paragraph 127 or paragraph 128 for treatment of an infection
130. A method as described in paragraph 127 or paragraph 128 for treatment of a chronic infection.
131. A method as described in paragraph 127 or paragraph 128 for prophylactic vaccination.
132. A method as described in paragraph 131 which confers protective immunity.
133. A pharmaceutical composition comprising:
i) a protein or polypeptide which comprises a Notch ligand DSL domain and either 0, 1 or 2, but no more than 2 Notch ligand EGF-like domains;
ii) a multimer of such a protein or polypeptide (wherein each monomer may be the same or different); or
iii) a polynucleotide coding for such a protein or polypeptide.
134. A method for modifying an immune response by administering a Notch ligand protein or polypeptide consisting essentially of the following components:
i) a Notch ligand DSL domain;
ii) optionally 1 or 2 EGF repeat domains;
iii) optionally all or part of a Notch ligand N-terminal domain; and
iv) optionally one or more heterologous amino acid sequences;
or by administering a polynucleotide coding for such a Notch ligand protein or polypeptide.
135. A method for increasing an immune response by administering a Notch ligand protein or polypeptide consisting essentially of the following components:
i) a Notch ligand DSL domain;
ii) optionally 1 or 2 EGF repeat domains;
iii) optionally all or part of a Notch ligand N-terminal domain; and
iv) optionally one or more heterologous amino acid sequences;
or by administering a polynucleotide coding for such a Notch ligand protein or polypeptide.
136. A method for reducing immune tolerance by administering a Notch ligand protein or polypeptide consisting essentially of the following components:
i) a Notch ligand DSL domain;
ii) optionally 1 or 2 EGF repeat domains;
iii) optionally all or part of a Notch ligand N-terminal domain; and
iv) optionally one or more heterologous amino acid sequences;
or by administering a polynucleotide coding for such a Notch ligand protein or polypeptide.
137. A method for modifying T cell activity by administering a Notch ligand protein or polypeptide consisting essentially of the following components:
i) a Notch ligand DSL domain;
ii) optionally 1 or 2 EGF repeat domains;
iii) optionally all or part of a Notch ligand N-terminal domain; and
iv) optionally one or more heterologous amino acid sequences;
or by administering a polynucleotide coding for such a Notch ligand protein or polypeptide.
138. A method for increasing helper (T_{H}) or cytotoxic (T_{C}) T-cell activity by administering a Notch ligand protein or polypeptide consisting essentially of the following components:
i) a Notch ligand DSL domain;
ii) optionally 1 or 2 EGF repeat domains;
iii) optionally all or part of a Notch ligand N-terminal domain; and
iv) optionally one or more heterologous amino acid sequences;
or by administering a polynucleotide coding for such a Notch ligand protein or polypeptide.
139. A method for reducing activity of regulatory T cells by administering a Notch ligand protein or polypeptide consisting essentially of the following components:
i) a Notch ligand DSL domain;
ii) optionally 1 or 2 EGF repeat domains;
iii) optionally all or part of a Notch ligand N-terminal domain; and
iv) optionally one or more heterologous amino acid sequences;
or by administering a polynucleotide coding for such a Notch ligand protein or polypeptide.
140. A method as described in paragraph 138 or paragraph 139 wherein the regulatory T cells are Tr1 regulatory T-cells.
141. A method as described in any one of the preceding paragraphs which comprises administering a Notch ligand protein or polypeptide consisting essentially of the following components:
i) a Notch ligand DSL domain;
ii) optionally all or part of a Notch ligand N-terminal domain; and
iii) optionally one or more heterologous amino acid sequences; or which comprises administering a polynucleotide coding for such a Notch ligand protein or polypeptide.
142. A method as described in any one of paragraphs 134 to 141 which comprises administering a Notch ligand protein or polypeptide consisting essentially of the following components:
i) a Notch ligand DSL domain;
ii) one Notch ligand EGF domain;
iii) optionally all or part of a Notch ligand N-terminal domain; and
iv) optionally one or more heterologous amino acid sequences; or which comprises administering a polynucleotide coding for such a Notch ligand protein or polypeptide.
143. A method as described in any one of paragraphs 134 to 142 which comprises administering a Notch ligand protein or polypeptide consisting essentially of the following components:
i) a Notch ligand DSL domain;
ii) two Notch ligand EGF domains;
iii) optionally all or part of a Notch ligand N-terminal domain; and
iv) optionally one or more heterologous amino acid sequences; or which comprises administering a polynucleotide coding for such a Notch ligand protein or polypeptide.
144. A method as described in any one of paragraphs 134 to 143 comprising administering a Notch ligand protein or polypeptide which is not bound to a cell or part of a cell.
145. A method as described in any of paragraphs 134 to 143 comprising administering a Notch ligand protein or polypeptide which is bound to a cell or part of a cell.
146. A method as described in any one of paragraphs 134 to 145 wherein the Notch ligand protein or polypeptide is a Notch receptor antagonist.
147. A method as described in any one of paragraphs 134 to 146 wherein the Notch ligand protein, polypeptide or polynucleotide comprises or codes for a heterologous amino acid sequence corresponding to all or part of an immunoglobulin Fc domain.
148. A method as described in any one of paragraphs 134 to 147 wherein the Notch ligand protein, polypeptide or polynucleotide comprises or codes for at least part of a mammalian Notch ligand sequence.
149. A method as described in any one of paragraphs 134 to 148 wherein the Notch ligand protein, polypeptide or polynucleotide comprises or codes for at least part of a human Notch ligand sequence.
150. A method as described in any one of paragraphs 134 to 149 wherein the Notch ligand protein, polypeptide or polynucleotide comprises or codes for Notch ligand domains from Delta, Serrate or Jagged or domains having at least 30% amino acid sequence similarity or
151. A method as described in any one of paragraphs 134 to 150 wherein the Notch ligand protein, polypeptide or polynucleotide comprises or codes for Notch ligand domains from Delta1, Delta 3, Delta 4, Jagged 1 or Jagged 2 or domains having at least 30% amino acid sequence similarity or identity thereto.
152. A method as described in any one of paragraphs 134 to 151 wherein the protein, polypeptide or polynucleotide is administered to a patient in vivo.
153. A method as described in any of paragraphs 134 to 151 wherein the protein, polypeptide or polynucleotide is administered to cells from a patient ex vivo.
154. A method as described in paragraph 153 wherein the cells are administered to a patient after administration of the protein, polypeptide or polynucleotide.
155. A Notch ligand protein or polypeptide consisting essentially of the following components:
i) a Notch ligand DSL domain;
ii) optionally 1 or 2 EGF domains;
iii) optionally all or part of a Notch ligand N-terminal domain; and
iv) optionally one or more heterologous amino acid sequences; or a polynucleotide coding for such a Notch ligand protein or polypeptide, for use to treat disease.
156. A Notch ligand protein or polypeptide or polynucleotide for a use as described in paragraph 155 wherein the Notch ligand protein or polypeptide consists essentially of the following components:
i) a Notch ligand DSL domain;
ii) optionally all or part of a Notch ligand N-terminal domain; and
iii) optionally one or more heterologous amino acid sequences; or wherein the polynucleotide codes for such a Notch ligand protein or polypeptide.
157. A Notch ligand protein or polypeptide or polynucleotide for a use as described in paragraph 155 wherein the Notch ligand protein or polypeptide consists essentially of the following components:
i) a Notch ligand DSL domain;
ii) one Notch ligand EGF domain;
iii) optionally all or part of a Notch ligand N-terminal domain; and
iv) optionally one or more heterologous amino acid sequences; or wherein the polynucleotide codes for such a Notch ligand protein or polypeptide.
158. A Notch ligand protein or polypeptide or polynucleotide for a use as described in paragraph 155 wherein the Notch ligand protein or polypeptide consists essentially of the following components:
i) a Notch ligand DSL domain;
ii) two Notch ligand EGF domains;
iii) optionally all or part of a Notch ligand N-terminal domain; and
iv) optionally one or more heterologous amino acid sequences; or wherein the polynucleotide codes for such a Notch ligand protein or polypeptide.
159. A Notch ligand protein or polypeptide or polynucleotide for a use as described in any one of paragraphs 155 to 158 wherein the Notch ligand protein or polypeptide is not bound to a cell or part of a cell.
160. A Notch ligand protein or polypeptide or polynucleotide for a use as described in any one of paragraphs 155 to 158 wherein the Notch ligand protein or polypeptide is bound to a cell or part of a cell.
161. A Notch ligand protein or polypeptide or polynucleotide for a use as described in any one of paragraphs 155 to 160 wherein the Notch ligand protein or polypeptide activates a Notch receptor.
162. A Notch ligand protein or polypeptide or polynucleotide for a use as described in any one of paragraphs 155 to 161 wherein the Notch ligand protein, polypeptide or polynucleotide comprises or codes for a heterologous amino acid sequence corresponding to all or part of an immunoglobulin F_{c} segment.
163. A Notch ligand protein or polypeptide or polynucleotide for a use as described in any one of paragraphs 155 to 162 wherein the Notch ligand protein, polypeptide or polynucleotide comprises or codes for at least part of a mammalian Notch ligand sequence.
164. A Notch ligand protein or polypeptide or polynucleotide for a use as described in any one of paragraphs 155 to 163 wherein the Notch ligand protein, polypeptide or polynucleotide comprises or codes for at least part of a human Notch ligand sequence.
165. A Notch ligand protein or polypeptide or polynucleotide for a use as described in any one of paragraphs 155 to 164 wherein the Notch ligand protein, polypeptide or polynucleotide comprises or codes for Notch ligand domains from Delta, Serrate or Jagged or domains having at least 30% amino acid sequence similarity thereto.
166. A Notch ligand protein or polypeptide or polynucleotide for a use as described in any one of paragraphs 155 to 165 wherein the Notch ligand protein, polypeptide or polynucleotide comprises or codes for Notch ligand domains from Delta1, Delta 3, Delta 4, Jagged 1 or Jagged 2 or domains having at least 30% amino acid sequence similarity thereto.
167. The use of a Notch ligand protein or polypeptide consisting essentially of the following components:
i) a Notch ligand DSL domain;
ii) optionally 1 or 2 EGF domains;
iii) optionally all or part of a Notch ligand N-terminal domain; and
iv) optionally one or more heterologous amino acid sequences; or a polynucleotide coding for such a Notch ligand protein or polypeptide, in the manufacture of a medicament for modification of an immune response.
168. The use of a Notch ligand protein or polypeptide consisting essentially of the following components:
i) a Notch ligand DSL domain;
ii) optionally 1 or 2 EGF domains; and
iii) optionally one or more heterologous amino acid sequences; or a polynucleotide coding for such a Notch ligand protein or polypeptide, in the manufacture of a medicament for modification of an immune response.
169. The use of a Notch ligand protein or polypeptide consisting essentially of the following components:
i) a Notch ligand DSL domain;
ii) optionally 1 or 2 EGF domains;
iii) optionally all or part of a Notch ligand N-terminal domain; and
iv) optionally one or more heterologous amino acid sequences; or a polynucleotide coding for such a Notch ligand protein or polypeptide, in the manufacture of a medicament for increasing an immune response.
170. The use of a Notch ligand protein or polypeptide consisting essentially of the following components:
i) a Notch ligand DSL domain;
ii) optionally 1 or 2 EGF domains;
iii) optionally all or part of a Notch ligand N-terminal domain; and
iv) optionally one or more heterologous amino acid sequences; or a polynucleotide coding for such a Notch ligand protein or polypeptide, in the manufacture of a medicament for reducing immune tolerance.
171. The use of a Notch ligand protein or polypeptide consisting essentially of the following components:
i) a Notch ligand DSL domain;
ii) optionally 1 or 2 EGF domains;
iii) optionally all or part of a Notch ligand N-terminal domain; and
iv) optionally one or more heterologous amino acid sequences; or a polynucleotide coding for such a Notch ligand protein or polypeptide, in the manufacture of a medicament for modification of T-cell activity.
172. The use of a Notch ligand protein or polypeptide consisting essentially of the following components:
i) a Notch ligand DSL domain;
ii) optionally 1 or 2 EGF domains;
iii) optionally all or part of a Notch ligand N-terminal domain; and
iv) optionally one or more heterologous amino acid sequences; or a polynucleotide coding for such a Notch ligand protein or polypeptide, in the manufacture of a medicament for increasing helper (T_{H}) or cytotoxic (T_{C}) T-cell activity.
173. The use of a Notch ligand protein or polypeptide consisting essentially of the following components:
i) a Notch ligand DSL domain;
ii) optionally 1 or 2 EGF domains;
iii) optionally all or part of a Notch ligand N-terminal domain; and
iv) optionally one or more heterologous amino acid sequences; or a polynucleotide coding for such a Notch ligand protein or polypeptide, in the manufacture of a medicament for reducing activity of regulatory T cells.
174. A use as described in paragraph 173 for reducing activity of Tr1 or Th3 regulatory T-cells.
175. A use as described in any one of paragraphs 167 to 174 wherein the Notch ligand protein or polypeptide consists essentially of the following components:
i) a Notch ligand DSL domain;
ii) optionally all or part of a Notch ligand N-terminal domain; and
iii) optionally one or more heterologous amino acid sequences; or wherein the polynucleotide codes for such a Notch ligand protein or polypeptide.
176. A use as described in any one of paragraphs 167 to 174 wherein the Notch ligand protein or polypeptide consists essentially of the following components:
i) a Notch ligand DSL domain;
ii) one EGF domain;
iii) optionally all or part of a Notch ligand N-terminal domain; and
iv) optionally one or more heterologous amino acid sequences; or wherein the polynucleotide codes for such a Notch ligand protein or polypeptide.
177. A use as described in any one of paragraphs 167 to 174 wherein the Notch ligand protein or polypeptide consists essentially of the following components:
i) a Notch ligand DSL domain;
ii) two EGF domains;
iii) optionally all or part of a Notch ligand N-terminal domain; and
iv) optionally one or more heterologous amino acid sequences; or wherein the polynucleotide codes for such a Notch ligand protein or polypeptide.
178. A use as described in any one of paragraphs 167 to 177 wherein the Notch ligand protein or polypeptide is not bound to a cell or part of a cell.
179. A use as described in any one of paragraphs 167 to 177 wherein the Notch ligand protein or polypeptide is bound to a cell or part of a cell.
180. A use as described in any one of paragraphs 167 to 179 wherein the Notch ligand protein or polypeptide inhibits a Notch receptor.
181. A use as described in any one of paragraphs 167 to 180 wherein the Notch ligand protein, polypeptide or polynucleotide comprises or codes for a heterologous amino acid sequence corresponding to all or part of an immunoglobulin F_{c} segment.
182. A use as described in any one of paragraphs 167 to 181 wherein the Notch ligand protein, polypeptide or polynucleotide comprises or codes for at least part of a mammalian Notch ligand sequence.
183. A use as described in any one of paragraphs 167 to 182 wherein the Notch ligand protein, polypeptide or polynucleotide comprises or codes for at least part of a human Notch ligand sequence.
184. A use as described in any one of paragraphs 167 to 183 wherein the Notch ligand protein, polypeptide or polynucleotide comprises or codes for Notch ligand domains from Delta, Serrate or Jagged or domains having at least 30% amino acid sequence similarity or identity thereto.
185. A use as described in any one of paragraphs 167 to 184 wherein the Notch ligand protein, polypeptide or polynucleotide comprises or codes for Notch ligand domains from Delta1, Delta 3, Delta 4, Jagged 1 or Jagged 2 or domains having at least 30% amino acid sequence similarity or identity thereto.
186. A pharmaceutical composition comprising a Notch ligand protein or polypeptide consisting essentially of the following components:
i) a Notch ligand DSL domain;
ii) optionally 1 or 2 EGF domains;
iii) optionally all or part of a Notch ligand N-terminal domain; and
iv) optionally one or more heterologous amino acid sequences; or a polynucleotide coding for such a Notch ligand protein or polypeptide, optionally in combination with a pharmaceutically acceptable carrier.
187. A pharmaceutical composition comprising a Notch ligand protein or polypeptide consisting essentially of the following components:
i) a Notch ligand DSL domain;
ii) optionally all or part of a Notch ligand N-terminal domain; and
iii) optionally one or more heterologous amino acid sequences; or a polynucleotide coding for such a Notch ligand protein or polypeptide, optionally in combination with a pharmaceutically acceptable carrier.
188. A pharmaceutical composition comprising a Notch ligand protein or polypeptide consisting essentially of the following components:
i) a Notch ligand DSL domain;
ii) one EGF repeat domain;
iii) optionally all or part of a Notch ligand N-terminal domain; and
iv) optionally one or more heterologous amino acid sequences; or a polynucleotide coding for such a Notch ligand protein or polypeptide, optionally in combination with a pharmaceutically acceptable carrier.
189. A pharmaceutical composition comprising a Notch ligand protein or polypeptide consisting essentially of the following components:
i) a Notch ligand DSL domain;
ii) two EGF domains;
iii) optionally all or part of a Notch ligand N-terminal domain; and
iv) optionally one or more heterologous amino acid sequences; or a polynucleotide coding for such a Notch ligand protein or polypeptide, optionally in combination with a pharmaceutically acceptable carrier.
190. A pharmaceutical composition as described in any of paragraphs 186 to 189 wherein the Notch ligand protein or polypeptide is not bound to a cell or part of a cell.
191. A pharmaceutical composition as described in any of paragraphs 186 to 189 wherein the Notch ligand protein or polypeptide is bound to a cell or part of a cell.
192. A pharmaceutical composition as described in any of paragraphs 186 to 191 wherein the Notch ligand protein or polypeptide inhibits a Notch receptor.
193. A pharmaceutical composition as described in any of paragraphs 186 to 192 wherein the Notch ligand protein, polypeptide or polynucleotide comprises or codes for a heterologous amino acid sequence corresponding to all or part of an immunoglobulin F_{c} segment.
194. A pharmaceutical composition as described in any of paragraphs 186 to 193 wherein the Notch ligand protein, polypeptide or polynucleotide comprises or codes for at least part of a mammalian Notch ligand sequence.
195. A pharmaceutical composition as described in any of paragraphs 186 to 194 wherein the Notch ligand protein, polypeptide or polynucleotide comprises or codes for at least part of a human Notch ligand sequence.
196. A pharmaceutical composition as described in any of paragraphs 186 to 195 wherein the Notch ligand protein, polypeptide or polynucleotide comprises or codes for Notch ligand domains from Delta, Serrate or Jagged or domains having at least 30% amino acid sequence similarity or identity thereto.
197. A pharmaceutical composition as described in any of paragraphs 186 to 196 wherein the Notch ligand protein, polypeptide or polynucleotide comprises or codes for Notch ligand domains from Delta1, Delta 3, Delta 4, Jagged 1 or Jagged 2 or domains having at least 30% amino acid sequence similarity thereto.
198. A Notch ligand protein or polypeptide which consists essentially of the following components:
i) a Notch ligand DSL domain;
ii) optionally all or part of a Notch ligand N-terminal domain;
iii) an immunoglobulin Fc domain; and
iv) optionally one or more further heterologous amino acid sequences; or a polynucleotide which codes for such a Notch ligand protein or polypeptide.
199. A Notch ligand protein or polypeptide which consists essentially of the following components:
i) a Notch ligand DSL domain;
ii) one EGF domain;
iii) optionally all or part of a Notch ligand N-terminal domain; and
iv) optionally one or more heterologous amino acid sequences; or a polynucleotide which codes for such a Notch ligand protein or polypeptide.
200. A Notch ligand protein or polypeptide which consists essentially of the following components:
i) a Notch ligand DSL domain;
ii) two EGF domains; and
iii) optionally one or more heterologous amino acid sequences; or a polynucleotide sequence which codes for such a Notch ligand protein or polypeptide.
201. A Notch ligand protein or polypeptide as described in any of paragraphs 198 to 200 which is not bound to a cell or part of a cell.
202. A Notch ligand protein or polypeptide as described in any of paragraphs 198 to 200 which is bound to a cell or part of a cell.
203. A Notch ligand protein or polypeptide or polynucleotide as described in any one of paragraphs 198 to 202 wherein the Notch ligand protein or polypeptide activates a Notch receptor.
204. A Notch ligand protein or polypeptide or polynucleotide as described in any one of paragraphs 198 to 203 which comprises or codes for a heterologous amino acid sequence corresponding to all or part of an immunoglobulin Fc segment.
205. A Notch ligand protein or polypeptide or polynucleotide as described in any one of paragraphs 198 to 204 which comprises or codes for at least part of a mammalian Notch ligand sequence.
206. A Notch ligand protein or polypeptide or polynucleotide as described in any one of paragraphs 198 to 205 which comprises or codes for at least part of a human Notch ligand sequence.
207. A Notch ligand protein or polypeptide or polynucleotide as described in any one of paragraphs 198 to 206 which comprises or codes for Notch ligand domains from Delta, Serrate or Jagged or domains having at least 30% amino acid sequence similarity thereto.
208. A Notch ligand protein or polypeptide or polynucleotide as described in any one of paragraphs 196 to 207 which comprises or codes for Notch ligand domains from Delta1, Delta 3, Delta 4, Jagged 1 or Jagged 2 or domains having at least 30% amino acid sequence similarity thereto.
209. A vector comprising a polynucleotide coding for a Notch ligand protein or polypeptide as described in any one of paragraphs 196 to 208.
210. A host cell transformed or transfected with a vector as described in paragraph 209.
211. A cell displaying a Notch ligand protein or polypeptide as described in any one of paragraphs 196 to 208 on its surface and/or transfected with a polynucleotide coding for such a protein or polypeptide.

### REFERENCES

Artavanis-Tsakonas S, et al. (1995) Science 268:225-232.
Artavanis-Tsakonas S, et al. (1999) Science 284:770-776.
Brucker K, et al. (2000) Nature 406:411-415.
Camilli et al. (1994) Proc Natl Acad Sci USA 91:2634-2638.
Chee M. et al. (1996) Science 274:601-614.
Hemmati-Brivanlou and Melton (1997) Cell 88:13-17.
Hicks C, et al. (2000) Nat. Cell. Biol. 2:515-520.
Iemura et al. (1998) PNAS 95:9337-9345.
Irvine K D (1999) Curr. Opin. Genet. Devel. 9:434-441.
Ju B J, et al. (2000) Nature 405:191-195.
Leimeister C. et al. (1999) Mech Dev 85(1-2):173-7.
L1 et al. (1998) Immunity 8(1):43-55.
Lieber, T. et al. (1993) Genes Dev 7(10):1949-65.
Lu, F. M. et al. (1996) Proc Natl Acad Sci 93(11):5663-7.
Matsuno K, et al. (1998) Nat. Genet. 19:74-78.
Matsuno, K. et al. (1995) Development 121(8):2633-44.
McGuinness T. et al (1996) Genomics 35(3):473-85.
Medhzhitov et al. (1997) Nature 388:394-397.
Meuer S. et al (2000) Rapid Cycle Real-time PCR, Springer-Verlag Berlin and Heidelberg GmbH & Co.
Moloney D J, et al. (2000) Nature 406:369-375.
Munro S, Freeman M. (2000) Curr. Biol. 10:813-820.
Ordentlich et al. (1998) Mol. Cell. Biol. 18:2230-2239.
Osborne B, Miele L. (1999) Immunity 11:653-663.
Panin V M, et al. (1997) Nature 387:908-912.
Sasai et al. (1994) Cell 79:779-790.
Schroeter, E. H. et al. (1998) Nature 393(6683):382-6.
Struhl G, Adachi A. (1998) Cell 93:649-660.
Takebayashi K. et al. (1994) J Biol Chem 269(7):150-6.
TamuraK, et al. (1995) Curr. Biol. 5:1416-1423.
Valenzuela et al. (1995) J. Neurosci. 15:6077-6084.
Weinmaster G. (2000) Curr. Opin. Genet. Dev. 10:363-369.
Wilson and Hemmati-Brivanlou (1997) Neuron 18:699-710.
Zhao et al. (1995) J. Immunol. 155:3904-3911.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

1. A product comprising:
i) an inhibitor of Notch signalling or a polynucleotide encoding the inhibitor; and
ii) a pathogen antigen or antigenic determinant or a polynucleotide encoding a pathogen antigen or antigenic determinant;
as a combined preparation for simultaneous, contemporaneous, separate or sequential administration for modulation of the immune system.

2. The product as claimed in claim 1, wherein the inhibitor of Notch signalling does not act by downregulating expression of Notch or a Notch ligand.

3. The product as claimed in claim 1, wherein the inhibitor of Notch signalling is a Notch antagonist or a polynucleotide encoding a Notch antagonist.

4. The product as claimed in claim 1, wherein the inhibitor of Notch signalling is an agent which inhibits Notch-Notch ligand interaction or a polynucleotide encoding the agent.

5. The product as claimed in claim 4, wherein the inhibitor of Notch signalling binds to a Notch ligand or Notch receptor so as to interfere with Notch-Notch ligand interaction.

6. The product as claimed in claim 1, wherein the inhibitor of Notch signalling is an agent capable of inhibiting the activity of a Notch receptor or a Notch ligand.

7. The product as claimed in claim 1, wherein the inhibitor of Notch signalling is an agent capable of inhibiting the activity or downregulating the expression of a downstream component of the Notch signalling pathway.

8. The product as claimed in claim 1, wherein the inhibitor of Notch signalling comprises or encodes an extracellular domain selected from the group consisting of: the extracellular domain of Delta, Serrate, Jagged, Notch, and a fragment thereof.

9. The product as claimed in claim 1, wherein the inhibitor of Notch signalling comprises:
i) a protein or polypeptide which comprises a Notch ligand DSL domain and at least one Notch ligand EGF-like domains;
ii) a multimer of the protein or polypeptide, wherein each monomer is the same or different; or
iii) a polynucleotide encoding the protein or polypeptide.

10. The product as claimed in claim 1, wherein the inhibitor of Notch signalling comprises:
i) a protein or polypeptide which comprises a Notch ligand DSL domain and 0, 1 or 2, but no more than 2, Notch ligand EGF-like domains;
ii) a multimer of the protein or polypeptide, wherein each monomer is the same or different; or
iii) a polynucleotide encoding the protein or polypeptide.

11. The product as claimed in claim 10, wherein the protein or polypeptide is substantially free of Notch ligand EGF-like domains.

12. The product as claimed in claim 9, wherein the protein or polypeptide has one Notch ligand EGF-like domain.

13. The product as claimed in claim 9, wherein the protein or polypeptide has two Notch ligand EGF-like domains.

14. The product as claimed in claim 9, wherein the protein or polypeptide comprises a Notch ligand DSL domain having at least 50% amino acid sequence identity to the DSL domain of human Delta1, Delta3 or Delta4 and at least one Notch ligand EGF-like domain having at least 50% amino acid sequence identity to an EGF-like domain of human Delta1, Delta3 or Delta4.

15. The product as claimed in claim 10, wherein the protein or polypeptide has a Notch ligand DSL domain having at least 50% amino acid sequence identity to the DSL domain of human Delta1, Delta3 or Delta4 and 1 or 2, but no more than 2, Notch ligand EGF-like domains having at least 50% amino acid sequence identity to an EGF-like domain of human Delta1, Delta3 or Delta4.

16. The product as claimed in claim 9, wherein the protein or polypeptide comprises a Notch EGF-like domain having at least 50% amino acid sequence identity to EGF11 of human Notch1, Notch2, Notch3 or Notch4 and a Notch EGF-like domain having at least 50% amino acid sequence identity to EGF12 of human Notch1, Notch2, Notch3 or Notch4.

17. The product as claimed in claim 9, wherein the protein or polypeptide comprises a Notch ligand DSL domain having at least 50% amino acid sequence identity to the DSL domain of human Jagged1 or Jagged2 and at least one Notch ligand EGF-like domain having at least 50% amino acid sequence identity to an EGF-like domain of human Jagged1 or Jagged2.

18. The product as claimed in claim 10, wherein the protein or polypeptide comprises a Notch ligand DSL domain having at least 50% amino acid sequence identity to the DSL domain of human Jagged1 or Jagged2 and either 0, 1 or 2, but no more than 2, Notch ligand EGF-like domains having at least 50% amino acid sequence identity to an EGF-like domain of human Jagged1 or Jagged2.

19. The product as claimed in claim 9, wherein the protein or polypeptide comprises a Notch ligand DSL domain having at least 70% amino acid sequence identity to the DSL domain of human Delta1, Delta3 or Delta4 and at least one Notch ligand EGF-like domain having at least 70% amino acid sequence identity to an EGF-like domain of human Delta1, Delta3 or Delta4.

20. The product as claimed in claim 10, wherein the protein or polypeptide comprises a Notch ligand DSL domain having at least 70% amino acid sequence identity to the DSL domain of human Delta1, Delta3 or Delta4 and either 0, 1 or 2, but no more than 2, Notch ligand EGF-like domains having at least 70% amino acid sequence identity to an EGF-like domain of human Delta1, Delta3 or Delta4.

21. The product as claimed in claim 9, wherein the protein or polypeptide comprises an EGF domain having at least 70% amino acid sequence identity to EGF11 of human Notch1, Notch2, Notch3 or Notch4 and an EGF domain having at least 70% amino acid sequence identity to EGF12 of human Notch1, Notch2, Notch3 or Notch4.

22. The product as claimed in claim 9, wherein the protein or polypeptide is fused to a heterologous amino acid sequence.

23. The product as claimed in claim 22, wherein the protein or polypeptide is fused to an immunoglobulin Fc (IgFc) domain.

24. The product as claimed in claim 23, wherein the IgFc domain is a human IgG1 or IgG4 Fc domain.

25. The product as claimed in claim 9, wherein the protein or polypeptide further comprises a Notch ligand N-terminal domain.

26. The product as claimed in claim 1, wherein the inhibitor of Notch signalling is an antibody, antibody fragment or antibody derivative or a polynucleotide encoding an antibody, antibody fragment or antibody derivative.

27. The product as claimed in claim 26 wherein the antibody, antibody fragment or antibody derivative binds to a Notch receptor or a Notch ligand so as to interfere with Notch ligand-receptor interaction.

28. A method for stimulating the immune system comprising administering an inhibitor of the Notch signalling pathway, wherein the method does not comprise reversing bacteria-induced, infection-induced or tumour-induced immunosuppression or treatment of a tumour.

29. A method for stimulating the immune system comprising administering an inhibitor of the Notch signalling pathway, wherein the inhibitor does not act by downregulating expression of Notch or a Notch ligand.

30. The method of claim 29, wherein administration of the inhibitor treats or prevents an infection.

31. The method of claim 30, wherein administration of the inhibitor treats or prevents an infection.

32. A method for vaccination against a pathogen comprising administering an inhibitor of the Notch signalling pathway.

33. A method for enhancing vaccination against a pathogen comprising administering an inhibitor of the Notch signalling pathway.

34. A method for treating a chronic pathogen infection comprising administering an inhibitor of the Notch signalling pathway.

35. A method of increasing the immune response of a subject to an antigen or antigenic determinant comprising administering an effective amount of an inhibitor of the Notch signalling pathway to said subject simultaneously, separately or sequentially with said antigen or antigenic determinant or simultaneously, separately or sequentially with a polynucleotide encoding the antigen or antigenic determinant.

36. The method as claimed in claim 35, wherein the inhibitor of the Notch signalling pathway comprises a protein or polypeptide or a polynucleotide encoding the protein or polypeptide.

37. The method as claimed in claim 35, wherein the agent comprises or encodes the extracellular domain of Delta or a fragment thereof.

38. The method as claimed in claim 35, wherein the inhibitor of the Notch signalling pathway comprises or encodes the extracellular domain of Serrate or Jagged or a fragment thereof.

39. The method as claimed in claim 35, wherein the inhibitor of the Notch signalling pathway comprises or encodes the extracellular domain of Notch or a fragment thereof.

40. The method as claimed in claim 35, wherein the inhibitor of Notch signalling comprises:
i) a protein or polypeptide which comprises a Notch ligand DSL domain and at least one Notch ligand EGF-like domain;
ii) a multimer of the protein or polypeptide; or
iii) a polynucleotide encoding the a protein or polypeptide.

41. The method as claimed in claim 40, wherein the protein or polypeptide comprises a Notch ligand DSL domain and at least two Notch ligand EGF-like domains.

42. The method as claimed in claim 35, wherein the inhibitor of Notch signalling comprises:
i) a protein or polypeptide which comprises a Notch ligand DSL domain and either 0, 1 or 2, but no more than 2, Notch ligand EGF-like domains;
ii) a multimer of such a protein or polypeptide (wherein each monomer may be the same or different); or
iii) a polynucleotide coding for such a protein or polypeptide.

43. The method as claimed in claim 40, wherein the protein or polypeptide comprises a Notch ligand DSL domain having at least 50% amino acid sequence identity to the DSL domain of human Delta1, Delta3 or Delta4 and at least one Notch ligand EGF-like domain having at least 50% amino acid sequence identity to an EGF-like domain of human Delta1, Delta3 or Delta4.

44. The method as claimed in claim 42, wherein the protein or polypeptide comprises a Notch ligand DSL domain having at least 50% amino acid sequence identity to the DSL domain of human Delta1, Delta3 or Delta4 and either 0, 1 or 2, but no more than 2, Notch ligand EGF-like domains having at least 50% amino acid sequence identity to an EGF-like domain of human Delta1, Delta3 or Delta4.

45. The method as claimed in claim 40, wherein the protein or polypeptide comprises an EGF domain having at least 50% amino acid sequence identity to EGF11 of human Notch1, Notch2, Notch3 or Notch4 and an EGF domain having at least 50% amino acid sequence identity to EGF12 of human Notch1, Notch2, Notch3 or Notch4.

46. The method as claimed in claim 40, wherein the protein or polypeptide comprises a Notch ligand DSL domain having at least 50% amino acid sequence identity to the DSL domain of human Jagged1 or Jagged2 and at least one Notch ligand EGF-like domain having at least 50% amino acid sequence identity to an EGF-like domain of human Jagged 1 or Jagged2.

47. The method as claimed in claim 42, wherein protein or polypeptide comprises a Notch ligand DSL domain having at least 50% amino acid sequence identity to the DSL domain of human Jagged1 or Jagged2 and either 0, 1 or 2, but no more than 2, Notch ligand EGF-like domains having at least 50% amino acid sequence identity to an EGF-like domain of human Jagged 1 or Jagged2.

48. The method as claimed in claim 40, wherein the protein or polypeptide comprises a Notch ligand DSL domain having at least 70% amino acid sequence identity to the DSL domain of human Delta1, Delta3 or Delta4 and at least one Notch ligand EGF-like domain having at least 70% amino acid sequence identity to an EGF-like domain of human Delta1, Delta3 or Delta4.

49. The method as claimed in claim 42, wherein the protein or polypeptide comprises a Notch ligand DSL domain having at least 70% amino acid sequence identity to the DSL domain of human Delta1, Delta3 or Delta4 and either 1 or 2, but no more than 2, Notch ligand EGF-like domains having at least 70% amino acid sequence identity to an EGF-like domain of human Delta1, Delta3 or Delta4.

50. The method as claimed in claim 40, wherein the protein or polypeptide comprises an EGF domain having at least 70% amino acid sequence identity to EGF11 of human Notch1, Notch2, Notch3 or Notch4 and an EGF domain having at least 70% amino acid sequence identity to EGF12 of human Notch1, Notch2, Notch3 or Notch4.

51. The method as claimed in claim 40, wherein the protein or polypeptide is fused to a heterologous amino acid sequence.

52. The method as claimed in claim 51, wherein the protein or polypeptide is fused to an immunoglobulin Fc (IgFc) domain.

53. The method as claimed in claim 52, wherein the IgFc domain is a human IgG4 Fc domain.

54. The method as claimed in claim 35, wherein the inhibitor of the Notch signalling pathway is a Notch signalling repressor or an agent which increases the expression or activity of a Notch signalling repressor.

55. The method as claimed in claim 35, wherein the inhibitor of the Notch signalling pathway is an agent capable of inhibiting the activity of a Notch receptor or a Notch ligand.

56. The method as claimed in 35, wherein the inhibitor of the Notch signalling pathway is an agent capable of inhibiting the activity or downregulating the expression of a downstream component of the Notch signalling pathway.

57. The method as claimed in claim 35, wherein the inhibitor of the Notch signalling pathway is an agent which binds to a Notch receptor or a Notch ligand so as to interfere with Notch-Notch ligand interaction.

58. The method as claimed in claim 57, wherein the agent is a protein or polypeptide or a polynucleotide which codes for such a protein or polypeptide.

59. The method as claimed in claim 35, wherein the inhibitor of the Notch signalling pathway is an antibody, antibody fragment or antibody derivative or a polynucleotide which codes for an antibody, antibody fragment or antibody derivative.

60. The method as claimed in claim 59, wherein the antibody, antibody fragment or antibody derivative binds to a Notch receptor or a Notch ligand so as to interfere with Notch-Notch ligand interaction.

61. The method as claimed in claim 29, wherein the inhibitor is an antibody or antibody derivative which binds to a Notch receptor or to a Notch ligand, or a polynucleotide encoding the antibody or antibody derivative.

62. An adjuvant composition comprising an inhibitor of the Notch signalling % pathway.

63. The composition as claimed in claim 62, wherein the inhibitor of the Notch signalling pathway is a Notch signalling repressor or an agent which increases the expression or activity of a Notch signalling repressor.

64. The composition as claimed in claim 62, wherein the inhibitor of the Notch signalling pathway is an agent capable of inhibiting the activity of a Notch receptor or a Notch ligand.

65. The composition as claimed in claim 62, wherein the inhibitor of the Notch signalling pathway is an agent capable of inhibiting the activity or downregulating the expression of a downstream component of the Notch signalling pathway.

66. The composition as claimed in claim 62, wherein the inhibitor of the Notch signalling pathway is an agent which binds to a Notch receptor or a Notch ligand so as to interfere with Notch-Notch ligand interaction.

67. The composition as claimed in claim 62, wherein the inhibitor is a protein or polypeptide or a polynucleotide encoding the protein or polypeptide.

68. The composition as claimed in claim 67, wherein the inhibitor of the Notch signalling pathway is an antibody, antibody fragment or antibody derivative or a polynucleotide encoding an antibody, antibody fragment or antibody derivative.

69. The composition as claimed in claim 68, wherein the antibody, antibody fragment or antibody derivative binds to a Notch receptor or a Notch ligand so as to interfere with Notch-Notch ligand interaction.

70. The composition as claimed in claim 62, wherein the inhibitor comprises or encodes the extracellular domain of Delta or a fragment thereof.

71. The composition as claimed in claim 62, wherein the inhibitor comprises or encodes the extracellular domain of Serrate or Jagged or a fragment thereof.

72. The composition as claimed in claim 62, wherein the inhibitor comprises or encodes the extracellular domain of Notch or a fragment thereof.

73. A vaccine composition comprising the adjuvant composition as claimed in claim 62, and a pathogen antigen or antigenic determinant or a polynucleotide encoding a pathogen antigen or antigenic determinant.

74. The vaccine composition as claimed in claim 73, comprising a viral, fungal, parasitic or bacterial antigen or antigenic determinant or a polynucleotide encoding a viral, fungal, parasitic or bacterial antigen or antigenic determinant.

75. The product as claimed in claim 1, wherein effector T cell activity is increased.

76. A method for modulating the immune system in a mammal comprising simultaneously, contemporaneously, separately or sequentially administering an effective amount of the product of claim 1.

77. A conjugate comprising first and second sequences, wherein the first sequence comprises a pathogen antigen or antigenic determinant or a polynucleotide sequence encoding a pathogen antigen or antigenic determinant, and the second sequence comprises a polypeptide or polynucleotide for Notch signalling modulation.

78. The conjugate as claimed in claim 77, which is a vector comprising a first polynucleotide sequence encoding a modulator of the Notch signalling pathway and a second polynucleotide sequence encoding a pathogen antigen or antigenic determinant.

79. The conjugate as claimed in claim 77, which is an expression vector.

80. The conjugate as claimed in claim 77, wherein the first polynucleotide sequence encodes a Notch ligand or a fragment, derivative, homologue, analogue or allelic variant thereof.

81. The conjugate as claimed in claim 80, wherein the first polynucleotide sequence encodes a Delta or Serrate/Jagged protein or a fragment, derivative, homologue, analogue or allelic variant thereof.

82. The conjugate as claimed in claim 77, wherein the first polynucleotide sequence encodes a protein or polypeptide which comprises a Notch ligand DSL domain and at least one Notch ligand EGF-like domain.

83. The conjugate as claimed in claim 82, wherein the first polynucleotide sequence encodes a protein or polypeptide which comprises a Notch ligand DSL domain and at least two Notch ligand EGF-like domains.

84. The conjugate as claimed in claim 82, wherein the first polynucleotide sequence encodes a protein or polypeptide which comprises a Notch ligand DSL domain and 1 or 2, but no more than 2, Notch ligand EGF-like domains.

85. The conjugate as claimed in claim 77, wherein the first and second sequences are operably linked to one or more promoters.

86. The method as claimed in claim 35 for treatment of an infection.

87. The method as claimed in claim 86, wherein the infection is a chronic infection.

88. A composition comprising:
i) a protein or polypeptide which comprises a Notch ligand DSL domain and either 0, 1 or 2, but no more than 2, Notch ligand EGF-like domains;
ii) a multimer of the protein or polypeptide, wherein each monomer may be the same or different; or
iii) a polynucleotide encoding the protein or polypeptide.

89. A Notch ligand protein or polypeptide consisting essentially of:
i) a Notch ligand DSL domain;
ii) optionally, 1 or 2 EGF domains;
iii) optionally, all or part of a Notch ligand N-terminal domain; and
iv) optionally, one or more heterologous amino acid sequences;
or a polynucleotide encoding the Notch ligand protein or polypeptide, for use to treat disease.

90. The Notch ligand protein or polypeptide or polynucleotide as claimed in claim 89, wherein the Notch ligand protein or polypeptide has one Notch ligand EGF domain.

91. The Notch ligand protein or polypeptide or polynucleotide as claimed in claim 89, wherein the Notch ligand protein or polypeptide has two Notch ligand EGF domains.

92. The Notch ligand protein or polypeptide or polynucleotide as claimed in claim 89, wherein the Notch ligand protein or polypeptide is not bound to a cell or part of a cell.

93. The Notch ligand protein or polypeptide or polynucleotide as claimed in claim 89, wherein the Notch ligand protein or polypeptide is bound to a cell or part of a cell.

94. The Notch ligand protein or polypeptide or polynucleotide as claimed in claim 89, wherein the Notch ligand protein or polypeptide activates a Notch receptor.

95. The Notch ligand protein or polypeptide or polynucleotide as claimed in claim 89, wherein the Notch ligand protein, polypeptide or polynucleotide comprises or encodes a heterologous amino acid sequence corresponding to all or part of an immunoglobulin F_{c} segment.

96. The Notch ligand protein or polypeptide or polynucleotide as claimed in claim 89, wherein the Notch ligand protein, polypeptide or polynucleotide comprises or encodes at least part of a mammalian Notch ligand sequence.

97. The Notch ligand protein or polypeptide or polynucleotide as claimed in claim 89, wherein the Notch ligand protein, polypeptide or polynucleotide comprises or encodes at least part of a human Notch ligand sequence.

98. The Notch ligand protein or polypeptide or polynucleotide as claimed in claim 89, wherein the Notch ligand protein, polypeptide or polynucleotide comprises or encodes Notch ligand domains from Delta, Serrate or Jagged or domains having at least 30% amino acid sequence similarity thereto.

99. The Notch ligand protein or polypeptide or polynucleotide as claimed in claim 89, wherein the Notch ligand protein, polypeptide or polynucleotide comprises or codes for Notch ligand domains from Delta1, Delta3, Delta4, Jagged1 or Jagged2 or domains having at least 30% amino acid sequence similarity thereto.

100. A method for modifying an immune response comprising administering, to a subject in need thereof, the Notch ligand protein or polypeptide as claimed in claim 89 or a polynucleotide encoding the Notch ligand protein or polypeptide.

101. The method as claimed in claim 100, wherein the immune response is increased.

102. The method as claimed in claim 100, wherein immune tolerance is reduced.

103. The method as claimed in claim 100, wherein T cell activity is modified.

104. The method as claimed in claim 100, wherein helper (T_{H}) or cytotoxic (T_{C}) T-cell activity is increased.

105. The method as claimed in claim 100, wherein activity of regulatory T cells is reduced.

106. The method as claimed in claim 105, wherein the regulatory T cells are Tr1 regulatory T-cells.

107. The method as claimed in claim 100, wherein the Notch ligand protein or polypeptide has one Notch ligand EGF domain.

108. The method as claimed in claim 100, wherein the Notch ligand protein or polypeptide has two Notch ligand EGF domains.

109. The method as claimed in claim 100, wherein the Notch ligand protein or polypeptide is not bound to a cell or part of a cell.

110. The method as claimed in claim 100, wherein the Notch ligand protein or polypeptide is bound to a cell or part of a cell.

111. The method as claimed in claim 100, wherein the Notch ligand protein or polypeptide is a Notch receptor antagonist.

112. The method as claimed in claim 100, wherein the Notch ligand protein, polypeptide or polynucleotide comprises or encodes a heterologous amino acid sequence corresponding to all or part of an immunoglobulin F_{c} domain.

113. The method as claimed in claim 100, wherein the Notch ligand protein, polypeptide or polynucleotide comprises or encodes at least part of a mammalian Notch ligand sequence.

114. The method as claimed in claim 100, wherein the Notch ligand protein, polypeptide or polynucleotide comprises or encodes at least part of a human Notch ligand sequence.

115. The method as claimed in claim 100, wherein the Notch ligand protein, polypeptide or polynucleotide comprises or encodes Notch ligand domains from Delta, Serrate or Jagged or domains having at least 30% amino acid sequence similarity or identity thereto.

116. The method as claimed in claim 100, wherein the Notch ligand protein, polypeptide or polynucleotide comprises or encodes Notch ligand domains from Delta1, Delta3, Delta4, Jagged1 or Jagged2 or domains having at least 30% amino acid sequence similarity or identity thereto.

117. The method as claimed in claim 100, wherein the protein, polypeptide or polynucleotide is administered to a patient in vivo.

118. The method as claimed in claim 100, wherein the protein, polypeptide or polynucleotide is administered to cells from a patient ex vivo.

119. The method as claimed in claim 118, wherein the cells are administered to a patient after the protein, polypeptide or polynucleotide is administered to the cells.

120. A composition comprising the Notch ligand protein or polypeptide as claimed in claim 89 or a polynucleotide encoding the Notch ligand protein or polypeptide, optionally in combination with a pharmaceutically acceptable carrier.

121. A Notch ligand protein or polypeptide consisting essentially of:
i) a Notch ligand DSL domain;
ii) optionally, all or part of a Notch ligand N-terminal domain;
iii) an immunoglobulin Fc domain; and
iv) optionally, one or more further heterologous amino acid sequences;
or a polynucleotide which codes for such a Notch ligand protein or polypeptide.

122. The Notch ligand protein or polypeptide as claimed in claim 121, wherein the Notch ligand protein or polypeptide is not bound to a cell or part of a cell.

123. The Notch ligand protein or polypeptide as claimed in claim 121, wherein the Notch ligand protein or polypeptide is bound to a cell or part of a cell.

124. The Notch ligand protein or polypeptide as claimed in claim 121, wherein the Notch ligand protein or polypeptide activates a Notch receptor.

125. The Notch ligand protein or polypeptide or polynucleotide as claimed in claim 121, wherein the Notch ligand protein or polypeptide comprises or encodes a heterologous amino acid sequence corresponding to all or part of an immunoglobulin FC segment.

126. The Notch ligand protein or polypeptide or polynucleotide as claimed in claim 121, wherein the Notch ligand protein or polypeptide comprises or encodes at least part of a mammalian Notch ligand sequence.

127. The Notch ligand protein or polypeptide or polynucleotide as claimed in claim 121, wherein the Notch ligand protein or polypeptide comprises or encodes at least part of a human Notch ligand sequence.

128. The Notch ligand protein or polypeptide or polynucleotide as claimed in claim 121, wherein the Notch ligand protein or polypeptide comprises or encodes Notch ligand domains from Delta, Serrate or Jagged or domains having at least 30% amino acid sequence similarity thereto.

129. The Notch ligand protein or polypeptide or polynucleotide as claimed in claim 121, wherein the Notch ligand protein or polypeptide comprises or encodes Notch ligand domains from Delta1, Delta 3, Delta4, Jagged1 or Jagged2 or domains having at least 30% amino acid sequence similarity thereto.

130. A vector comprising a polynucleotide encoding for the Notch ligand protein or polypeptide as claimed in claim 121.

131. A host cell transformed or transfected with the vector as claimed in claim 130.

132. A cell displaying the Notch ligand protein or polypeptide as claimed in claim 121 in its surface.

133. The cell as claimed in claim 132, wherein the cell is transfected with a polynucleotide encoding the Notch ligand protein or polypeptide.


SEQUENCE LISTING (72 sequences)

SEQ ID NO: 1 - DNA, 24 bp, Artificial sequence
  oligonucleotide
tcgtcgttttgtcgttttgtcgtt

SEQ ID NO: 2 - DNA, 66 bp, Artificial sequence
  Plasmid
agcttgcggccgcgggcccagcggtggtggacctcactgagaagctagaggcttccaccaaaggcc

SEQ ID NO: 3 - DNA, 50 bp, Artificial sequence
  Kozak sequence top strand
agcttgccgccaccatgggcagtcggtgcgcgctggccctggcggtgctc

SEQ ID NO: 4 - DNA, 38 bp, Artificial sequence
  Kozak sequence -top strand
tcggccttgctgtgtcaggtctggagctctggggtgtt

SEQ ID NO: 5 - protein, 864 aa, Artificial sequence
  fusion protein
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHGYVCECARGYGGPNCQFLLPELPPGPAVVDLTEKLEASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - DNA, 20 bp, Artificial sequence
  oligonucleotide - top strand
gtgctgttacccgtacggta

SEQ ID NO: 7 - DNA, 40 bp, Artificial sequence
  Kozak sequence - top strand
agcttgccgccaccatgggttccccacggacacgcggccg

SEQ ID NO: 8 - DNA, 27 bp, Artificial sequence
  oligonucleotide - top strand
gtccgcaccttgtgggtacccgtacgg

SEQ ID NO: 9 - DNA, 22 bp, Artificial sequence
  linker oligonucleotide - top strand
gatctcgcttccaccaagggcc

SEQ ID NO: 10 - protein, 626 aa, Artificial sequence [sequence omitted]

SEQ ID NO: 11 - DNA, 26 bp, Artificial sequence
  TATA box sequence - top strand
gatctggggggctataaaagggggta

SEQ ID NO: 12 - DNA, 50 bp, Artificial sequence
  TPI motif sequence - top strand
gatcccgactcgtgggaaaatgggcggaagggcaccgtgggaaaatagta

SEQ ID NO: 13 - DNA, 33 bp, Artificial sequence
  PCR primer
ccaggcaagcttatgggttccccacggacgcgc

SEQ ID NO: 14 - DNA, 37 bp, Artificial sequence
  PCR primer
cagctctgtgacaaagatctcaattacctcgagatcg

SEQ ID NO: 15 - DNA, 39 bp, Artificial sequence
  adaptor oligonucleotide
agctttcagttctcgagggatcggcttccaccaagggcc

SEQ ID NO: 16 - protein, 302 aa, Artificial sequence
  fusion protein
MGSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYEGS

SEQ ID NO: 17 - DNA, 26 bp, Artificial sequence
  PCR primer
caccatgggcagtcggtgcgcgctgg

SEQ ID NO: 18 - DNA, 22 bp, Artificial sequence
  PCR primer
gtagttcaggtcctggttgcag

SEQ ID NO: 19 - DNA, 26 bp, Artificial sequence
  PCR primer
caccatgggcagtcggtgcgcgctgg

SEQ ID NO: 20 - DNA, 45 bp, Artificial sequence
  PCR primer
ggatatgggcccttggtggaagcgtagttcaggtcctggttgcag

SEQ ID NO: 21 - protein, 291 aa, Artificial sequence
  fusion protein
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNY

SEQ ID NO: 22 - DNA, 26 bp, Artificial sequence
  PCR primer
caccatgggcagtcggtgcgcgctgg

SEQ ID NO: 23 - DNA, 45 bp, Artificial sequence
  PCR primer
ggatatgggcccttggtggaagcctcgtcaatccccagctcgcag

SEQ ID NO: 24 - protein, 331 aa, Artificial sequence
  Fusion protein
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDE

SEQ ID NO: 25 - DNA, 26 bp, Artificial sequence
  PCR primer
caccatgggcagtcggtgcgcgctgg

SEQ ID NO: 26 - DNA, 22 bp, Artificial sequence
  PCR primer
ggtcatggcactcaattcacag

SEQ ID NO: 27 - DNA, 26 bp, Artificial sequence
  PCR primer
caccatgggcagtcggtgcgcgctgg

SEQ ID NO: 28 - DNA, 45 bp, Artificial sequence
  PCR primer
ggatatgggcccttggtggaagcggtcatggcactcaattcacag

SEQ ID NO: 29 - protein, 369 aa, Artificial sequence
  fusion protein
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMT

SEQ ID NO: 30 - DNA, 26 bp, Artificial sequence
  PCR primer
caccatgggcagtcggtgcgcgctgg

SEQ ID NO: 31 - DNA, 25 bp, Artificial sequence
  PCR primer
cctgctgacgggggcactgcagttc

SEQ ID NO: 32 - DNA, 26 bp, Artificial sequence
  PCR primer
caccatgggcagtcggtgcgcgctgg

SEQ ID NO: 33 - DNA, 48 bp, Artificial sequence
  PCR primer
ggatatgggcccttggtggaagccctgctgacgggggcactgcagttc

SEQ ID NO: 34 - protein, 484 aa, Artificial sequence
  fusion protein
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSR

SEQ ID NO: 35 - protein, 63 aa, Drosophila sp.
WKTNKSESQYTSLEYDFRVTCDLNYYGSGCAKFCRPRDDSFGHSTCSETGEIICLTGWQGDYC

SEQ ID NO: 36 - protein, 63 aa, Homo sapiens
WSQDLHSSGRTDLKYSYRFVCDEHYYGEGCSVFCRPRDDAFGHFTCGERGEKVCNPGWKGPYC

SEQ ID NO: 37 - protein, 63 aa, Mus musculus
WSQDLHSSGRTDLRYSYRFVCDEHYYGEGCSVFCRPRDDAFGHFTCGDRGEKMCDPGWKGQYC

SEQ ID NO: 38 - protein, 63 aa, Rattus rattus
WSQDLHSSGRTDLRYSYRFVCDEHYYGEGCSVFCRPRDDAFGHFTCGERGEKMCDPGWKGQYC

SEQ ID NO: 39 - protein, 63 aa, Mus musculus
WRTDEQNDTLTRLSYSYRVICSDNYYGESCSRLCKKRDDHFGHYECQPDGSLSCLPGWTGKYC

SEQ ID NO: 40 - protein, 63 aa, Homo sapiens
WLLDEQTSTLTRLRYSYRVICSDNYYGDNCSRLCKKRNDHFGHYVCQPDGNLSCLPGWTGEYC

SEQ ID NO: 41 - protein, 63 aa, Rattus rattus
WQTLKQNTGIAHFEYQIRVTCDDHYYGFGCNKFCRPRDDFFGHYACDQNGNKTCMEGWMGPEC

SEQ ID NO: 42 - protein, 63 aa, Mus musculus
WQTLKQNTGIAHFEYQIRVTCDDHYYGFGCNKFCRPRDDFFGHYACDQNGNKTCMEGWMGPDC

SEQ ID NO: 43 - protein, 63 aa, Homo sapiens
WQTLKQNTGVAHFEYQIRVTCDDYYYGFGCNKFCRPRDDFFGHYACDQNGNKTCMEGWMGREC

SEQ ID NO: 44 - protein, 63 aa, Gallus sp.
WQTLKHNTGAAHFEYQIRVTCAEHYYGFGCNKFCRPRDDFFTHHTCDQNGNKTCLEGWTGPEC

SEQ ID NO: 45 - protein, 63 aa, Gallus sp.
WKTLQFNGPVANFEVQIRVKCDENYYSALCNKFCGPRDDFVGHYTCDQNGNKACMEGWMGEEC

SEQ ID NO: 46 - protein, 63 aa, Mus musculus
WKSLHFSGHVAHLELQIRVRCDENYYSATCNKFCRPRNDFFGHYTCDQYGNKACMDGWMGKEC

SEQ ID NO: 47 - protein, 63 aa, Homo sapiens; identical to SEQ ID NO: 46

SEQ ID NO: 48 - protein, 63 aa, Rattus rattus; identical to SEQ ID NO: 46

SEQ ID NO: 49 - protein, 63 aa, Homo sapiens; identical to SEQ ID NO: 46

SEQ ID NO: 50 - protein, 63 aa, Drosophila sp.
WKTLDHIGRNARITYRVRVQCAVTYYNTTCTTFCRPRDDQFGHYACGSEGQKLCLNGWQGVNC

SEQ ID NO: 51 - protein, 942 aa, Homo sapiens [sequence omitted]

SEQ ID NO: 52 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 53 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 54 - protein, 1218 aa, Homo sapiens
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 55 - protein, 1238 aa, Homo sapiens [sequence omitted]

SEQ ID NO: 56 - protein, 2556 aa, Homo sapiens
  UNSURE (1)..(2556): X ia any amino acid
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPRLCRHGGTCHNEVGSYRCVCRATHTGPNCERPYVPCSPSPCQNGGTCRPTGDVTHECACLPGFTGQNCEENIDDCPGNNCKNGGACVDGVNTYNCPCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCMCMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRLRGTCQDPDNAYLCFCLKGTTGPNCEINLDDCASSPCDSGTCLDKIDGYECACEPGYTGSMCNSNIDECAGNPCHNGGTCEDGINGFTCRCPEGYHDPTCLSEVNECNSNPCVHGACRDSLNGYKCDCDPGWSGTNCDINNNECESNPCVNGGTCKDMTSGIVCTCREGFSGPNCQTNINECASNPCLNKGTCIDDVAGYKCNCLLPYTGATCEVVLAPCAPSPCRNGGECRQSEDYESFSCVCPTAGAKGQTCEVDINECVLSPCRHGASCQNTHGXYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHVVNECDSRPCLLGGTCQDGRGLHRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 57 - protein, 2471 aa, Homo sapiens
MPALRPALLWALLALWLCCAAPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCNQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 58 - protein, 43 aa, Artificial sequence
  DSL Domain
CXXXXXXXXCXXXCXXXXXXXXXXXCXXXXXXXCXXXXXXXXC

SEQ ID NO: 59 - protein, 43 aa, Artificial sequence; identical to SEQ ID NO: 58
  DSL consensus sequence 2

SEQ ID NO: 60 - protein, 43 aa, Artificial sequence
  DSL consensus sequence 3
CXXXYYXXXCXXXCRPRXDXFGHXXCXXXGXXXCXXGWXGXXC

SEQ ID NO: 61 - protein, 18 aa, Artificial sequence
  EGF- like domain
XCXCXCXCXCXGXXGXCX

SEQ ID NO: 62 - DNA, 12 bp, Artificial sequence
  low complexity filtering example
nnnnnnnnnnnn

SEQ ID NO: 63 - protein, 9 aa, Artificial sequence
  Low complexity filtering example
XXXXXXXXX

SEQ ID NO: 64 - DNA, 58 bp, Artificial sequence
  Plasmid
acgccggcgcccgggtcgccaccacctggagtgactcttcgatctccgaaggtggttt

SEQ ID NO: 65 - DNA, 40 bp, Artificial sequence
  Kozak sequence bottom strand
acggcggtggtacccgtcagccacgcgcgaccgggaccgc

SEQ ID NO: 66 - DNA, 46 bp, Artificial sequence
  Kozak sequence - bottom strand
cacgagagccggaacgacacagtccagacctcgagaccccacaagc

SEQ ID NO: 67 - DNA, 20 bp, Artificial sequence
  oligonucleotide - bottom strand
gaacacgacaatgggcatgc

SEQ ID NO: 68 - DNA, 39 bp, Artificial sequence
  Kozak sequence - bottom strand
acggcggtggtacccaaggggtgcctgtgcgccggccag

SEQ ID NO: 69 - DNA, 28 bp, Artificial sequence
  oligonucleotide - bottom strand
gcgtggaacacccatgggcatgccttaa

SEQ ID NO: 70 - DNA, 14 bp, Artificial sequence
  linker oligonucleotide - bottom strand
agcgaaggtggttc

SEQ ID NO: 71 - DNA, 26 bp, Artificial sequence
  TATA box sequence - bottom strand
accccccgatattttcccccattcga

SEQ ID NO: 72 - DNA, 50 bp, Artificial sequence
  TP1motif sequence - bottom strand
ggctgagcacccttttacccgccttcccgtggcacccttttatcatctag